(19) United States
(12) Patent Application Publication
(10) Pub. No.: US 2022/0160664 A1
(43) Pub. Date: May 26, 2022

(54) TAZ ACTIVATORS AND WNT AGONISTS FOR TREATING EAR DISORDERS

(71) Applicant: Frequency Therapeutics, Inc., Lexington, MA (US)

(72) Inventors: Will MCLEAN, North Haven, CT (US); Megan S. HARRISON, Middletown, CT (US); Bradley TAIT, North Andover, MA (US)

(21) Appl. No.: 17/429,153

(22) PCT Filed: Feb. 7, 2020
(86) PCT No.: PCT/US2020/017353

§ 371 Date: Aug. 6, 2021

Related U.S. Application Data
(60) Provisional application No. 62/803,354, filed on Feb. 8, 2019.

(51) Int. Cl. A61K 31/19; A61K 31/5377; A61K 31/444; A61K 31/4545; A61K 38/17; A61K 31/4725; A61K 31/443; A61K 31/4439; A61K 31/496; A61K 31/4409; A61K 31/195; A61K 31/495; A61K 31/4465; A61K 31/135; A61K 31/15; A61K 31/7064; A61K 31/7076; A61K 31/5517; A61K 31/506; A61P 27/16

(52) U.S. Cl. CPC A61K 31/19; A61P 27/16; A61K 31/444; A61K 31/4545; A61K 38/1709; A61K 31/4725; A61K 31/443; A61K 31/4439; A61K 31/496; A61K 31/4409; A61K 31/195; A61K 31/495; A61K 31/4465; A61K 31/135; A61K 31/15; A61K 31/7064; A61K 31/7076; A61K 31/5517; A61K 31/506; A61K 31/5377

(57) ABSTRACT

Provided are compositions and methods comprising a TAZ activator and a Wnt agonist for increasing proliferation of cochlear supporting cells or vestibular supporting cells, and related methods of treating inner ear hearing or balance disorders.

### CROSS-REFERENCE TO RELATED APPLICATION

This application claims priority to U.S. Provisional Application No. 62/803,354 filed Feb. 8, 2019, entitled “COMPOSITIONS AND METHODS FOR GENERATING HAIR CELLS BY ACTIVATING TAZ”, the disclosures of which are incorporated by reference herein.

### FIELD OF THE INVENTION

The present disclosure relates to compositions and methods comprising a TAZ activator and a Wnt agonist for increasing proliferation of cochlear supporting cells or vestibular supporting cells, production of an expanded population of cochlear or vestibular cells, in particular Lgr5+ cells, and related methods of treating an inner hearing or balance disorder, in particular sensorineural hearing loss.

### BACKGROUND OF THE INVENTION

Generation of sensory hair cells from undifferentiated cell populations is likely to provide a therapy for several inner ear hearing and balance disorders that arise from damage and loss of sensory hair cells in the inner ear. Replacement hair cells could be produced in situ, in the damaged sensory epithelium of the inner ear, or grown in vitro and then delivered to the inner ear, and so strategies for generation of sensory cells in vitro and in vivo are of interest.

Sensorineural hearing loss (SNHL), which is largely due to the loss of sensory hair cells and their neural connections is a widespread problem. It is estimated that over one billion young people are at risk for noise-related sensorineural hearing loss. SNHL accounts for about 90% of all hearing loss (Li et al., Adv. Drug Deliv. Rev. 108, 2-12, 2017), and leading causes include advanced age, ototoxic medications, and noise exposure (Liberman & Kujawa, Hear. Res. 349, 138-147, 2017). The majority of children and adults with SNHL are managed with hearing aids or cochlear implants, as there is currently no therapeutic option to restore function in the damaged inner ear (see, for example, Ramakers et al., Laryngoscope 125, 2584-92, 2015; Raman et al., Effectiveness of Cochlear Implants in Adults with Sensorineural Hearing Loss. Agency for Healthcare Research and Quality (US), 2011; and Roche & Hansen, Otolaryngol. Clin. North Am. 48, 1097-116, 2015). Loss or damage of hair cells in the vestibular system of inner ear can lead to balance disorders (for example, dizziness and vertigo), incidences of which also increase with age. Like the cochlea, there is currently no therapeutic option to restore function in damaged vestibular epithelia, and regeneration of hair cells may also be an effective therapeutic approach for balance disorders.

The underlying pathophysiologic changes of sensory epithelia of the inner ear in patients with inner ear hearing loss or balance disorders includes damage and loss of sensory transducers of the cochlear and vestibular systems called hair cells. Hair cells are susceptible to damage, and although other species such as birds, fish, and amphibians can regenerate these cells throughout life, mammals lack this ability (Fujioka et al., Trends Neurosci. 38, 139-44, 2015).

Several approaches are being investigated to replace damaged or absent hair cells in mammalian inner ear sensory epithelia (reviewed in Mittal et al. Front Mol Neurosci. (2017); 10: 236). These include cell-based approaches (which aim to deliver exogenous cells to the inner ear to restore the sensory epithelia) and gene-based approaches (which aim to deliver exogenous genes to the sensory epithelia and reprogram endogenous cells to generate hair cells). For example, adenovirus-mediated delivery of Atoh1 can stimulate cells within the sensory epithelia to differentiate into hair cells. One drawback with these approaches is the requirement to deliver cells or vectors into the inner ear of the patient, which can be challenging in the complex system of the inner ear. Molecular approaches, in which the endogenous signaling pathways of inner ear cells are modulated by exogenous agents are therefore attractive, as the delivery of such agents for prolonged periods of time is likely to be more straightforward than cell-based or gene-based approaches.

Using molecular agents to initiate transdifferentiation, in which existing supporting cells of the cochlear are stimulated to differentiate into replacement hair cells, is one area of interest. However, transdifferentiation alone (i.e. without proliferation) may not provide sufficient hair cells to regenerate a functioning cochlea or vestibular system, especially as an associated depletion of the supporting cell population could also negatively impact the functioning of the cochlea or vestibular organs. Focus has therefore been placed on activation of proliferative response in the supporting cells, in order to provide a new population of cells that could differentiate into hair cells, thereby replacing lost or damaged hair cells.

A subset of supporting cells that express Lgr5 have been shown to be endogenous hair cell progenitors with stimulation via the Wnt/beta-catenin pathway leading to proliferation and differentiation of these cells into sensory hair cells (Bramhall et al., 2014 Stem Cell Repotrs 2, 311-322). More recently, a combination of a Wnt pathway agonist (a GSKβ inhibitor) in combination with a histone deacetylase complex (HDAC) inhibitor has been found to stimulate expansion of an Lgr5+ supporting cell population in the inner ear (McLean et al., Cell Rep. 2017 February 21; 18(8): 1917-1929).

There remains a need for the development of effective hair cell regeneration strategies in the inner ear, both in vitro and in vivo which may include boosting the proliferation of supporting cells of sensory epithelium of the inner ear beyond that which has been achieved previously.

### SUMMARY OF THE INVENTION

The disclosure provides a method for increasing proliferation of a cochlear supporting cell or a vestibular supporting cell, by contacting the supporting cell with: a) a transcriptional coactivator with PDZ-binding motif (TAZ) activator; and b) a Wnt agonist; wherein (a) and (b) can occur in any order or simultaneously.

The disclosure provides a method for producing an expanded population of cochlear or vestibular cells, by contacting a population of cochlear supporting cells or vestibular supporting cells with: a) a transcriptional coactivator with PDZ-binding motif (TAZ) activator and; b) a Wnt agonist wherein (a) and (b) can occur in any order or simultaneously.

In some embodiments of the methods of the disclosure, the cochlear supporting cell(s) or vestibular supporting cell(s) express(es) leucine-rich repeat-containing G-protein coupled receptor 5 (Lgr5).

In some embodiments of the methods of the disclosure, the cochlear supporting cell(s) or vestibular supporting cell(s) are/is a mature cell(s).

In some embodiments of the methods of the disclosure, the expanded population of cochlear or vestibular cells expresses leucine-rich repeat-containing G-protein coupled receptor 5 (Lgr5).

In some embodiments of the methods of the disclosure, the cochlear supporting cell(s) or vestibular supporting cell(s) are/is a cochlear supporting cell(s).

In some embodiments of the methods of the disclosure, the expanded population of cochlear or vestibular cells are cochlear cells.

In some embodiments of the methods of the disclosure, the TAZ activator in combination with the Wnt agonist increases the Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay

The disclosure provides a method of treating a subject who has, or is at risk of, developing an inner ear hearing or balance disorder, by administering to the subject: a) a transcriptional coactivator with PDZ-binding motif (TAZ) activator; and b) a Wnt agonist wherein (a) and (b) can occur in any order or simultaneously.

In some embodiments of the methods of the disclosure, the subject has an inner ear hearing or balance disorder.

In some embodiments of the methods of the disclosure, the disorder is an inner ear hearing disorder.

In some embodiments of the methods of the disclosure, the disorder is a balance disorder

In some embodiments of the methods of the disclosure, the inner ear hearing or balance disorder is sensorineural hearing loss.

In some embodiments of the methods of the disclosure, wherein the treatment results in improved auditory function when assessed by behavioural audiometry or auditory brainstem response (ABR) testing or any other measure of hearing loss as defined herein.

In some embodiments of the methods of the disclosure, the TAZ activator is IBS008738, TM-25659, FHZ-000706, or TT10.

In some embodiments of the methods of the disclosure, the 1BS008738 is at a concentration of about between 1 μM to 30 μM.

In some embodiments of the methods of the disclosure, the TM-25659 is at a concentration of about between 10 μM to 100 μM.

In some embodiments of the methods of the disclosure, the TT10 is at a concentration of about between 1 μM to 10 μM.

In some embodiments of the methods of the disclosure, the FHZ-000706 is at a concentration of about between 1 μM to 1000 μM.

In some embodiments of the methods of the disclosure, the Wnt agonist is a GSK3 inhibitor.

In some embodiments of the methods of the disclosure, the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.

In some embodiments of the methods of the disclosure, methods further include contacting the cochlear or vestibular supporting cell(s) with, or administering to the subject, an epigenetic agent.

In some embodiments of the methods of the disclosure, the epigenetic agent is an HDAC inhibitor, an EZH2 inhibitor, a DOT1L inhibitor a KDM inhibitor or an LSD1 inhibitor.

In some embodiments of the methods of the disclosure, the HDAC inhibitor is Valproic Acid (VPA)

In some embodiments of the methods of the disclosure, the VPA is at a concentration of about between 100 μM to 4,000 μM.

In some embodiments of the methods of the disclosure, the EZH2 inhibitor is an enzymatic inhibitor.

In some embodiments of the methods of the disclosure, the EZH2 inhibitor is selected from the group consisting of; CPI-1205, CPI-169, E11, PF-06821497, tazemetostat, valemetostat, CPI-360, EPZ011989, UNC 2399, and PF 06726304.

In some embodiments of the methods of the disclosure, the CPI-1205 is at a concentration of about between 10 nM to 1000 nM.

In some embodiments of the methods of the disclosure, the E11 is at a concentration of about between 1 μM to 10 μM.

In some embodiments of the methods of the disclosure, the PF-06821497 is at a concentration of about between 1 nM to 100 nM.

In some embodiments of the methods of the disclosure, the tazemetostat is at a concentration of about between 0.1 μM to 1.5 μM.

In some embodiments of the methods of the disclosure, the valemetostat is at a concentration of about between 10 nM to 1000 nM.

In some embodiments of the methods of the disclosure, the CPI-169 is at a concentration of about between 1 μM to 10 μM.

In some embodiments of the methods of the disclosure, the CPI-360 is at a concentration of about between 0.100 nM to 100 μM.

In some embodiments of the methods of the disclosure, the EPZ011989 is at a concentration of about between 10 nM to 10 μM.

In some embodiments of the methods of the disclosure, the UNC 2399 is at a concentration of about between 1 μM to 1000 μM.

In some embodiments of the methods of the disclosure, the PF-06726304 is at a concentration of about between 10 nM to 10 μM.

In some embodiments of the methods of the disclosure, the DOT1L inhibitor is an S-adenosyl methionine (SAM) competitive inhibitor.

In some embodiments of the methods of the disclosure, the DOT1L inhibitor is selected from the group consisting of EPZ004777, pinometostat and SGC0946.

In some embodiments of the methods of the disclosure, the EPZ004777 is at a concentration of about between 0.5 μM to 45 μM.

In some embodiments of the methods of the disclosure, the pinometostat is at a concentration of about between 0.1 μM to 10 μM.

In some embodiments of the methods of the disclosure, the SGC0946 is at a concentration of about between 0.5 μM to 5 μM.

In some embodiments of the methods of the disclosure, the KDM inhibitor is AS 8351, TC-E 5002 or EPT-103182.

In some embodiments of the methods of the disclosure, the AS 8351 is at a concentration of about between 0.5 μM to 5 μM.

In some embodiments of the methods of the disclosure, the TC-E 5002 is at a concentration of about between 0.1 μM to 10 μM.

In some embodiments of the methods of the disclosure, the EPT-103182 is at a concentration of about 1 nM to 100 nM.

In some embodiments of the methods of the disclosure, the LSD1 inhibitor is irreversible.

In some embodiments of the methods of the disclosure, the LSD1 inhibitor is selected from the group consisting of GSK-2879552, GSK-LSD1, Tranylcypromine, Phenelzine sulfate, RN-1, or ORY-1001.

In some embodiments of the methods of the disclosure, GSK2879552 is at a concentration of about between 4 nM to 30 μM.

In some embodiments of the methods of the disclosure, GSK-LSD1 is at a concentration of about between 4 nM to 50 μM.

In some embodiments of the methods of the disclosure, Tranylcypromine is at a concentration of about between 0.1 μM to 20 μM.

In some embodiments of the methods of the disclosure, Phenelzine sulfate at a concentration of about between 0.1 μM to 10 μM.

In some embodiments of the methods of the disclosure, RN-1 is at a concentration of about between 1 nM to 1000 nM.

In some embodiments of the methods of the disclosure, ORY-1001 at a concentration of about between 1 nM to 1000 nM.

In some embodiments of the methods of the disclosure, the TAZ activator is administered locally and/or systemically.

In some embodiments of the methods of the disclosure, the Wnt agonist is administered locally and/or systemically.

In some embodiments of the methods of the disclosure, the epigenetic agent is administered locally and/or systemically.

In some embodiments of the methods of the disclosure, the local administration is to the tympanic membrane, the middle ear or the inner ear.

In some embodiments of the methods of the disclosure, the local administration is to the middle ear

In some embodiments of the methods of the disclosure, the systemic administration is oral or parenteral.

In some embodiments of the methods of the disclosure, the systemic administration is oral.

In some embodiments of the methods of the disclosure, the TAZ activator is 1BS008738, TM-25659 or TT10.

In some embodiments of the methods of the disclosure, wherein the TAZ activator is IBS008738 and is administered locally at a dose of 10 μM.

In some embodiments of the methods of the disclosure, the TAZ activator is IBS008738 and is administered systemically at a dose of 25 mg.

In some embodiments of the methods of the disclosure, the TAZ activator is TM-25659 and is administered systemically at a dose of 25 mg.

In some embodiments of the methods of the disclosure, the TAZ activator is TT10 and is administered systemically at a dose of 25 mg.

In some embodiments of the methods of the disclosure, the TAZ activator is FHZ-000706 and is administered systemically at a dose of 25 mg.

In some embodiments of the methods of the disclosure, the Wnt agonist is CHIR99021 and is administered locally at a dose of 4 μM.

In some embodiments of the methods of the disclosure, the epigenetic agent is valproic acid (VPA) and is administered locally at a dose of 1 mM

In some embodiments of the methods of the disclosure, the epigenetic agent is valproic acid (VPA) and is administered systemically at a unit dose of 500 mg.

The disclosure provides a pharmaceutical composition including a TAZ activator, a Wnt agonist and a pharmaceutically acceptable carrier.

In some embodiments of the pharmaceutical compositions of the disclosure, the TAZ activator is IBS008738, TM-25659, FHZ-000706, or TT10.

In some embodiments of the pharmaceutical compositions of the disclosure, the IBS008738 is at a concentration of about between 1 mM to 30 mM.

In some embodiments of the pharmaceutical compositions of the disclosure, the TM-25659 is at a concentration of about between 1 mM to 100 mM.

In some embodiments of the pharmaceutical compositions of the disclosure, the TT10 is at a concentration of about between 1 mM to 100 mM.

In some embodiments of the pharmaceutical compositions, the FHZ-000706 is at a concentration of about between 1 mM to 1000 mM.

In some embodiments of the pharmaceutical compositions of the disclosure, the Wnt agonist is a GSK3 inhibitor.

In some embodiments of the pharmaceutical compositions of the disclosure, the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.

In some embodiments of the pharmaceutical compositions of the disclosure, the composition further includes an epigenetic agent.

In some embodiments of the pharmaceutical compositions of the disclosure, the epigenetic agent is an HDAC inhibitor, an EZH2 inhibitor, a DOT1L inhibitor a KDM inhibitor or a LSD1 inhibitor.

In some embodiments of the pharmaceutical compositions of the disclosure, the HDAC inhibitor is Valproic Acid (VPA)

In some embodiments of the pharmaceutical compositions of the disclosure, the VPA is at a concentration of about between 100 mM to 4,000 mM.

In some embodiments of the pharmaceutical compositions of the disclosure, the EZH2 inhibitor an enzymatic inhibitor.

In some embodiments of the pharmaceutical compositions of the disclosure, the EZH2 inhibitor is selected from the group consisting of: CPI-1205, CPI-169, E11, PF-06821497, tazemetostat, valemetostat, CPI-360, EPZ011989, UNC 2399, and PF 06726304.

In some embodiments of the pharmaceutical compositions of the disclosure, wherein the CPI-1205 is at a concentration of about between 10 μM to 1000 μM.

In some embodiments of the pharmaceutical compositions of the disclosure, wherein the E11 is at a concentration of about between 1 mM to 10 mM.

In some embodiments of the pharmaceutical compositions of the disclosure, wherein the PF-06821497 is at a concentration of about between 1 μM to 10 μM.

In some embodiments of the pharmaceutical compositions of the disclosure, wherein the tazemetostat is at a concentration of about between 0.1 mM to 10 mM.

In some embodiments of the pharmaceutical compositions of the disclosure, the valemetostat is at a concentration of about between 10 μM to 1000 μM.

In some embodiments of the pharmaceutical compositions of the disclosure, the CPI-169 is at a concentration of about between 1 mM to 10 mM.

In some embodiments of the pharmaceutical compositions, the CPI-360 is at a concentration of about between 100 μM to 100 mM.

In some embodiments of the pharmaceutical compositions, the EPZ011989 is at a concentration of about between 10 μM to 10 mM.

In some embodiments of the pharmaceutical compositions, the UNC 2399 is at a concentration of about between 1 mM to 1000 mM.

In some embodiments of the pharmaceutical compositions, the PF-06726304 is at a concentration of about between 10 μM to 10 mM.

In some embodiments of the pharmaceutical compositions of the disclosure, the DOT1L inhibitor and S-adenosyl methionine (SAM) competitive inhibitor.

In some embodiments of the pharmaceutical compositions of the disclosure, the DOT1L inhibitor is selected from the group consisting of EPZ004777, pinometostat and SGC0946.

In some embodiments of the pharmaceutical compositions of the disclosure, the EPZ004777 is at a concentration of about between 0.5 mM to 45 mM.

In some embodiments of the pharmaceutical compositions of the disclosure, the pinometostat is at a concentration of about between 0.1 mM to 10 mM.

In some embodiments of the pharmaceutical compositions of the disclosure, the SGC0946 is at a concentration of about between 0.5 mM to 5 mM.

In some embodiments of the pharmaceutical compositions of the disclosure, the KDM inhibitor is AS 8351, TC-E 5002 and EPT-103182.

In some embodiments of the pharmaceutical compositions of the disclosure, the AS 8351 is at a concentration of about between 0.5 μM to 5 μM.

In some embodiments of the pharmaceutical compositions of the disclosure, the TC-E 5002 is at a concentration of about between 0.1 μM to 10 μM.

In some embodiments of the pharmaceutical compositions of the disclosure, the EPT-103182 is at a concentration of about between 1 μM to 100 μM.

In some embodiments of the pharmaceutical compositions of the disclosure, the LSD1 inhibitor is irreversible.

In some embodiments of the pharmaceutical compositions of the disclosure, wherein the LSD1 inhibitor is selected from the group consisting of GSK-2879552, GSK-LSD1, Tranylcypromine, Phenelzine sulfate, RN-1, or ORY-1001.

In some embodiments of the pharmaceutical compositions of the disclosure, wherein GSK2879552 is at a concentration of about between 4 nM to 30 μM.

In some embodiments of the pharmaceutical compositions of the disclosure, GSK-LSD1 is at a concentration of about between 4 nM to 50 μM.

In some embodiments of the pharmaceutical compositions of the disclosure, Tranylcypromine is at a concentration of about between 0.1 μM to 20 μM.

In some embodiments of the pharmaceutical compositions of the disclosure, Phenelzine sulfate at a concentration of about between 0.1 μM to 10 μM.

In some embodiments of the pharmaceutical compositions, RN-1 is at a concentration of about between 1 μM to 1000 μM.

In some embodiments of the pharmaceutical compositions, ORY-1001 is at a concentration of about between 1 μM to 1000 μM.

In some embodiments of the pharmaceutical compositions of the disclosure, the pharmaceutical composition is in a biocompatible matrix.

In some embodiments of the pharmaceutical compositions of the disclosure, the biocompatible matrix includes hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.

In some embodiments of the pharmaceutical compositions of the disclosure, the pharmaceutical composition is formulated for administration as defined in any of claims 73-94

In some embodiments of the pharmaceutical compositions of the disclosure, the composition is for use in treating or preventing an inner ear hearing or balance disorder.

In some embodiments of the pharmaceutical compositions of the disclosure, the composition is for use according to claim 167, wherein the inner ear hearing or balance disorder is sensorineural hearing loss.

In some embodiments of the use of the pharmaceutical compositions of the disclosure, the composition is for use in the manufacture of a medicament for the treatment or prevention of an inner ear hearing or balance disorder.

In some embodiments of the use of the pharmaceutical compositions of the disclosure, the inner ear hearing or balance disorder is sensorineural hearing loss.

The disclosure provides a transcriptional coactivator with PDZ-binding motif (TAZ) activator for use in treating or preventing an inner ear hearing or balance disorder in a subject, wherein the subject has been, or will be, administered a Wnt agonist.

The disclosure provides a Wnt agonist for use in treating or preventing an inner ear hearing or balance disorder in a subject, wherein the subject has been, or will be, administered a transcriptional coactivator with PDZ-binding motif (TAZ) activator.

The disclosure provides a epigenetic agent for use in treating or preventing an inner ear hearing or balance disorder in a subject, wherein the subject has been, or will be, administered a transcriptional coactivator with PDZ-binding motif (TAZ) activator and a Wnt agonist.

In some embodiments, the TAZ activator, Wnt agonist or epigenetic agent for use according to any of the embodiments of the disclosure, wherein the inner ear hearing or balance disorder is sensorineural hearing loss.

In some embodiments, the TAZ activator, Wnt agonist or epigenetic agent for use according to any of the embodiments of the disclosure, wherein the treatment is as defined in any of the embodiments of the disclosure.

The disclosure provides a container including a transcriptional coactivator with PDZ-binding motif (TAZ) activator and instructions, where those instructions describe the TAZ activator use for treating or preventing an inner ear hearing or balance disorder in a subject, wherein the instructions require that the subject has been, or will be, administered a Wnt agonist.

The disclosure provides a container including a Wnt agonist and instructions, where those instructions describe the Wnt agonist's use in treating or preventing an inner ear hearing or balance disorder in a subject, wherein the instructions require that the subject has been, or will be, administered a transcriptional coactivator with PDZ-binding motif (TAZ) activator.

The disclosure provides a container including an epigenetic agent and instructions, where those instructions describe the epigenetic agent's use in treating or preventing an inner ear hearing or balance disorder in a subject, wherein the instructions require that the subject has been, or will be, administered a transcriptional coactivator with PDZ-binding motif (TAZ) activator and a Wnt agonist.

In some embodiments, the container according to any of the embodiments of the disclosure, wherein the inner ear hearing or balance disorder is sensorineural hearing loss.

In some embodiments, the container according to any of the embodiments of the disclosure, wherein the treatment is as defined in any of the embodiments of the disclosure.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present invention, suitable methods and materials are described below. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE DRAWINGS

FIG. 1A is a graph depicting that the TAZ activator IBS008738 enhances Lgr5 GFP(+) progenitor cell proliferation when combined with CHIR in a background of growth factors. The y-axis depicts Lgr5 GFP(+) cell count and the x-axis depicts control conditions (background growth factors plus CHIR (EFI-C) or CHIR and VPA (EFI-CV)) versus CHIR+IBS008738 (EFI-C-IBS). Media components include 50 ng/mL EGF, 50 ng/mL bFGF, 50 ng/mL IGR1, 4 μM CHIR99021, 1 mM VPA and 10 μM IBS008738.

FIG. 1B is a graph depicting that the TAZ activator IBS008738 enhances enrichment of Lgr5 GFP(+) cochlear progenitor cells when combined with CHIR in a background of growth factors. The y-axis depicts Lgr5 GFP(+) cell proliferation percentage and the x-axis depicts control conditions (EFI-C) or (EFI-CV) versus CHIR+IBS008738 (EFI-C-IBS). Media components include 50 ng/mL EGF, 50 ng/mL bFGF, 50 ng/mL IGR1, 4 μM CHIR99021, 1 mM VPA and 10 μM IBS008738.

FIG. 2A is a graph depicting that the TAZ activator IBS008738 does not enhance Lgr5 GFP(+) progenitor cell proliferation when combined with CHIR and VPA in a background of growth factors. The y-axis depicts Lgr5 GFP(+) cell count and the x-axis depicts control conditions (background growth factors plus CHIR (EFI-C) or CHIR and VPA (EFI-CV)) versus CHIR+IBS008738 (EFI-C-IBS). Media components include 50 ng/mL EGF, 50 ng/mL bFGF, 50 ng/mL IGR1, 4 μM CHIR99021, 1 mM VPA and 10 μM IBS008738.

FIG. 2B is a graph depicting that the TAZ activator IBS008738 enhances enrichment of Lgr5 GFP(+) cochlear progenitor cells when combined with CHIR and VPA in a background of growth factors. The y-axis depicts Lgr5 GFP(+) cell proliferation percentage and the x-axis depicts control conditions (EFI-C) or (EFI-CV) versus CHIR+IBS008738 (EFI-C-IBS). Media components include 50 ng/mL EGF, 50 ng/mL bFGF, 50 ng/mL IGR1, 4 μM CHIR99021, 1 mM VPA and 10 μM IBS008738.

FIG. 3A is a graph depicting that the TAZ activator FHZ-000706 does not proliferate Lgr5 GFP(+) cochlear progenitor cells in a background of growth factors. The y-axis depicts Lgr5 GFP(+) cell area and the x-axis depicts concentration of FHZ-000706. Media components include 50 ng/mL EGF, 50 ng/mL bFGF, 50 ng/mL IGR1, 4 μM CHIR99021, 1 mM VPA and 0-30 μM FHZ-000706.

FIG. 3B is a graph depicting that the TAZ activator FHZ-000706 does not enrich for Lgr5 GFP(+) cochlear progenitor cells in a background of growth factors. The y-axis depicts Lgr5 GFP(+) cell area and the x-axis depicts concentration of FHZ-000706. Media components include 50 ng/mL EGF, 50 ng/mL bFGF, 50 ng/mL IGR1, 4 μM CHIR99021, 1 mM VPA and 0-30 μM FHZ-000706.

FIG. 4A is a graph depicting that the TAZ activator FHZ-000706 enhances Lgr5 GFP(+) progenitor cell proliferation when combined with CHIR in a background of growth factors compared to CHIR alone. The y-axis depicts Lgr5 GFP(+) cell area and the x-axis depicts media conditions: E=50 ng/mL EGF, F=50 ng/mL bFGF, I=50 ng/mL IGR1, C═CHIR=4 μM CHIR99021, FHZ-706=10 μM FHZ-000706.

FIG. 4B is a graph depicting that the TAZ activator FHZ-000706 enhances enrichment of Lgr5 GFP(+) cochlear progenitor cells when combined with CHIR in a background of growth factors compared to CHIR alone. The y-axis depicts Lgr5 GFP(+) cell area percentage and the x-axis depicts media conditions: E=50 ng/mL EGF, F=50 ng/mL bFGF, 1=50 ng/mL IGR1, C=CHIR=4 μM CHIR99021, FHZ-706=10 μM FHZ-000706.

### DETAILED DESCRIPTION

The invention is based upon the discovery that activating TAZ motif (also called WWTR1) a transcriptional coactivator with a PDZ-binding domain with a TAZ activator in combination with a Wnt agonist results in the proliferation of cochlear supporting cells or vestibular supporting cells while maintaining, in the daughter cells, the capacity to differentiate into cochlear hair cells or vestibular hair cells. Wnt agonists have previously been used to stimulate proliferation of supporting cells with some success. However, the combination of TAZ activation and Wnt agonist resulted in a surprising level of proliferation and/or enrichment of cells in these contexts. In some embodiments, the combination of TAZ activation and a Wnt agonist results in cell populations where the expanded cells are enriched for Lgr5 expression (i.e. a greater percentage of the expanded cell population express Lgr5 compared to the starting cell population) compared to either Wnt agonist or TAZ activation alone. Indeed, the combination of TAZ activation and a Wnt agonist increased proliferation of cochlear supporting cells or vestibular supporting cells relative to stimulation with either Wnt agonist or TAZ activation alone. The combination of TAZ activation and Wnt agonist therefore produces a larger population of expanded cochlear cells or vestibular cells compared to either Wnt agonist or TAZ activation alone. In other words, the combination of TAZ activation and Wnt agonist is more effective at inducing self-renewal of cochlear supporting cells and vestibular supporting cells than either Wnt agonist or TAZ activation alone. By self-renewal of cochlear supporting cells or vestibular supporting cells, it is meant inducing the a cochlear supporting cell or vestibular supporting cell to proliferate while maintaining, in the daughter cells, the capacity to differentiate into cochlear hair cells, thus providing a therapy for treating a subject who has, or is at risk of, developing an inner ear hearing or balance disorder.

The methods described herein can increase the proliferation of cochlear supporting cells or vestibular supporting cells. Typically, the cochlear supporting cell or vestibular supporting cell in which proliferation is stimulated expresses Lgr5 (Leucine-rich repeat-containing G-protein coupled receptor 5). However the methods described herein may also stimulate proliferation of supporting cells with little or no Lgr5 expression.

The methods described herein can produce an expanded population of cochlea or vestibular cells. In some embodiments, the expanded cells are enriched for Lgr5 expression (i.e. a greater percentage of the expanded cell population express Lgr5 compared to the starting cell population).

Lgr5 is a member of GPCR class A receptor proteins that is expressed across a diverse range of tissues such as in the muscle, placenta, spinal cord and brain, and particularly as a biomarker of adult stem cells in certain tissues. Lgr5+ stem cells are the precursors for sensory hair cells that are present in cochlea and vestibular organs of the inner ear. Increasing the population of Lgr5+ cochlear or vestibular cells is therefore beneficial because it increases the population of precursor cells which may differentiate into sensory hair cells.

The present invention provides compositions and methods for inducing the self-renewal of a cochlear supporting cells and vestibular supporting cells by increasing TAZ expression or activity in combination with a Wnt agonist.

Thus, in various aspects the invention provides compositions and methods for increasing proliferation of a cochlear supporting cell or vestibular supporting cell; producing an expanded population of cochlear or vestibular cells and treating an inner ear hearing or balance disorder in a subject by contacting a cochlear supporting cell or vestibular supporting cell, or administering to a subject, a TAZ activator and a Wnt Agonist.

In another aspect of the invention, the cochlear supporting cell or vestibular supporting cell is further contacted with, or a subject is further administered with, an epigenetic agent. In some embodiments, the epigenetic agent is an HDAC inhibitor, for example valproic acid (VPA), an LSD1 inhibitor, an EZH2 inhibitor, a DOT1L inhibitor, or a KDM inhibitor. The addition of an epigenetic agent to the TAZ activator and Wnt agonist is advantageous because proliferation of the supporting cell population can be increased compared to the combination of either TAZ activator and Wnt agonist or Wnt agonist and valproic acid. In some embodiments, the expanded population of cells that can be produced following treatment with and TAZ activator, a Wnt agonist and an epigenetic agent is larger than the expanded population of cells that is produced compared to the combination of either TAZ activator and Wnt agonist or Wnt agonist and valproic acid. The Lgr5+ cell population can be more enriched when an epigenetic agent is used compared to the combination of a TAZ activator and a Wnt agonist, or the combination of a Wnt agonist and an HDAC inhibitor.

### TAZ Activators

TAZ motif (also called WWTR1) a transcriptional coactivator with a PDZ-binding was identified as a 14-3-3-binding protein. It is similar to Yes-associated protein 1 (YAP1) in its molecular structure, which consists of an N-terminal TEAD binding domain, one or two WW domains, and a transcriptional activation domain.

TAZ is phosphorylated at four sites by large tumor suppressor kinase 1 (LATS1) and LATS2, which are core kinases of the Hippo pathway. Phosphorylated TAZ is trapped by 14-3-3, is recruited from the nucleus to the cytoplasm, and undergoes protein degradation. In this way, the Hippo pathway negatively regulates TAZ. Accordingly, in some embodiments, TAZ is activated via a compound that affects a member of the HIPPO pathway, e.g., YAP, MST1, MST2, LATS1, LATS2, MOB1, and SAV1.

In addition to the Hippo pathway, TAZ is regulated by cell junction proteins such as ZO-1, ZO-2, and angiomotin. Recent studies have revealed that TAZ is under the control of the actin cytoskeleton and the mechanical stretch. Moreover, Wnt signaling stabilizes. Conversely, cytoplasmic TAZ binds -catenin and Dishevelled (DVL) and inhibits -catenin nuclear localization and DVL phosphorylation to negatively regulate the Wnt pathway.

TAZ activators are chemical compounds that stabilizes and increases unphosphorylated TAZ levels.

Thus, “TAZ activator” refers to an agent capable of the increasing the stability or activity of TAZ. For example, an TAZ activator results in a decrease in TAZ phoshorylation and/or TAZ protein degradation.

In certain embodiments, the TAZ activator increases the stability or activity of TAZ by at least 5, 10, 20, 30, 40, 50, 60, 70, 80, 90, or 100% relative to a control, for example relative to a baseline level of activity.

In certain embodiments, the TAZ activator increases the expression of a target gene by at least 5, 10, 20, 30, 40, 50, 60, 70, 80, 90, or 100% relative to a control, for example relative to a baseline level of activity.

In some embodiments, the TAZ activator increases the stability or activity of TAZ by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, increases the expression of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary TAZ Activators are provided in Table 1.

TABLE 1

| | | | | | | | | | |
|---|---|---|---|---|---|---|---|---|---|
| | | | | | | | | Human | |
| | | Chemo- | | Lit | Lgr5+ | Perilymph | Formulation | In Vive | Human |
| Agent | CAS | type | Mechanism | Cell | Assay | Conc | Conc | Conc | Dosage |

| | | | | | | | | | | | | |
|---|---|---|---|---|---|---|---|---|---|---|---|---|
| IBS008738 | 371128-48-2 | Hydrazone | TAZ Activ. | | | 1.1-30 | μM | 1.1-30 | μM | 1.1-30 | mM | 25-500 mg |
| TM-25659 | 260553-97-7 | AT II | TAZ Activ. | 10-100 | μM | 10-100 | μM | 10-100 | μM | 10-100 | mM | 25-500 mg |
| TT10 | 2230640-94-3 | Thiazole | TAZ Activ. | 1 | μM | 1-10 | μM | 1-10 | μM | 1-10 | mM | 25-500 mg |
| FHZ-000706 | 304871-19-0 | Thiazole | TAZ Activ. | | | 1-100 | μM | 1-100 | μM | 1-100 | mM | 25-500 mg |

| IBS003031 | 381177-81-7 | Acridine | YAP Activ. | | |
|---|---|---|---|---|---|
| TAZ12 | 371128-48-2 | Thiazole | TAZ Activ. | | |
| TM-53 | 1257247-76-9 | AT II | TAZ Activ. | 10-100 | μM |
| TM-54 | 1257247-77-0 | AT II | TAZ Activ. | 10-100 | μM |

| | | | |
|---|---|---|---|
| (—)- | 1257-08-5 | Nat Prod | |
| epicatechin | | | |
| gallate | | | |
| Ethacridine | 1837-57-6 | Acridine | Activity |
| kaempferol | 520-18-3 | Nat Prod | |
| KR 62980 | 867187-61-9 | N-Oxide | |
| phorbaketal | 1196507-03-5 | Nat Prod | |
| A | | | |

In some embodiments the TAZ activator is IBS008738, TM-25659, FHZ-000706, or TT10.

In some embodiments, TAZ is activated via a compound that affects a member of the HIPPO pathway, e.g., YAP, MST1, MST2, LATS1, LATS2, MOB1, and SAV1

Modulators of LATS1 and LATS2 can be found in the following references, the contents of which are herein incorporated by reference in their entirety:
Qiao, Jingxin; Lin, Guifeng; Xia, Anjie; Xiang, Zhiyu; Chen, Pei; Zhang, Guo; Li, Linli; Yang, Shengyong. Bioorganic & Medicinal Chemistry Letters (2019), 29(18), 2595-260.

Modulators of MST1 and MST2 can be found in the following references, the contents of which are herein incorporated by reference in their entirety:
Ardestani Amin; Annamalai Karthika; Lupse Blaz; Geravandi Shirin; Dobrowolski Aleksandra; Oetjen Janina; Herranz Raquel; Awal Sushil; Altenhofen Delsi; Maedler Kathrin; et al Nature communications (2019), 10(1), 5015.
Triastuti, Efta; Nugroho, Ardiansah Bayu; Zi, Min; Prehar, Sukhpal; Kohar, Yulia Suciati; Bui, Thuy Anh; Stafford, Nicholas; Cartwright, Elizabeth J.; Abraham, Sabu; Oceandy, Delvac British Journal of Pharmacology (2019), 176(20), 3956-3971.
Liang, F., Shi, L., Zheng, J., Chen, S., Wang, Y., & Zhang, J. Scientific Reports 2017, 7(1), 9201.
Cao, Jingwen; Huang, Wenlong Two faces of Hippo: activate or suppress the Hippo pathway in cancer Anti-Cancer Drugs (2017), 28(10), 1079-1085.
Fan, Fuqin; He, Zhixiang; Kong, Lu-Lu; Chen, Qinghua; Yuan, Quan; Zhang, Shihao; Ye, Jinjin; Liu, Hao; Sun, Xiufeng; Geng, Jing; et al. Science Translational Medicine (2016), 8(352), 352.
Juan, Wen Chun; Hong, Wanjin. Genes (2016), 7(9), 55/1.
Kotha, S.; Goyal, D.; Bitra, A.; Thota, N.; Kruger, G.; Anand, R. RSC Advances (2013), 3(46), 24447-2445.

The following are references discussing the HIPPO pathways and modulators of the HIPPO pathway, the contents of which are herein incorporated by reference in their entirety:
Juan, Wen Chun; Hong, Wanjin. Genes (2016), 7(9), 55/1.
Guo, Liwen; Teng, Lisong. Review International Journal of Oncology (2015), 46(4), 1444-1452.
Johnson, Randy; Halder, Georg. Nature Reviews Drug Discovery (2014), 13(1), 63-79.
Gibault, Floriane; Sturbaut, Manon; Bailly, Fabrice; Melnyk, Patricia; Cotelle, Philippe. Journal of Medicinal Chemistry (2018), 61(12), 5057-5072.
Nagashima, Shunta; Bao, Yijun; Hata, Yutaka. Therapy and Regenerative Medicine Current Drug Targets (2017), 18(4), 447-454.
Santucci, Matteo; Vignudelli, Tatiana; Ferrari, Stefania; Mor, Marco; Scalvini, Laura; Bolognesi, Maria Laura: Uliassi, Elisa; Costi, Maria Paola. Journal of Medicinal Chemistry (2015), 58(12), 4857-4873.

### Wnt Agonists

A Wnt agonist refers to an agent that increases the expression, levels, and/or activity of a Wnt gene, protein, or signaling pathway (e.g. TCF/LEF, Frizzled receptor family, Wif1, Lef1, Axin2, β-catenin) in a cell, for example, a cochlear cell. A Wnt agonist includes a GSK3 inhibitor, such as a GSK3-α or a GSK3-β inhibitor. In preferred embodiments, the GSK3 inhibitor is a GSK3-β inhibitor.

The TCF/LEF family is a group of transcription factors that bind to DNA through a high mobility group domain, and which are involved in the Wnt signaling pathway where they recruit the coactivator β-catenin to enhancer elements of targeted genes. Frizzled is a family of G protein-coupled receptor proteins that serves as receptors in the Wnt signaling pathway. Frizzled receptors inhibit intracellular β-catenin degradation and activate TCF/LEF-mediated transcription.

In some embodiments, the Wnt agonist increases Wnt signaling in a cochlear or vestibular cell by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, the Wnt agonist increases TCF/LEF-mediated transcription in a cochlear or vestibular cell, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, the Wnt agonist binds and activates a Frizzled receptor family member, for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) or more relative to a control, for example relative to a baseline level of activity.

In some embodiments, the Wnt agonist inhibits GSK3 for example, by about or at least about 10, 20, 30, 40, 50, 60, 70, 80, 90, 100, 200, 300, 400, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more or more relative to a control, for example relative to a baseline level of activity.

In some embodiments, the Wnt agonist preferentially upregulates Jag-1, Deltex-1 or Hif-1 more that the Wnt agonist upregulates Hes or Hey. In some embodiments, the Wnt agonist increases the expression of Jag-1, Deltex-1 and/or Hif-1 10%, 25%, 50%, 75%, 100%, 125%, 150%, 175%, 200%, 250% or more than it increases the expression or activity of Hes and Hey.

Exemplary agents having activity as a Wnt agonist are provided in Table 2 and 3 below, including pharmaceutically-acceptable salts thereof.

TABLE 2

| | | | | | | | | Formul. | |
|---|---|---|---|---|---|---|---|---|---|
| | | GSK-3 | GSK-3 | Lgr5+ | | Perilymph | | Conc. | |
| Agent | CAS | alpha | alpha | Assay | | Conc. | | Intraymp | |
| CHIR99021 | 252917-06-9 | 4.4 nM | 6.6 nM | 2-6 | μM | 2-6 | μM | 4 | mM |
| AZD 1080 | 612487-72-6 | 6.9 nM | 31 nM | 1-5 | μM | 1-5 | μM | 1-5 | mM |
| GSK XX1I | 1195901-31-5 | 2.3 nM | 2.0 nM | 0.2-1 | μM | 0.2-1 | μM | 0.2-1 | mM |
| LY2090314 | 603288-22-8 | 2.1 nM | 0.9 nM | 5-20 | nM | 5-20 | nM | 5-20 | μM |

TABLE 3

| Class | Agent | CAS |
|---|---|---|
| WNT | | |
| ARFGAP1 | QS 11 | 944328-88-5 |
| ARFGAP1 | WASP-1, ZINC00087877 | 352328-82-6 |
| Axin | Cpd1 | 1357473-75-6 |
| Axin | Cpd2 | 1228659-47-9 |
| Axin | HLY78 | 854847-61-3 |
| Axin | SKL2001 | 909089-13-0 |
| beta-catenin | DCA | 56-47-3 |
| Disrupts the Axin | Compound 2 | 1360540-82-4 |
| Complex | | |
| Disrupts the Axin | Compound 71 | 1622429-71-3 |
| Complex | | |
| Disrupts the Axin | ISX 9 | 832115-62-5 |
| Complex | | |
| DKK1 inhibitor | WAY-262611 | 1123231-07-1 |
| MEK | Radicicol | 12772-57-5 |
| MEK | Selumetinib (AZD6244) | 606143-52-6 |
| PP2A | IQ 1 | 331001-62-8 |
| sFRP-1 inhibitor | (Dimethylamino)propyl)-2-ethyl-5- | 915754-88-0 |
| | (phenylsulfonyl)benzenesulfonamide | |
| sFRP-1 inhibitor | Cyclosporine A (CsA) | 59865-13-3 |
| sFRP-1 inhibitor | Cyclosporine analogs | |
| sFRP-1 inhibitor | PSC833 (Valspodar) | 121584-18-7 |
| sFRP-1 inhibitor | WAY 316606 | 915759-45-4 |
| Target Undetermined | Diketones | WO 2016029021 A1; |
| | | WO 2012024404 A1 |
| Target Undetermined | Diketones | 1622429-56-4 |
| Target Undetermined | Diketones | 1360540-88-0 |
| Target Undetermined | Diketones | 1360540-89-1 |
| Target Undetermined | Diketones | 1622429-79-1 |
| Target Undetermined | Diketones | 1622429-75-7 |
| Target Undetermined | Diketones | 1622429-74-6 |
| Target Undetermined | Diketones | 1622430-76-5 |
| Target Undetermined | Diketones | 1622430-31-2 |
| Target Undetermined | Diketones | 1622430-52-7 |
| Target Undetermined | Diketones | 1622429-67-7 |
| Target Undetermined | Diketones | 1622429-65-5 |
| Target Undetermined | Diketones | 1622429-69-9 |
| van-Gogh-like receptor | Compound 109 | 1314885-81-8 |
| proteins (Vangl) | | |
| Wnt Ligand | Wnt-1 | Protein |
| Wnt Ligand | Wnt-10a | Protein |
| Wnt Ligand | Wnt-10b/12 | Protein |
| Wnt Ligand | Wnt-11 | Protein |
| Wnt Ligand | Wnt-16 | Protein |
| Wnt Ligand | Wnt-2/Irp (Int-I-related protein) | Protein |
| Wnt Ligand | Wnt-2b/13 | Protein |
| Wnt Ligand | Wnt-3/Int-4 | Protein |
| Wnt Ligand | Wnt-3a | Protein |
| Wnt Ligand | Wnt-4 | Protein |
| Wnt Ligand | Wnt-5a | Protein |
| Wnt Ligand | Wnt-5b | Protein |
| Wnt Ligand | Wnt-6 | Protein |
| Wnt Ligand | Wnt-7a | Protein |
| Wnt Ligand | Wnt-7b | Protein |
| Wnt Ligand | Wnt-8a/8d | Protein |
| Wnt Ligand | Wnt-8b | Protein |
| Wnt Ligand | Wnt-9a/14 | Protein |
| Wnt Ligand | Wnt-9b/14b/15 | Protein |
| Wnt Related Protein | Norrin | Protein |
| Wnt Related Protein | R-Spondin 1/2/3/4 | Protein |
| Wnt-3a/Dkk-1 | BML-284 | 853220-52-7 |
| Wnt-3a/Dkk-1 | Compound 1 | 1084833-94-2 |
| Wnt-3a/Dkk-1 | Compound 25 | 1084834-05-8 |
| GSK3 alpha | | |
| CREB knockdown | 666-15 | 1433286-70-4 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | I5 | 264217-24-5 |
| Maleimide | Tivantinib | 905854-02-6 |
| Organometallic | Compound (R)-DW12 | 1047684-07-0 |
| Organometallic | Compound 3 | 1498285-39-4 |
| | | 1498285-48-5 |
| Organometallic | Compound lambda-OS1 | 1291104-51-2 |
| | | 1292843-11-8 |
| Oxadiazoles | Compound 14d | 1374671-64-3 |
| Oxadiazoles | Compound 15b | 1374671-66-5 |
| Oxadiazoles | Compound 27 | 1820758-44-8 |
| Oxindole | AZD1080 | 612487-72-6 |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3b XXII | 1195901-31-5 |
| Pyrazolopyridazines | Compound 18 | 405223-20-3 |
| Pyrazolopyridazines | Compound 19 | 405223-71-4 |
| Pyrazolopyridines | Compound 14 | 583038-63-5 |
| Pyrazolopyridines | Compound 23 | 583038-76-0 |
| Pyrazolopyridines | Pyrazolopyridine 34 | 583039-27-4 |
| Pyrazolo- | BRD1172 | 1597438-86-2 |
| tetrahydroquinolinone | | |
| Pyrazolo- | BRD1652 | 1597438-93-1 |
| tetrahydroquinolinone | | |
| Pyrazolo- | BRD4003 chiral | 1597439-60-5 |
| tetrahydroquinolinone | | |
| Pyrazolo- | BRD4003 chiral | 1597439-59-2 |
| tetrahydroquinolinone | | |
| Pyrazolo- | Compound 11 | 1597439-12-7 |
| tetrahydroquinolinone | | |
| Pyrazolo- | Compound 16 | 1597440-17-9 |
| tetrahydroquinolinone | | |
| Pyrazolo- | Compound 8 | 1597439-01-4 |
| tetrahydroquinolinone | | |
| Pyrazolo- | Compound 9 | 1597439-02-5 |
| tetrahydroquinolinone | | |
| Triazolpyrimidine | Compound 90 | 91322-11-1 |
| Triazolpyrimidine | Compound 92 | 1043429-30-6 |
| Urea | AR-A014418 | 487021-52-3 |
| GSK3-beta | | |
| Acid | Bikinin | 188011-69-0 |
| Acid | Valproic Acid, Sodium Salt | 99-66-1 |
| Aloisines | Aloisine A | 496864-16-5 |
| Aloisines | Aloisine B | 496864-14-3 |
| Aloisines | TWS119 | 1507095-58-0 |
| Aminopyrimidine | CHIR98014 (CT98014) | 252935-94-7 |
| Aminopyrimidine | CHIR98023 (CT98023) | 252904-84-0 |
| Aminopyrimidine | CHIR98024 (CT98024) | 556813-39-9 |
| Aminopyrimidine | CHIR99021 (CT99021) | 252917-06-9 |
| Aminopyrimidine | CT20026 | 403808-63-9 |
| Aminopyrimidinyl | CGP60474 | 164658-13-3 |
| Aminopyrimidinyl | GSK-3β Inhibitor XVIII | 1139875-74-3 |
| Azaindolylmaleimide | Compound 29 | 436866-61-4 |
| Azaindolylmaleimide | Compound 46 | 682807-74-5 |
| Bisindolylmaleimide | Bisindolylmaleimide X HCl | 131848-97-0 |
| Bisindolylmaleimide | Compound 5a | 436866-54-5 |
| Bisindolylmaleimide | Enzastaurin (LY317615) | 170364-57-5 |
| Bisindolylmaleimide | GF109203x | 176504-36-2 |
| Bisindolylmaleimide | Ro318220 | 125314-64-9 |
| Dihydropyridine | ML320 | 1597438-84-0 |
| Flavone | Flavopiridol | 146426-40-6 |
| Furanosesquiterpenes | Palinurin | 254901-27-4 |
| Furanosesquiterpenes | Tricantin | 853885-55-9 |
| Furopyrimidine | Compound 100 | 744255-19-4 |
| Halomethylketones | Compound 17 | 62673-69-2 |
| Halomethylketones | GSK-3β Inhibitor VI | 62673-69-2 |
| Halomethylketones | GSK-3β Inhibitor VII | 99-73-0 |
| Hymenidin | Hymenidin | 107019-95-4 |
| Indirubins | 5-Iodo-indirubin-3′-monoxime | 331467-03-9 |
| Indirubins | 6-Bromoindirubin-3-acetoxime | 667463-85-6 |
| Indirubins | GSK-3 Inhibitor IX | 667463-62-9 |
| Indirubins | GSK-3 Inhibitor X | 740841-15-0 |
| Indirubins | Indirubin | 479-41-4 |
| Indirubins | Indirubin-3′-monoxime | 160807-49-8 |
| Indirubins | Indirubin-5-sulfonic acid sodium salt | 331467-05-1 |
| Inorganic atom | Beryllium | |
| Inorganic atom | Lithium Chloride | |
| Inorganic atom | Tungstate | |
| Inorganic atom | Zinc | |
| Isoindolone | Staurosporine | 62996-74-1 |
| Isonicotinamides | Compound 29 | 1772823-37-6 |
| Isonicotinamides | Compound 33 | 1772823-64-9 |
| Isonicotinamides | Compound 39 | 1772824-10-8 |
| Maleimide | 3F8 | 159109-11-2 |
| Maleimide | 603281-31-8 | 603281-31-8 |
| Maleimide | BIP-135 | 941575-71-9 |
| Maleimide | Compound 34 | 396091-16-0 |
| Maleimide | CP21R7 | 125314-13-8 |
| Maleimide | GSK-3 inhibitor 1 | 603272-51-1 |
| Maleimide | GSK-3β Inhibitor XI | 626604-39-5 |
| Maleimide | I5 | 264217-24-5 |
| Maleimide | IM-12 | 1129669-05-1 |
| Maleimide | Isogranulatimide | 244148-46-7 |
| Maleimide | KT 5720 | 108068-98-0 |
| Maleimide | LY2090314 | 603288-22-8 |
| Maleimide | SB-216763 | 280744-09-4 |
| Maleimide | SB-415286 (SB-41528) | 264218-23-7 |
| Maleimide | TCS 21311 | 1260181-14-3 |
| Maleimide | Tivantinib | 905854-02-6 |
| Manzamines | Manzamine A | 104196-68-1 |
| Miscellaneous | AZD2858 (AR28) | 486424-20-8 |
| Miscellaneous | CID 755673 | 521937-07-5 |
| Miscellaneous | Dibromocantharelline | 101481-34-9 |
| Miscellaneous | TCS 2002 | 1005201-24-0 |
| Organometallic | (RRu)-HB1229 | |
| Organometallic | (RRu)-NP549 | |
| Organometallic | Compound (R)-DW12 | 1047684-07-0 |
| Organometallic | Compound 3 | 1498285-39-4, |
| | | 1498285-48-5 |
| Organometallic | Compound lambda-OS1 | 1291104-51-2, |
| | | 1292843-11-8 |
| Organometallic | DW12 | 861251-33-4 |
| Organometallic | HB12 | 800384-87-6 |
| Organometallic | NP309 | 937810-13-4 |
| Oxadiazol | Compound 14d | 1374671-64-3 |
| Oxadiazol | Compound 15b | 1374671-66-5 |
| Oxadiazol | Compound 20x | 1005201-80-8 |
| Oxadiazol | GSK-3 Inhibitor II | 478482-75-6 |
| Oxadiazol | GSK3 Inhibitor, 2 | 1377154-01-2 |
| Oxadiazol | TC-G 24 | 1257256-44-2 |
| Oxindole | AZD1080 | 612487-72-6 |
| Oxindole | SU9516 | 77090-84-1 |
| Patent | CN 101341138 B | |
| Patent | CN 1319968 C | |
| Patent | CP-70949 | |
| Patent | CT118637 | |
| Patent | EP 1739087 A1 | |
| Patent | EP 1961748 A2 | |
| Patent | EP 2765188 A1 | |
| Patent | GI179186X | |
| Patent | GW784752X | |
| Patent | GW784775X | |
| Patent | US 20070088080 A1 | |
| Patent | US 20100292205 A1 | |
| Patent | U.S. Pat. No. 7,514,445 B2 | |
| Patent | U.S. Pat. No. 8,071,591 B2 | |
| Patent | U.S. Pat. No. 8,207,216 B2 | |
| Patent | U.S. Pat. No. 8,686,042 B2 | |
| Patent | U.S. Pat. No. 8,771,754 B2 | |
| Patent | WO 2001085685 A1 | |
| Patent | WO 2003037891 A1 | |
| Patent | WO 2006018633 A1 | |
| Patent | WO 2007102770 A1 | |
| Patent | WO 2008077138 A1 | |
| Patent | WO 2009017453 A1 | |
| Patent | WO 2010075551 A1 | |
| Patent | WO 2010104205 A1 | |
| Patent | WO 2011089416 A1 | |
| Patent | WO 2013124413 A1 | |
| Patent | WO 2014003098 A1 | |
| Patent | WO 2014013255 A1 | |
| Patent | WO 2014050779 A1 | |
| Patent | WO 2014059383 A1 | |
| Patent | WO 2014083132 A1 | |
| Patent | WO2006100490A1/EP 1863904 A1 | |
| Patent | WO2009017455 A1 | |
| Paullone | Cmpd 17b | 408532-42-3 |
| Paullone | Kenpaullone | 142273-20-9 |
| Paullones | Alsterpaullone | 237430-03-4 |
| Paullones | Alsterpaullone CN Ethyl | 852529-97-0 |
| Paullones | Azakenpaullone | 676596-65-9 |
| Paullones | Cazpaullone | 914088-64-5 |
| Peptide | FRATtide | |
| Peptide | L803 | |
| Peptides | L803-mts | |
| Publication | 705701 | |
| Publication | 708244 | |
| Publication | 709125 | |
| Publication | AR79 | |
| Publication | AZ13282107 | No Structure |
| Publication | AZ13282107 | |
| Publication | CEP-16805 | No Structure |
| Publication | CG-301338 | No Structure |
| Publication | CT73911 | |
| Publication | LY2064827 | |
| Publication | NP-103 | No Structure |
| Publication | SAR 502250 | No Structure |
| Publication | SAR 502250 (Sanofi) | 1073653-58-3 |
| Publication | XD-4241 | No Structure |
| Pyrazole | AT 7519 | 844442-38-2 |
| Pyrazole | Compound 4a | 1627557-91-8 |
| Pyrazole | Compound 4t | 1627558-10-4 |
| Pyrazole | Compound 4z | 1627558-16-0 |
| Pyrazole | GSK-3 Inhibitor XXII | 1195901-31-5 |
| Pyrazolone | GSK-3beta Inhibitor XXVI | 871843-09-3 |
| Pyrazolopyridazines | Compound 18 | 405223-20-3 |
| Pyrazolopyridazines | Compound 19 | 405223-71-4 |
| Pyrazolopyridine | Pyrazolopyridine 18 | 405221-39-8 |
| Pyrazolopyridine | Pyrazolopyridine 34 | 583039-27-4 |
| Pyrazolopyridine | Pyrazolopyridine 9 | 923029-74-7 |
| Pyrazolopyridines | Compound 14 | 583038-63-5 |
| Pyrazolopyridines | Compound 14 | 583038-63-5 |
| Pyrazolopyridines | Compound 23 | 583038-76-0 |
| Pyrazoloquinoxaline | NSC 693868 (Compound 1) | 40254-90-8 |
| Pyrazoloquinoxaline | NSC 693868 (Compound 1) | 40254-90-8 |
| Pyridinone | Compound 150 | 1282042-18-5 |
| Pyrrolopyridinyl | Compound 12 | 2025388-10-5 |
| Pyrrolopyridinyl | Compound 27 | 2025388-25-2 |
| Pyrroloazepine | Hymenialdisine | 82005-12-7 |
| Quinazolin | GSK-3 Inhibitor XIII | 404828-08-6 |
| Quinolinecarb | VP0.7 | 331963-23-6 |
| Quinolinecarboxamide | | 1132813-46-7 |
| Quinolinecarboxamide | | 1132812-98-6 |
| Quinolinecarboxamide | | 950727-66-9 |
| Thiadiazolidindiones | GSK-3β Inhibitor I | 327036-89-5 |
| Thiadiazolidindiones | NP031112 (Tideglusib) | 865854-05-3 |
| Thiadiazolidindiones | NP031115 | 1400575-57-6 |
| Triazolpyrimidine | Compound 90 | 91322-11-1 |
| Triazolpyrimidine | Compound 92 | 1043429-30-6 |
| Urea | GSK-3β Inh. VIII AR-A014418 | 487021-52-3 |
| Urea | A-1070722 | 1384424-80-9 |

In some embodiments, an agent of having activity as a Wnt agonist is a GSK3 inhibitor. In some embodiments, the GSK3 inhibitor is AZD1080, GSK3 inhibitor XXII, CHIR99021 or LY20903 14. In a preferred embodiment, the Wnt agonist is CHR99021. In other preferred embodiments, Wnt agonist and/or GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione. (Formula A.)

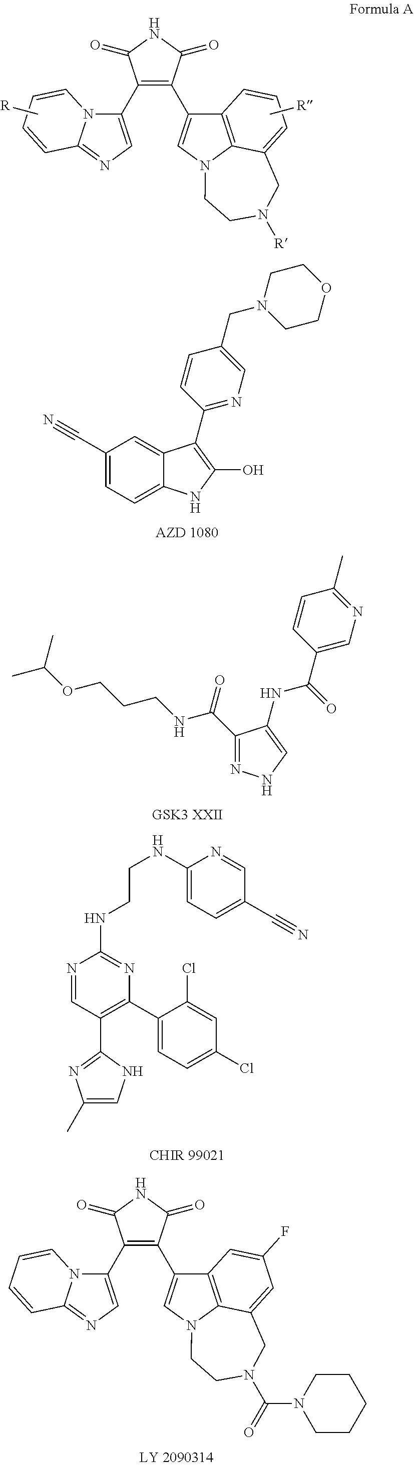

The Wnt agonist can be any selected from WO 2018/125746, which is hereby incorporated by reference. In some embodiments, the Wnt agonist can be the compound as defined in claim 1 of WO 2018/125746. In some embodiments, the Wnt agonist can be the compound as defined in claim 12 of WO 2018/125746.”

Exemplary, substituted 3-Imidazo[1,2-a]pyridin-3-yl-4-(1,2,3,4-tetrahydro-[1,4]diazepino-[6,7,1-hi]indol-7-yl)pyrrole-2,5-dione include: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 1-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide; 3-(9-(difluoromethvl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-((1R,4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(8-oxa-3-azabicyclo[3.2.1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(4,4-difluoropiperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-aminopiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4-(dimethylamino)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione (LY20900314).

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is: 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione; 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile; 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(4-(hydroxymethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile; 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione; 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione; 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide; 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione; 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione; 3-(8,9-difluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione; or 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione. (LY2090314).

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl)-4-(imidazo[1,2-a]pyridin-3-yl)-IH-pyrrole-2,5-dione. (LY2090314).

The structures of the substituted 3-Imidazo[1,2-a]pyridin-3-yl-4-(1,2,3,4-tetrahydro-[1,4]diazepino-[6,7,1-hi]indol-7-yl)pyrrole-2,5-dione are shown below in Table 4.

TABLE 4

| | | |
|---|---|---|
| Compound I-1 | 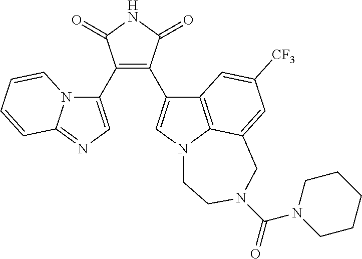 [SMILES: O=C1NC(=O)C(c2cnc3ccccn23)=C1c1cn2c3c(cc(C(F)(F)F)cc13)CN(C(=O)N1CCCCC1)CC2] | 3-(imidazo[1,2-a]pyridin-3-yl)-4-(2- (piperidine-1-carbonyl)-9-(trifluoromethyl)- 1,2,3,4-tetrahydro-[1,4]diazepino[6,7,1- hi]indol-7-yl)-1H-pyrrole-2,5-dione |
| Compound I-2 | 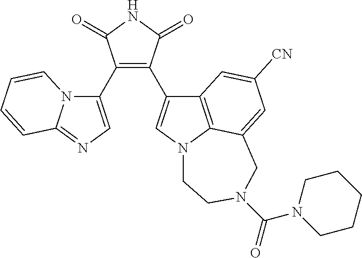 [SMILES: N#Cc1cc2c3c(c1)c(C1=C(c4cnc5ccccn45)C(=O)NC1=O)cn3CCN(C(=O)N1CCCCC1)C2] | 7-(4-(imidazo[1,2-a]pyridin-3-yl)-2,5-dioxo- 2,5-dihydro-1H-pyrrol-3-yl)-2-(piperidine-1- carbonyl)-1,2,3,4-tetrahydro- [1,4]diazepino[6,7,1-hi]indole-9-carbonitrile |
| Compound I-3 | 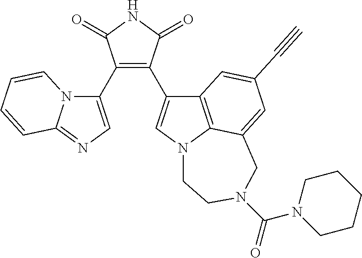 [SMILES: C#Cc1cc2c3c(c1)c(C1=C(c4cnc5ccccn45)C(=O)NC1=O)cn3CCN(C(=O)N1CCCCC1)C2] | 3-(9-ethynyl-2-(piperidine-1-carbonyl)- 1,2,3,4-tetrahydro-[1,4]diazepino[6,7,1- hi]indol-7-yl)-4,-(imidazo[1,2-a]pyridin-3-yl)- 1H-pyrrole-2,5-dione |
| Compound I-4 | 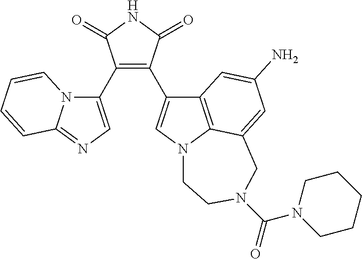 [SMILES: Nc1cc2c3c(c1)c(C1=C(c4cnc5ccccn45)C(=O)NC1=O)cn3CCN(C(=O)N1CCCCC1)C2] | 3-(9-amino-2-(piperidine-1-carbonyl)-1,2,3,4- tetrahydra-[1,4]diazepino[6,7,1-hi]indol-7-yl)- 4-(imidazo[1,2-a]pyridin-3-yl)-1H-pyrrole-2,5- dione |
| Compound I-5 | 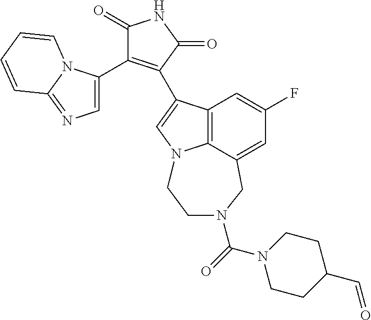 [SMILES: O=CC1CCN(C(=O)N2CCn3cc(C4=C(c5cnc6ccccn56)C(=O)NC4=O)c4cc(F)cc(c43)C2)CC1] | 1-(9-fluoro-7-(4-(imidazo[1,2-a]pyridin-3-yl)- 2,5-dioxo-2,5-dihydro-1H-pyrrol-3-yl)-1,2,3,4- tetrahydro-[1,4]diazepino[6,7,1-hi]indole-2- carbonyl)piperidine-4-carbaldehyde |
| Compound I-6 | 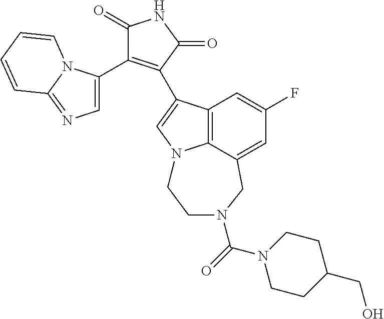 [SMILES: O=C1NC(=O)C(c2cnc3ccccn23)=C1c1cn2c3c(cc(F)cc13)CN(C(=O)N1CCC(CO)CC1)CC2] | 3-(9-fluoro-2-(4-(hydroxymethyl)piperidine-1- carbonyl)-1,2,3,4-tetrahydro- [1,4]diazepino[6,7,1-hi]-7-yl)-4- (imidazo[1,2-a]pyridin-3-yl)-1H-pyrrole-2,5- dione |
| Compound I-7 | 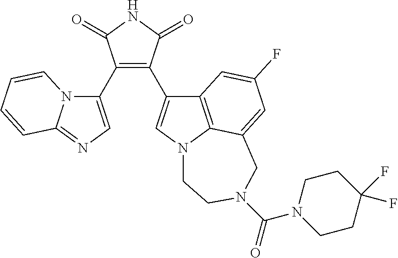 [SMILES: O=C1NC(=O)C(c2cnc3ccccn23)=C1c1cn2c3c(cc(F)cc13)CN(C(=O)N1CCC(F)(F)CC1)CC2] | 3-(2-(4,4-difluoropiperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-8 | 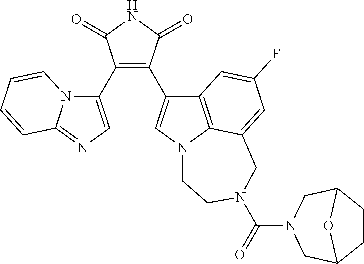 [SMILES: O=C1NC(=O)C(c2cnc3ccccn23)=C1c1cn2c3c(cc(F)cc13)CN(C(=O)N1CC3CCC(C1)O3)CC2] | 3-(2-(8-oxa-3-azabicyclo[3.2.1]octane-3- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I-9 | 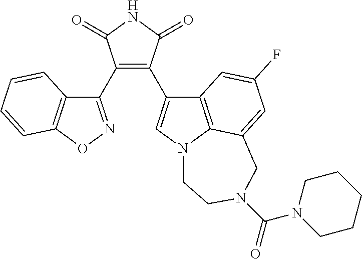 [SMILES: O=C1NC(=O)C(c2cn3c4c(cc(F)cc24)CN(C(=O)N2CCCCC2)CC3)=C1c1noc2ccccc12] | 3-(benzo[d]isoxazol-3-yl)-4-(9-fluoro-2- (piperidine-1-carbonyl)-1,2,3,4-tetrahydro- [1,4]diazepino[6,7,1-hi]indol-7-yl)-1H-pyrrole- 2,5-dione |
| Compound I- 10 | 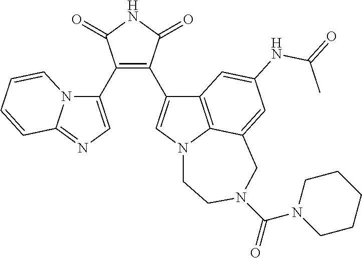 [SMILES: CC(=O)Nc1cc2c3c(c1)c(C1=C(c4cnc5ccccn45)C(=O)NC1=O)cn3CCN(C(=O)N1CCCCC1)C2] | N-(7-(4-(imidazo[1,2-a]pyridin-3-yl)-2,5-dioxo- 2,5-dihydro-1H-pyrrol-3-yl)-2-(piperidine-1- carbonyl)-1,2,3,4-tetrahydro- [1,4]diazepino[6,7,1-hi]indol-9-yl)acetamide |
| Compound I- 11 | 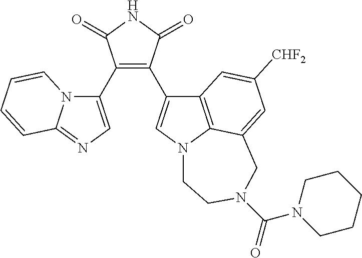 [SMILES: O=C1NC(=O)C(c2cnc3ccccn23)=C1c1cn2c3c(cc(C(F)F)cc13)CN(C(=O)N1CCCCC1)CC2] | 3-(9-(difluoromethyl)-2-(piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 12 | 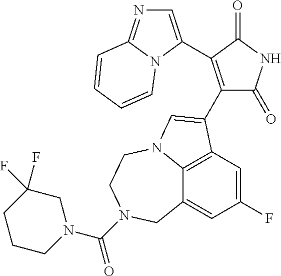 [SMILES: O=C1NC(=O)C(c2cnc3ccccn23)=C1c1cn2c3c(cc(F)cc13)CN(C(=O)N1CCCC(F)(F)C1)CC2] | 3-(2-(3,3-difluoropiperidine-1-carbonyl)-9- fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 13 | 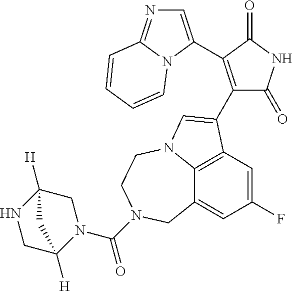 [SMILES: O=C1NC(=O)C(c2cnc3ccccn23)=C1c1cn2c3c(cc(F)cc13)CN(C(=O)N1C[C@H]3C[C@@H]1CN3)CC2] | 3-(2-((1R,4R)-2,5-diazabicyclo[2.2.1]heptane- 2-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 14 | 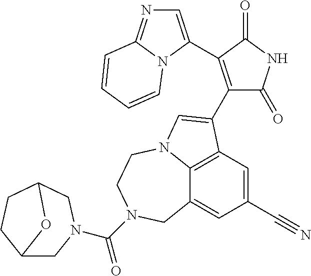 [SMILES: N#Cc1cc2c3c(c1)c(C1=C(c4cnc5ccccn45)C(=O)NC1=O)cn3CCN(C(=O)N1CC3CCC(C1)O3)C2] | 2-(8-oxa-3-azabicyclo[3.2.1]octane-3- carbonyl)-7-(4-(imidazo[1,2-a]pyridin-3-yl)- 2,5-dioxo-2,5-dihydro-1H-pyrrol-3-yl)-1,2,3,4- tetrahydro-[1,4]diazepino[6,7,1-hi]indole-9- carbonitrile |
| Compound I- 15 | 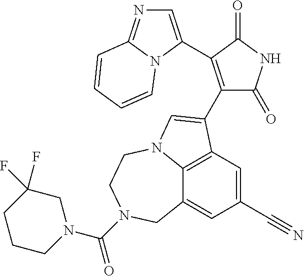 [SMILES: N#Cc1cc2c3c(c1)c(C1=C(c4cnc5ccccn45)C(=O)NC1=O)cn3CCN(C(=O)N1CCCC(F)(F)C1)C2] | 2-(3,3-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I- 16 | 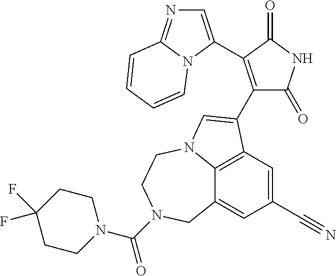 [SMILES: N#Cc1cc2c3c(c1)c(C1=C(c4cnc5ccccn45)C(=O)NC1=O)cn3CCN(C(=O)N1CCC(F)(F)CC1)C2] | 2-(4,4-difluoropiperidine-1-carbonyl)-7-(4- (imidazo[1,2-a]pyridin-3-yl)-2,5-dioxo-2,5- dihydro-1H-pyrrol-3-yl)-1,2,3,4-tetrahydro- [1,4]diazepino[6,7,1-hi]indole-9-carbonitrile |
| Compound I- 17 | 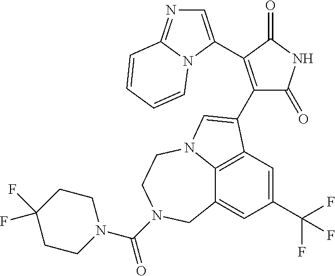 [SMILES: O=C1NC(=O)C(c2cnc3ccccn23)=C1c1cn2c3c(cc(C(F)(F)F)cc13)CN(C(=O)N1CCC(F)(F)CC1)CC2] | 3-(2-(4,4-difluoropiperidine-1-carbonyl)-9- (trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 18 | 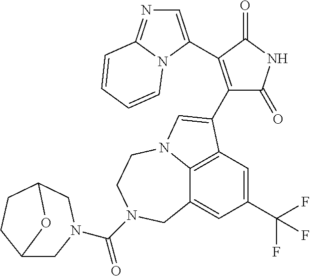 [SMILES: O=C1NC(=O)C(c2cnc3ccccn23)=C1c1cn2c3c(cc(C(F)(F)F)cc13)CN(C(=O)N1CC3CCC(C1)O3)CC2] | 3-(2-(8-oxa-3-azabicyclo[3.2.1]octane-3- carbonyl)-9-(trifluoromethyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I- 19 | 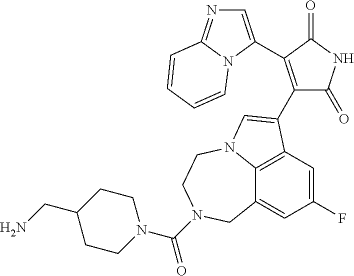 [SMILES: NCC1CCN(C(=O)N2CCn3cc(C4=C(c5cnc6ccccn56)C(=O)NC4=O)c4cc(F)cc(c43)C2)CC1] | 3-(2-(4-(aminomethyl)piperidine-1-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 20 | 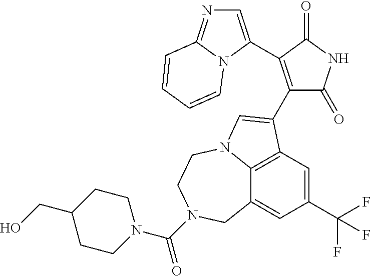 [SMILES: O=C1NC(=O)C(c2cnc3ccccn23)=C1c1cn2c3c(cc(C(F)(F)F)cc13)CN(C(=O)N1CCC(CO)CC1)CC2] | 3-(2-(4-(hydroxymethyl)piperidine-1- carbonyl)-9-(trifluoromethyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I- 21 | 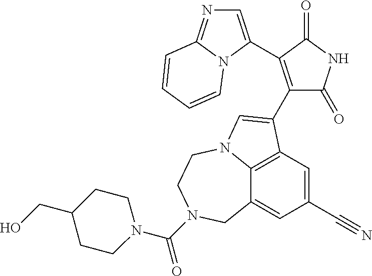 [SMILES: N#Cc1cc2c3c(c1)c(C1=C(c4cnc5ccccn45)C(=O)NC1=O)cn3CCN(C(=O)N1CCC(CO)CC1)C2] | 2-(4-(hydroxymethyl)piperidine-1-carbonyl)- 7-(4-(imidazo[1,2-a]pyridin-3-yl)-2,5-dioxo- 2,5-dihydro-1H-pyrrol-3-yl)-1,2,3- tetrahydro-[1,4]diazepino[6,7,1-hi]indole-9- carbonitrile |
| Compound I- 22 | 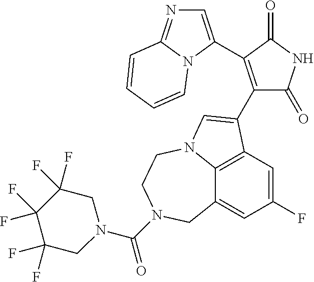 [SMILES: O=C1NC(=O)C(c2cnc3ccccn23)=C1c1cn2c3c(cc(F)cc13)CN(C(=O)N1CC(F)(F)C(F)(F)C(F)(F)C1)CC2] | 3-(9-fluoro-2-(3,3,4,4,5,5- hexafluoropiperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole-2,3- dione |
| Compound I- 23 | 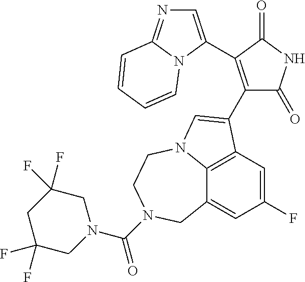 [SMILES: O=C1NC(=O)C(c2cnc3ccccn23)=C1c1cn2c3c(cc(F)cc13)CN(C(=O)N1CC(F)(F)CC(F)(F)C1)CC2] | 3-(9-fluoro-2-(3,3,5,5-tetrafluoropiperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| 1Compound I- 24 | 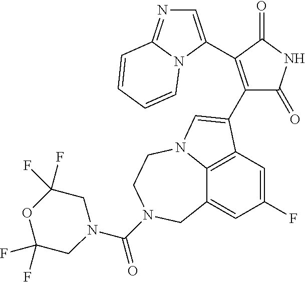 [SMILES: O=C1NC(=O)C(c2cnc3ccccn23)=C1c1cn2c3c(cc(F)cc13)CN(C(=O)N1CC(F)(F)OC(F)(F)C1)CC2] | 3-(9-fluoro-2-(2,2,6,6-tetrafluoromorpholine- 4-carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 25 | 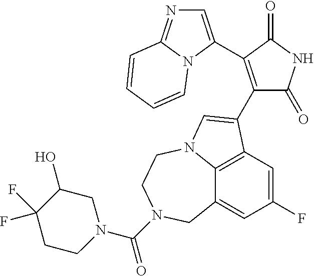 [SMILES: O=C1NC(=O)C(c2cnc3ccccn23)=C1c1cn2c3c(cc(F)cc13)CN(C(=O)N1CCC(F)(F)C(O)C1)CC2] | 3-(2-(4,4-difluoro-3-hydroxypiperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 26 | 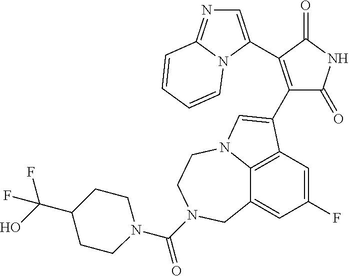 [SMILES: O=C1NC(=O)C(c2cnc3ccccn23)=C1c1cn2c3c(cc(F)cc13)CN(C(=O)N1CCC(C(O)(F)F)CC1)CC2] | 3-(2-(4-(difluoro(hydroxy)methyl)piperidine- 1-carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 27 | 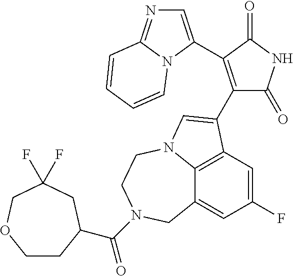 [SMILES: O=C1NC(=O)C(c2cnc3ccccn23)=C1c1cn2c3c(cc(F)cc13)CN(C(=O)C1CCOCC(F)(F)C1)CC2] | 3-(2-(6,6-difluoro-1,4-oxazepane-4-carbonyl)- 9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 28 | 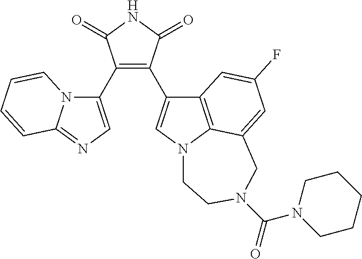 [SMILES: O=C1NC(=O)C(c2cnc3ccccn23)=C1c1cn2c3c(cc(F)cc13)CN(C(=O)N1CCCCC1)CC2] | 3-([1,2,4]triazolo[4,3-a]pyridin-3-yl)-4-(9- fluoro-2-(piperidine-1-carbonyl)-1,2,3,4- tetrahydro-[1,4]diazepino[6,7,1-hi]indol-7-yl)- 1H-pyrrole-2,5-dione |
| Compound I- 29 | 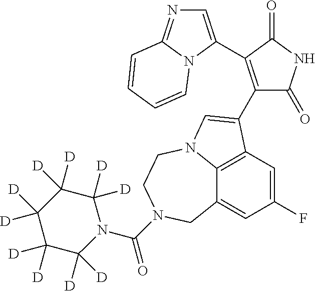 [SMILES: [2H]C1([2H])N(C(=O)N2CCn3cc(C4=C(c5cnc6ccccn56)C(=O)NC4=O)c4cc(F)cc(c43)C2)C([2H])([2H])C([2H])([2H])C([2H])([2H])C1([2H])[2H]] | 3-(9-fluoro-2-(piperidine-1-carbonyl-d10)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I- 30 | 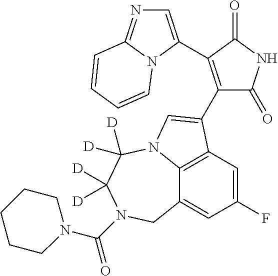 [SMILES: [2H]C1([2H])N(C(=O)N2CCCCC2)Cc2cc(F)cc3c(C4=C(c5cnc6ccccn56)C(=O)NC4=O)cn(c23)C1([2H])[2H]] | 3-(9-fluoro-2-(pipendine-1-carbonyl)-1,2,3,4- tetrahydro-[1,4]diazepino[6,7,1-hi]indol-7-yl- 3,3,4,4-d4)-4-(imidazo[1,2-a]pyridin-3-yl)-1H- pyrrole-2,5-dione |
| Compound I- 31 | 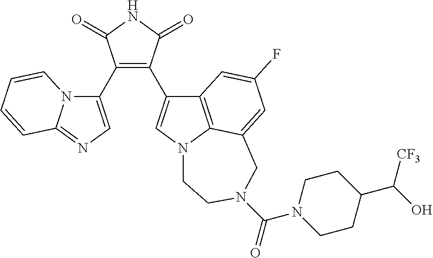 [SMILES: O=C1NC(=O)C(c2cnc3ccccn23)=C1c1cn2c3c(cc(F)cc13)CN(C(=O)N1CCC(C(O)C(F)(F)F)CC1)CC2] | 3-(9-fluoro-2-(4-(2,2,2-trifluoro-1- hydroxyethyl)piperidine-1-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I- 32 | 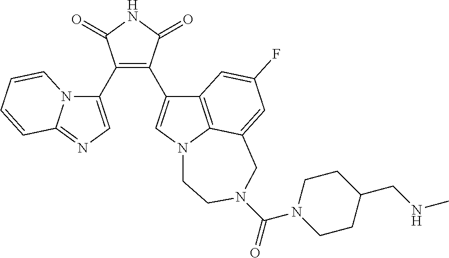 [SMILES: CNCC1CCN(C(=O)N2CCn3cc(C4=C(c5cnc6ccccn56)C(=O)NC4=O)c4cc(F)cc(c43)C2)CC1] | 3-(9-fluoro-2-(4- ((methylamino)methyl)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 33 | 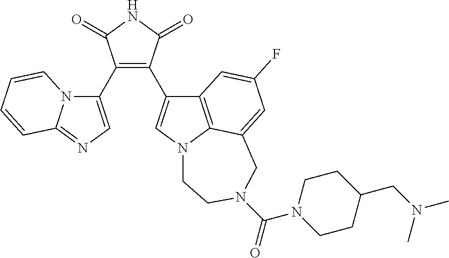 [SMILES: CN(C)CC1CCN(C(=O)N2CCn3cc(C4=C(c5cnc6ccccn56)C(=O)NC4=O)c4cc(F)cc(c43)C2)CC1] | 3-(2-(4-((dimethylamino)methyl)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 34 | 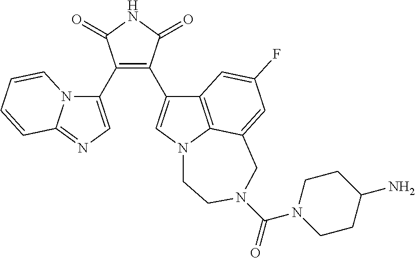 [SMILES: NC1CCN(C(=O)N2CCn3cc(C4=C(c5cnc6ccccn56)C(=O)NC4=O)c4cc(F)cc(c43)C2)CC1] | 3-(2-(4-aminopiperidine-1-carbonyl)-9-fluoro- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I- 35 | 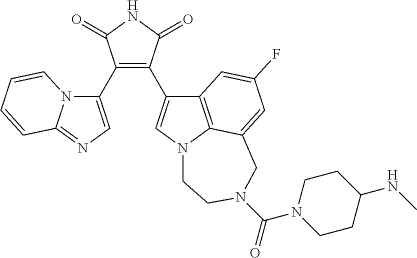 [SMILES: CNC1CCN(C(=O)N2CCn3cc(C4=C(c5cnc6ccccn56)C(=O)NC4=O)c4cc(F)cc(c43)C2)CC1] | 3-(9-fluoro-2-(4-(methylamino)piperidine-1- carbonyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 36 | 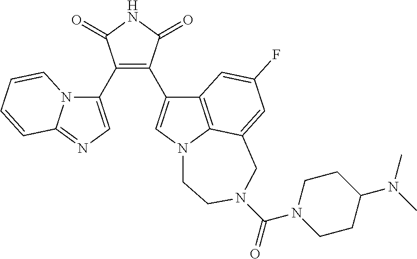 [SMILES: CN(C)C1CCN(C(=O)N2CCn3cc(C4=C(c5cnc6ccccn56)C(=O)NC4=O)c4cc(F)cc(c43)C2)CC1] | 3-(2-(4-(dimethylamino)piperidine-1- carbonyl)-9-fluoro-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 37 | 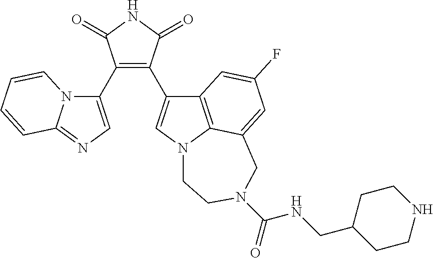 [SMILES: O=C1NC(=O)C(c2cnc3ccccn23)=C1c1cn2c3c(cc(F)cc13)CN(C(=O)NCC1CCNCC1)CC2] | 9-fluoro-7-(4-(imidazo[1,2-a]pyridin-3-yl)-2,5- dioxo-2,5-dihydro-1H-pyrrol-3-yl)-N- (piperidin-4-ylmethyl)-3,4-dihydro- [1,4]diazepino[6,7,1-hi]indole-2(1H)- carboxamide |
| Compound I- 38 | 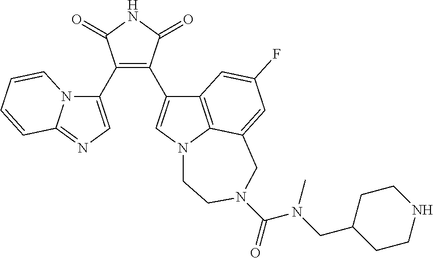 [SMILES: CN(CC1CCNCC1)C(=O)N1CCn2cc(C3=C(c4cnc5ccccn45)C(=O)NC3=O)c3cc(F)cc(c32)C1] | 9-fluoro-7-(4-(imidazo[1,2-a]pyridin-3-yl)-2,5- dioxo-2,5-dihydro-1H-pyrrol-3-yl)-N-methyl- N-(piperidin-4-ylmethyl)-3,4-dihydro- [1,4]diazepino[6,7,1-hi]indole-2(1H)- carboxamide |
| Compound I- 39 | 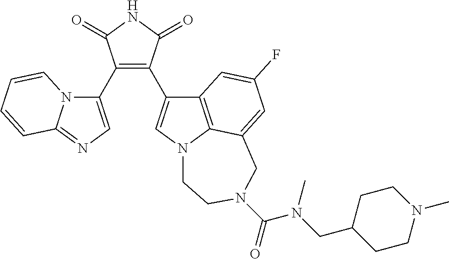 [SMILES: CN1CCC(CN(C)C(=O)N2CCn3cc(C4=C(c5cnc6ccccn56)C(=O)NC4=O)c4cc(F)cc(c43)C2)CC1] | 9-fluoro-7-(4-(imidazo[1,2-a]pyridin-3-yl)-2,5- dioxo-2,5-dihydro-1H-pyrrol-3-yl)-N-methyl- N-((1-methylpiperidin-4-yl)methyl)-3,4- dihydro-[1,4]diazepino[6,7,1-hi]indole-2(1H)- carboxamide |
| Compound- 40 | 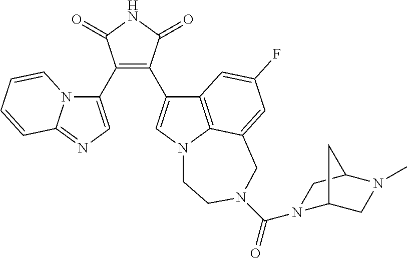 [SMILES: CN1CC2CC1CN2C(=O)N1CCn2cc(C3=C(c4cnc5ccccn45)C(=O)NC3=O)c3cc(F)cc(c32)C1] | 3-(9-fluoro-2-((1R,4R)-5-methyl-2,5- diazabicyclo[2.2.1]heptane-2-carbonyl)- 1,2,3,4-tetrahydro-[1,4]diazepino[6,7,1- hi]indol-7-yl)-4-(imidazo[1,2-a]pyridin-3-yl)- 1H-pyrrole-2,5-dione |
| Compound I- 41 | 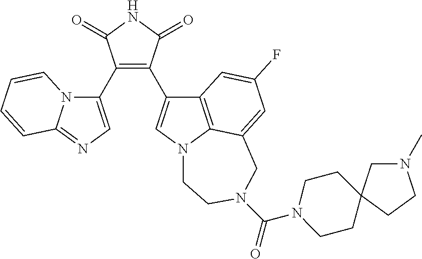 [SMILES: CN1CCC2(CCN(C(=O)N3CCn4cc(C5=C(c6cnc7ccccn67)C(=O)NC5=O)c5cc(F)cc(c54)C3)CC2)C1] | 3-(9-fluoro-2-(2-methyl-2,8- diazaspiro[4.5]decane-8-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound 42 | 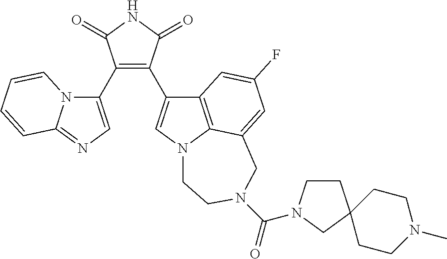 [SMILES: CN1CCC2(CC1)CCN(C(=O)N1CCn3cc(C4=C(c5cnc6ccccn56)C(=O)NC4=O)c4cc(F)cc(c43)C1)C2] | 3-(9-fluoro-2-(8-methyl-2,8- diazaspiro[4.5]decane-2-carbonyl)-1,2,3,4- tetrahydro-[1,4]diazepino[6,7,1-hi]indol-7-yl)- 4-(imidazo[1,2-a]pyridin-3-yl)-1H-pyrrole-2,5- dione |
| Compound I- 43 | 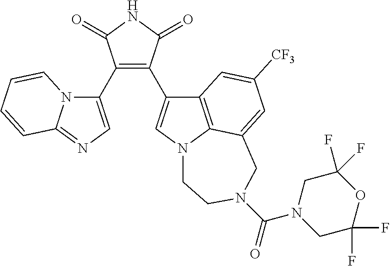 [SMILES: O=C1NC(=O)C(c2cnc3ccccn23)=C1c1cn2c3c(cc(C(F)(F)F)cc13)CN(C(=O)N1CC(F)(F)OC(F)(F)C1)CC2] | 3-(imidazo[1,2-a]pyridin-3-yl)-4-(2-(2,2,6,6- tetrafluoromorpholine-4-carbonyl)-9- (trifluoromethyl)-1,2,3,4-tetrahydro- [1,4]diazepino[6,7,1-hi]indol-7-yl)-1H-pyrrole- 2,5-dione |
| Compound I- 44 | 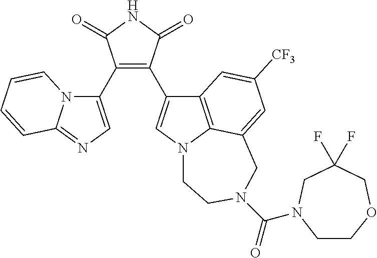 [SMILES: O=C1NC(=O)C(c2cnc3ccccn23)=C1c1cn2c3c(cc(C(F)(F)F)cc13)CN(C(=O)N1CCOCC(F)(F)C1)CC2] | 3-(2-(6,6-difluoro-1,4-oxazepane-4-carbonyl)- 9-(trifluoromethyl)-1,2,3,4-tetrahydro- [1,4]diazepino[6,7,1-hi]indol-7-yl)-4- (imidazo[1,2-a]pyridin-3-yl)-1H-pyrrole-2,5- dione |
| Compound I- 45 | 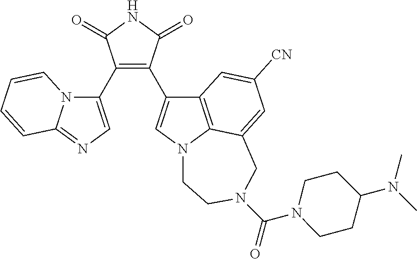 [SMILES: CN(C)C1CCN(C(=O)N2CCn3cc(C4=C(c5cnc6ccccn56)C(=O)NC4=O)c4cc(C#N)cc(c43)C2)CC1] | 2-(4-(dimethylamino)piperidine-1-carbonyl)- 7-(4-(imidazo[1,2-a]pyridin-3-yl)-2,5-dioxo- 2,5-dihydro-1H-pyrrol-3-yl)-1,2,3,4- tetrahydro-[1,4]diazepino[6,7,1-hi]indole-9- carbonitrile |
| Compound I- 46 | 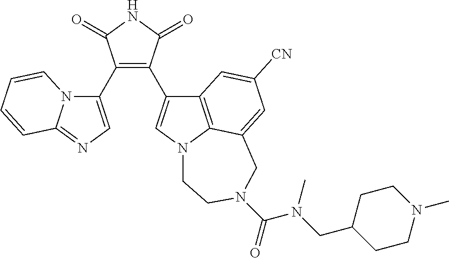 [SMILES: CN1CCC(CN(C)C(=O)N2CCn3cc(C4=C(c5cnc6ccccn56)C(=O)NC4=O)c4cc(C#N)cc(c43)C2)CC1] | 9-cyano-7-(4-(imidazo[1,2-a]pyridin-3-yl)-2,5- dioxo-2,5-dihydro-1H-pyrrol-3-yl)-N-methyl- N-((1-methylpiperidin-4-yl)methyl)-3,4- dihydro-[1,4]diazepino[6,7,1-hi]indole-2(1H)- carboxamide |
| Compound I- 47 | 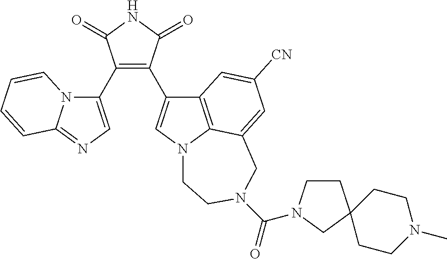 [SMILES: CN1CCC2(CC1)CCN(C(=O)N1CCn3cc(C4=C(c5cnc6ccccn56)C(=O)NC4=O)c4cc(C#N)cc(c43)C1)C2] | 7-(4-(imidazo[1,2-a]pyridin-3-yl)-2,5-dioxo- 2,5-dihydro-1H-pyrrol-3-yl)-2-(8-methyl-2,8- diazaspiro[4.5]decane-2-carbonyl)-1,2,3,4- tetrahydro-[1,4]diazepino[6,7,1-hi]indole-9- carbonitrile |
| Compound I- 48 | 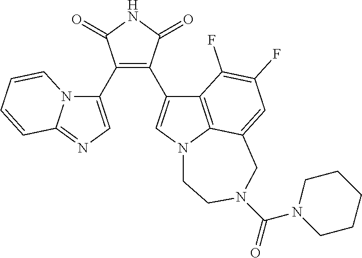 [SMILES: O=C1NC(=O)C(c2cnc3ccccn23)=C1c1cn2c3c(cc(F)c(F)c13)CN(C(=O)N1CCCCC1)CC2] | 3-(8,9-difluoro-2-(piperidine-1-carbonyl)- 1,2,3,4-tetrahydro-[1,4]diazepino[6,7,1- hi]indol-7-yl)-4-(imidaze[1,2-a]pyridin-3-yl)- 1H-pyrrole-2,5-dione |

In other embodiments, Wnt agonist and/or GSK3 inhibitor as described in WO 2018/125746, US 20180214458 and U.S. Ser. No. 62/608,663 the contents of which are each incorporated by reference in their entireties.

Epigenetic Agents

Epigenetic agents are agents that can modulate activity of epigenetic modifiers, mediators and modulators. Epigenetic modifiers are genes whose products modify the epigenome directly through DNA methylation, the post-translational modification of chromatin or the alteration of the structure of chromatin. Epigenetic mediators, are often the target of epigenetic modification, although they are rarely mutated themselves. The epigenetic mediators largely overlap with the genes involved in stem cell reprogramming and their role in cancer followed directly from the discovery of their reprogramming role. Epigenetic mediators are those genes whose products are the targets of the epigenetic modifiers. Epigenetic modulators are the as genes lying upstream of the modifiers and mediators in signalling and metabolic pathways

In some embodiments, an agent of having activity as an epigenetic agents is an HDAC inhibitor, an EZH2 inhibitor, a DOT1L inhibitor, KDM inhibitor or an LSD1 inhibitor.

HDAC Inhibitors

Histone deacetylases (HDAC) are a class of enzymes that remove acetyl groups (O═C—CH3) from an ε-N-acetyl lysine amino acid on a histone, allowing the histones to wrap the DNA more tightly. This is important because DNA is wrapped around histones, and DNA expression is regulated by acetylation and de-acetylation.

HDACs are classified in four classes depending on sequence homology to the yeast original enzymes and domain organization. The HDAC classes include HDACI, HDAC IIA, HDAC IIB, HDAC III and HDAC IV.

Histone deacetylase (HDAC) inhibitors (HDACi, HDIs) are chemical compounds that inhibit histone deacetylases.

Thus, “HDAC inhibitor” refers to an agent capable of the decreasing the expression or enzymatic activity of HDAC. For example HDAC inhibitor results in a decrease in histone deacetylation of a target gene in a cell.

In certain embodiments, the HDAC inhibitor decreases the expression or enzymatic activity of HDAC by at least 5, 10, 20, 30, 40, 50, 60, 70, 80, 90, or 100% relative to a control, for example relative to a baseline level of activity.

In certain embodiments, the HDAC inhibitor decreases histone deacetylation of a target gene by at least 5, 10, 20, 30, 40, 50, 60, 70, 80, 90, or 100% relative to a control, for example relative to a baseline level of activity.

In some embodiments, the HDAC inhibitor increases expression or activity of a target gene by at least 5, 10, 20, 30, 40, 50, 60, 70, 80, 90, or 100% relative to a control, for example relative to a baseline level of activity.

In some embodiments, the HDAC inhibitor decreases expression or enzymatic activity of HDAC by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the HDAC inhibitor decreases histone deacetylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the HDAC inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

TABLE 5

| | | Chemo- | Mechanism | Class | HDAC | | Lgr5+ | Perilymph | Formulation |
|---|---|---|---|---|---|---|---|---|---|
| Agent | CAS | type | HDAC Inhib | selectivity | Potene | | Assay | Conc | Conc |
| Sodium | 1069-66-5 | Acid | 1, 2, 3, 8 | Class I | 39-161 | μM | 100 μM- | 100 μM- | 100 mM- |
| Valproate | | | | | | | 4 mM | 4 mM | 4000 mM |
| 2-hexyl-4- | 96017-59-3 | Acid | 1, 2, 3, 8 | Class I | 13 | μM | 100 μM- | 100 μM- | 100 mM- |
| pentynoic acid | | | | | | | 4 mM | 4 mM | 4000 mM |
| Na | 1716-12-7 | Acid | 1, 2, 3, 8 | Class I > | 9-16 | μM | 100 μM- | 100 μM- | 100 mM- |
| phenylbutyrate | | | | Class IIb | | | 4 mM | 4 mM | 4000 mM |

In various embodiments, the methods and compositions of the invention include use an HDAC inhibitor. Exemplary HDAC inhibitors are provide in Table 6

TABLE 6

| Class | Agent | CAS |
|---|---|---|
| Aliphatic Acid | Butyrate | 107-92-6 |
| Aliphatic Acid | Phenyl butyrate | 1821-12-1 |
| Aliphatic Acid | Valproic Acid | 99-66-1 |
| Aliphatic Acid Ester | AN-9 | 122110-53-6 |
| Amine | 932718-22-4 | 932718-22-4 |
| Benzamide | 4SC-202 | 1186222-89-8 |
| Benzamide | BML-210 | 537034-17-6 |
| Benzamide | Chidamide | 743438-44-0 |
| Benzamide | Entinostat (MS-275) | 209783-80-2 |
| Benzamide | HDAC Inhibitor IV | 537034-15-4 |
| Benzamide | Mocetinostat (MGCD0103) | 726169-73-9 |
| Benzamide | NKL 22 | 537034-15-4 |
| Benzamide | RGFP109 | 1215493-56-3 |
| Benzamide | RGFP136 | 1215493-97-2 |
| Benzamide | RGFP966 | 1357389-11-7 |
| Benzamide | Tacedinaline | 112522-64-2 |
| Benzamide | TC-H 106, HDAC Inhibitor VII | 937039-45-7 |
| Cyclic peptide | Apicidin | 183506-66-3 |
| Cyclic peptide | Dihydrochlamydocin | 52574-64-8 |
| Cyclic peptide | HC Toxin | 83209-65-8 |
| Cyclic peptide | Romidepsin | 128517-07-7 |
| Cyclic Peptide | Thailandepsin A | 1269219-30-8 |
| Cyclic peptide | Trapoxin A | 133155-89-2 |
| Epoxide | (−)-Depudecin | 139508-73-9 |
| Epoxide | Parthenolide | 20554-84-1 |
| Hydroxamate | (S)-HDAC-42 | 935881-37-1 |
| Hydroxamate | 4-(dimethylamino)-N-[6- | 193551-00-7 |
| | (hydroxyamino)-6-oxohexyl]- | |
| | benzamide | |
| Hydroxamate | 4-iodo-SAHA | 1219807-87-0 |
| Hydroxamate | 4SC-201 (Resminostat) | 864814-88-0 |
| Hydroxamate | ACY1215 | 1316214-52-4 |
| Hydroxamate | APHA Compound 8 | 676599-90-9 |
| Hydroxamate | BRD9757 | 1423058-85-8 |
| Hydroxamate | Bufexamac | 2438-72-4 |
| Hydroxamate | Butyrylhydroxamic acid | 4312-91-8 |
| Hydroxamate | CAY10603 | 1045792-66-2 |
| Hydroxamate | CBHA | 174664-65-4 |
| Hydroxamate | CG200745 | 936221-33-9 |
| Hydroxamate | CHR-3996 | 1256448-47-1 |
| Hydroxamate | CUDC-101 | 1012054-59-9 |
| Hydroxamate | Droxinostat | 99873-43-5 |
| Hydroxamate | HDAC Inhibitor II | 174664-65-4 |
| Hydroxamate | HDAC Inhibitor VI | 926908-04-5 |
| Hydroxamate | HDAC Inhibitor XXIV | 854779-95-6 |
| Hydroxamate | HDAC6 Inhibitor III | 1450618-49-1 |
| Hydroxamate | HDAC-IN-1 | 1239610-44-6 |
| Hydroxamate | HNHA | 926908-04-5 |
| Hydroxamate | HPOB | 1429651-50-2 |
| Hydroxamate | ITF2357 | 497833-27-9 |
| Hydroxamate | ITF2357 (Givinostat) | 497833-27-9 |
| Hydroxamate | LAQ-824 | 591207-53-3 |
| Hydroxamate | LBH-589 (panobinostat) | 404950-80-7 |
| Hydroxamate | LMK235 | 1418033-25-6 |
| Hydroxamate | M 344 | 251456-60-7 |
| Hydroxamate | MC 1568 | 852475-26-4 |
| Hydroxamate | Nexturastat A | 1403783-31-2 |
| Hydroxamate | NSC 57457 | 6953-61-3 |
| Hydroxamate | Oxamflatin | 151720-43-3 |
| Hydroxamate | PCI-24781 (Abexinostat) | 783355-60-2 |
| Hydroxamate | PCI-34051 | 950762-95-5 |
| Hydroxamate | PDX-101 (belinostat) | 866323-14-0 |
| Hydroxamate | Pyroxamide | 382180-17-8 |
| Hydroxamate | SAHA (Zolinza, vorinostat) | 149647-78-9 |
| Hydroxamate | SB939 (Pracinostat) | 929016-96-6 |
| Hydroxamate | SBHA | 38937-66-5 |
| Hydroxamate | Scriptaid | 287383-59-9 |
| Hydroxamate | Tefinostat (CHR-2845) | 914382-60-8 |
| Hydroxamate | Trichostatin A (TSA) | 58880-19-6 |
| Hydroxamate | Tubacin | 537049-40-4 |
| Hydroxamate | Tubastatin A | 1252003-15-8 |
| Hydroxamate | VAHA | 106132-78-9 |
| Ketone | Compound 43 | 891259-76-0 |
| Ketone - a-ketoamides | 436150-82-2 | 436150-82-2 |
| Ketone - CF3 | Compound 27 | 946499-86-1 |
| Ketone - CF3 | Compound 6e | 946500-31-8 |
| Ketone - CF3 | Compound 6H | 946500-39-6 |
| Non classical | Tasquinimod | 254964-60-8 |
| Non classical | TMP269 | 1314890-29-3 |
| Polyketide | Ratjadone A | 163564-92-9 |
| Silylalcohol | 1587636-32-5 | 1587636-32-5 |
| Sulphonamide | 1587636-33-6 | 1587636-33-6 |
| Sulphonamide | 329967-25-1 | 329967-25-1 |
| Sulphonyl Urea | 960130-17-0 | 960130-17-0 |
| Thioester | HDAC Inhibitor XXII | 848354-66-5 |
| Thioester | KD 5170 | 940943-37-3 |
| Thioester | PTACH | 848354-66-5 |
| Thioester | TCS HDAC6 20b | 956154-63-5 |
| Thioketone | SIRT1/2 Inhibitor VII | 143034-06-4 |
| Thiol | 1368806-68-1 | 1368806-68-1 |
| Thiol | 1428536-05-3 | 1428536-05-3 |
| Thiol | 827036-76-0 | 827036-76-0 |
| Thiol | 828920-13-4 | 828920-13-4 |
| Thiol | 908860-21-9 | 908860-21-9 |
| Tropones | 1411673-95-4 | 1411673-95-4 |
| Tropones | 46189-88-2 | 46189-88-2 |

In some embodiments the HDAC inhibitor is a class I HDAC inhibitor. In these embodiments, the class I HDAC inhibitor is a short chain carboxylic acid. In a preferred embodiment, the HDAC inhibitor is valproic acid (VPA), 2-hexyl-4-pentynoic acid, or Na phenylbutyrate. In some embodiments, the HDAC inhibitor is valproic acid (VPA).

As used herein the terms “valproic acid”, “VPA” and “sodium valproate” are used interchangably to refer to the same compound.

Ezh2 Inhibitors

Enhancer of zeste homolog 2 (EZH2) is a histone-lysine N-methyltransferase enzyme encoded by EZH2 gene, that participates in histone methylation and, ultimately, transcriptional repression. EZH2 catalyzes the addition of methyl groups to histone H3 at lysine 27, by using the cofactor S-adenosyl-L-methionine. Methylation activity of EZH2 facilitates heterochromatin formation thereby silences gene function. Remodeling of chromosomal heterochromatin by EZH2 is also required during cell mitosis.

EZH2 is the functional enzymatic component of the Polycomb Repressive Complex 2 (PRC2), which is responsible for healthy embryonic development through the epigenetic maintenance of genes responsible for regulating development and differentiation EZH2 is responsible for the methylation activity of PRC2, and the complex also contains proteins required for optimal function (EED, SUZ12, JARID2, AEBP2, RbAp46/48, and PCL).

EZH2 inhibitors are chemical compounds that inhibit histone-lysine N-methyltransferase enzyme encoded by EZH2 gene

Thus, “EZH2 inhibitor” refers to an agent capable of the decreasing the expression or enzymatic activity of EZH2. For example, an EZH2 inhibitor results in a decrease in histone methylation of a target gene in a cell.

In certain embodiments, the EZH2 inhibitor decreases the expression or enzymatic activity of EZH2 by at least 5, 10, 20, 30, 40, 50, 60, 70, 80, 90, or 100% relative to a control, for example relative to a baseline level of activity.

In certain embodiments, the EZH2 inhibitor decreases histone methylation of a target gene by at least 5, 10, 20, 30, 40, 50, 60, 70, 80, 90, or 100% relative to a control, for example relative to a baseline level of activity.

In some embodiments, the EZH2 inhibitor increases expression or activity of a target gene by at least 5, 10, 20, 30, 40, 50, 60, 70, 80, 90, or 100% relative to a control, for example relative to a baseline level of activity.

In some embodiments, the EZH2 inhibitor decreases expression or enzymatic activity of EZH2 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the EZH2 inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the EZH2 inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary EZH2 inhibitors are provide in Table 7

TABLE 7

| | | pKi | | | | Select | | Lit | |
|---|---|---|---|---|---|---|---|---|---|
| | | or | | Enzymatic/ | | vs | | Cell | |
| Agent | CAS | IC50 | | Non-enzymatic | Chemo-type | EZH-1 | | Poten | |
| PF-06821497 | 1844849-10-0 | <1 | nM | enzymatic | 2-Pyridone | 70 | nM | 4-6 | nM |
| PF-06726304 | 1616287-82-1 | 1.1 | nM | enzymatic | 2-Pyridone | | | 10-25 | nM |
| CPI-1205 | 1621862-70-1 | 2.2 | nM | enzymatic | 2-Pyridone | 24x | | 32 | nM |
| valemetostat | 1809336-39-7 | 2.5 | nM | enzymatic | 2-Pyridone | 8.4 | nM | 25-250 | nM |
| (DS-3201b, | | | | | | | | | |
| (R)-OR-S2) | | | | | | | | | |
| tazemetostat | 1403254-99-8 | 2.5 | nM | enzymatic | 2-Pyridone | 35x | | | |
| (EPZ-6438) | | | | | | | | | |
| E11 | 1418308-27-6 | 13 | nM | enzymatic | 2-Pyridone | 90x | | | |
| CPI-169 | 1450655-76-1; | 0.24 | nM | enzymatic | 2-Pyridone | 6 | nM | | |
| | 1802175-07-0 | | | | | | | | |
| CPI-360 | 1802175-06-9 | 0.5 | nM | enzymatic | 2-Pyridone | ~50 | nM | | |
| EPZ-011989 | 1598383-40-4 | <3 | nM | enzymatic | 2-Pyridone | | | 94 | nM |
| UNC 2399 | 1433200-53-3 | | | enzymatic | 2-Pyridone | | | | |
| (R)-OR-S1 | 1809336-19-3 | 10 | nM | enzymatic | 2-Pyridone | 7.4 | nM | | |
| A-395 | 2089148-72-9 | 0.3 | nM | EED Inhibit | Amino | | | 90 | nM |
| | | | | | pyrrolidines | | | | |
| Astemizole | 68844-77-9 | 94 | μM | EED Inhibit | Benzimidazole | | | | |
| Compound 19 | 2079895-22-8 | 1.3 | μM | EED Inhibit | Imidazole | | | 1.9 | μM |
| Compound 22 | 1802175-07-0 | 2 | nM | enzymatic | 2-Pyridone | | | | |
| Compound 24 | 1659298-29-9 | 40 | nM | enzymatic | 2-Pyridone | | | | |
| Compound 34 | 2055347-72-1 | 29 | nM | enzymatic | 2-Pyridone | >100x | | | |
| Compound 41 | 2055347-94-7 | 11 | nM | enzymatic | 2-Pyridone | >100x | | | |
| CPI-0209 | | | | | | | | | |
| CPI-360 | 1802175-06-9 | 0.5 | nM | enzymatic | 2-Pyridone | ~50 | nM | | |
| EBI-2511 | 2098546-05-3 | 4 | nM | enzymatic | 2-Pyridone | | | | |
| EED162 | 1010897-73-0 | 30 | nM | EED Inhibit | Triazo | | | 80 | nM |
| EED226 | 2083627-02-3 | | | EED Inhibit | Triazo | | | | |
| EPZ-005687 | 1396772-26-1 | 24 | nM | enzymatic | 2-Pyridone | 50X | | | |
| EPZ-011989 | 1598383-40-4 | <3 | nM | enzymatic | 2-Pyridone | | | 94 | nM |
| GSK126 | 1346574-57-9 | <3 | nM | enzymatic | 2-Pyridone | 150x | | | |
| GSK343 | 1346704-33-3 | 1.2 | nM | enzymatic | 2-Pyridone | 60x | | 174 | nM |
| GSK503 | 1346572-63-1 | <10 | nM | enzymatic | 2-Pyridone | | | | |
| GSK926 | 1346704-13-9 | 7.9 | nM | enzymatic | 2-Pyridone | | | 324 | nM |
| MAK683 | 1951408-58-4 | | | EED inhibitor | Triazo | | | | |
| (EED162) | (likely patent) | | | | | | | | |
| SHR2554 | 2098545-98-1 | | | enzymatic | 2-Pyridone | | | | |
| SKLB1049 | 1826865-42-2 | 7.2 | nM | enzymatic | 2-Pyridone | | | 12 | μM |
| ZLD1039 | 1826865-46-6 | <15 | nM | enzymatic | 2-Pyridone | | | | |
| ZLD1122 | 1826865-51-3 | <15 | nM | enzymatic | 2-Pyridone | | | | |
| | 1404094-15-0 | 74 | nM | enzymatic | 2-Pyridone | | | 2510 | nM |
| | 1404094-16-1 | 14 | nM | enzymatic | 2-Pyridone | | | 1995 | nM |
| DZNep | 102052-95-9 | | | SAH-hydrolase | SAH derived | | | 1 | μM |
| | | | | inhibitor | | | | | |
| Cmpd 44 | 1378002-93-7 | 32 | nM | SAM Comp | Benzamide | | | 9 | μM |
| Compound 27 | 1676100-59-6 | 270 | nM | SAH-hydrolase | SAH derived | | | | |
| | | | | inhibitor | | | | | |
| Sinefungin | 58944-73-3 | 20 | nM | SAH-hydrolase | SAH derived | | | 33 | nM |
| | | | | inhibitor | | | | | |
| Tanshindiol B | 97465-70-8 | 520 | nM | enzymatic | Tanshindiols | | | | |
| Tanshindiol C | 97465-71-9 | 550 | nM | enzymatic | Tanshindiols | | | | |
| UN C1999 | 1431612-23-5 | 10 | nM | enzymatic | 2-Pyridone | 10x | | 124 | nM |
| (−)- | 989-51-5 | | | enzymatic | a, b-unsat | | | | |
| Epigallocatechin- | | | | | | | | | |
| 3-gallate (EGCG) | | | | | | | | | |
| Curcumin | 458-37-7 | | | enzymatic | a, b-unsat | | | | |
| MC1945 | 169903-68-8 | | | enzymatic | a, b-unsat | | | | |
| MC1947 | 949090-12-4 | | | Non-enzymatic | | | | | |
| MC1948 | 949090-20-4 | | | Non-enzymatic | | | | | |
| SAH-EZH2 | | | | Non-enzymatic | reactive | | | | |
| Sulforaphane | 4478-93-7 | | | EED Inhibit | Stapled | | | | |
| | | | | | Peptide | | | | |

| | | | | Formul. | Human | | |
|---|---|---|---|---|---|---|---|
| | Lgr5+ | Perilymph | | Conc. | In Vivo | | Human |
| Agent | Assay | Conc | | Intraymp | Conc | | Dosage |
| PF-06821497 | | 10-1000 | nM | 10-1000 | 5-50 | nM | 75 mg to |
| | | | | μM | | | 625 mg |
| | | | | | | | BID PO |
| PF-06726304 | 100 nM- | 100 nM- | | 100 μM- | 100 nM- | | 100 to |
| | 3 μM | 3 μM | | 3 mM | 30 μM | | 1000/day |
| | | | | | | | mg PO |
| CPI-1205 | 100 nM- | 0.10-30 | μM | 100- | 25-250 | μM | 800 mg |
| | 30 μM | | | 30,000 μM | | | BID and |
| | | | | | | | subsequently |
| | | | | | | | TID - PO |
| valemetostat | | 10-1000 | nM | 10- | 25-250 | nM | PO |
| (DS-3201b, | | | | 1000 μM | | | dose |
| (R)-OR-S2) | | | | | | | starting |
| | | | | | | | of 100 mg |
| | | | | | | | QD with |
| | | | | | | | dose escal |
| | | | | | | | dep on tox |
| tazemetostat | 0.1-10 μM | 0.1-10 | μM | 0.1-10 mM | 100-800 ng/ml | | PO 100 |
| (EPZ-6438) | | | | | (200-1600 nM) | | BID to |
| | | | | | | | 800 mg |
| | | | | | | | BID |
| E11 | 0.1-10 μM | 0.1-10 | μM | 0.1-10 mM | 1-10 | μM | (100 to |
| | | | | | | | 1000/day |
| | | | | | | | mg PO) |
| CPI-169 | 0.1-10 μM | 0.1-10 | μM | 0.1-10 mM | 1-10 | μM | 100 to |
| | | | | | | | 1000/day |
| | | | | | | | mg PO |
| CPI-360 | 0.1-10 μM | 0.1-10 | μM | 0.1-10 mM | 1-10 | μM | 100 to |
| | | | | | | | 1000/day |
| | | | | | | | mg PO |
| EPZ-011989 | 100 nM- | 0.10-30 | μM | 100- | 0.10-30 | μM | 100 to |
| | 30 μM | | | 30,000 μM | | | 1000/day |
| | | | | | | | mg PO |
| UNC 2399 | 0.1-10 μM | 0.1-10 | μM | 0.1-10 mM | 0.1-100 | μM | 100 to |
| | | | | | | | 1000/day |
| | | | | | | | mg PO |
| (R)-OR-S1 | | | | | | | IV 50 mg - |
| | | | | | | | poor oral |
| | | | | | | | bio. |
| A-395 | | | | | | | 50 mg and |
| | | | | | | | 200 mg PO |
| Astemizole | | | | | | | |
| Compound 19 | | | | | | | |
| Compound 22 | | | | | | | |
| Compound 24 | | | | | | | |
| Compound 34 | | | | | | | |
| Compound 41 | | | | | | | |
| CPI-0209 | | | | | | | |
| CPI-360 | | | | | | | |
| EBI-2511 | | | | | | | |
| EED162 | | | | | | | |
| EED226 | Active | | | | | | |
| EPZ-005687 | | | | | | | |
| EPZ-011989 | | | | | | | |
| GSK126 | | | | | | | |
| GSK343 | | | | | | | |
| GSK503 | | | | | | | |
| GSK926 | | | | | | | |
| MAK683 | | | | | | | |
| (EED162) | | | | | | | |
| SHR2554 | | | | | | | |
| SKLB1049 | | | | | | | |
| ZLD1039 | | | | | | | |
| ZLD1122 | | | | | | | |
| DZNep | Active | | | | | | |
| Cmpd 44 | | | | | | | |
| Compound 27 | | | | | | | |
| Sinefungin | | | | | | | |
| Tanshindiol B | | | | | | | |
| Tanshindiol C | | | | | | | |
| UN C1999 | Active | | | | | | |
| (−)- | | | | | | | |
| Epigallocatechin- | | | | | | | |
| 3-gallet (EGCG) | | | | | | | |
| Curcumin | | | | | | | |
| MC1945 | | | | | | | |
| MC1947 | | | | | | | |
| MC1948 | | | | | | | |
| SAH-EZH2 | | | | | | | |
| Sulforaphane | | | | | | | |

In some embodiments the EZH2 inhibitor is PF-06821497, CPI-120, valemetostat, tazemetostat, E11, CPI-169, CPI-360, EPZ011989, UNC 2399, or PF-06726304.

### Dot1L Inhibitors

DOT1-like (Disruptor of telomeric silencing 1-like), histone H3K79 methyltransferase (S. cerevisiae), also known as DOT1L, is a protein found in humans, as well as other eukaryotes. The methylation of histone H3 lysine 79 (H3K79) by DOT1L which is a conserved epigenetic mark in many eukaryotic epigenomes, increases progressively during the aging process.

DOT1L inhibitors are chemical compounds that inhibits histone H3K79 methyltransferase.

Thus, “DOT1L inhibitor” refers to an agent capable of the decreasing the expression or enzymatic activity of DOT1L. For example, an EZH2 inhibitor results in a decrease in histone methylation of a target gene in a cell.

In certain embodiments, the DOT1L inhibitor decreases the expression or enzymatic activity of DOT1L by at least 5, 10, 20, 30, 40, 50, 60, 70, 80, 90, or 100% relative to a control, for example relative to a baseline level of activity.

In certain embodiments, the DOT1L inhibitor decreases histone methylation of a target gene by at least 5, 10, 20, 30, 40, 50, 60, 70, 80, 90, or 100% relative to a control, for example relative to a baseline level of activity.

In some embodiments, the DOT1L inhibitor increases expression or activity of a target gene by at least 5, 10, 20, 30, 40, 50, 60, 70, 80, 90, or 100% relative to a control, for example relative to a baseline level of activity.

In some embodiments, the DOT1L inhibitor decreases expression or enzymatic activity of DOT1L by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the DOT1L inhibitor decreases histone methylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the DOT1L inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary DOT1L inhibitors are provide in Table 8.

TABLE 8

| | | pKi | | | | | | | Formulation | Human | |
|---|---|---|---|---|---|---|---|---|---|---|---|
| | | or | | Chemo- | Lit | | Lgr5+ | Perilymph | Conc. | In Vivo | Human |
| Agent | CAS | IC50 | | type | Cell | | Assay | Conc | Intratypm | Conc | Dosage |
| EPZ004777 | 1338466-77-5 | 0.3 | nM | Adenosine | 11 | nM | 0.6-45 μm | 0.6-45 μM | 0.1-45 mM | 0.1-45 uM | 10-100 |
| | | | | | | | | | | | mg/m2 |
| | | | | | | | | | | | per day |
| | | | | | | | | | | | IV |
| pinometostat | 1380288-87-8 | 0.08 | nM | Adenosine | 2.7 | nM | | 0.1-10 μM | 0.1-10 mM | Total | 54-90 |
| (EPZ-5676) | | | | | | | | | | plasma Css | mg/m2 |
| | | | | | | | | | | 800-1600 ng/mL | per day by |
| | | | | | | | | | | (1.42-2.94 uM) | continuous |
| | | | | | | | | | | (1-10 uM) | IV, |
| | | | | | | | | | | | Potential |
| | | | | | | | | | | | for SC |
| | | | | | | | | | | | dosing |
| SGC0946 | 1561178-17-3 | 0.3 | nM | Adenosine | 10 | mM | 0.6-5 μM | 0.6-5 μM | 0.6-5 mM | 0.1-5 μM | 10-100 |
| | | | | | | | | | | | mg/m2 |
| | | | | | | | | | | | per day |
| | | | | | | | | | | | IV |
| Bromo- | 1428254-21-0 | 77 | nM | Adenosine | | | | | | | |
| deaza- | | | | | | | | | | | |
| SAH | | | | | | | | | | | |
| CNSAH | 1985669-27-9 | 13 | nM | Adenosine | | | | | | | |
| Compound 10 | 1645266-99-4 | 29 | nM | Adenosine | | | | | | | |
| Compound 13 | 1940206-71-2 | 0.4 | nM | Amino- | | | | | | | |
| | | | | pyrimidine | | | | | | | |
| Compound 7 | 2088518-50-5 | <1 | nM | pyrrolo- | | | | | | | |
| | | | | pyrimidine | | | | | | | |
| Compound 8 | 1940224-84-9 | 14 | nM | Acetylene | | | | | | | |
| EPZ002696 | 1381760-94-6 | 13 | nM | Adenosine | | | | | | | |
| EPZ004450 | 1380315-97-8 | 4 | nM | Adenosine | | | | | | | |
| SAH | 979-92-0 | 600 | nM | Adenosine | | | | | | | |
| SYC-522 | 1381761-52-9 | 0.76 | mM | Adenosine | 6 | μM | | | | | |
| SYC-687 | 1440509-94-3 | 1.1 | mM | Non-Ribose | 200 | nM | | | | | |
| | 1440510-03-1, | 1.1 | nM | Adenosine | 200 | nM | | | | | |
| | 1440509-94-3 | | | | | | | | | | |
| Compound 21 | | | | Peptides | | | | | | | |
| Compound 28 | | | | Peptides | | | | | | | |
| Compound 6 | 167558-34-1 | 8.3 | μM | triazolo- | | | | | | | |
| | | | | thiadiazol | | | | | | | |
| Compound 8H | | | | pyrimidyl- | | | | | | | |
| | | | | aminoquinoline | | | | | | | |
| | 1163729-79-0 | 1.5 | μM | pyrimidine | | | | | | | |

In some embodiments the DOT1L inhibitor is EPZ004777, pinometostat or SGC0946.

### LSD1 Inhibitors

LSD1 mediated H3K4 demethylation can result in a repressive chromatin environment that silences gene expression. LSD1 has been shown to play a role in development in various contexts. LSD1 can interact with pluripotency factors in human embryonic stem cells and is important for decommissioning enhancers in stem cell differentiation. Beyond embryonic settings, LSD1 is also critical for hematopoietic differentiation. LSD1 is overexpressed in multiple cancer types and recent studies suggest inhibition of LSD1 reactivates the all-trans retinoic acid receptor pathway in acute myeloid leukemia (AML). These studies implicate LSD1 as a key regulator of the epigenome that modulates gene expression through post-translational modification of histones and through its presence in transcriptional complexes.

Thus, a “LSD1 inhibitor” refers to an agent capable of the decreasing the expression or enzymatic activity of LSD1. For example a LSD1 inhibitor results in a decrease in H3K4 demethylation of a target gene in a cell, for instance, in a cochlear cell or a vestibular. cell

In certain embodiments, a LSD1 inhibitor decreases the expression or enzymatic activity of LSD1 by at least 5, 10, 20, 30, 40, 50, 60, 70, 80, 90, or 100% relative to a control, for example relative to a baseline level of activity.

In certain embodiments, a LSD1 inhibitor decreases H3K4 demethylation by at least 5, 10, 20, 30, 40, 50, 60, 70, 80, 90, or 1⁰⁰% relative to a control, for example relative to a baseline level of activity.

In some instances, a LSD1 inhibitor decreases H3K4 demethylation by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some instances, a LSD1 inhibitor modulates (i.e. increases or decreases) expression or activity of a target gene by at least 5, 10, 20, 30, 40, 50, 60, 70, 80, 90, or 100% relative to a control, for example relative to a baseline level of activity.

In some instances, a LSD1 inhibitor modulates (i.e. increases or decreases) expression or enzymatic activity of LSD1 by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some instances a LSD1 inhibitor is reversible. In other instances the LSD1 inhibitor is irreversible.

Exemplary agents having activity as a LSD1 inhibitor are provided in Table 9 below, including pharmaceutically-acceptable salts thereof.

TABLE 9

| | | pKi | | Reversible | | | | Select | |
|---|---|---|---|---|---|---|---|---|---|
| | | or | | or | Chemo- | Select | | MAOs | |
| Agent | CAS | IC50 | | Irreversible | type | KDM1b | | A and B | |
| GSK-2879552 | 1401966-69-5 | 1.7 | μM | Irreversible | Cyproylamine | | | 20 | μM |
| | | (0.11 | μM) | | | | | | |
| GSK-LSD1 | 1431368-48-7 | 16 | nM | Irreversible | Cyproylamine | >1000X | | >1000X | |
| Phenelzine | 51-71-8 | 5.6 | μM | Irreversible | Hydrazine | | | MAO | |
| sulfate | | | | | | | | inhib | |
| | | | | | | | | 900 nM | |
| | | | | | | | | in Cell | |
| TCP | 155-09-9 | 11-477 | μM | Ineversible | Cyproylamine | 186 | μM | 1 | μM |
| (Tranylcypromine) | | | | | | | | | |
| ORY-1001 | 1431326-61-2 | <20 | nM | Irreversible | Cyproylamine | >100 | μM | >100 | μM |
| (RG6016, | | | | | | | | >100 | μM |
| RO7051790, | | | | | | | | | |
| Iadademstat) | | | | | | | | | |
| RN-1 | 1781835-13-9 | 70 | nM | Irreversible | Cyproylamine | | | 0.51 | μM |
| | | | | | | | | 2.78 | μM |
| CC-90011 | 2179319-65-2 | | | Reversible | Likely | | | | |
| | | | | | pyrimidinyl | | | | |
| GCG-11047 | 308145-19-9 | | | Reversible | Polyamine | | | | |
| (PG-11047) | | | | | | | | | |
| IMG-7289 | 2229826-41-7 | | | Ineversible | Likely | | | | |
| | | | | | Cyproylamine | | | | |
| INCB059872 | 1802909-49-4 | | | Ineversible | Likely | | | | |
| | | | | | Cyproylamine | | | | |
| ORY-2001 | 1357362-02-7 | | | Ineversible | Cyproylamine | | | | |
| (Vafidemstat) | | | | | | | | | |
| Osimertinib | 1421373-65-0 | 3.98 | μM | Reversible | Pyrimidinyl | | | | |
| (AZD9291) | | | | | | | | | |
| SP-2577 | 1423715-37-0 | | | Ineversible | Hydrazone | | | | |
| (Seclidemstat) | 1821307-10-1 | | | | | | | | |
| TCP Trans | 3721-28-6 | 284 | μM | Ineversible | Cyproylamine | 137 | μM | B: 4.4 | μM |
| Chiral | | | | | | | | | |
| TCP Trans | 3721-26-4 | 168 | μM | Ineversible | Cyproylamine | 127 | μM | B: 89 | μM |
| Chiral | | | | | | | | | |
| TCP Cis | 13531-35-6 | | | Ineversible | Cyproylamine | | | 11 | μM |
| | | | | | | | | 19 | μM |
| TCP Cis Chiral | 69684-88-4 | | | Ineversible | Cyproylamine | | | | |
| TCP Cis Chiral | 69684-89-5 | | | Ineversible | Cyproylamine | | | | |
| Compound 1 | 1221595-26-1 | 10 | nM | Ineversible | Cyproylamine | | | | |
| Compound 45 | 1667721-01-8 | 9 | nM | Ineversible | Cyproylamine | | | 15 | μM |
| | | | | | | | | >40 | μM |
| RN-7 | 1352345-82-4 | 31 | nM | Ineversible | Cyproylamine | | | | |
| Compound 5A | 1613476-09-7 | 12 | nM | Ineversible | Cyproylamine | | | | |
| Compound 2 | 1235863-51-0 | 67 | nM | Ineversible | Cyproylamine | | | >37 | μM |
| Compound 43 | 1784703-61-2 | 610 | nM | Ineversible | Cyproylamine | | | | |
| Compound 12f | 1802319-25-0 | 86 | nM | Ineversible | Cyproylamine | | | 460 | nM, |
| | | | | | | | | >70 | μM |
| T-3775440 | 1422620-34-5 | 2.1 | nM | Ineversible | Cyproylamine | | | 110 | μM, |
| | | | | | | | | 17 | μM |
| OG-L002 | 1357299-45-6 | 20 | nM | Ineversible | Cyproylamine | | | | |
| S2101 | 1239262-36-2 | 990 | nM | Ineversible | Cyproylamine | | | | |
| NCL-1 | 1196119-03-5 | 1.6 | μM | Ineversible | Cyproylamine | | | | |
| Compound 9A | 2095849-74-2 | 1.2 | μM | Ineversible | Cyproylamine | | | | |
| Compound 19l | 2173543-81-0 | 0.97 | μM | Ineversible | Cyproylamine | | | | |
| NCD-25 | 1456972-46-5 | 480 | nM | Ineversible | Cyproylamine | | | | |
| NCD-38 | 2078047-42-2 | 590 | nM | Ineversible | Cyproylamine | | | | |
| Compound 14A | 2247939-53-1 | 2.2 | nM | Ineversible | Cyproylamine | | | | |
| Compound 15A | 2247939-55-3 | 70 | nM | Ineversible | Cyproylamine | | | | |
| Compound 15B | 2247939-56-4 | 11 | nM | Ineversible | Cyproylamine | | | | |
| Compound 4 | 2226461-60-3 | 43 | nM | Ineversible | Cyproylamine | | | | |
| Pargyline | 555-57-7 | 1000 | μM | Ineversible | Amino-propyne | | | | |
| Peptide | 945548-35-6 | | | Ineversible | Amino-propyne | | | | |
| Bizine | 1591932-50-1 | 59 | nM | Ineversible | Hydrazine | | | | |
| Compound 5a | 1990536-90-7 | 1.4 | nM | Reversible | Hydrazone | | | | |
| Compound 5n | 1990537-03-5 | 1.7 | nM | Reversible | Hydrazone | | | | |
| SP-2509 | 1423715-09-6 | 13 | nM | Reversible | Hydrazone | | | >300 | μM |
| (HCI-2509) | | | | | | | | | |
| LSD1IN-32 | 2137044-49-4 | 83 | nM | Reversible | Amide | | | | |
| LSD1IN-11p | 2101951-67-9 | 20-80 | nM | Reversible | Pyrazole | | | | |
| Resveratrol | 501-36-0 | 15 | μM | Reversible | Resveratrol | | | | |
| Hydroxylamine | 2035912-55-9 | 121 | nM | Reversible | Resveratrol | | | | |
| Compound 8c | 2170023-28-4 | 283 | nM | Reversible | Resveratrol | | | | |
| CBB-1007 | 1379573-92-8 | 2.1 | μM | Reversible | Polyamine | | | | |
| Namoline | 342795-11-3 | 51 | μM | Reversible | Benzopyran-4-one | | | | |
| GSK-354 | 1841508-96-0 | 29-80 | nM | Reversible | Diphpyridine | | | A > 50 μM | |
| | | | | | | | | B = 19 μM | |
| GSK-690 | 2101305-84-2 | 37 | nM | Reversible | Diphpyridine | | | | |
| E11 | 1239589-91-3 | 243 | nM | Reversible | 2,4- | | | | |
| | | | | | Quinazolinediamine | | | | |
| MC2694 | 1435055-66-5 | 1 | μM | Reversible | 2,4- | | | | |
| | | | | | Quinazolinediamine | | | | |
| Alpha- | 11/1/6147 | 2.8 | μM | Reversible | mangostin | | | | |
| mangostin | | | | | | | | | |
| Compound 12 A | 1923750-07-5 | 0.41 | μM | Reversible | Barbituate | | | | |
| Compound 4 | 126118-57-8 | 6.4 | μM | Reversible | Purine-2,6-dione | | | | |
| Compound 10d | 2226997-31-3 | 4 | μM | Reversible | Carboxamide | | | | |
| Compound 90 | 1884266-15-2 | 162 | nM | Reversible | Carboxamide | | | | |
| Compound 46 | 1884266-36-7 | 8 | nM | Reversible | Carboxamide | | | | |
| Compound 49 | 1884266-49-2 | 7 | nM | Reversible | Carboxamide | | | | |
| Compound 50 | 1884266-48-1 | 8 | nM | Reversible | Carboxamide | | | | |
| Polymyxin B | 1404-26-8 | 157 | nM | Reversible | Polymyxin B | | | | |
| Polymyxin E | 1066-17-7 | 193 | nM | Reversible | Polymyxin E | | | | |
| Baicalin | 21967-41-9 | 3.0 | μM | Reversible | Baicalin | | | | |
| Compound 16Q | 1612870-90-2 | 9.5 | uM | Reversible | Benzenesulfonamide | | | >500 | μM |
| LSD1 inhibitor | 1853269-07-4 | 7 | nM | Reversible | Imidazole | | | | |
| 24 | | | | | | | | | |
| geranylgeranoic | 35750-48-2 | 120 | μM | Reversible | Geranyl | | | | |
| acid | | | | | | | | | |
| Geranylgeraniol | 24034-73-9 | 80 | μM | Reversible | Geranyl | | | | |
| Thiocarbamate | 1430852-56-4 | 390 | nM | Reversible | Thiocarbamate | | | >1250 | μM |
| Thiourea | 1637373-61-5 | 650 | nM | Reversible | Thiourea | | | >1250 | μM |
| Thiourea | 2035417-23-1 | 154 | nM | Reversible | Thiourea | | | | |
| Thienopyrrole | 1206028-57-0 | 2.9 | μM | Reversible | Thienopyrrole | >100 | μM | >100 | uM, |
| | | | | | | | | 57 | μM |
| Thienopyrrole | 1884266-15-2 | 162 | nM | Reversible | Thienopyrrole | | | | |
| Thienopyrrole | 1884266-48-1 | 7.8 | nM | Reversible | Thienopyrrole | 13 | μM | 41 | uM, |
| | | | | | | | | 100 | μM |
| 4SC-202 | 910462-43-0 | 1-10 | μM | Reversible | o-aminoph | | | | |
| ORY-3001 | 2179325-30-3 | | | | | | | | |
| JL1037 | | | | | | | | | |
| FLI-06 | 313967-18-9 | 92 | nM | Inhibits | Dihydropyridine | | | | |
| | | | | expression | | | | | |
| | | | | of ESD1 | | | | | |
| Rhodium | | 40 | nM | | Rhodium | | | | |
| Complex 1 | | | | | | | | | |

| | | | | | Formul. | Human | |
|---|---|---|---|---|---|---|---|
| | Literat. | | Lgr5+ | Perilymph | Conc. | Plasma | Human |
| Agent | Cell | | Assay | Conc. | Intraymp. | Conc. | Dosage |
| GSK-2879552 | 2-240 | nM | 40 nM- | 40 nM- | 40 μM to | 1-100 nM | 1 or 2 mg |
| | | | 30 μM | 30 μM | 30 mM | | QD PO |
| GSK-LSD1 | | | 4 nM- | 4 nM- | 4 μM to | 1-100 nM | 10-100 mg |
| | | | 50 μM | 50 μM | 50 mM | | PO |
| Phenelzine | 900 | | | 0.1-10 μM | 0.1-10 | Cmax | 15-90 mg/day |
| sulfate | nM in | | | | mM | 10 to | PO |
| | Cell | | | | | 60 ng/mL | |
| | | | | | | (73-440 nM) | |
| TCP | | | 0.1-20 μM | 0.1-20 μM | 0.1-20 | Cmax | 15-150\mg/day |
| (Tranylcypromine) | | | | | mM | 30-200 ng/ml | PO |
| | | | | | | (225-1500 nM) | |
| ORY-1001 | 0.5-3 | nM | 41 nM | 4 nM- | 4 uM to | 1-100 nM | 1-100 mg |
| (RG6016, | | | | 50 μM | 50 mM | | PO |
| RO7051790, | | | | | | | |
| Iadademstat) | | | | | | | |
| RN-1 | | | 41 nM | 4 nM- | 4 uM to | 1-100 nM | 10-100 mg |
| | | | | 50 μM | 50 mM | | PO |
| CC-90011 | | | | | | | |
| GCG-11047 | | | | | | | |
| (PG-11047) | | | | | | | |
| IMG-7289 | | | | | | Cmax | 80 mg |
| | | | | | | 63 ng/ml | QD PO |
| INCB059872 | | | | | | | |
| ORY-2001 | | | | | | | |
| (Vafidemstat) | | | | | | | |
| Osimertinib | 43 | nM | | | | | 10-80 mg |
| (AZD9291) | | | | | | | |
| SP-2577 | | | | | | | |
| (Seclidemstat) | | | | | | | |
| TCP Trans | | | | | | | |
| Chiral | | | | | | | |
| TCP Trans | | | | | | | |
| Chiral | | | | | | | |
| TCP Cis | | | | | | | |
| TCP Cis Chiral | | | | | | | |
| TCP Cis Chiral | | | | | | | |
| Compound 1 | | | | | | | |
| Compound 45 | | | | | | | |
| RN-7 | | | | | | | |
| Compound 5A | | | | | | | |
| Compound 2 | | | | | | | |
| Compound 43 | | | | | | | |
| Compound 12f | | | | | | | |
| T-3775440 | | | | | | | |
| OG-L002 | | | | | | | |
| S2101 | | | | | | | |
| NCL-1 | | | | | | | |
| Compound 9A | | | | | | | |
| Compound 19l | | | | | | | |
| NCD-25 | | | | | | | |
| NCD-38 | | | | | | | |
| Compound 14A | | | | | | | |
| Compound 15A | | | | | | | |
| Compound 15B | | | | | | | |
| Compound 4 | 3.8 | μM | | | | | |
| Pargyline | | | | | | | |
| Peptide | | | | | | | |
| Bizine | | | | | | | |
| Compound 5a | | | | | | | |
| Compound 5n | | | | | | | |
| SP-2509 | 350-650 | nM | | | | | |
| (HCI-2509) | | | | | | | |
| LSD1IN-32 | 670 | nM | | | | | |
| LSD1IN-11p | 0.52 | μM | | | | | |
| Resveratrol | | | | | | | |
| Hydroxylamine | | | | | | | |
| Compound 8c | 5 to 9 | μM | | | | | |
| CBB-1007 | IC50 ≤ 5 μM | | | | | | |
| Namoline | | | | | | | |
| GSK-354 | 1.3 | μM | | | | | |
| GSK-690 | | | | | | | |
| E11 | | | | | | | |
| MC2694 | | | | | | | |
| Alpha- | | | | | | | |
| mangostin | | | | | | | |
| Compound 12 A | | | | | | | |
| Compound 4 | | | | | | | |
| Compound 10d | | | | | | | |
| Compound 90 | | | | | | | |
| Compound 46 | 1-4 | μM | | | | | |
| Compound 49 | 1-4 | μM | | | | | |
| Compound 50 | 1-4 | μM | | | | | |
| Polymyxin B | | | | | | | |
| Polymyxin E | | | | | | | |
| Baicalin | | | | | | | |
| Compound 16Q | | | | | | | |
| LSD1 inhibitor | | | | | | | |
| 24 | | | | | | | |
| geranylgeranoic | | | | | | | |
| acid | | | | | | | |
| Geranylgeraniol | | | | | | | |
| Thiocarbamate | | | | | | | |
| Thiourea | | | | | | | |
| Thiourea | | | | | | | |
| Thienopyrrole | | | | | | | |
| Thienopyrrole | | | | | | | |
| Thienopyrrole | | | | | | | |
| 4SC-202 | | | | | | | 25-400 mg/Day |
| ORY-3001 | | | | | | | |
| JL1037 | | | | | | | |
| FLI-06 | | | | | | | |
| Rhodium | | | | | | | |
| Complex 1 | | | | | | | |

In some embodiments, an agent of having activity as a LSD1 inhibitor is GSK-2879552, GSK-LSD1, Osimertinib (AZD9291), Phenelzine sulfate, Tranylcypromine (TCP), RN-1, ORY-1001, Seclidemstat (SP-2577), Vafidemstat (ORY-2001), CC-90011, IMG-7289 or, INCB059872. In some embodiments, the LSD1 inhibitor is GSK-2879552, GSK-LSD1, Tranylcypromine, Phenelzine sulfate, RN-1, or ORY-1001.

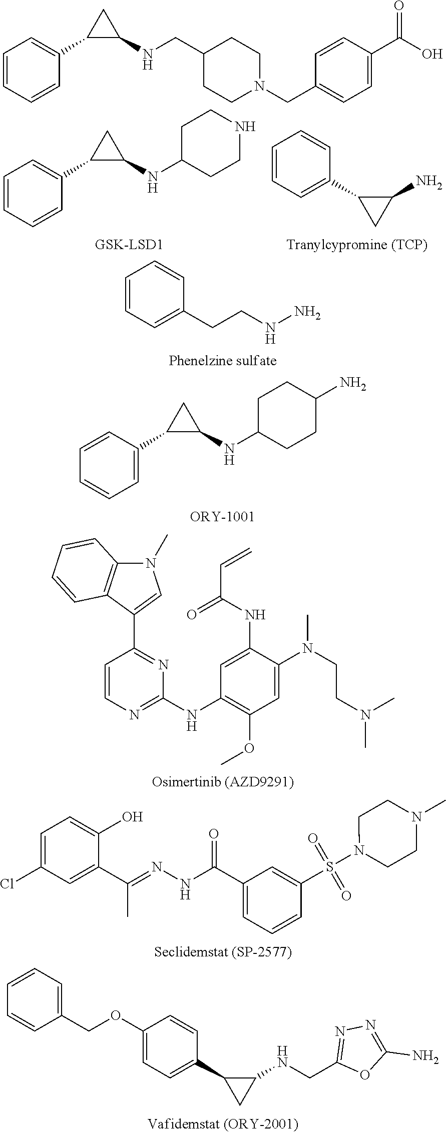

In some embodiments, the LSD1 inhibitor is GSK-2879552, ORY-1001, RN-1, or Tranylcypromine (TCP).

### KDM Inhibitors

About 30 JmjC domain-containing proteins have been identified as lysine demethylases in the human genome. Based on histone lysine sites and demethylation states, the JmjC domain-containing protein family is divided into six subfamilies: KDM2, KDM3, KDM4, KDM5, KDM6 and PHF. The JmjC domain-containing proteins belong to the Fe(II) and 2-oxoglutarate (2-OG)-dependent dioxygenases, which demethylate a variety of targets, including histones (H3K4, H3K9, H3K27, H3K36 as well as H1K26) and non-histone proteins. Unlike the LSD family, the JmjC-domain-containing histone demethylases (JHDMs) are able to erase all three kinds of histone lysine-methylation states since the JHDMs do not require protonated nitrogen for demethylation.

The KDM2 (also named FBXL) subfamily includes two members: KDM2A and KDM2B. KDM4 gene family, first identified in silico, consists of six members, including KDM4A, KDM4B, KDM4C, KDM4D, KDM4E and KDM4F. The KDM5 subfamily contains four enzymes: KDM5A, KDM5B, KDM5C and KDM5D, which specifically remove methyl marks from H3K4me2/3. In the human genome, the KDM6 subfamily is comprised of KDM6A, KDM6B and UTY, which share a well-conserved JmjC histone catalytic domain.

KDM inhibitors are chemical compounds that inhibits lysine demethylases.

Thus, “KDM inhibitor” refers to an agent capable of the decreasing the expression or enzymatic activity of KDM. For example, an KDM inhibitor results in a decrease in histone demethylation of a target gene in a cell.

In certain embodiments, the KDM inhibitor decreases the expression or enzymatic activity of KDM by at least 5, 10, 20, 30, 40, 50, 60, 70, 80, 90, or 100% relative to a control, for example relative to a baseline level of activity.

In certain embodiments, the KDM inhibitor decreases histone demethylation of a target gene by at least 5, 10, 20, 30, 40, 50, 60, 70, 80, 90, or 100% relative to a control, for example relative to a baseline level of activity.

In some embodiments, the KDM inhibitor increases expression or activity of a target gene by at least 5, 10, 20, 30, 40, 50, 60, 70, 80, 90, or 100% relative to a control, for example relative to a baseline level of activity.

In some embodiments, the KDM inhibitor decreases expression or enzymatic activity of KDM by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the KDM inhibitor decreases histone demethylation of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

In some embodiments, the KDM inhibitor increases expression or activity of a target gene by at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more relative to a control, for example relative to a baseline level of activity.

Exemplary KDM inhibitors are provide in Table 10.

TABLE 10

| | | | Covalent | Select | Select | Select | Select | | Select | | Select |
|---|---|---|---|---|---|---|---|---|---|---|---|
| Agent | CAS | Chemo-type | or not | KDM1 | KDM2 | KDM3 | KDM4 | | KDM5 | | KDM6 |
| AS 8351 | 796-42-9 | Hydrzone | | | | | | | | | |
| TC-E | 1453071-47-0 | Hydroamate | No | | 6.8 μM | | 83 | μM | 55 | μM | >100 μM |
| 5002 | | | | | | | | | | | |
| EPT-103182 | | | | | | | 20-50 | | <100 | nM | 3K |
| Compound 54k | 1844064-06-7 | pyrimidin-4-one | No | | | | 102/31 | nM | 23 | nM | |
| Cmpd 1 | 1546899-38-9 | Cyproamine | Yes | | 220 nM | | | | 190 | nM | |
| | | isonicotinic acid | | | | | | | | | |
| Cmpd 105 | 1613514-89-8 | Isonicotinic acid | No | | | | | | <100 | nM | |
| Compound 34 | 1461602-86-7 | Isonicotinic acid | No | | | | <100 | nM | <100 | nM | |
| Compound 41 | 1628332-52-4 | pyridopyrimidinone | No | | | | | | <100 | nM | |
| Compound 48 | 16282.10-26-3 | cyanopyrazole | No | | | | | | 15 | nM | |
| Compound 18 | 1993438-65-5 | naphthyridones | No | | | | | | 206 | nM | |
| Compound 33 | 1613410-75-5 | pyrazolylpyridines | No | | | | | | 10 | nM | |
| Compound 48 | 1905482-57-6 | Amide | No | | | | | | | | |
| Compound 48 | 1905482-57-6 | Pyrazole | No | | | | | | | | |
| Compound 49 | 1905481-35-7 | Pyrazole | No | | | | | | | | |
| Compound 50 | 1905481-36-8 | Pyrazole | No | | | | | | | | |
| Compound 6 | 2169272-46-0 | 1-H-Indole | No | | | | | | 50 | nM | |
| Compound R-35 | 1807514-47-1 | Triazole | No | | | | | | 65 | nM | |
| CPI-455 | 1628208-23-0 | cyanopyiazole | No | | | | 41 | nM | 13.2 | nM | |
| CPI-4203 | 1628214-07-2 | cyanopyrazol | No | | | | | | 1.1 | μM | |
| E67-2 | 1364914-62-4 | Quinazoline | No | | | | | | | | |
| GSK-467 | 1628332-52-4 | Pyrazole | No | | | | | | 14 | nM | |
| GSK-J1 | 1373422-53-7 | Acid | No | | | | | | | | |
| GSK-J4 | 1373423-53-0 | Ethyl Ester | No | | | | | | 170 | nM | 28 nM |
| KDM5-C49 | 1596348-16-1 | Pyridine | No | | | | | | | | |
| KDM5-C50 | 1596348-32-1 | Pyridine | No | | | | | | | | |
| KDOAM25 | 2230731-99-2 | Amide | No | | | | | | | | |
| N11 | 1613515-45-9 | isonicotinic | No | | | | | | 65 | nM | |
| | 1807514-47-1 | Amide | No | | | | | | 90 | nM | |
| | 1844064-07-8 | Pyridopyrimidinone | No | | | | | | 45 | nM | |
| Compound 1 | | Rh Complex | | | | | | | | | |
| Compound 15e | 1498996-89-6 | Hydrazine | | | | | | | X | | |
| Daminozide | 1596-84-5 | Hydrazine | | | X | | | | | | |
| JIB-04 | 99596-05-9 | Hydrazine | | | | | | | 220 | μM | |
| Methylstat | 1310877-95-2 | Unsat amide | | | | | | | | | |
| Compound 10r | 2098902-68-0 | cyanopyrazole | No | | | | | | | | |
| N71 | | | Yes | | | | X | | | | |
| NSC | | | | | | | 410 | nM | | | |
| 6369819 | | | | | | | | | | | |

| | | | | | | | | | | | Human | | |
|---|---|---|---|---|---|---|---|---|---|---|---|---|---|
| | Select | Select | Lit | | Lgr5+ | | Perilymph | | Formulation | | In Vivo | | Human |
| Agent | KDM7 | KDM8 | Cell | | Assay | | Conc | | Conc | | Conc | | Dosage |
| AS 8351 | | | | | 1-3 | μM | 1-3 | μM | 1-3 | mM | 1-3 | mM | 100-2000 |
| | | | | | | | | | | | | | mg/day |
| TC-E | 0.2, | | 16-40 | μM | 0.12-10 | μM | 0.12-10 | μM | 0.12-10 | mM | 0.12-10 | μM | 100-1000 |
| 5002 | 1, 2 μM | | | | | | | | | | | | mg/day |
| EPT-103182 | | | 1.8 | nM | 1-100 | nM | 1-100 | nM | 1-100 | μM | 5-50 | nM | 10 mg to |
| | | | | | | | | | | | | | 1000 mg/day |
| Compound 54k | | | | | | | | | | | | | |
| Cmpd 1 | 220 nM | | | | | | | | | | | | |
| Cmpd 105 | | | 0.1-1 | μM | | | | | | | | | |
| Compound 34 | | | | | | | | | | | | | |
| Compound 41 | | | | | | | | | | | | | |
| Compound 48 | | | 340 | nM | | | | | | | | | |
| Compound 18 | | | >10 | μM | | | | | | | | | |
| Compound 33 | | | ~1 | μM | | | | | | | | | |
| Compound 48 | | | 1-10 | μM | | | | | | | | | |
| Compound 48 | | | | | | | | | | | | | |
| Compound 49 | | | | | | | | | | | | | |
| Compound 50 | | | | | | | | | | | | | |
| Compound 6 | | | | | | | | | | | | | |
| Compound R-35 | | | 1.5 | μM | | | | | | | | | |
| CP1-455 | | | 90 | nM | | | | | | | | | |
| CPI-4203 | | | | | | | | | | | | | |
| E67-2 | | | | | | | | | | | | | |
| GSK-467 | | | | | | | | | | | | | |
| GSK-J1 | | | 50 | μM | | | | | | | | | |
| GSK-J4 | | | | | | | | | | | | | |
| KDM5-C49 | | | | | | | | | | | | | |
| KDM5-C50 | | | | | | | | | | | | | |
| KDOAM25 | | | | | | | | | | | | | |
| N11 | | | 1.6 | μM | | | | | | | | | |
| | | | 960 | μM | | | | | | | | | |
| Compound 1 | | | | | | | | | | | | | |
| Compound 15e | | | | | | | | | | | | | |
| Daminozide | X | | | | | | | | | | | | |
| JIB-04 | | | | | | | | | | | | | |
| Methyl slat | | | | | | | | | | | | | |
| Compound 10r | | | | | | | | | | | | | |
| N71 | | | | | | | | | | | | | |
| NSC | | | | | | | | | | | | | |
| 6369819 | | | | | | | | | | | | | |

In some embodiments the KDM inhibitor is AS 8351, TC-E 5002, or EPT-103182.

### Measurement of Sensorineural Hearing Loss

Hearing loss can be assessed by several different tests. Such tests may determine the audibility of a sound to a patient and/or the intelligibility of the sound to a patient prior to or after treatment. The audibility of a sound is a measure of a patient's ability to detect the sound (i.e. whether the patient can determine the presence or absence of a sound). The intelligibility of a sound is a measure of a patient's ability to correctly identify the sound. For instance, hearing is assessed according to whether a patient can correctly identify a word or not. A patient with hearing loss may therefore neither be able to detect a sound nor correctly identify it (i.e. the sound is inaudible and unintelligible). However, audibility is not necessarily associated with intelligibility, and a patient may, for example, be able detect a sound, but not correctly identify it (i.e. the sound is audible but unintelligible).

Pure Tone Audiometry

Assessment of a patient's audibility function is typically carried out by an audiologist using an audiometer in a hearing test known as pure tone audiometry. Pure tone audiometry is a standard test used to assess the audibility of a sounds and is described in detail elsewhere (see, for example, Katz, J., Medwetsky, L., Burkard, R, & Hood, L. (2009) Handbook of Clinical Audiology. Philadelphia, Pa.: Lippincott Williams and Wilkins). Pure tone audiometry is typically carried out in a sound-treated booth, which reduces ambient noise levels that may interfere with the detection of low-level sound stimuli.

In pure tone audiometry, a patient is exposed to pure tone stimuli at specific frequencies to determine the patient's hearing threshold at each frequency. Standard audiometry measures a patient's pure tone hearing threshold at each of the following frequencies 0.25 kHz, 0.5 kHz, 1 kHz, 2 kHz, 3 kHz, 4 kHz, 6 kHz and 8 kHz. However, a patient's hearing threshold does not need to be determined at all of these frequencies to ascertain whether or not the patient has sensorineural hearing loss. For instance, a subset frequencies, or a single frequency is tested to identify a patient with sensorineural hearing loss.

To determine the hearing threshold, the volume of the pure tone is altered to determine the lowest level of stimuli that the patient is able to detect. The lowest level of stimuli (corresponding to the quietest sound) is the pure tone hearing threshold at a given frequency. The pure tone threshold is typically measured in a patient using according decibels in hearing level (dB HL) on an audiometer. However, hearing thresholds may also be determined using other methods known to the person skilled in the art. For example, hearing function is measured by Auditory Brainstem Response (ABR) testing or Auditory Steady State Response (ASSR) testing. Other tests can also be used to determine hearing function in a patient. For instance, Distortion product otoacoustic emissions (DPOAEs) can be used to measure outer hair cell function and loss and is used in differential diagnosis of hearing loss arising from hair cell loss from hearing loss associated with higher level processing (e.g. auditory neuropathy).

Pure tone thresholds are plotted on a graph to produce an audiogram for the patient.

Pure tone thresholds measured across different frequencies may also be averaged to provide a pure tone average. For instance, a patient that has pure tone hearing thresholds of 50 dB HL at 0.5 Hz, 60 dB HL at 1 kHz, 65 dB HL at 2 kHz and 70 dB at 4 kHz would have a pure tone average of 61.25 dB HL, when measured across 0.5 kHz, 1 kHz, 2 kHz and 4 kHz.

Pure tone averages are calculated across different frequencies. Pure tone thresholds at any subset of frequencies are used to calculate pure tone averages. In some embodiments, the average of the patient hearing threshold is measured across 0.5 kHz, 1 kHz, 2 kHz and 4 kHz. In some embodiments, pure tone average is measured across 4 kHz, 6 kHz and 8 kHz. Measurement of pure tone average across 4 kHz, 6 kHz and 8 kHz is useful when seeking to assess the patient's hearing function at the higher frequencies within the standard audiometric frequencies.

Sensorineural hearing loss can be categorized according to its severity. The severity of hearing loss is determined by the hearing levels at which a threshold level is obtained in a patient by pure tone audiometry. Severity of hearing loss is classified according to hearing thresholds using the following definitions:
Normal: 25 dB HL or less
Mild: at least 25 dB HL and no more than 40 dB HL
Moderate: at least 40 dB HL and no more than 55 dB HL
Moderately Severe: at least 55 dB HL and no more than 70 dB HL
Severe: at least 70 dB HL and no more than 90 dB HL
Profound: at least 90 dB HL or more
   These measures of severity are standard measures in the field (see Goodman, A. (1965). Reference zero levels for pure tone audiometer. ASHA, 7, 262-263). In some embodiments, the severity of hearing loss is classified according to a patient's hearing threshold at a single frequency (for example, 0.25 kHz, 0.5 kHz, 1 kHz, 2 kHz, 3 kHz, 4 kHz, 6 kHz or 8 kHz). For instance, a patient may have mild hearing loss at 8 kHz, and normal hearing at the other standard audiometric frequencies. In some embodiments, the severity of hearing loss is classified according to pure tone average, when measured across a subset of frequencies. In certain such embodiments, the severity of hearing loss is classified according to the pure tone average across 0.5 kHz, 1 kHz, 2 kHz and 4 kHz. For example, a patient may have moderate hearing loss according to their pure tone average across 0.5 kHz, 1 kHz, 2 kHz and 4 kHz, but have moderately severe hearing loss at a single frequency (e.g. 8 kHz). In other embodiments, the severity of hearing loss is classified according to the pure tone average across 4 kHz, 6 kHz and 8 kHz.

A patient that has hearing threshold of 25 dB HL or less at standard audiometric frequencies (i.e. 0.25 kHz, 0.5 kHz, 1 kHz, 2 kHz, 3 kHz, 4 kHz, 6 kHz and 8 kHz) has normal hearing. The patient's audiogram is also a normal audiogram.

### Word Recognition Tests

Alternatively, or in addition to pure tone audiometry, hearing loss is assessed using a word recognition test. A word recognition test measures the patient's ability to correctly identify a word, thereby providing a measure of sound intelligibility (in particular, speech intelligibility) that may not be provided by pure tone audiometry. In some embodiments, a word recognition score is used to determine the patient's ability to correctly identify words prior to treatment.

A standard word recognition in quiet test, also referred to herein as a standard word recognition test, is a test administered by an audiologist that measures a patient's speech intelligibility in recognizing words in a quiet environment. A quiet environment is an environment with little to no background noise.

A standard word recognition test is used to determine a person's ability to recognize words selected from a word list and presented to the patient at a given decibel (dB) level. In some embodiments, the standard word recognition test is used to determine a patient's ability to recognize words at more than one decibel level.

In some embodiments, the standard word recognition test assesses the patient's ability to identify 50 words. However, the number of words presented to the patient is more or less than 50. For example, in some embodiments, the standard word recognition test is for 25 words. In other embodiments, the standard word recognition test is for 10 words.

A standard word recognition test is used to generate a standard word recognition (%) score which is calculated using the formula:

$$standard word recogntion score ( \% ) = 100 \times ( \frac{\begin{matrix}words recognised in standard \\ word recognition test\end{matrix}}{total words} )$$

In some embodiments, the standard word recognition score is expressed as the number of words that are correctly recognized in the test.

In some embodiments, a list of words is administered to each ear, and a standard word recognition score is calculated for each ear. Herein the results of the standard word recognition score refer to the ear that has been/will be treated.

A standard word recognition test is carried out using any list of words. However, standard word lists are typically used in a standard word recognition test. In some embodiments, each test word is embedded in a carrier phrase. Example of carrier phrases are: “Say the word_again”, “You will say_”, or “Say the word__”.

In some embodiments, the standard word recognition test is the Maryland consonant-vowel nucleus-consonant (CNC) word test. The Maryland CNC word test has been described, for example, in Mendel, L. L., Mustain, W. D., & Magro, J. (2014). Normative data for the Maryland CNC Test. Journal of the American Academy of Audiology, 25, 775-781.

The Maryland CNC word test is a standard word recognition test that uses phonemically balanced word lists comprising words that are consonant-nucleus-consonant (CNC) monosyllables. These CNC lists are balanced so that each initial consonant, each vowel, and each final consonant appears with the same frequency within each list. The Maryland CNC test has 10 lists of 50 words.

In some embodiments, the Maryland CNC Test uses words from Lehiste and Peterson's phonemically balanced word lists, all of which were CNC monosyllables, for example as described in Lehiste I, Peterson GE. (1959) Linguistic considerations in the study of speech intelligibility. Journal of the Acoustical Society of America 31(3): 280-286.

In some embodiments, the Maryland CNC Test uses words from revised CNC lists that eliminate rare literary words and proper names, for example as described in Peterson G E, Lehiste I. (1962) Revised CNC lists for auditory tests. Journal of Speech and Hearing Disorders 27:62-70.

In some embodiments, the Maryland CNC Test uses words from modified CNC word lists that take into consideration the effects of coarticulation, where the acoustic properties of phonemes are influenced by those phonemes that immediately precede and follow them, for example as described in Causey G D, Hood L J, Hermanson C L, Bowling L S. (1984) The Maryland CNC Test: normative studies. Audiology 23(6): 552-568. The words of the Maryland CNC test are spoken within the carrier phrase: ‘Say the_again,’

In some embodiments, the standard word recognition test is the C.I.D Auditory Test W-22 (CID W-22) test. The CID W-22 test has been described, for example, in Hirsh, I. J., Davis, H. Silverman, S_{R}., Reynolds, E. G., Eldert, E., & Benson, R. W. (1952). Development of Materials for Speech Audiometry. Journal of Speech, Language, and Hearing Research, 17(3), 321-337.

The CID W-22 test uses 200 monosyllabic words which are divided into four lists of 50 words each. Each list is phonetically balanced. The speech sounds within the list occur with the same relative frequency as they do in a representative sample of English speech. There are three criteria for the vocabulary in the phonetically balanced word lists. First, all the words must be one-syllable words with no repetition of words in the different lists. Second, any word chosen should be a familiar word. This second criterion is to minimize the effect of differences in the educational background of subjects. Third, the phonetic composition of each word list should correspond to that of English as a whole as closely as possible. The words of the CID W-22 test are spoken with the carrier phrase: “You will say_”.

In some embodiments the standard word recognition test is the NU No. 6 test. The NU No. 6 has been described, for example, in Tillman, T. W., & Carhart, R. (1966). An expanded test for speech discrimination utilizing CNC monosyllabic words: Northwestern University Auditory Test No. 6. Northwestern Univ Evanston I1 Auditory Research Lab.

In some embodiments, the NU No. 6 test uses 4 lists of 50 words, for example, as described in Table 28-2 of Tillman, T. W., & Carhart, R. (1966). The words of the NU No. 6 test are spoken with the carrier phrase: “Say the word_”.

In some embodiments the standard word recognition test is the Maryland CNC test, using the words list and carrier phrases as defined in Causey G D, Hood L J, Hermanson C L, Bowling L S. (1984) The Maryland CNC Test: normative studies. Audiology 23(6): 552-568. In certain such embodiments, the word signal is provided to the patient at 40 dB above speech perception level.

### Words-In-Noise (WIN) Test

A “Words-in-Noise (WIN) Test” is a test administered by an audiologist to measure a patient's speech intelligibility in recognizing words in the presence of background noise.

The WIN test consists of administering words to an ear at a varying signal-to-noise ratio (SNR) level. The signal-to-noise ratio is the ratio of the strength of the signal carrying information (e.g. the test word signal), relative to the signal of interference (e.g. noise), and is typically expressed in decibels. In some embodiments, the background noise is multi-talker babble at a fixed decibel level.

In some embodiments the multi-talker babble is comprised of six talkers (three female, three male) at a fixed level, for example, as described in Wilson, R. H., Abrams, H. B., & Pillion, A. L. (2003). A word-recognition task in multi-talker babble using a descending presentation mode from 24 dB to 0 dB signal to babble. Journal of Rehabilitation Research and Development, 40(4), 321-328.

In some embodiments, the background noise is maintained at a fixed decibel level, and the variation in the SNR decibel level is achieved by varying the decibel level of the test word signal. The SNR decibel level is therefore the SNR above the background noise. For example if the level of multi-talker babble is fixed at 70 dB SPL, and the level of the test word signal varied from 70 dB SPL to 94 dB SPL, this would give a SNR decibel level variation of 0 dB to 24 dB.

In some embodiments, the test words that are used are from any list described herein for the word recognition tests. In some embodiments, the word-in-noise test is for 70 words. In other embodiments, the words-in-noise test is for 35 words.

In some embodiments, the test consists of administering 35 or 70 monosyllabic words from the NU No. 6 word lists. The test words are spoken with the carrier phrase: “Say the word_”.

In some embodiments, the WIN test is administered in a descending-level SNR paradigm. In these embodiments, the test words at the high SNR decibel level are presented first, followed by test words at gradually lower SNR decibel levels, with words at the lowest SNR decibel level administered last. The high SNR decibel level is the easiest setting for the patient to identify the signal words. The low SNR decibel levels is the most difficult setting for the patient to identify the signal words. In other embodiments, the WIN test is administered in a randomized-level SNR paradigm. In these embodiments, the test words are presented at different SNR decibel levels in a randomized order.

In some embodiments the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR, 12 dB SNR, 8 dB SNR, 4 dB SNR and 0 dB SNR).

In some embodiments the WIN test consists of administering 70 monosyllabic words from the NU No. 6 word lists, where the SNR decibel level of the test words varies from 24 dB SNR (easiest condition) to 0 dB SNR (most difficult condition) in 4 dB decrements, for a total of seven SNR levels (i.e. 24 dB SNR, 20 dB SNR, 16 dB SNR, 12 dB SNR, 8 dB SNR, 4 dB SNR and 0 dB SNR). In this embodiment, the level of multi-talker babble is fixed at 70 dB SPL, and the level of the test word signal varies from 70 dB SPL to 94 dB SPL.

The ‘words-in-noise’ test is used to generate a words-in-noise score.

In some embodiments the words-in-noise score is given as a percentage of the total correct words recognized by the patient in the test and calculated using the formula:

$$words n noise score ( \% ) = 100 \times ( \frac{\begin{matrix}words recognised in standard \\ words in noise test\end{matrix}}{total words} )$$

### Methods of Use

In certain embodiments, the present disclosure relates to inducing, promoting, or enhancing the growth, proliferation or regeneration of inner ear tissue, particularly inner ear supporting cells and hair cells. Some embodiments relate to methods for controlled proliferation of stem cells comprising an initial phase of inducing stemness while inhibiting differentiation and a subsequent phase of differentiation of the stem cells into tissue cells.

When cochlear supporting cell or vestibular supporting cell populations are treated with an agent in accordance to the methods of the invention, whether the population is in vivo or in vitro, the treated supporting cells exhibit stem-like behavior in that the treated supporting cells have the capacity to proliferate and differentiate and, more specifically, differentiate into cochlear hair cells or vestibular hair cells. In some instances, an agent induces and maintains the supporting cells to produce daughter stem cells that can divide for many generations and maintain the ability to have a high proportion of the resulting cells differentiate into hair cells. In certain embodiments, the proliferating stem cells express stem cell marker(s) selected from one or more of Lgr5, Sox2, Opem1, Phex, lin28, Lgr6, cyclin D1, Msx1, Myb, Kit, Gdnf3, Zic3, Dppa3, Dppa4, Dppa5, Nanog, Esrrb, Rex1, Dnmt3a, Dnmt3b, Dnmt3l, Utf1, Tcl1, Oct4, Klf4, Pax6, Six2, Zic1, Zic2, Otx2, Bmi1, CDX2, STAT3, Smad1, Smad2, smad2/3, smad4, smad5, and smad7. In some embodiments, the proliferating stem cells express stem cell marker(s) selected from one or more of Lgr5, the

In some embodiments, the methods are used to maintain, or even transiently increase stemness (i.e. self-renewal) of a pre-existing supporting cell population prior to significant hair cell formation. In some embodiments, the pre-existing supporting cell population comprises inner pillar cells, outer pillar cells, inner phalangeal cells, Deiter cells, Hensen cells, Boettcher cells, and/or Claudius cells. Morphological analyses with immunostaining (including cell counts) and lineage tracing across a Representative Microscopy Samples are/is used to confirm expansion of one or more of these cell-types. In some embodiments, the pre-existing supporting cells comprise Lgr5+ cells. Morphological analyses with immunostaining (including cell counts) and qPCR and RNA hybridization are/is used to confirm Lgr5 upregulation amongst the cell population.

Advantageously, methods described herein can achieve these goals without the use of genetic manipulation. Germ-line manipulation used in many academic studies is not a therapeutically desirable approach to treating hearing loss. In some embodiments, the therapy involves the administration of a small molecule, peptide, antibody, or other non-nucleic acid molecule or nucleic acid delivery vector unaccompanied by gene therapy. In certain embodiments, the therapy involves the administration of a small organic molecule. In some instances, hearing protection or restoration is achieved through the use of a (non-genetic) therapeutic that is injected in the middle ear and diffuses into the cochlea.

The cochlea relies heavily on all present cell types, and the organization of these cells is important to their function. As supporting cells play an important role in neurotransmitter cycling and cochlear mechanics. Thus, maintaining a rosette patterning within the organ of Corti is important for function. Cochlear mechanics of the basilar membrane activate hair cell transduction. Due to the high sensitivity of cochlear mechanics, it is also desirable to avoid masses of cells. In all, maintaining proper distribution and relation of hair cells and supporting cells along the basilar membrane, even after proliferation, is likely a desired feature for hearing as supporting cell function and proper mechanics is necessary for normal hearing.

In some embodiments, the cell density of hair cells in a cochlear cell population is expanded in a manner that maintains, or even establishes, the rosette pattern characteristic of cochlear epithelia.

In certain embodiments, the cell density of hair cells is increased in a population of cochlear cells comprising both hair cells and supporting cells. The cochlear cell population is an in vivo population (i.e. comprised by the cochlear epithelium of a subject) or the cochlear cell population is an in vitro (ex vivo) population. If the population is an in vitro population, the increase in cell density is determined by reference to a Representative Microscopy Sample of the population taken prior and subsequent to any treatment. If the population is an in vivo population, the increase in cell density is determined indirectly by determining an effect upon the hearing of the subject with an increase in hair cell density correlating to an improvement in hearing.

In some embodiments, supporting cells placed in a Stem Cell Proliferation Assay in the absence of neuronal cells form ribbon synapses.

In a native cochlea, patterning of hair cells and supporting cells occurs in a manner parallel to the basilar membrane. In some embodiments, the proliferation of supporting cells in a cochlear cell population is expanded in a manner that the basilar membrane characteristic of cochlear epithelia.

In some embodiments, the number of supporting cells in an initial cochlear cell population is selectively expanded by treating the initial cochlear cell population with a composition of the present disclosure to form an intermediate cochlear cell population, wherein the ratio of supporting cells to hair cells in the intermediate cochlear cell population exceeds the ratio of supporting cells to hair cells in the initial cochlear cell population. The expanded cochlear cell population is, for example, an in vivo population, an in vitro population or even an in vitro explant. In some embodiments, the ratio of supporting cells to hair cells in the intermediate cochlear cell population exceeds the ratio of supporting cells to hair cells in the initial cochlear cell population. For example, in some embodiments, the ratio of supporting cells to hair cells in the intermediate cochlear cell population exceeds the ratio of supporting cells to hair cells in the initial cochlear cell population by a factor of 1.1, 1.5, 2, 3, 4, 5 or more. In some instances, the capacity of a composition to expand a cochlear cell population is be determined by means of a Stem Cell Proliferation Assay.

In some embodiments, the number of stem cells in a cochlear cell population is expanded to form an intermediate cochlear cell population by treating a cochlear cell population with a composition of the present disclosure wherein the cell density of stem cells in the intermediate cochlear cell population exceeds the cell density of stem cells in the initial cochlear cell population. The treated cochlear cell population is, for example, an in vivo population, an in vitro population or even an in vitro explant. In one such embodiment, the cell density of stem cells in the treated cochlear cell population exceeds the cell density of stem cells in the initial cochlear cell population by a factor of at least 1.1, 1.25, 1.5, 2, 3, 4, 5 or more. In vitro cochlear cell populations may expand significantly more than in vivo populations; for example, in certain embodiments the cell density of stem cells in an expanded in vitro population of stem cells is at least 4, 5, 6, 7, 8, 9, 10, 15, 20, 25, 30, 35, 40, 45, 50, 75, 100, 200, 300, 400, 500, 600, 700, 800, 900, 1000, 2000 or even 3000 times greater than the cell density of the stem cells in the initial cochlear cell population. In some instances, the capacity of a composition to expand a cochlear cell population is determined by means of a Stem Cell Proliferation Assay.

In some embodiments, a cochlear supporting cell population or a vestibular supporting cell population is treated with a composition of the present disclosure to increase the Lgr5 activity of the population. For example, in some instances a TAZ activator and a Wnt agonist has the capacity to increase and maintain the Lgr5 activity of an in vitro population of cochlear supporting cells or vestibular supporting cells by factor of at least 1.2, 1.5, 2, 3, 4, 5, or more. In some embodiments, the TAZ activator and a Wnt agonist has the capacity to increase the Lgr5 activity of an in vitro population of cochlear supporting cells or vestibular supporting cells by factor of 2, 3, 5 10, 100, 500, 1000, 2000 or even 3000. Increases in Lgr5 activity may also be observed for in vivo populations but the observed increase is less than in vitro populations. In some instances, the TAZ activator and a Wnt agonist inhibitor has the capacity to increase the Lgr5 activity of an in vivo population of cochlear supporting cells or vestibular supporting cells by about or at least about 5%, 10%, 20%, 30% or more. In some instances, the capacity of the TAZ activator and a Wnt agonist for such an increase in Lgr5 activity is demonstrated, for example, in an In vitro Lgr5+ Activity Assay, and in an in vivo population is demonstrated, for example, in an in Vivo Lgr5+ Activity Assay, as measured by isolating the organ and performing morphological analyses using immunostaining, endogenous fluorescent protein expression of Lgr5, and qPCR for Lgr5.

In some embodiments, the TAZ activator in combination with a Wnt agonist has the capacity to increase the Lgr5 Activity of an in vitro population of cochlear supporting cells or vestibular supporting cells by a factor of 10, 20, 30, 40, 50, 75, 100 or 200% compared to a Wnt agonist alone as measured for example in an In vitro Lgr5+ Activity Assay.

In some embodiments, the TAZ activator in combination with CHIR99021 has the capacity to increase the Lgr5 Activity of an in vitro population of cochlear supporting cells or vestibular supporting cells by a factor of 10, 20, 30, 40, 50, 75, 100 or 200% compared to CHIR99021 in combination with VPA, as measured for example in an In vitro Lgr5+ Activity Assay.

In some embodiments, the TAZ activator in combination with a Wnt agonist has the capacity to increase the Lgr5 proliferation of an in vitro population of cochlear supporting cells or vestibular supporting cells by factor of 10, 20, 30, 40, 50, 75, 100 or 200% compared to a Wnt agonist alone as measured for example in a Stem Cell Proliferation Assay.

In some embodiments, the TAZ activator in combination with a Wnt agonist has the capacity to increase the Lgr5 proliferation of an in vitro population of cochlear supporting cells or vestibular supporting cells by factor of 10, 20, 30, 40, 50, 75, 100 or 200% compared to a Wnt agonist in combination with a VPA as measured for example in a Stem Cell Proliferation Assay.

In some embodiments, the TAZ activator in combination with a Wnt agonist and VPA has the capacity to increase the Lgr5 proliferation of an in vitro population of cochlear supporting cells or vestibular supporting cells by factor of 10, 20, 30, 40, 50, 75, 100 or 200% compared to a in combination with a VPA as measured for example in a Stem Cell Proliferation Assay.

In addition to increasing the Lgr5 activity of the population, the number of Lgr5+ supporting cells in a cochlear or vestibular cell population is increased by treating a cochlear or vestibular cell population containing Lgr5+ supporting cells (whether in vivo or in vitro) with a composition of the present disclosure. In general, the cell density of the stem/progenitor supporting cells may expand relative to the initial cell population via one or more of several mechanisms. For example, in some embodiments, newly generated Lgr5+ supporting cells are generated that have increased stem cell propensity (i.e. greater capacity to differentiate into hair cell). By way of further example, in some embodiments no daughter Lgr5+ cells are generated by cell division, but pre-existing Lgr5+ supporting cells are induced to differentiate into hair cells. By way of further example, in some embodiments no daughter cells are generated by cell division, but Lgr5− supporting cells are activated to a greater level of Lgr5 activity and the activated supporting cells are then able to differentiate into hair cells. Regardless of the mechanism, in some embodiment a composition of the present disclosure (e.g. a composition comprising a TAZ activator and a Wnt agonist and optionally a epigenetic agent) has the capacity to increase the cell density of Lgr5+ supporting cells in an in vitro isolated cell population of cochlear supporting cells or vestibular supporting cells by factor of at least 5, 10, 50, 100, 500, 1000, or 2000. Increases in the cell density of Lgr5+ supporting cells are also observed for in vivo populations but the observed increase is somewhat more modest. For example, in some embodiments the composition has the capacity to increase the cell density of Lgr5+ supporting cells in an in vivo population of cochlear supporting cells or vestibular supporting cells by about or at least about 5%, 10%, 20%, 30% or more. The capacity of the composition for such an increase in Lgr5+ supporting cells in an in vitro population is demonstrated, for example, in a Stem Cell Proliferation Assay or in an appropriate in vivo assay. In some embodiments, a composition of the present disclosure has the capacity to increase the number of Lgr5+ cells in the cochlea by inducing expression of Lgr5 in cells with absent or low detection levels of the protein, while maintaining Native Morphology. In some embodiments, a composition has the capacity to increase the number of Lgr5⁺ cells in the cochlea or vestibular organ by inducing expression of Lgr5 in cells with absent or low detection levels of the protein, while maintaining Native Morphology and without producing Cell Aggregates.

Included in the invention are methods of increasing proliferation of a Lgr5+ cochlear supporting cell by contacting a cochlear supporting cell with a TAZ activator and a Wnt agonist. Optionally, the cell is further contacted with an epigenetic agent such as an HDAC inhibitor, an LSD1 Inhibitor, an EZH2 inhibitor, a DOT1L inhibitor, or a KDM inhibitor. In some embodiments, the HDAC inhibitor is VPA.

Included in the invention are methods of increasing proliferation of a vestibular supporting cell by contacting a vestibular supporting cell with a TAZ activator and a Wnt agonist. Optionally, the cell is further contacted with an epigenetic agent such as an HDAC inhibitor. In some embodiments, the HDAC inhibitor is VPA.

In the various methods Lgr5+ cochlear cell or vestibular cell proliferation is increased compared to a vehicle control.

In some embodiments, the TAZ activator and the Wnt agonist increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more), relative to a vehicle control.

In some embodiments, the TAZ activator and the Wnt agonist in combination with an epigenetic agent increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to a Wnt agonist alone in a Stem Cell Proliferation Assay.

In some embodiments, the TAZ activator and the Wnt agonist in combination with an epigenetic agent increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more) relative to Wnt agonist in combination with VPA in a Stem Cell Proliferation Assay.

In some embodiments, the TAZ activator and the Wnt agonist increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more), relative to a Wnt agonist alone, as measured in a Stem Cell Proliferation Assay.

In some embodiments, the TAZ activator and the Wnt agonist increases Lgr5+ cochlear supporting cell or vestibular supporting cell proliferation by at least 10, 20, 30, 40, 50, 60, 70, 80, 90, 100, 150, 200, 250, 300, 350, 400, 450, or 500% or more (or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more), relative to a Wnt agonist in combination with VPA, as measured in a Stem Cell Proliferation Assay.

Also included are methods for expanding a population of cochlear cells in a cochlear tissue comprising a parent population of cells by contacting the cochlear tissue with a TAZ activator and a Wnt agonist to form an expanded population of cells in the cochlear tissue. Optionally, the cell is further contacted with an epigenetic agent such as an HDAC inhibitor such as an class I HDAC inhibitor In some embodiment, the class I HDAC inhibitor is a short chain carboxylic acid such as for example, valproic acid (VPA).

The TAZ activator and the Wnt agonist (optionally in combination with an epigenetic agent) is capable of (i) forming a proliferation assay final cell population from a proliferation assay initial cell population over a proliferation assay time period in a stem cell proliferation assay, and/or (ii) forming a differentiation assay final cell population from a differentiation assay initial cell population over a differentiation assay time period in a Stem Cell differentiation assay wherein: (a) the proliferation assay initial cell population has (i) a Proliferation assay initial number of total cells, (ii) a proliferation assay initial number of Lgr5+ cells, (iii) a proliferation assay initial number of hair cells, (iv) a proliferation assay initial Lgr5+ cell fraction that equals the ratio of the proliferation assay initial number of Lgr5+ cells to the proliferation assay initial number of total cells, and (v) a proliferation assay initial hair cell fraction that equals the ratio of the proliferation assay initial number of hair cells to the proliferation assay initial number of total cells; (b) the proliferation assay final cell population has (i) a proliferation assay final number of total cells, (ii) a proliferation assay final number of Lgr5+ cells, (iii) a proliferation assay final number of hair cells, (iv) a proliferation assay final Lgr5+ cell fraction that equals the ratio of the proliferation assay final number of Lgr5+ cells to the proliferation assay final number of total cells and (v) a proliferation assay final hair cell fraction that equals the ratio of the proliferation assay final number of hair cells to the proliferation assay final number of total cells; (c) the differentiation assay initial cell population has (i) a differentiation assay initial number of total cells, (ii) a differentiation assay initial number of Lgr5+ cells, (iii) a differentiation assay initial number of hair cells, (iv) a differentiation assay initial Lgr5+ cell fraction that equals the ratio of the differentiation assay initial number of Lgr5+ cells to the differentiation assay initial number of total cells, and (v) a differentiation assay initial hair cell fraction that equals the ratio of the differentiation assay initial number of hair cells to the differentiation assay initial number of total cells; (d) the differentiation assay final cell population has (i) a differentiation assay final number of total cells, (ii) a differentiation assay final number of Lgr5+ cells, (iii) a differentiation assay final number of hair cells, (iv) a differentiation assay final Lgr5+ cell fraction that equals the ratio of the differentiation assay final number of Lgr5+ cells to the differentiation assay final number of total cells, and (v) a differentiation assay final hair cell fraction that equals the ratio of the differentiation assay final number of hair cells to the differentiation assay final number of total cells; (e) the proliferation assay final number of Lgr5+ cells exceeds the proliferation assay initial number of Lgr5+ cells by a factor of at least 10; and/or (f) the differentiation assay final number of hair cells is a non-zero number.

The invention also includes methods of producing an expanded population of Lgr5+ cochlear cells by contacting the cell population with a TAZ activator and Wnt agonist to form an expanded population of cells in the cochlear tissue. Optionally, the cell is further contacted with an epigenetic agent such as an HDAC inhibitor. In some embodiments, the HDAC inhibitor is VPA.

The expanded population is capable of differentiating into hair cells as measured in a stem cell differentiation assay.

In some embodiments, the cochlear cell is in a cochlear tissue. In some embodiments, the cochlear tissue is in a subject.

Some embodiments relate to methods of treating a subject who has, or is at risk for developing, hearing loss or reduced auditory function. The prophylaxis and/or treatment of acute and chronic ear disease and hearing loss, dizziness and balance problems especially of sudden hearing loss, acoustic trauma, hearing loss due to chronic noise exposure, presbycusis, trauma during implantation of the inner ear prosthesis (insertion trauma), dizziness due to diseases of the inner ear area, dizziness related and/or as a symptom of Meniere's disease, vertigo related and/or as a symptom of Meniere's disease, tinnitus, hypercusis and hearing loss due to antibiotics and cytostatics and other drugs.

Some embodiments include methods to prevent, reduce, or treat the incidence and/or severity of inner ear disorders and hearing impairments involving inner ear tissue, particularly inner ear hair cells, their progenitors, and optionally, the stria vascularis, and associated auditory nerves. Of particular interest are those conditions that lead to permanent hearing loss where reduced number of hair cells is responsible and/or decreased hair cell function. Also of interest are those arising as an unwanted side-effect of ototoxic therapeutic drugs including cisplatin and its analogs, aminoglycoside antibiotics, salicylate and its analogs, or loop diuretics.

Hearing loss or reduced auditory function is treated or prevented in a subject by contacting a Lgr5+ cochlear cell or administering to the subject a TAZ activator and Wnt agonist to form an expanded population of cells in the cochlear tissue. Optionally, the cell is further contacted with an epigenetic agent such as an HDAC inhibitor. In some embodiments, the HDAC inhibitor is VPA.

In various embodiments the TAZ activator and Wnt agonist and optionally, the one or more additional epigenetic agents are administered to the subject systemically or locally. Systemic administration includes, but is not limited, to oral or parenteral administration. Parenteral routes include for example intramuscular (IM), subcutaneous (SC) and intravenous (IV). Local administration includes for example, intratympanic or intracochlear administration. More specific methods of local delivery are described herein. In some embodiments, both the TAZ activator and Wnt agonist are administered locally. In other embodiments, both the TAZ activator and Wnt agonist are administered systemically. In some embodiments the TAZ activator is administered locally and the Wnt agonist is administered systemically. In other embodiments the TAZ activator is administered systemically and the Wnt agonist is administered locally.

In some embodiments, the TAZ activator and Wnt agonist are administered at the same time. In other embodiments, the TAZ activator and Wnt agonist are administered at different times. In some embodiments the TAZ activator is administered a period of time before the WNT agonist. In other embodiments, the TAZ activator is administered at a period of time after the Wnt agonist. For example, the TAZ activator is administered 1, 2, 3, 4, 5, 6, 7, 8, 9, 10, 12, 13, 14, 14, 15, 17, 18, 19, 20, 21. 22, 23, 24 hours or 1, 2, 3, 4, 5, 6, 7 or more days before the Wnt agonist. Alternatively, the TAZ activator is administered 1, 2, 3, 4, 5, 6, 7, 8, 9, 10, 12, 13, 14, 14, 15, 17, 18, 19, 20, 21. 22, 23 or 24 hours or 1, 2, 3, 4, 5, 6, 7 or more days before the Wnt agonist after the Wnt agonist.

Hearing loss or reduced auditory function is treated or prevented utilizing the various methods described herein to increase Lgr5+ cochlear cell proliferation. The cochlear cell is contacted with a TAZ activator and Wnt agonist at a “cell effective concentration” to form an expanded population of cells in the cochlear tissue. Optionally, the cell is further contacted with an epigenetic agent such as an HDAC inhibitor. In some embodiments, the HDAC inhibitor is VPA.

A “cell effective concentration” is the minimum concentration of the compound that induces at least an 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more in gene expression and/or about a 1.5-fold increase in number of Lgr5+ cells in a Stem Cell Proliferation Assay compared to a vehicle control.

In some embodiments, the Lgr5+ cochlear cell is contacted in vitro with the compound(s) at the “cell effective concentration”, such as for example, in a cell culture (and then implanted into the cochlea). In other embodiments, the Lgr5+ cochlear cell is contacted with the compound(s) at the “cell effective concentration”, in situ (i.e. within the cochlea). In some embodiments, sufficient compound is delivered to achieve the “cell effective concentration” throughout the speech region of the human cochlea. In order to achieve this target concentration, a higher concentration of drug is instilled in the cochlea and diffuse throughout the speech region. In other embodiments, the Lgr5+ cochlear cell is contacted with the compound(s) at 2, 3, 4, 5, 10, 20, 50, 100, 200, 300, 400, 500, 600, 700, 800, 900, 1000-fold more than the “cell effective concentration”, in situ (i.e. within the cochlea).

Alternatively, hearing loss or reduced auditory function is treated by administering the compound(s) at the “formulation effective concentration”. A “formulation effective concentration” is a higher concentration than the “cell effective formulation”. For example, the “formulation effective concentration” is at least about 100 to 5000 fold higher than the “cell effective concentration”, or about 20 100, 250, 500, 750, 1000, 1250, 1500, 1750, 2000 fold higher than the “cell effective concentration”, or about 100, 200, 300, 400, 500, 600, 700, 800, 900 or 1000 fold higher than the “cell effective concentration”. Typically, the “formulation effective concentration” is at least about 1000 fold higher than the “cell effective concentration”.

Alternatively, hearing loss or reduced auditory function is treated by administering the compound(s) at a set daily dose.

The compound(s) are formulated at the “cell effective concentration” and the “formulation effective concentration” as described supra.

In some embodiments, the “cell effective concentration” of the compound(s) is about 0.01 μM to 1000 nM, about 1 μM to 100 nM, about 10 μM to 10 nM, about 1 μM to 10 μM, about 10 nM to 100 nM, about 100 nM to 1000 nM, about 1 nM to 10 nM, 0.01 μM to 1000 μM, about 1 μM to 100 μM, about 10 μM to 10 μM, about 1 μM to 1 mM, or about 10 mM to 100 mM.

In some embodiment the compound is administered to the subject systemically at a daily dose of about 0.01 mg to 1000 mg/day; about 0.01 mg to 500 mg/day; about 0.01 mg to 250 mg/day; about 0.01 mg to 100 mg/day; about 0.01 mg to 50 mg/day; about 0.01 mg to 25 mg/day; about 0.01 mg to 10 mg/day; about 0.01 mg to 5 mg/day; 0.1 mg to 100 mg/day; about 0.1 mg to 50 mg/day; about 0.01 mg to 25 mg/day; about 0.01 mg to 10 mg/day; about 0.01 mg to 5 mg/day; about 0.01 mg to 2.5 mg/day; about 0.1 mg to 10 mg/day; about 0.1 mg to 5 mg/day about 0.1 mg to 4 mg/day; about 0.1 mg to 3 mg/day; about 0.1 mg to 2 mg/day; about 0.1 mg to 2 mg/day or about 1 mg to 5 mg/day.

In some embodiments, compound is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration. In some embodiments, compound administered to the subject at about 0.01×. 0.1×, 2×, 3×, 5× or 10×, relative to an FDA approved concentration.

In some embodiments the additional agent is a TAZ activator.

In some embodiments, the TAZ activator is IBS008738 and is administered for example to a cochlear cell in amount sufficient to achieve a concentration of about 0.01 nM to 1 mM, about 0.1 nM to 100 μM, about 1 nM to 100 μM, about 10 nM to 100 μM, about 100 nM to 100 μM, 1 μM to 100 μM, about 10 nM to 100 nM, about 100 nM to 1 μM, about 1 μM to 10 μM or about 10 μM to 100 μM, in the perilymph fluid in the inner ear.

In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the TAZ activator is IBS008738 is administered to a subject, for example to the middle ear at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the TAZ activator is IBS008738 and is administered systemically at a daily dose of about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the TAZ activator is IBS008738 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, TAZ activator is IBS008738 and is administered to the subject at about 0.01×. 0.1×, 1×, 2×, 3×, 4×, 5× or 10×, relative to an FDA approved dose. An IBS008738 dose is for example the concentration listed on Table 1, column titled “Human Dosage”.

In some embodiments, the TAZ activator is TT-10 and is administered for example to a cochlear cell in amount sufficient to achieve a concentration of about 0.01 nM to 1 mM, about 0.1 nM to 100 μM, about 1 nM to 100 μM, about 10 nM to 100 μM, about 100 nM to 100 μM, 1 μM to 100 μM, about 10 nM to 100 nM, about 100 nM to 1 μM, about 1 μM to 10 μM or about 10 μM to 100 μM, in the perilymph fluid in the inner ear.

In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the TAZ activator is TT-10 is administered to a subject, for example to the middle ear at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the TAZ activator is TT-10 and is administered systemically at a daily dose of about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the TAZ activator is TT-10 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, TAZ activator is TT-10 and is administered to the subject at about 0.01×. 0.1×, lx, 2×, 3×, 4×, 5× or 10×, relative to an FDA approved dose. A TT-10 dose is for example the concentration listed on Table 1, column titled “Human Dosage”.

In some embodiments, the TAZ activator is TM-25659 and is administered for example to a cochlear cell in amount sufficient to achieve a concentration of about 0.01 nM to 1 mM, about 0.1 nM to 100 μM, about 1 nM to 100 μM, about 10 nM to 100 μM, about 100 nM to 100 μM, 1 μM to 100 μM, about 10 nM to 100 nM, about 100 nM to 1 μM, about 1 μM to 10 μM or about 10 μM to 100 μM, in the perilymph fluid in the inner ear.

In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear.

In some embodiments, the TAZ activator is TM-25659 is administered to a subject, for example to the middle ear at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the TAZ activator is TM-25659 and is administered systemically at a daily dose of about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the TAZ activator is TM-25659 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, TAZ activator is TM-25659 and is administered to the subject at about 0.01×. 0.1×, 1×, 2×, 3×, 4×, 5× or 10×, relative to an FDA approved dose. An TM-25659 dose is for example the concentration listed on Table 1, column titled “Human Dosage”

In some embodiments, the TAZ activator is FHZ-000706 and is administered for example to a cochlear cell in amount sufficient to achieve a concentration of about 0.01 nM to 1 mM, about 0.1 nM to 100 μM, about 1 nM to 100 μM, about 10 nM to 100 μM, about 100 nM to 100 μM, 1 μM to 100 μM, about 10 nM to 100 nM, about 100 nM to 1 μM, about 1 μM to 10 sM or about 10 μM to 100 μM, in the perilymph fluid in the inner ear.

In some embodiments, the FHZ-000706 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM or about 100 μM in the perilymph fluid in the inner ear.

In some embodiments, the TAZ activator is FHZ-000706 is administered to a subject, for example to the middle ear at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the FHZ-000706 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 m, 3 mM, 4 mM, 5 mM, 6 μM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM or about 100 mM.

In some embodiments, the TAZ activator is FHZ-000706 and is administered systemically at a daily dose of about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the GSK3 Inhibitor is AZD1080, and is administered for example to a cochlear cell in amount sufficient to achieve a concentration of about 0.001 μM to 10 mM, about 0.01 uM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM in the perilymph fluid in the inner ear.

In some embodiments, the AZD1080 is administered, is administered, in amount sufficient to achieve a concentration of about is about 1 μM, 2 μM, 3 μM, 4 μM, 5 μM, 6 μM, 7 μM, 8 μM, 9 μM, or 10 μM in the perilymph fluid in the inner ear.

In some embodiments, the GSK3 Inhibitor is AZD1080, and is administered to a subject, for example to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the AZD1080 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments, the GSK3 Inhibitor is AZD1080 and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is AZD1080 and is administered to the subject at about 0.01×. 0.1×, 2×, 3×, 5× or 10×, relative to an FDA approved concentration

In some embodiments, the GSK3 Inhibitor is LY2090314, and is administered for example to a cochlear cell in amount sufficient to achieve a concentration of about 0.001 nM to 10 mM, about 0.01 nM to 1 μM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, or about 1 μM to 10 μM, in the perilymph fluid in the inner ear.

In some embodiments, the LY2090314 is administered, in amount sufficient to achieve a concentration of about 1 nM, 5 nM, 10 nM, 15 nM, 20 nM, or 40 nM, in the perilymph fluid in the inner ear.

In some embodiments, the GSK3 Inhibitor is LY2090314, and is administered to a subject, for example to the middle ear at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, LY2090314 the is administered to a subject, for example to the middle ear at a concentration of about 1 μM, 5 μM, 10 μM, 15 μM, 20 μM, or 40 μM.

In some embodiments, the GSK3 Inhibitor is LY2090314 and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is LY2090314 and is administered to the subject at about 0.01×. 0.1×, 2×, 3×, 5× or 10×, relative to an FDA approved concentration.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione, and is administered for example to a cochlear cell in amount sufficient to achieve a concentration of about 0.001 nM to 10 mM, about 0.01 nM to 1 μM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1 μM, or about 1 μM to 10 μM, in the perilymph fluid in the inner ear.

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione, is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione, and is administered to a subject, for example to the middle ear at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione, the is administered to a subject, for example to the middle ear at a concentration of about 1 μM, 5 μM, 10 μM, 15 μM, 20 μM, 50 μM, 100 μM, 250 μM, or 500 μM.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione, and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to the subject at about 0.01×. 0.1×, 2×, 3×, 5× or 10×, relative to an FDA approved concentration

In some embodiments, the GSK3 Inhibitor is GSK3-inhibitor XXII, and is administered for example to a cochlear cell in amount sufficient to achieve a concentration of about 0.1 nM to 1 mM, about 1 nM to 100 μM, about 10 nM to 10 μM, about 0.1 nM to 1 nM, about 1 nM to 10 nM, about 10 nM to 100 nM, about 100 nM to 1 μM, about 1 μM to 10 μM, about 10 μM to 100 μM, or about 100 μM to 1000 μM, in the perilymph fluid in the inner ear.

In some embodiments, the GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear.

In some embodiments, the GSK3 Inhibitor is GSK3-inhibitor XXII, is administered to a subject, for example to the middle ear at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM. In some embodiments, the GSK3-inhibitor XXII is administered, to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, or 1.0 mM

In some embodiments, the GSK3 Inhibitor is GSK3-inhibitor XXII and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is GSK3-inhibitor XXII and is administered to the subject at about 0.01×. 0.1×, 2×, 3×, 5× or 10×, relative to an FDA approved concentration.

In some embodiments, the GSK3 Inhibitor is CHIR99021, and is administered for example to a cochlear cell in amount sufficient to achieve a concentration of about 0.001 mM to 10 mM, about 0.01 mM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM, in the perilymph fluid in the inner ear.

In some embodiments, the CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear.

In some embodiments, the GSK3 Inhibitor is CHIR99021, is administered to a subject, for example to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments, the GSK3 Inhibitor is CHIR99021 and is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration.

In some embodiments, the GSK3 Inhibitor is CHIR99021 and is administered to the subject at about 0.01×. 0.1×, 2×, 3×, 5× or 10×, relative to an FDA approved concentration.

In various embodiments, the methods further comprise administering one more additional epigenetic agents, such as an HDAC inhibitor, an EZH2 inhibitor, a DOT1L inhibitor, a KDM inhibitor, or a TAZ activator as described herein.

In some embodiments the additional epigenetic agent is an HDAC inhibitor and is administered for example to a cochlear cell in amount sufficient to achieve a concentration of about is about 0.01 uM to 1000 mM, about 1 uM to 100 mM, about 10 uM to 10 mM, about 1 uM to 10 uM, about 10 uM to 100 uM, about 100 uM to 1000 uM, about 1 mM to 10 mM, or about 10 mM to 100 mM in the perilymph fluid in the inner ear.

In some embodiments the HDAC inhibitor is administered, to a subject, for example to the middle ear at a concentration about 10 uM to 1,000,000 mM, about 1000 uM to 100,000 mM, about 10,000 uM to 10,000 mM, about 1000 uM to 10,000 uM, about 10,000 uM to 100,000 uM, about 100,000 uM to 1,000,000 uM, about 1,000 mM to 10,000 mM, or about 10,000 mM to 100,000 mM.

In some embodiments, the HDAC inhibitor is VPA and is administered for example to a cochlear cell in amount sufficient to achieve a concentration of about is about 10 μM to 4 mM in the perilymph fluid in the inner ear.

In some embodiments VPA is administered, to a subject, for example to the middle ear at a concentration about 100 mM to 4,000 mM.

In some embodiments, the HDAC inhibitor is VPA and is administered to a subject systemically at a daily dose of about 50 mg, about 100 mg, about 125 mg, about 250 mg, about 500 mg, 1000 mg, 2000 mg, 3000 mg, 4000 mg, or about 5000 mg. In some embodiments, the VPA is administered as an oral dosage form of about 50 mg, about 100 mg, about 125 mg, about 250 mg, about 500 mg, 1000 mg, 2000 mg, 3000 mg, 4000 mg, or about 5000 mg

In some embodiments, the HDAC inhibitor is 2-hexyl-4-pentynoic acid and is administered for example to a cochlear cell in amount sufficient to achieve a concentration of about is about 10 μM to 4 mM in the perilymph fluid in the inner ear.

In some embodiments 2-hexyl-4-pentynoic acid is administered, to a subject, for example to the middle ear at a concentration about 100 mM to 4,000 mM.

In some embodiments, the HDAC inhibitor is 2-hexyl-4-pentynoic acid and is administered to a subject systemically at a daily dose of about 50 mg, about 100 mg, about 125 mg, about 250 mg, about 500 mg, 1000 mg, 2000 mg, 3000 mg, 4000 mg, or about 5000 mg. In some embodiments, the VPA is administered as an oral dosage form of about 50 mg, about 100 mg, about 125 mg, about 250 mg, about 500 mg, 1000 mg, 2000 mg, 3000 mg, 4000 mg, or about 5000 mg

In some embodiments, the HDAC inhibitor is Na phenylbutyrate and is administered for example to a cochlear cell in amount sufficient to achieve a concentration of about is about 10 μM to 4 mM in the perilymph fluid in the inner ear.

In some embodiments V Na phenylbutyrate is administered, to a subject, for example to the middle ear at a concentration about 100 mM to 4,000 mM.

In some embodiments, the HDAC inhibitor is Na phenylbutyrate and is administered to a subject systemically at a daily dose of about 50 mg, about 100 mg, about 125 mg, about 250 mg, about 500 mg, 1000 mg, 2000 mg, 3000 mg, 4000 mg, or about 5000 mg. In some embodiments, the VPA is administered as an oral dosage form of about 50 mg, about 100 mg, about 125 mg, about 250 mg, about 500 mg, 1000 mg, 2000 mg, 3000 mg, 4000 mg, or about 5000 mg.

In some embodiments the additional epigenetic agent is an EZH2 inhibitor

In some embodiments, the EZH2 inhibitor is PF-06821497 and is administered for example to a cochlear cell in amount sufficient to achieve a concentration of about 0.001 nM to 100 μM about 0.01 nM to 10 μM, about 0.1 nM to 1 s M, about 1 nM to 100 nM, about 1 nM to 10 nM, about 10 nM to 100 nM, or about 100 nM to 1 μM, in the perilymph fluid in the inner ear.

In some embodiments, the PF-06821497 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear.

In some embodiments, the EZH2 inhibitor is PF-06821497 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the PF-06821497 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the EZH2 inhibitor is PF-06821497 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is PF-06821497 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is PF-06821497 and is administered to the subject at about 0.01×. 0.1×, lx, 2×, 3×, 4×, 5× or 10×, relative to an FDA approved dose. A PF-06821497 dose is for example the concentration listed on Table 7, column titled “Human Dosage”.

In some embodiments, the EZH2 inhibitor is CPI-1205 and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, or about 100 nM to 1 μM, in the perilymph fluid in the inner ear.

In some embodiments, the CPI-1205 is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM, in the perilymph fluid in the inner ear.

In some embodiments, the EZH2 inhibitor is CPI-1205 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, about 10 μM to 100 μM, or about 100 μM to 1000 μM.

In some embodiments, the CPI-1205 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the EZH2 inhibitor is CPI-1205 and is administered systemically at a daily dose of about 100 to 5,000 mg/day, about 100 mg to 4000 mg/day, about 100 mg to 3000 mg/day, about 100 mg to 2000 mg/day, about 500 to 5,000 mg/day, about 500 mg to 4000 mg/day, about 500 mg to 3000 mg/day, about 750 to 5,000 mg/day, about 750 mg to 4000 mg/day, about 750 mg to 3000 mg/day, about 800 mg to 2400 mg/day, about 400 mg/day, about 600 mg/day, about 800 mg/day, about 1000 mg/day, about 1200 mg/day, about 1400 mg/day, about 1600 mg/day, about 1800 mg/day, about 2000 mg/day, about 2200 mg/day, about 2400 mg/day, about 2600 mg/day, about 2800 mg/day, about 3000 mg/day, about 3250 mg/day, about 3500 mg/day, about 4000 mg/day, about 4500 mg/day, or about 5000 mg/day.

In some embodiments, the EZH2 inhibitor is CPI-1205 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is CPI-1205 and is administered to the subject at about 0.01×. 0.1×, 1×, 2×, 3×, 4×, 5× or 10×, relative to an FDA approved dose. A CPI-1205 dose is for example the concentration listed on Table 7, column titled “Human Dosage”.

In some embodiments, the EZH2 inhibitor is valemetostat and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, or about 100 nM to 1 μM, in the perilymph fluid in the inner ear.

In some embodiments, the valemetostat is administered, in amount sufficient to achieve a concentration of about 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or 1 μM, in the perilymph fluid in the inner ear.

In some embodiments, the EZH2 inhibitor is valemetostat is administered to a subject, for example to the middle ear at a concentration of about 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1000 μM.

In some embodiments, the valemetostat is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or 1 mM.

In some embodiments, the EZH2 inhibitor is valemetostat and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2000 mg/day, about 100 mg to 1500 mg/day, about 100 mg to 1000 mg/day, about 100 mg to 500 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is valemetostat and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is valemetostat and is administered to the subject at about 0.01×. 0.1×, 1×, 2×, 3×, 4×, 5× or 10×, relative to an FDA approved dose. A valemetostat dose is for example the concentration listed on Table 7, column titled “Human Dosage”

In some embodiments, the EZH2 inhibitor is tazemetostat and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, 100 nM to 1 μM, or about 1 μM to 10 μM, in the perilymph fluid in the inner ear.

In some embodiments, the tazemetostat is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the EZH2 inhibitor is tazemetostat is administered to a subject, for example to the middle ear at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the tazemetostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the EZH2 inhibitor is tazemetostat and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is tazemetostat and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is tazemetostat and is administered to the subject at about 0.01×. 0.1×, lx, 2×, 3×, 4×, 5× or 10×, relative to an FDA approved dose. A tazemetostat dose is for example the concentration listed on Table 7, column titled “Human Dosage”.

In some embodiments, the EZH2 inhibitor is E11 and is administered for example to a cochlear cell in amount sufficient to achieve a concentration of about 0.1 nM to 1 mM, about 1 nM to 100 μM, about 10 nM to 10 μM, about 100 nM to 10 μM, about 10 nM to 100 nM, about 100 nM to 1 μM about 1 μM to 10 μM, or about 10 μM to 100 μM, in the perilymph fluid in the inner ear.

In some embodiments, the E11 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, or about 50 μM, in the perilymph fluid in the inner ear.

In some embodiments, the EZH2 inhibitor is E11 is administered to a subject, for example to the middle ear at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the E11 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the EZH2 inhibitor is E11 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is E11 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is E11 and is administered to the subject at about 0.01×. 0.1×, 1×, 2×, 3×, 4×, 5× or 10×, relative to an FDA approved dose. An E11 dose is for example the concentration listed on Table 7, column titled “Human Dosage”.

In some embodiments, the EZH2 inhibitor is CPI-169 and is administered for example to a cochlear cell in amount sufficient to achieve a concentration of about 0.1 nM to 1 mM, about 1 nM to 100 μM, about 10 nM to 10 μM, about 100 nM to 10 μM, about 10 nM to 100 nM, about 100 nM to 1 μM, about 1 μM to 10 μM, or about 10 μM to 100 μM, in the perilymph fluid in the inner ear.

In some embodiments, the CPI-169 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, or about 50 μM, in the perilymph fluid in the inner ear.

In some embodiments, the EZH2 inhibitor is CPI-169 is administered to a subject, for example to the middle ear at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the CPI-169 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the EZH2 inhibitor is CPI-169 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is CPI-169 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is CPI-169 and is administered to the subject at about 0.01×. 0.1×, 1×, 2×, 3×, 4×, 5× or 10×, relative to an FDA approved dose. An CPI-169 dose is for example the concentration listed on Table 7, column titled “Human Dosage”

In some embodiments, the EZH2 inhibitor is CPI-360 and is administered for example to a cochlear cell in amount sufficient to achieve a concentration of about 0.001 nM to 1000 μM, about 0.01 nM to 100 μM, about 0.1 nM to 10 μM, about 1 nM to 1000 nM, about 1 nM to 10 nM, about 10 nM to 100 nM, about 100 nM to 1 μM, about 1000 nM to 10 μM, or about 10 μM to 100 μM, in the perilymph fluid in the inner ear.

In some embodiments, the CPI-360 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, or about 20 μM in the perilymph fluid in the inner ear.

In some embodiments, the EZH2 inhibitor is CPI-360 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1 mM, 1 mM to 10 mM, or about 10 mM to 100 mM.

In some embodiments, the CPI-360 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, or about 40 mM.

In some embodiments, the EZH2 inhibitor is CPI-360 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is CPI-360 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the EZH2 inhibitor is EPZ011989 and is administered for example to a cochlear cell in amount sufficient to achieve a concentration of about 0.001 nM to 100 μM, about 0.01 nM to 10 μM, about 0.1 nM to 1 μM, about 1 nM to 100 nM, about 1 nM to 10 nM, about 10 nM to 100 nM, or about 100 nM to 1 μM, in the perilymph fluid in the inner ear.

In some embodiments, the EPZ011989 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear.

In some embodiments, the EZH2 inhibitor is EPZ011989 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the EPZ011989 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the EZH2 inhibitor is EPZ011989 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is EPZ011989 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the EZH2 inhibitor is UNC 2399 and is administered for example to a cochlear cell in amount sufficient to achieve a concentration of about 0.001 nM to 100 μM, about 0.01 nM to 10 μM, about 0.1 nM to 1 μM, about 1 nM to 100 nM, about 1 nM to 10 nM, about 10 nM to 100 nM, about 100 nM to 1 μM about 1 μM to 10 μM or about 10 μM to 100 μM, in the perilymph fluid in the inner ear.

In some embodiments, the UNC 2399 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 15 μM, 20 μM, 30 μM or about 40 μM in the perilymph fluid in the inner ear.

In some embodiments, the EZH2 inhibitor is UNC 2399 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1 mM, 1 mM to 10 mM, or about 10 mM to 100 mM.

In some embodiments, the UNC 2399 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, or about 40 mM.

In some embodiments, the EZH2 inhibitor is UNC 2399 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is UNC 2399 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, the EZH2 inhibitor is PF-06726304 and is administered for example to a cochlear cell in amount sufficient to achieve a concentration of about 0.01 nM to 1 mM, about 0.1 nM to 100 μM, about 1 nM to 10 μM, about 10 nM to 1 μM, about 1 nM to 10 nM, about 10 nM to 100 nM, 100 nM to 1 μM, or about 1 μM to 10 μM, in the perilymph fluid in the inner ear.

In some embodiments, the PF-06726304 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the EZH2 inhibitor is PF-06726304 is administered to a subject, for example to the middle ear at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the PF-06726304 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the EZH2 inhibitor is PF-06726304 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the EZH2 inhibitor is PF-06726304 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, EZH2 inhibitor is PF-06726304 and is administered to the subject at about 0.01×. 0.1×, Ix, 2×, 3×, 4×, 5× or 10×, relative to an FDA approved dose. A PF-06726304 dose is for example the concentration listed on Table 7, column titled “Human Dosage”.

In some embodiments the additional epigenetic agent is a DOTL1 inhibitor.

In some embodiments, the DOT1L inhibitor is EPZ004777 and is administered for example to a cochlear cell in amount sufficient to achieve a concentration of about 0.01 nM to 1 mM, about 0.1 nM to 100 μM, about 1 nM to 100 μM, about 10 nM to 100 μM, about 1 nM to 10 nM, about 10 nM to 100 nM, about 100 nM to 1 μM, about 1 μM to 10 μM or about 10 μM to 100 μM, in the perilymph fluid in the inner ear.

In some embodiments, the EPZ004777 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the DOT1L inhibitor is EPZ004777 is administered to a subject, for example to the middle ear at a concentration of about 0.01 μM to 1000 mM, about 0.1 μM to 100 mM, about 1 μM to 10 mM, about 10 μM to 1 mM, 10 μM to 100 μM, about 100 sM to 1000 μM, about 1 mM to 10 mM, or about 10 mM to 100 mM.

In some embodiments, the EPZ004777 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the DOT1L inhibitor is EPZ004777 and is administered systemically at a daily dose of about 1-1000 mg/m2 per day IV, about 10-100 mg/m2 per day IV, about 10 mg/m2 per day IV, about 15 mg/m2 per day IV, about 20 mg/m2 per day IV, about 25 mg/m2 per day IV, about 30 mg/m2 per day IV, about 35 mg/m2 per day IV, about 40 mg/m2 per day IV, about 45 mg/m2 per day IV, about 50 mg/m2 per day IV, about 55 mg/m2 per day IV, about 60 mg/m2 per day IV, about 65 mg/m2 per day IV, about 70 mg/m2 per day IV, about 75 mg/m2 per day IV, about 80 mg/m2 per day IV, about 85 mg/m2 per day IV, about 90 mg/m2 per day IV, about 95 mg/m2 per day IV, about 100 mg/m2 per day IV, about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the DOT1L inhibitor is EPZ004777 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, DOT1L inhibitor is EPZ004777 and is administered to the subject at about 0.01×. 0.1×, Ix, 2×, 3×, 4×, 5× or 10×, relative to an FDA approved dose. An EPZ004777 dose is for example the concentration listed on Table 8, column titled “Human Dosage”.

In some embodiments, the DOT1L inhibitor is SGC0946 and is administered for example to a cochlear cell in amount sufficient to achieve a concentration of about 0.01 nM to 1 mM, about 0.1 nM to 100 μM, about 1 nM to 100 μM, about 10 nM to 100 μM, about 1 nM to 10 nM, about 10 nM to 100 nM, about 100 nM to 1 μM, about 1 μM to 10 μM or about 10 μM to 100 μM, in the perilymph fluid in the inner ear.

In some embodiments, the SGC0946 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the DOT1L inhibitor is SGC0946 is administered to a subject, for example to the middle ear at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the SGC0946 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 μM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the DOT1L inhibitor is SGC0946 and is administered systemically at a daily dose of about 1-1000 mg/m2 per day IV, about 10-100 mg/m2 per day IV, about 10 mg/m2 per day IV, about 15 mg/m2 per day IV, about 20 mg/m2 per day IV, about 25 mg/m2 per day IV, about 30 mg/m2 per day IV, about 35 mg/m2 per day IV, about 40 mg/m2 per day IV, about 45 mg/m2 per day TV, about 50 mg/m2 per day IV, about 55 mg/m2 per day IV, about 60 mg/m2 per day IV, about 65 mg/m2 per day IV, about 70 mg/m2 per day IV, about 75 mg/m2 per day IV, about 80 mg/m2 per day IV, about 85 mg/m2 per day IV, about 90 mg/m2 per day IV, about 95 mg/m2 per day IV, about 100 mg/m2 per day IV, about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the DOT1L inhibitor is SGC0946 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, DOT1L inhibitor is SGC0946 and is administered to the subject at about 0.01×. 0.1×, 1×, 2×, 3×, 4×, 5× or 10×, relative to an FDA approved dose. A SGC0946 dose is for example the concentration listed on Table 8, column titled “Human Dosage”.

In some embodiments, the DOT1L inhibitor is pinometostat and is administered for example to a cochlear cell in amount sufficient to achieve a concentration of about 0.01 nM to 1 mM, about 0.1 nM to 100 μM, about 1 nM to 100 μM, about 10 nM to 100 μM, about 1 nM to 10 nM, about 10 nM to 100 nM, about 100 nM to 1 μM, about 1 μM to 10 μM or about 10 μM to 100 μM, in the perilymph fluid in the inner ear.

In some embodiments, the pinometostat is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear.

In some embodiments, the DOT1L inhibitor is pinometostat is administered to a subject, for example to the middle ear at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pinometostat is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the DOT1L inhibitor is pinometostat and is administered systemically at a daily dose of about 1-1000 mg/m2 per day IV, about 10-100 mg/m2 per day IV, about 10 mg/m2 per day IV, about 15 mg/m2 per day IV, about 20 mg/m2 per day IV, about 25 mg/m2 per day IV, about 30 mg/m2 per day IV, about 35 mg/m2 per day IV, about 40 mg/m2 per day IV, about 45 mg/m2 per day TV, about 50 mg/m2 per day IV, about 55 mg/m2 per day IV, about 60 mg/m2 per day TV, about 65 mg/m2 per day IV, about 70 mg/m2 per day IV, about 75 mg/m2 per day TV, about 80 mg/m2 per day IV, about 85 mg/m2 per day IV, about 90 mg/m2 per day IV, about 95 mg/m2 per day IV, about 100 mg/m2 per day IV, about 10 mg to 5,000 mg/day, about 10 mg to 3000 mg/day, about 10 mg to 1000 mg/day, about 10 mg to 500 mg/day, 20 mg to 5,000 mg/day, about 20 mg to 1000 mg/day, about 20 mg to 500 mg/day, about 10 mg/day, about 25 mg/day, about 50 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, or about 1000 mg/day.

In some embodiments, the DOT1L inhibitor is pinometostat and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, DOT1L inhibitor is pinometostat and is administered to the subject at about 0.01×. 0.1×, Ix, 2×, 3×, 4×, 5× or 10×, relative to an FDA approved dose. A pinometostat dose is for example the concentration listed on Table 8, column titled “Human Dosage”.

In some embodiments, the additional epigenetic agent is an LSD1 inhibitor.

In some embodiments, the LSD-1 inhibitor “cell effective concentration” is about 0.01 μM to 100 μM, about 0.1 μM to 10 μM about 1 μM to 1 μM, about 0.01 μM to 10 μM, about 0.1 μM to 10 μM, about 1 μM to 10 μM, 10 μM to 100 μM, or about 100 μM to 1000 mM.

In some embodiments, the LSD-1 inhibitor “formulation effective concentration” is about 0.01 μM to 100 mM, about 0.1 μM to 10 mM, about 1 μM to 1 mM, about 0.01 mM to 10 mM, about 0.1 mM to 10 mM, about 1 μM to 10 μM, 10 μM to 100 μM, or about 100 μM to 1000 mM.

In some embodiment the LSD-1 is to a subject administered systemically at a daily dose of about 0.01 mg to 1000 mg/day; about 0.01 mg to 500 mg/day; about 0.01 mg to 250 mg/day; about 0.01 mg to 100 mg/day; about 0.01 mg to 50 mg/day; about 0.01 mg to 25 mg/day; about 0.01 mg to 10 mg/day; about 0.01 mg to 5 mg/day; 0.1 mg to 100 mg/day; about 0.1 mg to 50 mg/day; about 0.01 mg to 25 mg/day; about 0.01 mg to 10 mg/day; about 0.01 mg to 5 mg/day; about 0.01 mg to 2.5 mg/day; about 0.1 mg to 10 mg/day; about 0.1 mg to 5 mg/day about 0.1 mg to 4 mg/day; about 0.1 mg to 3 mg/day; about 0.1 mg to 2 mg/day; about 0.1 mg to 2 mg/day or about 1 mg to 5 mg/day.

In some embodiments, the LSD1 inhibitor is administered to the subject at a concentration ratio of about 0.001 to 10 fold relative to an FDA approved concentration or about 0.1 to 50 fold relative to an FDA approved concentration, or about 0.1 to 5 fold relative to an FDA approved, or about 1 to 5 fold relative to an FDA approved concentration. In some embodiments, LSD1 inhibitor is administered to the subject at about 0.01×. 0.1×, 2×, 3×, 5× or 10×, relative to an FDA approved concentration.

In some embodiments, the LSD1 inhibitor is GSK-2879552 and is administered for example to a cochlear cell in amount sufficient to achieve a concentration of about 0.001 nM to 1 mM, about 0.01 nM to 100 μM, about 0.1 nM to 10 μM, about 1 nM to 1 μM, about 1 nM to 10 nM, about 10 nM to 100 nM, about 100 nM to 1 μM, or about 1 μM to 10 μM in the perilymph fluid in the inner ear.

In some embodiments, the GSK-2879552 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7,μM, 8 μM, 9 μM, 10 μM, 12 μM, 14 μM, 16 μM, 18 μM, 20 μM, 25 μM, or about 30 μM in the perilymph fluid in the inner ear.

In some embodiments, the LSD1 inhibitor is GSK-2879552 is administered to a subject, for example to the middle ear at a concentration of 0.001 μM to 1,000 mM, about 0.01 sM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the GSK-2879552 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the LSD-1 is GSK-2879552 and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, or about 5-10 mg/day.

In some embodiments, the LSD1 inhibitor is GSK-2879552 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, LSD1 inhibitor is GSK-2879552 and is administered to the subject at about 0.01×. 0.1×, 1×, 2×, 3×, 4×, 5× or 10×, relative to an FDA approved concentration. A GSK-2879552 FDA approved concentration is for example the concentration listed on Table 9, column titled “Human Dosage”.

In some embodiments, the LSD1 inhibitor is GSK-LSD1 and is administered for example to a cochlear cell in amount sufficient to achieve a concentration of about 0.001 nM to 10 uM, about 0.01 nM to 1 uM, about 0.1 nM to 100 nM, about 0.001 nM to 0.01 nM, about 0.01 nM to 0.1 nM, about 0.1 nM to 1 nM, about 1 nM to 10 nM, about 10 nM to 100 nM, about 100 nM to 1,000 nM, 1 μM to 10 μM or about 10 μM to 100 μM in the perilymph fluid in the inner ear.

In some embodiments, the GSK-LSD1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 1 μM, 5 μM, 10 μM, or 50 μM in the perilymph fluid in the inner ear.

In some embodiments, the LSD1 inhibitor is GSK-LSD1 is administered to a subject, for example to the middle ear at a concentration of 0.001 μM to 10 mM, about 0.01 μM to 1 mM, about 0.1 μM to 100 μM, about 0.001 μM to 0.01 μM, about 0.01 μM to 0.1 μM, about 0.1 sM to 1 μM, about 1 μM to 10 μM, about 10 μM to 100 μM, about 100 μM to 1,000 μM or about 1 mM to 50 mM.

In some embodiments, the GSK-LSD1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the LSD-1 inhibitor is GSK-LSD1 and is administered to a subject systemically at a daily dose of about 0.01 mg to 500 mg/day, about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, about 5-10 mg/day, about 10-25 mg/day, about 25-50 mg/day, or about 50-100 mg/day.

In some embodiments, the LSD1 inhibitor is GSK-LSD1 and is administered to the subject at a concentration ratio of about 0.001 to 100 fold relative to an FDA approved concentration or about 0.01 to 50 fold relative to an FDA approved concentration, or about 0.1 to 10 fold relative to an FDA approved concentration, or about 0.1 to 5 fold relative to an FDA approved concentration, or about 1 to 5 fold relative to an FDA approved concentration.

In some embodiments, LSD1 inhibitor is GSK-LSD1 and is administered to the subject at about 0.01×. 0.1×, 2×, 3×, 4×, 5× or 10×, relative to an FDA approved concentration. A GSK-LSD1 FDA approved concentration is for example the concentration listed on Table 9, column tided “Human Dosage”.

In some embodiments, the LSD-1 inhibitor is Tranylcypromine, and is administered for example to a cochlear cell in amount sufficient to achieve a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, about 100 μM to 1,000 μM, or about 1 mM to 10 mM in the perilymph fluid in the inner ear.

In some embodiments, the Tranylcypromine is administered, for example to a cochlear cell in amount sufficient to achieve a concentration of about 0.1 μM, 1 μM, 2 μM, 3 μM, 4 μM, 5 μM, 6 μM, 7 μM, 8 μM, 9 μM, 10 μM, 12 μM, 14 μM, 16 μM, 18 μM or 20 μM in the perilymph fluid in the inner ear.

In some embodiments, the LSD-1 inhibitor is Tranylcypromine, and is administered to a subject, for example to the middle ear at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the Tranylcypromine to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM.

In some embodiments, the LSD-1 inhibitor is Tranylcypromine and is administered to a subject systemically at a daily dose of about 1.5 mg to 750 mg/day, about 5 mg to 500 mg/day, about 10 mg to 250 mg/day, about 15 mg to 150 mg/day, about 1.5 mg to 10 mg/day, about 10 mg to 20 mg/day, about 20 mg to 30 mg/day, about 30 mg to 40 mg/day, about 40 mg to 50 mg/day, about 50 mg to 60 mg/day, about 60 mg to 70 mg/day, about 70 mg to 80 mg/day, about 90 mg to 100 mg/day, about 100 mg to 120 mg/day, or about 120 mg to 150 mg/day.

In some embodiments, the LSD1 inhibitor is Tranylcypromine and is administered to the subject at a concentration ratio of about 0.001 to 100 fold relative to an FDA approved concentration or about 0.01 to 50 fold relative to an FDA approved concentration, or about 0.1 to 5 fold relative to an FDA approved, or about 1 to 5 fold relative to an FDA approved concentration.

In some embodiments, LSD1 inhibitor is Tranylcypromine and is administered to the subject at about 0.01×. 0.1×, 2×, 3×, 4×, 5× or 10×, relative to an FDA approved concentration. A Tranylcypromine FDA approved concentration is for example the concentration listed on Table 9, column titled “Human Dosage”.

In some embodiments, the LSD-1 inhibitor is Phenelzine sulfate, and is administered for example to a cochlear cell in amount sufficient to achieve a concentration of 0.001 μM to 100 mM, about 0.01 μM to 10 mM, about 0.1 μM to 1 mM, about 1 μM to 100 μM, about 0.001 μM to 0.01 μM, about 0.01 μM to 0.1 μM, about 0.1 μM to 1 μM, about 1 μM to 10 μM, about 10 sM to 100 μM, about 100 μM to 1,000 μM, or about 1 mM to 10 mM in the perilymph fluid in the inner ear.

In some embodiments, the Phenelzine sulfate is administered, for example to a cochlear cell in amount sufficient to achieve a concentration of about 0.1 μM, 0.2 μM, 0.3 μM, 0.4 uM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM or 10 μM in the perilymph fluid in the inner ear.

In some embodiments, the LSD-1 inhibitor is Phenelzine sulfate, and is administered to a subject, for example to the middle ear at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the Phenelzine sulfate is administered to a subject, for example to the middle ear at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the LSD-1 inhibitor is Phenelzine sulfate and is administered to a subject systemically at a daily dose of about 1.5 mg to 750 mg/day, about 5 mg to 500 mg/day, about 10 mg to 250 mg/day, about 15 mg to 150 mg/day, about 1.5 mg to 10 mg/day, about 10 mg to 20 mg/day, about 20 mg to 30 mg/day; about 30 mg to 40 mg/day; about 40 mg to 50 mg/day about 50 mg to 60 mg/day; about 60 mg to 70 mg/day; about 70 mg to 80 mg/day; or about 90 mg to 100 mg/day

In some embodiments, the LSD1 inhibitor is Phenelzine sulfate and is administered to the subject at a concentration ratio of about 0.001 to 100 fold relative to an FDA approved concentration or about 0.01 to 50 fold relative to an FDA approved concentration, or about 0.1 to 5 fold relative to an FDA approved, or about 1 to 5 fold relative to an FDA approved concentration.

In some embodiments, LSD1 inhibitor is Phenelzine sulfate and is administered to the subject at about 0.01×. 0.1×, 2×, 3×, 4×, 5× or 10×, relative to an FDA approved concentration. A Tranylcypromine FDA approved concentration is for example the concentration listed on Table 9, column titled “Human Dosage”.

In some embodiments, the LSD1 inhibitor is ORY-1001 and is administered for example to a cochlear cell in amount sufficient to achieve a concentration of about 0.001 nM to 1 mM, about 0.01 nM to 100 μM, about 0.1 nM to 10 μM, about 1 nM to 1 μM, about 1 nM to 10 nM, about 10 nM to 100 nM, about 100 nM to 1 μM, or about 1 μM to 10 μM in the perilymph fluid in the inner ear.

In some embodiments, the ORY-1001 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear.

In some embodiments, the LSD1 inhibitor is ORY-1001 is administered to a subject, for example to the middle ear at a concentration of 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the ORY-1001 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the LSD-1 inhibitor is ORY-1001 and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, or about 5-10 mg/day.

In some embodiments, the LSD1 inhibitor is ORY-1001 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, LSD1 inhibitor is ORY-1001 and is administered to the subject at about 0.01×. 0.1×, lx, 2×, 3×, 4×, 5× or 10×, relative to an FDA approved concentration. An ORY-1001 FDA approved concentration is for example the concentration listed on Table 1, column titled “Human Dosage”.

In some embodiments, the LSD1 inhibitor is RN-1 and is administered for example to a cochlear cell in amount sufficient to achieve a concentration of about 0.001 nM to 1 mM, about 0.01 nM to 100 μM, about 0.1 nM to 10 μM, about 1 nM to 1 μM, about 1 nM to 10 nM, about 10 nM to 100 nM, about 100 nM to 1 μM, or about 1 μM to 10 μM in the perilymph fluid in the inner ear.

In some embodiments, the RN-1 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 12 μM, 14 μM, 16 μM, 18 μM, 20 μM, 25 μM, or about 30 μM in the perilymph fluid in the inner ear.

In some embodiments, the LSD1 inhibitor is RN-1 is administered to a subject, for example to the middle ear at a concentration of 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the RN-1 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM or about 30 mM.

In some embodiments, the LSD-1 inhibitor is RN-1 and is administered to a subject systemically at a daily dose of about 0.01 mg to 500 mg/day about 0.1 mg to 100 mg/day, about 1 mg to 50 mg/day, about 1 mg to 25 mg/day, about 1 mg to 10 mg/day, about 1 mg to 5 mg/day, about 0.01 mg to 0.1 mg/day, about 0.1 mg to 1 mg/day, about 1 mg to 10 mg/day, about 10 mg to 100 mg/day, about 100 mg to 500 mg/day, about 0.5 mg to 1 mg/day, about 1 mg to 2 mg/day, about 2 mg to 3 mg/day, about 3 mg to 4 mg/day, about 4 mg to 5 mg/day, or about 5-10 mg/day.

In some embodiments, the LSD1 inhibitor is RN-1 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, LSD1 inhibitor is RN-1 and is administered to the subject at about 0.01×. 0.1×, Ix, 2×, 3×, 4×, 5× or 10×, relative to an FDA approved concentration. An RN-1 2879552 FDA approved concentration is for example the concentration listed on Table 1, column titled “Human Dosage”.

In some embodiments, the KDM inhibitor is AS 8351 and is administered for example to a cochlear cell in amount sufficient to achieve a concentration of about 0.01 nM to 1 mM, about 0.1 nM to 100 μM, about 1 nM to 10 μM, about 10 nM to 10 μM, about 1 nM to 10 nM, about 10 nM to 100 nM, 100 nM to 1 μM, or about 1 μM to 10 μM, in the perilymph fluid in the inner ear.

In some embodiments, the AS 8351 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the KDM inhibitor is AS 8351 is administered to a subject, for example to the middle ear at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the AS 8351 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9n M, or 10 mM.

In some embodiments, the KDM inhibitor is AS 8351 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the KDM inhibitor is AS 8351 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, KDM inhibitor is AS 8351 and is administered to the subject at about 0.01×. 0.1×, 1×, 2×, 3×, 4×, 5× or 10×, relative to an FDA approved dose. An AS 8351 dose is for example the concentration listed on Table 10 column titled “Human Dosage”.

In some embodiments, the KDM inhibitor is TC-E 5002 and is administered for example to a cochlear cell in amount sufficient to achieve a concentration of about 0.01 nM to 1 mM, about 0.1 nM to 100 μM, about 1 nM to 10 μM, about 10 nM to 10 μM, about 1 nM to 10 nM, about 10 nM to 100 nM, 100 nM to 1 μM, or about 1 μM to 10 μM, in the perilymph fluid in the inner ear.

In some embodiments, the TC-E 5002 is administered, in amount sufficient to achieve a concentration of about 10 nM, 50 nM, 75 nM, 100 nM, 110 nM, 120 nM, 130 nM, 140 nM, 150 nM, 160 nM, 170 nM, 180 nM, 190 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, or about 10 μM, in the perilymph fluid in the inner ear.

In some embodiments, the KDM inhibitor is TC-E 5002 is administered to a subject, for example to the middle ear at a concentration of about 0.01 μM to 1000 mM, about 0.1 μM to 100 mM, about 1 μM to 10 mM, about 10 μM to 1000 μM, about 1 μM to 10 μM, 10 μM to 100 s M, about 100 μM to 1000 μM or about 1 mM to 10 mM.

In some embodiments, the AS TC-E 5002 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the KDM inhibitor is TC-E 5002 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1600 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the KDM inhibitor is TC-E 5002 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, KDM inhibitor is TC-E 5002 and is administered to the subject at about 0.01×. 0.1×, 1×, 2×, 3×, 4×, 5× or 10×, relative to an FDA approved dose. An TC-E 5002 dose is for example the concentration listed on Table 10, column titled “Human Dosage”.

In some embodiments, the KDM inhibitor is EPT-103182 and is administered for example to a cochlear cell in amount sufficient to achieve a concentration of about 0.001 nM to 100 μM, about 0.01 nM to 10 μM, about 0.1 nM to 1 s M, about 1 nM to 100 nM, about 1 nM to 10 nM, about 10 nM to 100 nM, or about 100 nM to 1 μM, in the perilymph fluid in the inner ear

In some embodiments, the EPT-103182 is administered, in amount sufficient to achieve a concentration of about 0.1 nM, 0.2 nM, 0.3 nM, 0.4 nM, 0.5 nM, 0.6 nM, 0.7 nM, 0.8 nM, 0.9 nM, 1.0 nM, 2.0 nM, 3.0 nM, 4.0 nM, 5.0 nM, 6.0 nM, 7.0 nM, 8.0 nM, 9.0 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, or about 1 μM in the perilymph fluid in the inner ear.

In some embodiments, the KDM inhibitor is EPT-103182 is administered to a subject, for example to the middle ear at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 μM to 1 mM.

In some embodiments, the EPT-103182 is administered to a subject, for example to the middle ear at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the KDM inhibitor is EPT-103182 and is administered systemically at a daily dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 150 mg to 2500 mg/day, about 150 mg to 2000 mg/day, about 150 mg to 1500 mg/day, about 150 mg to 1250 mg/day, about 75 mg/day, about 100 mg/day, about 150 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the KDM inhibitor is EPT-103182 and is administered to the subject at a concentration ratio of about 0.001 to 100 fold relative to an FDA approved concentration or about 0.01 to 50 fold relative to an FDA approved concentration or about 0.1 to 10 fold relative to an FDA approved concentration, or about 0.1 to 5 fold relative to an FDA approved, or about 1 to 5 fold relative to an FDA approved concentration.

In some embodiments, KDM inhibitor is EPT-103182 and is administered to the subject at about 0.01×. 0.1×, lx, 2×, 3×, 4×, 5× or 10×, relative to an FDA approved dose. An EPT-103182 dose is for example the concentration listed on Table 10, column titled “Human Dosage”.

In some embodiments the TAZ activator is IBS008738 and the Wnt agonist is AZD1080. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the TAZ activator is IBS008738 and the Wnt agonist is LY2090314. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and LY2090314, and is administered to a subject, for example to the middle ear at a concentration of about 1 μM, 5 μM, 10 μM, 15 μM, 20 μM, or 40 μM.

In some embodiments the TAZ activator is IBS008738 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM. 3 μM, 4 μM. 5 μM, 6 μM. 7 μM, 8 μM. 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the TAZ activator is IBS008738 and the Wnt agonist is GSK3 inhibitor XXII. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the 1BS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the TAZ activator is IBS008738 and the Wnt agonist is CHIR99021. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the TAZ activator is TT-10 and the Wnt agonist is AZD1080. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the TAZ activator is T-10 and the Wnt agonist is LY2090314. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and LY2090314, and is administered to a subject, for example to the middle ear at a concentration of about 1 μM, 5 μM, 10 μM, 15 μM, 20 μM, or 40 μM.

In some embodiments the TAZ activator is TT-10 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the TAZ activator is TT-10 and the Wnt agonist is GSK3 inhibitor XXII. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 nM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the TAZ activator is TT-10 and the Wnt agonist is CHIR99021. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear and CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the TAZ activator is TM-25659 and the Wnt agonist is AZD1080. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 IM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear and AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear.

Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 μM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the TAZ activator is TM-25659 and the Wnt agonist is LY2090314. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear and LY2090314 is administered, in amount sufficient to achieve a concentration of about 1 nM, 5 nM, 10 nM, 15 nM, 20 nM, or 40 nM in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and LY2090314, and is administered to a subject, for example to the middle ear at a concentration of about 1 μM, 5 μM, 10 μM, 15 μM, 20 μM, or 40 μM.

In some embodiments the TAZ activator is TM-25659 and the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear and the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM.

In some embodiments the TAZ activator is TM-25659 and the Wnt agonist is GSK3 inhibitor XXII. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear and GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear.

In some embodiments the TAZ activator is TM-25659 and the Wnt agonist is CHIR99021. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear and CHR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM.

In some embodiments the TAZ activator is IBS008738; the Wnt agonist is AZD1080 and the epigenetic agent is VPA. In some embodiments, the 1BS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the TAZ activator is IBS008738; the Wnt agonist is LY2090314 and the epigenetic agent is VPA. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; LY2090314 is administered, in amount sufficient to achieve a concentration of about 1 nM, 5 nM, 10 nM, 15 nM, 20 nM, or 40 nM in the perilymph fluid in the inner ear and VPA is administered in amount sufficient to achieve a concentration of about is about 100 μM to 4 mM in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; LY2090314, and is administered to a subject, for example to the middle ear at a concentration of about 1 μM, 5 μM, 10 μM, 15 μM, 20 μM, or 40 μM and VPA to a subject, for example to the middle ear at a concentration about 100 mM to 4,000 mM.

In some embodiments the TAZ activator is IBS008738; the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the epigenetic agent is VPA. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the 1BS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 sM and VPA to a subject, for example to the middle ear at a concentration about 100 mM to 4,000 mM.

In some embodiments the TAZ activator is IBS008738; the Wnt agonist is GSK3 inhibitor XXII and the epigenetic agent is VPA. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA to a subject, for example to the middle ear at a concentration about 100 mM to 4,000 mM.

In some embodiments the TAZ activator is IBS008738; the Wnt agonist is CHIR99021 and the epigenetic agent is VPA. In some embodiments, the IBS008738 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the IBS008738 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the TAZ activator is TT-10; the Wnt agonist is AZD1080 and the epigenetic agent is VPA. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the TAZ activator is TT-10, the Wnt agonist is LY2090314 and the epigenetic agent is VPA. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 4 μM, 5 μM, 6 μM, 6 μM, 7 μM, 8 μM, 9 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; LY2090314 is administered, in amount sufficient to achieve a concentration of about 1 nM, 5 nM, 10 nM, 15 nM, 20 nM, or 40 nM in the perilymph fluid in the inner ear and VPA is administered in amount sufficient to achieve a concentration of about is about 100 μM to 4 mM in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; LY2090314, and is administered to a subject, for example to the middle ear at a concentration of about 1 μM, 5 μM, 10 μM, 15 μM, 20 μM, or 40 μM and VPA to a subject, for example to the middle ear at a concentration about 100 mM to 4,000 mM.

In some embodiments the TAZ activator is TT-10, the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the epigenetic agent is VPA. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA to a subject, for example to the middle ear at a concentration about 100 mM to 4,000 mM.

In some embodiments the TAZ activator is TT-10, the Wnt agonist is GSK3 inhibitor XXII and the epigenetic agent is VPA. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA to a subject, for example to the middle ear at a concentration about 100 mM to 4,000 mM.

In some embodiments the TAZ activator is TT-10; the Wnt agonist is CIR99021 and the epigenetic agent is VPA. In some embodiments, the TT-10 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 4 μM, 5 μM, 6 μM, 6 μM, 7 μM, 8 μM, 9 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the TT-10 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the TAZ activator is TM-25659; the Wnt agonist is AZD1080 and the epigenetic agent is VPA. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM; AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the TAZ activator is TM-25659; the Wnt agonist is LY2090314 and the epigenetic agent is VPA. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear; LY2090314 is administered, in amount sufficient to achieve a concentration of about 1 nM, 5 nM, 10 nM, 15 nM, 20 nM, or 40 nM in the perilymph fluid in the inner ear and VPA is administered in amount sufficient to achieve a concentration of about is about 100 μM to 4 mM in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM; LY2090314, and is administered to a subject, for example to the middle ear at a concentration of about 1 μM, 5 μM, 10 μM, 15 μM, 20 μM, or 40 μM and VPA to a subject, for example to the middle ear at a concentration about 100 mM to 4,000 mM.

In some embodiments the TAZ activator is TM-25659; the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the epigenetic agent is VPA. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM; the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA to a subject, for example to the middle ear at a concentration about 100 mM to 4,000 mM.

In some embodiments the TAZ activator is TM-25659, the Wnt agonist is GSK3 inhibitor XXII and the epigenetic agent is VPA. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM; the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA to a subject, for example to the middle ear at a concentration about 100 mM to 4,000 mM.

In some embodiments the TAZ activator is TM-25659; the Wnt agonist is CHIR99021 and the epigenetic agent is VPA. In some embodiments, the TM-25659 is administered, in amount sufficient to achieve a concentration of about 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, or 100 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the TM-25659 is administered to a subject, for example to the middle ear at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM; CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the TAZ activator is FHZ-000706; the Wnt agonist is AZD1080 and the epigenetic agent is VPA. In some embodiments, the FHZ-000706 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; AZD1080 is administered, in amount sufficient to achieve a concentration of about is about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the FHZ-000706 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM AZD1080, and is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mM.

In some embodiments the TAZ activator is FHZ-000706; the Wnt agonist is LY2090314 and the epigenetic agent is VPA. In some embodiments, the FHZ-000706 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; LY2090314 is administered, in amount sufficient to achieve a concentration of about 1 nM, 5 nM, 10 nM, 15 nM, 20 nM, or 40 nM in the perilymph fluid in the inner ear and VPA is administered in amount sufficient to achieve a concentration of about is about 100 μM to 4 mM in the perilymph fluid in the inner ear. Alternatively, the FHZ-000706 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 LM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM: LY2090314, and is administered to a subject, for example to the middle ear at a concentration of about 1 μM, 5 μM, 10 μM, 15 μM, 20 μM, or 40 μM and VPA to a subject, for example to the middle ear at a concentration about 100 mM to 4,000 mM.

In some embodiments the TAZ activator is FHZ-000706; the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione and the epigenetic agent is VPA. In some embodiments, the FHZ-000706 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; the substituted 3-Imidazo[1,2-a]pyridin-3-yl-4-(1,2,3,4-tetrahydro-[1,4]diazepino-[6,7,1-hi]indol-7-yl)pyrrole-2,5-dione is administered, in amount sufficient to achieve a concentration of about 1 nM, 5 nM, 10 nM, 15 nM, 20 nM, 50 nM, 100 nM, 250 nM, or 500 nM, in the perilymph fluid in the inner ear and VPA is administered in amount sufficient to achieve a concentration of about is about 100 μM to 4 mM in the perilymph fluid in the inner ear. Alternatively, the FHZ-000706 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; the substituted 3-Imidazo[1,2-a]pyridin-3-yl-4-(1,2,3,4-tetrahydro-[1,4]diazepino-[6,7,1-hi]indol-7-yl)pyrrole-2,5-dione and is administered to a subject, for example to the middle ear at a concentration of about 1 μM, 5 μM, 10 μM, 15 μM, 20 μM, 50 μM, 100 μM, 250 μM, or 500 μM and VPA to a subject, for example to the middle ear at a concentration about 100 mM to 4,000 mM.

In some embodiments the TAZ activator is FHZ-000706; the Wnt agonist is GSK3 inhibitor XXII and the epigenetic agent is VPA. In some embodiments, the FHZ-000706 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 20 μM, 25 μM, 30 μM, 35 μM, 40 μM, 45 μM, or about 50 μM in the perilymph fluid in the inner ear; GSK3-inhibitor XXII is administered, in amount sufficient to achieve a concentration of about 0.1 μM, 0.2 μM, 0.3 μM, 0.4 μM, 0.5 μM, 0.6 μM, 0.7 μM, 0.8 μM, 0.9 μM, or 1.0 μM, in the perilymph fluid in the inner ear and VPA is administered in amount sufficient to achieve a concentration of about is about 100 μM to 4 mM in the perilymph fluid in the inner ear. Alternatively, the FHZ-000706 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; the GSK3-inhibitor XXII is administered, in amount sufficient to achieve a concentration of about 0.1 mM, 0.2 mM, 0.3 mM, 0.4 mM, 0.5 mM, 0.6 mM, 0.7 mM, 0.8 mM, 0.9 mM, or 1.0 mM, in the perilymph fluid in the inner ear and VPA to a subject, for example to the middle ear at a concentration about 100 mM to 4,000 mM.

In some embodiments the TAZ activator is FHZ-000706; the Wnt agonist is CHIR99021 and the epigenetic agent is VPA. In some embodiments, the FHZ-000706 is administered, in amount sufficient to achieve a concentration of about 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 11 μM, 12 μM, 13 μM 14 μM, 15 μM, 20 μM, 25 μM, 30 μM, 35 μM, 40 μM, 45 μM, or about 50 μM in the perilymph fluid in the inner ear; CHIR99021 is administered, in amount sufficient to achieve a concentration of about 1 μM, 2 μM, 3 μM, 4 μM, 5 μM, 6 μM, 7 μM, 8 μM, 9 μM, or 10 μM, in the perilymph fluid in the inner ear and VPA is administered in amount sufficient to achieve a concentration of about is about 100 μM to 4 mM in the perilymph fluid in the inner ear. Alternatively, the FHZ-000706 is administered to a subject, for example to the middle ear at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM; CHIR99021 is administered to a subject, for example to the middle ear at a concentration of about 1 mM, 2 mM, 3 mM, 4 mM, 5 mM, 6 mM, 7 mM, 8 mM, 9 mM, or 10 mM and VPA to a subject, for example to the middle ear at a concentration about 100 mM to 4,000 mlv.

Some embodiments comprise administering the (i) TAZ activator and (ii) Wnt agonist together in the same pharmaceutical composition, as described herein. Some embodiments comprise administering the (i) TAZ activator and (ii) Wnt agonist separately in separate pharmaceutical compositions.

Some embodiments comprise administering the (i) TAZ activator, (ii) Wnt agonist, and (iii) the additional epigenetic agent(s) together in the same pharmaceutical composition, as described herein. Some embodiments comprise administering the (i) TAZ activator (ii) Wnt agonist and (iii) the additional epigenetic agent(s) Wnt agonist separately in separate pharmaceutical compositions.

Some embodiments comprise administering the (i) TAZ activator, (ii) Wnt agonist, and (iii) the additional epigenetic agent(s) together in the same pharmaceutical composition, as described herein and the (iii) epigenetic agent in a pharmaceutical composition.

### Pharmaceutical Compositions and Administration

Certain embodiments relate to pharmaceutical, prophylactic, and/or therapeutic compositions, comprising a pharmaceutically-acceptable carrier and an TAZ activator and a Wnt agonist (and optionally an epigenetic agent,) a pharmaceutically-acceptable salt thereof or combinations thereof as described herein (collectively referred to herein as the “compound(s)”).

Certain embodiments relate to pharmaceutical, prophylactic, and/or therapeutic compositions, comprising a pharmaceutically-acceptable carrier and a TAZ activator and a Wnt agonist (and optionally an epigenetic agent,) a pharmaceutically-acceptable salt thereof or combinations thereof as described herein (collectively referred to herein as the “compound(s)”). In some embodiments, the concentration of the compound(s) in the pharmaceutical compositions of the invention are at the “formulation effective concentration” as described supra. In some embodiments, the pharmaceutical composition comprises a TAZ activator at a concentration of about 0.01 nM to 1000 μM, about 1 nM to 100 μM, about 10 nM to 10 μM, about 1 nM to 10 μM, about 10 μM to 100 μM, about 100 μM to 1000 μM, about 1 μM to 10 μM, 0.01 mM to 1000 mM, about 1 mM to 100 mM, or about 10 mM to 100 mM.

In some embodiments, the pharmaceutical composition comprises a TAZ activator that is IBS008738 at a unit dose of about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a IBS008738 that is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the pharmaceutical composition comprises a IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a TAZ activator that is TT-10 at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the pharmaceutical composition comprises a TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a TAZ activator is TT-10 at a unit dose of about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a TAZ activator is TM-25659 at a concentration of about 0.01 μM to 1000 mM, about 0.1 μM to 100 mM, about 1 sM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the pharmaceutical composition comprises a TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM.

In some embodiments, the pharmaceutical composition comprises a TAZ activator is TM-25659 at a unit dose of about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a TAZ activator that is FHZ-000706 at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM.

In some embodiments, the pharmaceutical composition comprises a FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a TAZ activator is FHZ-000706 at a unit dose of about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a GSK3 Inhibitor that is AZD1080, at a concentration of about 0.001 mM to 10,000 mM, about 0.01 mM to 1,000 nM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. In some embodiments, the AZD1080 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a GSK3 Inhibitor that is LY2090314 at a concentration of about 0.001 μM to 10 mM, about 0.01 μM to 1 mM, about 0.1 μM to 100 uM, about 0.001 μM to 0.01 μM, about 0.01 μM to 0.1 s M, about 0.1 μM to 1 μM, about 1 μM to 10 μM, about 10 μM to 100 μM, about 100 μM to 1 mM, or about 1 mM to 10 mM. In some embodiments, LY2090314 the is at a concentration of about 1 μM, 5 μM, 10 μM, 15 μM, 20 μM, or 40 μM.

In some embodiments, the pharmaceutical composition comprises a GSK3 Inhibitor that is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. In some embodiments, the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM, or 500 μM.

In some embodiments, the pharmaceutical composition comprises a GSK3 Inhibitor that is GSK3-inhibitor XXII, at a concentration of about of about 0.1 μM to 1,000 mM, about 1 sM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM. In some embodiments, the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a GSK3 Inhibitor that is CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM. In some embodiments, the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises an epigenetic agent that is an HDAC inhibitor at a concentration about 10 uM to 1,000,000 mM, about 1000 uM to 100,000 mM, about 10,000 uM to 10,000 mM, about 1000 uM to 10,000 uM, about 10,000 uM to 100,000 uM, about 100,000 uM to 1,000,000 uM, about 1,000 mM to 10,000 mM, or about 10,000 mM to 100,000 mM.

In some embodiments, the pharmaceutical composition comprises a HDAC inhibitor that is VPA at a concentration about 100 mM to 4,000 mM.

In some embodiments, the pharmaceutical composition comprises VPA at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of VPA at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises a HDAC inhibitor that is 2-hexyl-4-pentynoic acid at concentration about 100 mM to 4,000 mM.

In some embodiments, the pharmaceutical composition comprises 2-hexyl-4-pentynoic acid at a unit dose of 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of 2-hexyl-4-pentynoic acid at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises, Na phenylbutyrate that is at a concentration about 100 mM to 4,000 mM.

In some embodiments, the pharmaceutical composition comprises Na phenylbutyrate at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an oral dosage form of the Na phenylbutyrate at a unit dose of about 50 mg, about 100 mg, about 125 mg, about 250 mg, about 500 mg, 1000 mg, 2000 mg, 3000 mg, 4000 mg, or about 5000 mg

In some embodiments, the pharmaceutical composition comprises an epigenetic agent that is an EZH2 inhibitor

In some embodiments, the pharmaceutical composition comprises an EZH2 inhibitor that is PF-06821497 at a concentration of 0.001 μM to 100 mM, about 0.01 μM to 10 mM, about 0.1 μM to 1 mM, about 1 μM to 100 μM, about 1 μM to 10 μM, 10 μM to 100 μM or about 100 μM to 1 mM.

In some embodiments, the pharmaceutical composition comprises an EZH2 inhibitor that is PF-06821497 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises PF-06821497 at a daily dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 150 mg to 2500 mg, about 150 mg to 2000 mg, about 150 mg to 1500 mg, about 150 mg to 1250 mg, about 75 mg, about 100 mg, about 150 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is CPI-1205 at a concentration of 0.001 μM to 100 mM, about 0.01 μM to 10 mM, about 0.1 μM to 1 mM, about 1 μM to 100 μM, about 1 μM to 10 μM, about 10 μM to 100 μM, or about 100 μM to 1000 μM.

In some embodiments, the pharmaceutical composition comprises CPI-1205 is that is at a concentration of about 0.1 μM, 0.2 μM, 0.3 μM, 0.4 μM, 0.5 μM, 0.6 μM, 0.7 μM, 0.8 μM 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises an EZH2 inhibitor that is CPI-1205 ay a unit dose about 100 to 5,000 mg, about 100 mg to 4000 mg, about 100 mg to 3000 mg, about 100 mg to 2000 mg, about 500 to 5,000 mg, about 500 mg to 4000 mg, about 500 mg to 3000 mg, about 750 to 5,000 mg, about 750 mg to 4000 mg, about 750 mg to 3000 mg, about 800 mg to 2400 mg, about 400 mg, about 600 mg, about 800 mg, about 1000 mg, about 1200 mg, about 1400 mg, about 1600 mg, about 1800 mg, about 2000 mg, about 2200 mg, about 2400 mg, about 2600 mg, about 2800 mg, about 3000 mg, about 3250 mg, about 3500 mg, about 4000 mg, about 4500 mg, or about 5000 mg.

In some embodiments, the pharmaceutical composition comprises an EZH2 inhibitor that is valemetostat at a concentration of about 0.001 μM to 100 mM, about 0.01 μM to 10 mM, about 0.1 μM to 1 mM, about 1 μM to 100 μM about 1 μM to 10 μM, 10 μM to 100 μM, or about 100 μM to 1000 μM.

In some embodiments, the pharmaceutical composition comprises Valemetost that is at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or 1 mM.

In some embodiments, the pharmaceutical composition comprises an EZH2 inhibitor is valemetostat at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2000 mg, about 100 mg to 1500 mg, about 100 mg to 1000 mg, about 100 mg to 500 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is tazemetostat at a concentration of about 0.001 μM to 100 mM, about 0.01 μM to 10 mM, about 0.1 μM to 1 mM, about 1 μM to 100 μM, about 1 μM to 10 μM, 10 μM to 100 μM, about 100 μM to 1000 μM or about 1 mM to 10 mM.

In some embodiments, the pharmaceutical composition comprises tazemetostat t at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises an EZH2 inhibitor that is tazemetostat at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is E11 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor is E11 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1500 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises an EZH2 inhibitor that is CPI-169 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises CPI-169 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is CPI-169 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises an EZH2 inhibitor that is CPI-360 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 sM to 1000 μM, 1 mM to 10 mM, or about 10 mM to 100 mM.

In some embodiments, the pharmaceutical composition comprises CPI-360 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is CPI-360 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises an EZH2 inhibitor that is EPZ011989 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises EPZ011989 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is EPZ011989 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments, the pharmaceutical composition comprises an EZH2 inhibitor that is UNC 2399 at a concentration of about 0.1 μM to 1000 mM, about 1 μM to 100 mM, about 10 μM to 10 mM, about 100 μM to 10 mM, about 1 μM to 10 μM, 10 μM to 100 μM, about 100 μM to 1000 μM, 1 mM to 10 mM, or about 10 mM to 100 mM.

In some embodiments, the pharmaceutical composition comprises UNC 2399 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 15 mM, 20 mM, 25 mM, 30 mM, 35 mM, 40 mM, 45 mM, or about 50 mM.

In some embodiments, the pharmaceutical composition comprises an EZH2 inhibitor that is UNC 2399 at a unit dose of about 50 mg to 5,000 mg/day, about 50 mg to 4000 mg/day, about 50 mg to 3000 mg/day, about 50 mg to 2000 mg/day, about 50 mg to 1000 mg/day, about 50 mg to 500 mg/day, about 100 mg to 2500 mg/day, about 100 mg to 2000 mg/day, about 100 mg to 1500 mg/day, about 100 mg to 1000 mg/day, about 100 mg to 500 mg/day, about 200 mg to 2500 mg/day, about 200 mg to 2000 mg/day, about 200 mg to 1500 mg/day, about 200 mg to 1000 mg/day, about 100 mg/day, about 200 mg/day, about 300 mg/day, about 400 mg/day, about 500 mg/day, about 600 mg/day, about 700 mg/day, about 800 mg/day, about 900 mg/day, about 1000 mg/day, about 1200 mg/day, about 1400 mg/day, about 1600 mg/day, about 1800 mg/day, or about 2000 mg/day.

In some embodiments the additional epigenetic agent is a DOTL1 inhibitor.

In some embodiments, the pharmaceutical composition comprises a DOT1L inhibitor that is EPZ004777 at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is EPZ004777 at a concentration of about 0.01 μM to 1000 mM, about 0.1 μM to 100 mM, about 1 μM to 10 mM, about 10 μM to 1 mM, 10 μM to 100 μM about 100 μM to 1000 μM about 1 mM to 10 mM, or about 10 mM to 100 mM.

In some embodiments, the pharmaceutical composition comprises EPZ004777 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor is EPZ004777 at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor is EPZ004777 formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is SGC0946 at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises SGC0946 that is at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor is SGC0946 at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor is SGC0946 formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is pinometostat at a concentration of about 0.01 μM to 1000 mM, about 0.1 μM to 100 mM, about 1 μM to 10 mM, about 10 μM to 1 mM, 10 μM to 100 μM, about 100 μM to 1000 μM, about 1 mM to 10 mM, or about 10 mM to 100 mM.

In some embodiments, the pharmaceutical composition comprises a pinometostat a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM.

In some embodiments, the pharmaceutical composition comprises a DOT1L inhibitor that is pinometostat at a unit dose of about 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the pharmaceutical composition comprises a DOT1L inhibitor that is pinometostat formulated for IV administration at a unit dose of 1-1000 mg, about 10-100 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 10 mg to 5,000 mg, about 10 mg to 3000 mg, about 10 mg to 1000 mg, about 10 mg to 500 mg, 20 mg to 5,000 mg, about 20 mg to 1000 mg, about 20 mg to 500 mg, about 10 mg, about 25 mg, about 50 mg, about 75 mg, about 100 mg, about 150 mg, about 200 mg, about 300 mg, about 400 mg, about 500 mg, about 600 mg, about 700 mg, about 800 mg, about 900 mg, or about 1000 mg.

In some embodiments, the additional epigenetic agent is an LSD1 inhibitor.

In some embodiments, the pharmaceutical composition comprises a LSD1-inhibitor that is GSK-2879552 at a concentration of about 0.001 μM to 1,000 mM, about 0.01 μM to 100,000 μM, about 0.1 μM to 10,000 μM, about 1 μM to 1,000 μM, about 1 μM to 10 μM, about 10 μM to 100 μM, about 100 μM to 1 mM, or about 1 mM to 10 mM.

In some embodiments, the pharmaceutical composition comprises a LSD1-inhibitor that is GSK-2879552 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 12 mM, 14 mM, 16 mM, 18 mM, 20 mM, 25 mM, or about 30 mM.

In some embodiments, the pharmaceutical composition comprises GSK-2879552 at a unit dose of about 0.01 mg to 500 mg about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, or about 5-10 mg.

In some embodiments, the pharmaceutical composition comprises a LSD1-inhibitor that is GSK-LSD1 at a concentration of about 0.001 μM to 10 mM, about 0.01 μM to 1 mM, about 0.1 μM to 100 μM, about 0.001 μM to 0.01 μM, about 0.01 μM to 0.1 μM, about 0.1 μM to 1 μM, about 1 μM to 10 μM, about 10 μM to 100 μM, or about 100 μM to 1,000 μM.

In some embodiments, the pharmaceutical composition comprises a LSD1-inhibitor that is GSK-LSD1 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 1 mM, 5 mM, 10 mM, or 50 mM.

In some embodiments, the pharmaceutical composition comprises GSK-LSD1 at a unit dose of about of about 0.01 mg to 500 mg, about 0.1 mg to 100 mg, about 1 mg to 50 mg, about 1 mg to 25 mg, about 1 mg to 10 mg, about 1 mg to 5 mg, about 0.01 mg to 0.1 mg, about 0.1 mg to 1 mg, about 1 mg to 10 mg, about 10 mg to 100 mg, about 100 mg to 500 mg, about 0.5 mg to 1 mg, about 1 mg to 2 mg, about 2 mg to 3 mg, about 3 mg to 4 mg, about 4 mg to 5 mg, about 5-10 mg, about 10-25 mg, about 25-50 mg, or about 50-100 mg.

In some embodiments, the pharmaceutical composition comprises a LSD1-inhibitor that is Tranylcypromine at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1-inhibitor that is Tranylcypromine at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 12 mM, 14 mM, 16 mM, 18 mM, or 20 mM.

In some embodiments, the pharmaceutical composition comprises Tranylcypromine at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg, about 30 mg to 40 mg, about 40 mg to 50 mg, about 50 mg to 60 mg, about 60 mg to 70 mg, about 70 mg to 80 mg, about 90 mg to 100 mg, about 100 mg to 120 mg, or about 120 mg to 150 mg.

In some embodiments, the pharmaceutical composition comprises a LSD1-inhibitor that is Phenelzine sulfate at a concentration of about 0.1 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1-inhibitor that is Phenelzine sulfate at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the pharmaceutical composition comprises Phenelzine sulfate at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg; about 30 mg to 40 mg; about 40 mg to 50 mg about 50 mg to 60 mg; about 60 mg to 70 mg; about 70 mg to 80 mg; or about 90 mg to 100 mg.

In some embodiments, the pharmaceutical composition comprises a LSD1 inhibitor that is ORY-1001 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1 inhibitor that is ORY-1001 at a concentration of about 1 μM, 2 μM, 3 μM, 4 μM, 5 μM, 6 μM, 7 μM, 8 μM, 9 μM, 10 μM, 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the pharmaceutical composition comprises ORY-1001 at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg; about 30 mg to 40 mg; about 40 mg to 50 mg about 50 mg to 60 mg; about 60 mg to 70 mg; about 70 mg to 80 mg; or about 90 mg to 100 mg.

In some embodiments, the pharmaceutical composition comprises a LSD1 inhibitor that is RN-1 at a concentration of about 0.001 mM to 100,000 mM, 0.01 mM to 10,000 mM, about 0.1 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the pharmaceutical composition comprises a LSD1 inhibitor that is RN-1 at a concentration of about 1 μM, 2 μM, 3 μM, 4 μM, 5 μM, 6 μM, 7 μM, 8 μM, 9 μM, 10 μM, 10 μM, 20 μM, 30 μM, 40 μM, 50 μM, 60 μM, 70 μM, 80 μM, 90 μM, 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, or 10 mM.

In some embodiments, the pharmaceutical composition comprises RN-1 at a unit dose of about 1.5 mg to 750 mg, about 5 mg to 500 mg, about 10 mg to 250 mg, about 15 mg to 150 mg, about 1.5 mg to 10 mg, about 10 mg to 20 mg, about 20 mg to 30 mg; about 30 mg to 40 mg; about 40 mg to 50 mg about 50 mg to 60 mg; about 60 mg to 70 mg; about 70 mg to 80 mg; or about 90 mg to 100 mg.

In some embodiments the additional epigenetic agent is a KDM inhibitor.

In some embodiments, the pharmaceutical composition comprises a KDM inhibitor that is AS 8351 at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the pharmaceutical composition comprises a AS 8351 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 nM, 7 mM, 8 mM, 9 mM, or 10 mM.

In some embodiments, the pharmaceutical composition comprises a KDM inhibitor that is AS 8351 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises a KDM inhibitor that is TC-E 5002 at a concentration of about 0.01 μM to 1000 mM, about 0.1 μM to 100 mM, about 1 μM to 10 mM, about 10 μM to 1000 μM, about 1 μM to 10 μM, 10 μM to 100 μM, about 100 μM to 1000 μM or about 1 mM to 10 mM.

In some embodiments, the pharmaceutical composition comprises a AS TC-E 5002 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, or 10 mM.

In some embodiments, the pharmaceutical composition comprises a KDM inhibitor is TC-E 5002 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 200 mg to 2500 mg, about 200 mg to 2000 mg, about 200 mg to 1600 mg, about 200 mg to 1000 mg, about 100 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg

In some embodiments, the pharmaceutical composition comprises a KDM inhibitor that is EPT-103182 at a concentration of 0.001 μM to 100 mM, about 0.01 μM to 10 mM, about 0.1 μM to 1 mM, about 1 μM to 100 μM, about 1 μM to 10 μM, 10 μM to 100 μM, or about 100 sM to 1 mM.

In some embodiments, the pharmaceutical composition comprises EPT-103182 at a concentration of about 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, or about 1 mM.

In some embodiments, the pharmaceutical composition comprises a KDM inhibitor is EPT-103182 at a unit dose of about 50 mg to 5,000 mg, about 50 mg to 4000 mg, about 50 mg to 3000 mg, about 50 mg to 2000 mg, about 50 mg to 1000 mg, about 50 mg to 500 mg, about 100 mg to 2500 mg, about 100 mg to 2000 mg, about 100 mg to 1500 mg, about 100 mg to 1000 mg, about 100 mg to 500 mg, about 150 mg to 2500 mg, about 150 mg to 2000 mg, about 150 mg to 1500 mg, about 150 mg to 1250 mg, about 75 mg, about 100 mg, about 150 mg, about 200 mg, about 300 mg, about 400 mg, about 500 mg, about 600 mg, about 700 mg, about 800 mg, about 900 mg, about 1000 mg, about 1200 mg, about 1400 mg, about 1600 mg, about 1800 mg, or about 2000 mg.

In some embodiments, the pharmaceutical composition comprises a TAZ activator that is IBS008738 and a GSK3 Inhibitor that is AZD1080. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a TAZ activator that is IBS008738 and a GSK3 Inhibitor that is LY2090314. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 the is at a concentration of about 1 μM, 5 μM, 10 μM, 15 μM, 20 μM or 40 μM.

In some embodiments, the pharmaceutical composition comprises a TAZ activator that is IBS008738 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a TAZ activator that is IBS008738 and a GSK3 Inhibitor that is GSK3-inhibitor XXII. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the 1BS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a TAZ activator that is IBS008738 and a GSK3 Inhibitor that is CHIR99021. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 nM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a TAZ activator that is TT-10 and a GSK3 Inhibitor that is AZD1080. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a TAZ activator that is TT-10 and a GSK3 Inhibitor that is LY2090314. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 nM 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 the is at a concentration of about 1 μM, 5 μM, 10 μM, 15 μM, 20 μM or 40 μM.

In some embodiments, the pharmaceutical composition comprises a TAZ activator that is TT-10 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15, 20 μM, 50 μM, 100 μM, 250 μM or 500 μM.

In some embodiments, the pharmaceutical composition comprises a TAZ activator that is TT-10 and a GSK3 Inhibitor that is GSK3-inhibitor XXII. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 sM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mL, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mL, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a TAZ activator that is TT-10 and a GSK3 Inhibitor that is CHIR99021. The TT-10 is at a concentration of about 0.01 μM to 1000 mM, about 0.1 μM to 100 mM, about 1 μM to 100 mM, about 10 μM to 100 mL, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mL, about 0.1 mM to 1 mM, about 1 mM to 10 mM, about 10 mM to 100 mM, about 100 mM to 1,000 mM, or about 1,000 mM to 10,000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a TAZ activator that is TM-25659 and a GSK3 Inhibitor that is AZD1080. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mv, 95 mM, or 100 mM and the AZ1090 is at a concentration of about 1 mM, 2 mM, 3 mM, 4 mM, 5 mM, 6 mM, 7 mM, 8 mM, 9 mM, or 10 mM

In some embodiments, the pharmaceutical composition comprises a TAZ activator that is TM-25659 and a GSK3 Inhibitor that is LY2090314. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the LY2090314 the is at a concentration of about 1 μM, 5 μM, 10 μM, 15 μM, 20 μM or 40 μM.

In some embodiments, the pharmaceutical composition comprises a TAZ activator that is TM-25659 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a TAZ activator that is TM-25659 and a GSK3 Inhibitor that is GSK3-inhibitor XXII. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 s M, about 100 μM to 1 mM, about 1 mM to 10 mM, about 10 mM to 100 mM, or about 100 mM to 1000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM

In some embodiments, the pharmaceutical composition comprises a TAZ activator that is TM-25659 and a GSK3 Inhibitor that is CHIR99021. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the CHIR99021 is at a concentration of about 1 mli, 2 mM, 3 mM, 4 mM, 5 mM, 6 mM, 7 mM, 8 mM, 9 mM, or 10 mM.

In some embodiments, the pharmaceutical composition comprises a TAZ activator that is FHZ-000706 and a GSK3 Inhibitor that is AZD1080. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a TAZ activator that is FHZ-000706 and a GSK3 Inhibitor that is LY2090314. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 μM to 100 uM, about 0.001 sM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 the is at a concentration of about 1 μM, 5 μM, 10 μM, 15 μM, 20 μM or 40 μM.

In some embodiments, the pharmaceutical composition comprises a TAZ activator that is FHZ-000706 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 sM to 10 μM, about 10 μM to 100 μM, about 100 μM to 1 mM, or about 1 mM to 10 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM.

In some embodiments, the pharmaceutical composition comprises a TAZ activator that is FHZ-000706 and a GSK3 Inhibitor that is GSK3-inhibitor XXII. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM.

In some embodiments, the pharmaceutical composition comprises a TAZ activator that is FHZ-000706 and a GSK3 Inhibitor that is CHIR99021. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM.

In some embodiments, the pharmaceutical composition comprises a TAZ activator that is IBS008738 and a GSK3 Inhibitor that is AZD1080 and a HDAC inhibitor that is VPA. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a TAZ activator that is IBS008738 and a GSK3 Inhibitor that is LY2090314 and a HDAC inhibitor that is VPA. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 sM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a TAZ activator that is IBS008738 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the IBS008738 at a concentration of about 1.0 μM, 2.0 μM 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a TAZ activator that is IBS008738 and a GSK3 Inhibitor that is GSK3-inhibitor XXII and a HDAC inhibitor that is VPA. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises a TAZ activator that is IBS008738 and a GSK3 Inhibitor that is CHIR99021 and a HDAC inhibitor that is VPA. The IBS008738 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the IBS008738 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a TAZ activator that is TT-10 and a GSK3 Inhibitor that is AZD1080 and a HDAC inhibitor that is VPA. The TT-10 is at a concentration of about 0.01 μM to 1000 mM, about 0.1 μM to 100 mM, about 1 sM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a TAZ activator that is TT-10 and a GSK3 Inhibitor that is LY2090314 and a HDAC inhibitor that is VPA. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a TAZ activator that is TT-10 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a TAZ activator that is TT-10 and a GSK3 Inhibitor that is GSK3-inhibitor XXII and a HDAC inhibitor that is VPA. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises a TAZ activator that is TT-10 and a GSK3 Inhibitor that is CHIR99021 and a HDAC inhibitor that is VPA. The TT-10 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the TT-10 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a TAZ activator that is TM-25659 and a GSK3 Inhibitor that is AZD1080 and a HDAC inhibitor that is VPA. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the AZ1090 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a TAZ activator that is TM-25659 and a GSK3 Inhibitor that is LY2090314 and a HDAC inhibitor that is VPA. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a TAZ activator that is TM-25659 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a TAZ activator that is TM-25659 and a GSK3 Inhibitor that is GSK3-inhibitor XXII and a HDAC inhibitor that is VPA. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises a TAZ activator that is TM-25659 and a GSK3 inhibitor that is CHIR99021 and a HDAC inhibitor that is VPA. The TM-25659 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the TM-25659 at a concentration of about 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, or 100 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a TAZ activator that is FHZ-000706 and a GSK3 Inhibitor that is AZD1080 and a HDAC inhibitor that is VPA. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the AZ1090 is at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the AZ1090 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, the pharmaceutical composition comprises a TAZ activator that is FHZ-000706 and a GSK3 Inhibitor that is LY2090314 and a HDAC inhibitor that is VPA. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the LY2090314 at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM and the VPA at a concentration about 100 mM to 4,000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the LY2090314 the is at a concentration of about 1 μM, 5 μM, 10 μM, 15 μM, 20 μM or 40 μM and the VPA at a concentration about 100 mM to 4,000 mM.

In some embodiments, the pharmaceutical composition comprises a TAZ activator that is FHZ-000706 and a GSK3 Inhibitor that is that is a substituted 3-Imidazo[1,2-a]pyridin-3-yl-4-(1,2,3,4-tetrahydro-[1,4]diazepino-[6,7,1-hi]indol-7-yl)pyrrole-2,5-dione and a HDAC inhibitor that is VPA. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 10 mM, about 0.01 μM to 1 mM, about 0.1 μM to 100 uM, about 0.001 μM to 0.01 μM, about 0.01 μM to 0.1 μM, about 0.1 μM to 1 μM, about 1 μM to 10 μM, about 10 μM to 100 μM, about 100 μM to 1 mM, or about 1 mM to 10 mM. and the VPA at a concentration about 100 mM to 4,000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the substituted 3-Imidazo[1,2-a]pyridin-3-yl-4-(1,2,3,4-tetrahydro-[1,4]diazepino-[6,7,1-hi]indol-7-yl)pyrrole-2,5-dione, is at a concentration of about 1 μM, 5 μM, 10 μM, 15 μM, 20 μM, 50 μM, 100 μM, 250 μM or 500 μM and the VPA at a concentration about 100 mM to 4,000 mM.

In some embodiments, the pharmaceutical composition comprises a TAZ activator that is FHZ-000706 and a GSK3 Inhibitor that is GSK3-inhibitor XXII and a HDAC inhibitor that is VPA. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the GSK3-inhibitor XXII, at a concentration of about of about 0.1 μM to 1,000 mM, about 1 μM to 100 mM, about 10 μM to 10 mM, about 0.1 μM to 1 μM, about 1 μM to 10 μM, about 10 μM to 100 μM, about 100 μM to 1 mM, about 1 mM to 10 mM, about 10 mM to 100 mM, or about 100 mM to 1000 mM and the VPA at a concentration about 100 mM to 4,000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, or about 50 mM and the GSK3-inhibitor XXII is at a concentration of about 0.1 mM, 0.2 mM, 0.3 mM, 0.4 mM, 0.5 mM, 0.6 mM, 0.7 mM, 0.8 mM, 0.9 mM, or 1.0 mM. and the VPA at a concentration about 100 mM to 4,000 mM.

In some embodiments, the pharmaceutical composition comprises a TAZ activator that is FHZ-000706 and a GSK3 Inhibitor that is CHIR99021 and a HDAC inhibitor that is VPA. The FHZ-000706 is at a concentration of about 0.01 μM to 1000 mM, about 0.1 μM to 100 mM, about 1 μM to 100 mM, about 10 μM to 100 mM, about 100 μM to 100 mM, 10 μM to 100 μM, about 100 μM to 1000 μM, about 1 mM to 10 mM, or about 10 mM to 100 mM and the CHIR99021 at a concentration of about 0.001 mM to 10,000 mM, about 0.01 mM to 1,000 mM, about 0.1 mM to 100 mM, about 0.001 mM to 0.01 mM, about 0.01 mM to 0.1 mM, about 0.1 mM to 1 mM, about 1 mM to 10 mM, about 10 mM to 100 mM, about 100 mM to 1,000 mM, or about 1,000 mM to 10,000 mM and the VPA at a concentration about 100 mM to 4,000 mM.

In some embodiments, the FHZ-000706 at a concentration of about 1.0 μM, 2.0 μM, 3.0 μM, 4.0 μM, 5.0 μM, 6.0 μM, 7.0 μM, 8.0 μM, 9.0 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 ml, 10 mM, 11 mM, 12 mM, 13 mM, 14 mM, 15 mM, 20 mM, 25 mM, 30 mM, 35 mM, 40 mM, 45 mM, or about 50 mM and the CHIR99021 is at a concentration of about 1 mM, 2 mM, 3 mM, 4 mM, 5 mM, 6 mM, 7 mM, 8 mM, 9 mM, or 10 mM and the VPA at a concentration about 100 mM to 4,000 mM.

In some embodiments, as noted above, a composition is adapted for administration to the inner ear and/or middle ear, for example, local administration to the round window membrane or intratympanic or transtympanic administration, for example, to cochlear tissue. Alternatively, as noted above, a composition is adapted for administration systemically for example, orally or parentally.

When administered locally, for example to the inner and/or middle ear, the compounds (s) are administered at a unit dose of about 25 μl to 500 μl, or about 50 μl to 200 μl.

The phrase “pharmaceutically-acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically-acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically-acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

Certain compositions comprise at least one biocompatible matrix. The term “biocompatible matrix” as used herein is a polymeric carrier that is acceptable for administration to humans for the release of therapeutic agents. In some instances, a biocompatible matrix is a biocompatible gel, foam, fiber, film, or mats. In some embodiments the biocompatible matrix is derived from silk.

In some embodiments the biocompatible matrix comprises hyaluronic acid, hyaluronates, lecithin gels, pluronics, poly(ethyleneglycol), polymers, poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate or a combination thereof.

Exemplary polymers suitable for formulating the biologically active compositions of the present disclosure include, but are not limited to polyamides, polycarbonates, polyalkylenes (polyethylene glycol (PEG)), polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In some embodiments, the polymer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the polymer is in a concentration between about 10 wt %/o and about 23 wt % relative to the composition. In some embodiments the polymer is in a concentration between about 15 wt % and about 20 wt % relative to the composition. In particular embodiments, the polymer is in a concentration is approximately 17 wt % relative to the composition.

In one embodiment, a biologically active composition of the present disclosure is formulated in a ABA-type or BAB-type triblock copolymer or a mixture thereof, wherein the A-blocks are relatively hydrophobic and comprise biodegradable polyesters or poly(orthoester), and the B-blocks are relatively hydrophilic and comprise polyethylene glycol (PEG). The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof.

In some embodiments, the copolymer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the copolymer is in a concentration between about 10 wt % and about 23 wt % relative to the composition. In some embodiments the copolymer is in a concentration between about 15 wt % and about 20 wt % relative to the composition. In particular embodiments, the copolymer is in a concentration is approximately 17 wt % relative to the composition.

Certain compositions comprise at least one poloxamer. Poloxamers are triblock copolymers formed of (i.e. hydrophilic poly(oxyethylene) blocks and hydrophobic poly(oxypropylene) blocks) configured as a triblock of poly(oxyethylene)-poly(oxypropylene)-poly(oxyethylene). Poloxamers are one class of block copolymer surfactants having a propylene oxide block hydrophobe and an ethylene oxide hydrophile. Poloxamers are commercially available (e.g. Pluronic® polyols are available from BASF Corporation). Alternatively, poloxamers can be synthesized by known techniques.

Exemplary poloxamers include Poloxamer 124, Poloxamer 188, Poloxamer 237, Poloxamer 338, and Poloxamer 407. In some embodiments, the poloxamer comprises mixtures of two or more of Poloxamer 124, Poloxamer 188, Poloxamer 237, Poloxamer 338 or Poloxamer 407. In some embodiments, the mixture of two or more poloxamers comprise Poloxamer 407 and Poloxamer 124. In certain embodiments the poloxamer comprises at least one of Poloxamer 188 and Poloxamer 407 or mixtures thereof. In some embodiments, the poloxamer is Poloxamer 407.

In some embodiments, the poloxamer is in a concentration between about 5 wt % and about 25 wt % relative to the composition, or about 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt % relative to the composition. In certain embodiments, the poloxamer is in a concentration between about 10 wt % and about 23 wt % relative to the composition. In some embodiments the poloxamer is in a concentration between about 15 wt % and about 20 wt % relative to the composition. In particular embodiments, the poloxamer is in a concentration is approximately 17 wt % relative to the composition.

In some embodiments, wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Certain compositions comprise at least one antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In specific embodiments, the viscosity of the composition at about body temperature is substantially different (e.g. lesser, greater) than the viscosity of the composition at room temperature.

In some embodiments, the composition comprises a buffer. For example, in certain instances, the buffer is physiological saline or phosphate-buffered saline (PBS).

In some embodiments, the composition is at or near physiological pH. For instance, in some embodiments, the composition has a pH of between about 6 and about 8, including all integers, decimals, and ranges in between, for example, about 6 to about 6.5 to about 7 to about 7.5 to about 8. In specific embodiments, the composition has a pH of about 7.4 (±0.2).

In some aspects, the present disclosure the pharmaceutical compositions are lyophilized. comprising one or more agents described herein and a gelling agent.

In some embodiments, the lyophilized pharmaceutical composition is in the form of a lyophilized cake.

In some embodiments, the lyophilized pharmaceutical composition has a higher stability to oxygen and/or light as compared to a comparable pharmaceutical composition comprising one or more solvents.

In some embodiments, the present disclosure provides a reconstituted solution of the lyophilized pharmaceutical compositions.

As used herein, the term “gelling agent” refers to an agent capable of imparting a gel-like or thickening quality to the pharmaceutical composition or reconstituted solution of the present disclosure upon being subjected to a gelling condition (e.g. a particular temperature or temperature range, the presence of an ion, a pH value or range, or a concentration of gelling agent that causes the gelling agent to undergoing a change or transition from low viscosity to high viscosity, or the reverse). In some embodiments, the gelling condition is a particular temperature (e.g. about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.). In some embodiments, the gelling condition is a particular temperature range (e.g. about 26° C. or higher, about 27° C. or higher, about 28° C. or higher, about 29° C. or higher, about 30° C. or higher, about 31° C. or higher, about 32° C. or higher, about 33° C. or higher, about 34° C. or higher, about 35° C. or higher, about 36° C. or higher, about 37° C. or higher, about 38° C. or higher, about 39° C. or higher, or about 40° C. or higher). In some embodiments, the gelling agent provides a viscosity of between about 1,000 and 10,000,000 centipoise, between about 5,000 and 5,000,000 centipoise, or between about 100,000 and 4,000,000 centipoise, to the pharmaceutical composition or reconstituted solution of the present disclosure. In some embodiments, the gelling agent provides a viscosity of between about 50,000 and 2,000,000 centipoise to the pharmaceutical composition or reconstituted solution of the present disclosure.

In some embodiments, prior to gelling (e.g. at ambient temperature (e.g. between about 20° C. and about 26° C.)), the gelling agent provides a viscosity of less than about 100,000 centipoise, less than about 50,000 centipoise, 20,000 centipoise, less than about 10,000 centipoise, less than about 8,000 centipoise, less than about 7,000 centipoise, less than about 6,000 centipoise, less than about 5,000 centipoise, less than about 4,000 centipoise, less than about 3,000 centipoise, less than about 2,000 centipoise, or less than about 1,000 centipoise to the pharmaceutical composition or reconstituted solution of the present disclosure.

In some embodiments, upon gelling (e.g. at the temperature of a human body (e.g. between about 35° C. to about 39° C., between about 36° C. to about 38° C., or at about 37° C.)), the gelling agent provides a viscosity of greater than about 1,000 centipoise, greater than about 5,000 centipoise, greater than about 10,000 centipoise, greater than about 20,000 centipoise, greater than about 50,000 centipoise, greater than about 60,000 centipoise, greater than about 70,000 centipoise, greater than about 80,000 centipoise, greater than about 90,000 centipoise, or greater than about 100,000 centipoise.

In some embodiments, upon gelling (e.g. at the temperature of a human body (e.g. between about 36° C. to about 39° C., or at about 37° C.)), the viscosity of the pharmaceutical composition or reconstituted solution of the present disclosure, as measured in units of centipoise, being about 2 fold or greater, about 5 fold or greater, about 10 fold or greater, about 20 fold or greater, about 50 fold or greater, about 60 fold or greater, about 7 fold or greater, about 80 fold or greater, about 90 fold or greater, about 100 fold or greater as compared to the viscosity of the pharmaceutical composition or reconstituted solution prior to gelling (e.g. at ambient temperature (e.g. at about 25° C.)).

It is understood that the gelling condition (e.g. gelling temperature) of the pharmaceutical composition or reconstituted solution of the present disclosure is measured with a variety of techniques in the art. In some embodiment, the gelling temperature is determined using a commercially available rheometer having a parallel plate geometry (e.g. with plate distance ranging from 0.5 mm to 1.0 mm). In some embodiments, the analysis is performed over a continuous temperature range (e.g. 15° C. to 40° C.) at a constant rate (e.g. 2 to 3° C./min) and a deformation frequency of 0.74 Hz to 1 Hz. The gelation temperature is determined at the temperature whereby the shear storage modulus (G′) and the shear loss modulus (G″) are equal.

In some embodiments, the gelling agent comprises acacia, alginic acid, bentonite, poly(acrylic acid) (Carbomer), carboxymethyl cellulose, ethylcellulose, gelatin, hydroxyethyl cellulose, hydroxypropyl cellulose, magnesium aluminum silicate (Veegum), methylcellulose, poloxamer, hyaluronic acid sodium, polylacticglycolic acid sodium, chitosan, polyvinyl alcohol, sodium alginate, tragacanth, xanthan gum, or any combination thereof. In some embodiment, the gelling agent comprises poloxamer.

In some embodiments, the gelling agent is a thermoreversible gelling agent.

As used herein, the term “thermoreversible” refers to a capability of being reversible by the application of heat. The “thermoreversible gelling agent” refers to an agent capable of reversibly imparting a gel-like or thickening quality to the pharmaceutical composition or reconstituted solution of the present disclosure upon application of heat.

In some embodiments, the thermoreversible gelling agent comprises a poloxamer.

It is understood that the gelling agent (e.g. the thermoreversible gelling agent) may also be a bulking agent of the pharmaceutical composition or reconstituted solution of the present disclosure. In some embodiments, a poloxamer (e.g. poloxamer 407) is the gelling agent and/or the bulking agent of the pharmaceutical composition or reconstituted solution of the present disclosure. Poloxomers are a general class of commercially available and pharmaceutically acceptable triblock copolymers of polyethylene oxide-polypropylene oxide-polyethylene oxide which exhibit relatively low viscosity at low temperatures (e.g. room temperature or below) but much high viscosities at elevated temperatures (e.g. body temperatures of approximately 37° C.) whereby compositions containing such thermoreversible gelling agents effectively solidify in place. Other thermoreversible gelling agents such as polyethylene oxide-polylactic acid-polyethylene oxide polymers are also suitable in various embodiments of the present invention.

In some embodiments, the poloxamer (e.g. poloxamer 407) is the gelling agent and the bulking agent of the pharmaceutical composition or reconstituted solution of the present disclosure. In some embodiments, the presence of the poloxamer (e.g. poloxamer 407) in the pharmaceutical composition (e.g. the lyophilized pharmaceutical composition) alleviates the need for any other excipient (e.g. additional bulking agent). Such alleviation may provide one or more advantages to the pharmaceutical composition (e.g. enhanced stability and/or reduced reconstitution time).

In some embodiments, the poloxamer is selected from the group consisting of Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, and Poloxamer 407.

In some embodiments, the poloxamer is Poloxamer 188 or Poloxamer 407.

In some embodiments, the poloxamer is Poloxamer 407.

In some embodiments, the poloxamer is a purified poloxamer (e.g. purified Poloxamer 407).

In some embodiments, the purified poloxamer (e.g. purified Poloxamer 407) has an average molecular weight of about 9 kDa or greater, about 9.2 kDa or greater, about 9.4 kDa or greater, about 9.6 kDa or greater, about 9.8 kDa or greater, about 10 kDa or greater, about 10.2 kDa or greater, about 10.4 kDa or greater, about 10.6 kDa or greater, about 10.8 kDa or greater, about 11 kDa or greater, about 11.2 kDa or greater, about 11.4 kDa or greater, about 11.6 kDa or greater, about 11.8 kDa or greater, about 12 kDa or greater, or about 12.1 kDa or greater.

In some embodiments, the purified poloxamer (e.g. purified Poloxamer 407) has a reduced level of polymer chains with molecular weight below 9 kDa as compared to the unpurified poloxamer (e.g. unpurified Poloxamer 407).

In some embodiments, the purified poloxamer (e.g. purified Poloxamer 407) has about 99% or less, about 98% or less, about 95% or less, about 90% or less, about 80% or less, about 70% or less, about 60% or less, about 50% or less, about 40% or less, about 30% or less, about 20% or less, or about 10% or less of polymer chains with molecular weight below 9 kDa as compared to the unpurified poloxamer (e.g. unpurified Poloxamer 407).

In some embodiments, the purified poloxamer (e.g. purified Poloxamer 407) is prepared by liquid-liquid extraction or size exclusion chromatography.

In some embodiments, about 10% or more, about 20% or more, about 30% or more, about 400% or more, about 50% or more, about 60% or more, about 70% or more, about 800% or more, about 90% or more, about 95% or more, about 98% or more, or about 99% or more of the one or more impurities having molecular weights below 9 kDa are removed from the poloxamer (e.g. Poloxamer 407) during the purification.

In some embodiments, about 10% or more, about 20% or more, about 30% or more, about 40% or more, about 50% or more, about 60% or more, about 70% or more, about 80%1 or more, about 90% or more, about 95% or more, about 98% or more, or about 99% or more of the one or more diblock copolymers (e.g. PEO-PPO), single block polymers (e.g. PEO), and/or aldehydes are removed from the poloxamer (e.g. Poloxamer 407) during the purification.

In some embodiments, the pharmaceutical composition, pharmaceutical composition, the lyophilized pharmaceutical composition or reconstituted solution of the present disclosure comprises a buffering agent. The buffer controls the pH of the reconstituted solution to a range of from about 4 to about 13, from about 5 to about 12, from about 6 to about 11, from about 6.5 to about 10.5, or from about 7 to about 10.

Examples of the buffering agent include, but are not limited to, citrate buffering agents, acetate buffering agents, phosphate buffering agents, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents are selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

In some embodiments, the buffering agent comprises phosphate buffered saline, TRIS, tris acetate, tris HCl-65, sodium citrate, histidine, arginine, sodium phosphate, tris base-65, hydroxyethyl starch, or any combination thereof.

In some embodiments, the pharmaceutical composition, pharmaceutical composition, the lyophilized pharmaceutical composition or reconstituted solution of the present disclosure comprises a bulking agent.

In some embodiments, the bulking agent comprises poloxamer (e.g. poloxamer 407), mannitol, sucrose, maltose, trehalose, dextrose, sorbitol, glucose, raffinose, glycine, histidine, polyvinylpyrrolidone (e.g. polyvinylpyrrolidone K12 or polyvinylpyrrolidone K17), lactose, or any combination thereof.

In some embodiments, the pharmaceutical composition, pharmaceutical composition, the lyophilized pharmaceutical composition or reconstituted solution of the present disclosure comprises a stabilizing agent.

In some embodiments, the stabilizing agent comprises a cryoprotectant. In some embodiments, the cryoprotectant is a polyol (e.g. a diol or a triol such as propylene glycol (i.e. 1,2-propanediol), 1,3-propanediol, glycerol, (+/−)-2-methyl-2,4-pentanediol, 1,6-hexanediol, 1,2-butanediol, 2,3-butanediol, ethylene glycol, or diethylene glycol), a nondetergent sulfobetaine (e.g. NDSB-201 (3-(1-pyridino)-1-propane sulfonate), an osmolyte (e.g. L-proline or trimethylamine N-oxide dihydrate), a polymer (e.g. polyethylene glycol 200 (PEG 200), PEG 400, PEG 600, PEG 1000, PEG 3350, PEG 4000, PEG 8000, PEG 10000, PEG 20000, polyethylene glycol monomethyl ether 550 (mPEG 550), mPEG 600, mPEG 2000, mPEG 3350, mPEG 4000, mPEG 5000, polyvinylpyrrolidone (e.g. polyvinylpyrrolidone K 15), pentaerythritol propoxylate, or polypropylene glycol P 400), an organic solvent (e.g. dimethyl sulfoxide (DMSO) or ethanol), a sugar (e.g. D-(+)-sucrose, D-sorbitol, trehalose, D-(+)-maltose monohydrate, meso-erythritol, xylitol, myo-inositol, D-(+)-raffinose pentahydrate, D-(+)-trehalose dihydrate, or D-(+)-glucose monohydrate), or a salt (e.g. lithium acetate, lithium chloride, lithium formate, lithium nitrate, lithium sulfate, magnesium acetate, sodium chloride, sodium formate, sodium malonate, sodium nitrate, sodium sulfate, or any hydrate thereof) or any combination thereof.

In some embodiments, the stabilizing agent comprises a salt. In some embodiment, the salt is selected from the group consisting of lithium salts (e.g. lithium acetate, lithium chloride, lithium formate, lithium nitrate, lithium sulfate, or any hydrate thereof), magnesium salts (e.g. magnesium acetate or a hydrate thereof), and sodium salts (e.g. sodium chloride, sodium formate, sodium malonate, sodium nitrate, sodium sulfate, or any hydrate thereof). For another example, the formulation comprises one or more sodium salts. For yet another example, the formulation comprises sodium chloride.

In some embodiment, the stabilizing agent comprises a surfactant. In some embodiments, the surfactant comprises one or more anionic surfactants (e.g. 2-acrylamido-2-methylpropane sulfonic acid, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium dodecylbenzenesulfonate, sodium laurate, sodium laureth sulfate, sodium lauroyl sarcosinate, sodium myreth sulfate, sodium nonanoyloxybenzenesulfonate, sodium pareth sulfate, sodium stearate, or sulfolipid), one or more cationic surfactants (e.g. behentrimonium chloride, benzalkonium chloride, benzethonium chloride, benzododecinium bromide, bronidox, carbethopendecinium bromide, cetalkonium chloride, cetrimonium bromide, cetrimonium chloride, cetylpyridinium chloride, didecyldimethylammonium chloride, dimethyldioctadecylammonium bromide, dimethyldioctadecylammonium chloride, domiphen bromide, lauryl methyl gluceth-10 hydroxypropyl diimonium chloride, octenidine dihydrochloride, olaflur, n-oleyl-1,3-propanediamine, pahutoxin, stearalkonium chloride, tetramethylammonium hydroxide, or thonzonium bromide), one or more zwitterionic surfactants (e.g. cocamidopropyl betaine, cocamidopropyl hydroxysultaine, dipalmitoylphosphatidylcholine, egg lecithin, hydroxysultaine, lecithin, myristamine oxide, peptitergents, or sodium lauroamphoacetate), and/or one or more non-ionic surfactants (e.g. alkyl polyglycoside, cetomacrogol 1000, cetostearyl alcohol, cetyl alcohol, cocamide dea, cocamide mea, decyl glucoside, decyl polyglucose, glycerol monostearate, igepal ca-630, isoceteth-20, lauryl glucoside, maltosides, monolaurin, mycosubtilin, narrow-range ethoxylate, nonidet p-40, nonoxynol-9, nonoxynols, np-40, octaethylene glycol monododecyl ether, n-octyl beta-d-thioglucopyranoside, octyl glucoside, oleyl alcohol, peg-10 sunflower glycerides, pentaethylene glycol monododecyl ether, polidocanol, α-tocopheryl polyethylene glycol succinate (TPGS), poloxamer (e.g. poloxamer 407), polyethoxylated tallow amine, polyglycerol polyricinoleate, polysorbate (e.g. polysorbate 20, polysorbate 40, polysorbate 60, or polysorbate 80), sorbitan, sorbitan monolaurate, sorbitan monostearate, sorbitan tristearate, stearyl alcohol, surfactin, triton x-100).

In some embodiments, the pharmaceutical composition, pharmaceutical composition, the lyophilized pharmaceutical composition or reconstituted solution of the present disclosure comprises a tonicity-adjusting agent.

In some embodiments, the tonicity-adjusting agent comprises NaCl, dextrose, dextran, ficoll, gelatin, mannitol, sucrose, glycine, glycerol, or any combination thereof.

In some embodiments, the pharmaceutical composition or reconstituted solution of the present disclosure comprises a soothing agent. In some embodiments, the soothing agent comprises lidocaine

In addition to these components, the pharmaceutical composition, pharmaceutical composition, the lyophilized pharmaceutical composition or reconstituted solution of the present disclosure includes any substance useful in pharmaceutical compositions.

In some embodiments, the pharmaceutical composition, pharmaceutical composition, the lyophilized pharmaceutical composition or reconstituted solution of the present disclosure includes one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents are selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent is starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Examples of preservatives may include, but are not limited to, antioxidants, chelating agents, antimicrobial preservatives, antifungal preservatives, alcohol preservatives, acidic preservatives, and/or other preservatives. Examples of antioxidants include, but are not limited to, alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Examples of chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Examples of antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Examples of antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, benzyl alcohol, phenol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, an/or silicone oil.

Compounds or compositions described herein can be formulated in any manner suitable for a desired delivery route, e.g. transtympanic injection, transtympanic wicks and catheters, cochlear implants, and injectable depots. In some instances, compositions or formulations include one or more physiologically-acceptable components, including derivatives or prodrugs, solvates, stereoisomers, racemates, or tautomers thereof with any physiologically acceptable carriers, diluents, and/or excipients.

As noted above, certain compositions are adapted for, and certain methods employ, administration to the middle ear or inner ear, for example, by local administration to the round window membrane. The membrane of the round window is the biological barrier to the inner ear space and represents the major obstacle for the local treatment of hearing impairment. The administered drug must overcome this membrane to reach the inner ear space. The drug can operatively (e.g. injection through the tympanic membrane) be placed locally to the round window membrane and can then penetrate through the round window membrane. Substances that penetrate the round window typically distribute in the perilymph and thus reach the hair cells and supporting cells.

The pharmaceutical compositions or formulations may also contain a membrane penetration enhancer, which supports the passage of the agents mentioned herein through the round window membrane. Accordingly, liquid, gel or foam formulations are used. It is also possible to apply the active ingredient orally or to employ a combination of delivery approaches.

Certain compositions are adapted for, and certain methods employ, administration to the middle ear or inner ear, for example, by intratympanic or transtympanic administration. Intratympanic (IT) delivery of drugs to the ear is increasingly used for both clinical and research purposes. Some groups have applied drugs in a sustained manner using microcatheters and microwicks, while the majority have applied them as single or as repeated IT injections (up to 8 injections over periods of up to 2 weeks).

Intratympanically applied drugs are thought to enter the fluids of the inner ear primarily by crossing the round window (RW) membrane. Calculations show that a major factor controlling both the amount of drug entering the ear and the distribution of drug along the length of the ear is the duration the drug remains in the middle ear space. Single, ‘one-shot’ applications or applications of aqueous solutions for few hours' duration result in steep drug gradients for the applied substance along the length of the cochlea and rapidly declining concentration in the basal turn of the cochlea as the drug subsequently becomes distributed throughout the ear.

In some embodiments, other injection approaches include by osmotic pump, or, by combination with implanted biomaterial, or, by injection or infusion. Biomaterials that can aid in controlling release kinetics and distribution of drug include hydrogel materials, degradable materials. One class of materials that can be used includes in situ gelling materials. All potential materials and methodologies mentioned in references (Almeida H, Amaral M H, Lobao P, Lobo J M, Drug Discov Today 2014; 19:400-12; Wise A K, Gillespie L N, J Neural Eng 2012; 9:065002; Surovtseva E V, Johnston A H, Zhang W, et al, Int J Pharmaceut 2012; 424:121-7; Roy S, Glueckert R, Johnston A H, et al., Nanomedicine 2012; 7:55-63; Rivera T, Sanz L, Camarero G, Varela-Nieto I., Curr Drug Deliv 2012; 9:231-42; Pararas E E, Borkholder D A, Borenstein J T, Adv Drug Deliv Rev 2012; 64:1650-60; Li M L, Lee L C, Cheng Y R, et al., IEEE T Bio-Med Eng 2013; 60:2450-60; Lajud S A, Han Z, Chi F L, et al., J Control Release 2013; 166:268-76; Kim D K, Park S N, Park K H, et al., Drug Deliv 2014; Engleder E, Honeder C, Klobasa J, Wirth M, Arnoldner C, Gabor F, Int J Pharmaceut 2014; 471:297-302; Bohl A, Rohm H W, Ceschi P, et al., J Mater Sci Mater Med 2012:23:2151-62; Hoskison E, Daniel M, Al-Zahid S, Shakesheff K M, Bayston R, Birchall J P, Ther Deliv 2013; 4:115-24; Staecker H, Rodgers B, Expert Opin Drug Deliv 2013; 10:639-50; Pritz C O, Dudas J, Rask-Andersen H, Schrott-Fischer A, Glueckert R, Nanomedicine 2013; 8:1155-72), which are included herein by reference in their entirety. Other materials include collagen or other natural materials including fibrin, gelatin, and decellularized tissues. Gelfoam may also be suitable.

Delivery may also be enhanced via alternate means including but not limited to agents added to the delivered composition such as penetration enhancers, or could be through devices via ultrasound, electroporation, or high-speed jet.

Methods described herein can also be used for inner ear cell types that are produced using a variety of methods know to those skilled in the art including those cell types described in PCT Application No. WO2012103012 A1.

With regard to human and veterinary treatment, the amount of a particular agent(s) that is/are administered is/are dependent on a variety of factors, including the disorder being treated and the severity of the disorder; activity of the specific agent(s) employed; the age, body weight, general health, sex and diet of the patient; the time of administration, route of administration, and rate of excretion of the specific agent(s) employed; the duration of the treatment; drugs used in combination or coincidental with the specific agent(s) employed; the judgment of the prescribing physician or veterinarian; and like factors known in the medical and veterinary arts.

The agents described herein are administered in a therapeutically effective amount to a subject in need of treatment. Administration of compositions described herein can be via any of suitable route of administration, for example, by intratympanic administration. Other routes include ingestion, or alternatively parenterally, for example intravenously, intra-arterially, intraperitoneally, intrathecally, intraventricularly, intraurethrally, intrasternally, intracranially, intramuscularly, intranasally, subcutaneously, sublingually, transdermally, or by inhalation or insufflations, or topical by ear instillation for absorption through the skin of the ear canal and membranes of the eardrum. Such administration is a single or multiple oral dose, defined number of ear drops, or a bolus injection, multiple injections, or as a short- or long-duration infusion. Implantable devices (e.g. implantable infusion pumps) may also be employed for the periodic parenteral delivery over time of equivalent or varying dosages of the particular formulation. For such parenteral administration, the compounds are formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

Compositions described herein can be administered by several methods sufficient to deliver the composition to the inner ear. Delivering a composition to the inner ear includes administering the composition to the middle ear, such that the composition may diffuse across the round window to the inner ear. It also includes administering a composition to the inner ear by direct injection through the round window membrane. Such methods include, but are not limited to auricular administration, by transtympanic wicks or catheters, or parenteral administration, for example, by intrarticular, transtympanic, or intracochlear injection.

In particular embodiments, the compounds, compositions and formulations of the disclosure are locally administered, meaning that they are not administered systemically.

In one embodiment, a syringe and needle apparatus is used to administer compounds or compositions to a subject using auricular administration. A suitably sized needle is used to pierce the tympanic membrane and a wick or catheter comprising the composition is inserted through the pierced tympanic membrane and into the middle ear of the subject. The device is inserted such that it is in contact with the round window or immediately adjacent to the round window. Exemplary devices used for auricular administration include, but are not limited to, transtympanic wicks, transtympanic catheters, round window microcatheters (small catheters that deliver medicine to the round window), and Silverstein Microwicks™ (small tube with a “wick” through the tube to the round window, allowing regulation by subject or medical professional).

In some embodiments, a syringe and needle apparatus is used to administer compounds or compositions to a subject using transtympanic injection, injection behind the tympanic membrane into the middle and/or inner ear. The formulation is administered directly onto the round window membrane via transtympanic injection or is administered directly to the cochlea via intracochlear injection or directly to the vestibular organs via intravestibular injection.

In some embodiments, the delivery device is an apparatus designed for administration of compounds or compositions to the middle and/or inner ear. By way of example only: GYRUS Medical GmbH offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. patent application Ser. No. 08/874,208, which is incorporated herein by reference for such disclosure, describes a surgical method for implanting a fluid transfer conduit to deliver compositions to the inner ear. U.S. Patent Application Publication 2007/0167918, which is incorporated herein by reference for such disclosure, further describes a combined otic aspirator and medication dispenser for transtympanic fluid sampling and medicament application.

In some embodiments, a compound or composition disclosed herein is administered to a subject in need thereof once. In some embodiments, a compound or composition disclosed herein is administered to a subject in need thereof more than once. In some embodiments, a first administration of a compound or composition disclosed herein is followed by a second, third, fourth, or fifth administration of a compound or composition disclosed herein.

The number of times a compound or composition is administered to a subject in need thereof depends on the discretion of a medical professional, the disorder, the severity of the disorder, and the subject's response to the formulation. In some embodiments, the compound or composition disclosed herein is administered once to a subject in need thereof with a mild acute condition. In some embodiments, a compound or composition disclosed herein is administered more than once to a subject in need thereof with a moderate or severe acute condition. In the case wherein the subject's condition does not improve, upon the doctor's discretion the compound or composition is administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the compound or composition may administered continuously; alternatively, the dose of drug being administered is temporarily reduced or temporarily suspended for a certain length of time (i.e. a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday is from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once the subject's hearing and/or balance has improved, a maintenance dose can be administered, if necessary. Subsequently, the dosage or the frequency of administration, or both, is optionally reduced, as a function of the symptoms, to a level at which the improved disease, disorder or condition is retained. In certain embodiments, subjects require intermittent treatment on a long-term basis upon any recurrence of symptoms.

Certain embodiments include is a pharmaceutical product comprising a sealed packaging and the compound(s) according to the invention in the container. The container size can be optimized to reduce head space in the container after packaging and any head space is filled with an inert gas such as nitrogen. Furthermore, container material of construction can be chosen to minimize the moisture and oxygen ingress inside the container after packaging.

### Embodiments

In further embodiments, enumerated as embodiments 1-2760 below, the present disclosure includes:
Embodiment 1. A method for increasing proliferation of a cochlear supporting cell or a vestibular supporting cell, comprising contacting the supporting cell with:
   a) a transcriptional coactivator with PDZ-binding motif (TAZ) activator; and
   b) a Wnt agonist;
   wherein (a) and (b) can occur in any order or simultaneously, thereby increasing cochlear supporting cell or vestibular supporting cell proliferation compared to a vehicle control.
Embodiment 2. A method for producing an expanded population of cochlear or vestibular cells, comprising contacting a population of cochlear supporting cells or vestibular supporting cells with:
   a) a transcriptional coactivator with PDZ-binding motif (TAZ) activator and;
   b) a Wnt agonist
   wherein (a) and (b) can occur in any order or simultaneously, thereby producing an expanded population of cochlear or vestibular cells compared to a vehicle control.
Embodiment 3. The method of any of embodiments 1 and 2, wherein the cells are cochlear cells.
Embodiment 4. The method of any of embodiments 1 and 2, wherein the cells are vestibular cells.
Embodiment 5. The method of any preceding embodiment wherein the method is in vitro.
Embodiment 6. The method of any preceding embodiment, wherein the cochlear supporting cell(s) or vestibular supporting cell(s) express(es) leucine-rich repeat-containing G-protein coupled receptor 5 (Lgr5).
Embodiment 7. The method of any preceding embodiment, wherein the cochlear supporting cell(s) or vestibular supporting cell(s) are/is a mature cell(s).
Embodiment 8. The method of any preceding embodiment, wherein the expanded population of cochlear or vestibular cells expresses leucine-rich repeat-containing G-protein coupled receptor 5 (Lgr5).
Embodiment 9. The method of any preceding embodiment, wherein the cochlear supporting cell(s) or vestibular supporting cell(s) are/is a cochlear supporting cell(s).
Embodiment 10. The method of any preceding embodiment, wherein the expanded population of cochlear or vestibular cells are cochlear cells.
Embodiment 11. The method of any preceding embodiment, wherein the TAZ activator in combination with the Wnt agonist increases the Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay
Embodiment 12. The method of any preceding embodiment, wherein the TAZ activator is IBS008738 at a concentration of about 10 μM.
Embodiment 13. The method of any preceding embodiment, wherein the Wnt agonist is CHIR99021 at a concentration of about 4 μM.
Embodiment 14. A method of treating a subject who has, or is at risk of, developing an inner ear hearing or balance disorder, comprising administering to the subject:
   a) a transcriptional coactivator with PDZ-binding motif (TAZ) activator; and
   b) a Wnt agonist
      wherein (a) and (b) can occur in any order or simultaneously.
Embodiment 15. The method of embodiment 14, wherein the subject has an inner ear hearing or balance disorder.
Embodiment 16. The method of embodiment 14 or 15, wherein the inner ear hearing or balance disorder is sensorineural hearing loss.
Embodiment 17. The method of any of embodiments 14-16, wherein the treatment results in improved auditory function when assessed by behavioural audiometry or auditory brainstem response (ABR) testing.
Embodiment 18. The method of any of embodiments 14-17, wherein the TAZ activator is administered locally and/or systemically.
Embodiment 19. The method of embodiment 18, wherein the TAZ activator is administered locally.
Embodiment 20. The method of embodiment 18, wherein the TAZ activator is administered systemically.
Embodiment 21. The method of embodiment 18, wherein the TAZ activator is administered locally and systemically.
Embodiment 22. The method of any preceding embodiment, wherein the Wnt agonist is administered locally and/or systemically.
Embodiment 23. The method of any preceding embodiment, wherein the Wnt agonist is administered locally.
Embodiment 24. The method of any preceding embodiment, wherein the Wnt agonist is administered systemically.
Embodiment 25. The method of any preceding embodiment, wherein the Wnt agonist is administered locally and systemically.
Embodiment 26. The method of any preceding embodiment, wherein the epigenetic agent is administered locally and/or systemically.
Embodiment 27. The method of any preceding embodiment, wherein the epigenetic agent is administered locally.
Embodiment 28. The method of any preceding embodiment, wherein the epigenetic agent is administered systemically.
Embodiment 29. The method of any preceding embodiment, wherein the epigenetic agent is administered locally and systemically.
Embodiment 30. The method of any preceding embodiment, wherein the local administration is to the tympanic membrane, the middle ear or the inner ear.
Embodiment 31. The method of any of the above enumerated embodiments wherein a TAZ activator results in a decrease in TAZ phosphorylation in a cell.
Embodiment 32. The method of any preceding embodiment, wherein the TAZ activator is selected from the group consisting of AS 8351 or TC-E 5002.
Embodiment 33. The method of any preceding embodiment, wherein the TAZ activator is IBS008738.
Embodiment 34. The method of any preceding embodiment, wherein the TAZ activator is TM-25659.
Embodiment 35. The method of any preceeding embodiment, wherein the TAZ activator is TT-10.
Embodiment 36. The method of embodiment 33, wherein IBS008738 is at a concentration of about between 1 μM to 30 μM.
Embodiment 37. The method of embodiment 34, wherein TM-25659 is at a concentration of about between 10 μM to 100 μM.
Embodiment 38. The method of embodiment 35, wherein TT-10 is at a concentration of about between 1 μM to 10 μM.
Embodiment 39. The method of any preceding embodiment, wherein the Wnt agonist is a GSK3 inhibitor.
Embodiment 40. The method of any preceding embodiment, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 41. The method of any preceding embodiment, wherein the GSK3 inhibitor is AZD1080.
Embodiment 42. The method of any preceding embodiment, wherein the GSK3 inhibitor is LY2090314.
Embodiment 43. The method of any preceding embodiment, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 44. The method of any preceding embodiment, wherein the GSK3 inhibitor is GSK3 inhibitor XXII.
Embodiment 45. The method of any preceding embodiment, wherein the GSK3 inhibitor is CHIR99021.
Embodiment 46. The method of any preceding embodiment, wherein AZD1080 is at a concentration of about between 0.5 μM to 5 μM.
Embodiment 47. The method of any preceding embodiment, wherein LY2090314 is at a concentration of about between 4 nM to 40 nM.
Embodiment 48. The method of any preceding claim, wherein the disorder is an inner ear hearing disorder.
Embodiment 49. The method of any preceding claim, wherein the disorder is a balance disorder.
Embodiment 50. The method of any preceding embodiment, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is as defined in Formula A.
Embodiment 51. The method of any preceding embodiment, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is selected from those disclosed in Table 4.
Embodiment 52. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(imidazo[1,2-a]pyridin-3-yl)-4-(2-(piperidine-1-carbonyl)-9-(trifluoromethyl)-1,2,3,4-tetrahydro-[1,4]diazepino[6,7,1-hi]indol-7-yl)-1H-pyrrole-2,5-dione.
Embodiment 53. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indole-9-carbonitrile.
Embodiment 54. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-ethynyl-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 55. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-amino-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 56. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 1-(9-fluoro-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-2-carbonyl)piperidine-4-carbaldehyde.
Embodiment 57. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(hydroxymethvl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 58. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 59. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(8-oxa-3-azabicyclo[3.2.1]octane-3-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 60. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(benzo[d]isoxazol-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione.
Embodiment 61. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is N-(7-(4-(imidazo[1,2-a]pyridin-3-yl)-2,5-dioxo-2,5-dihydro-1H-pyrrol-3-yl)-2-(piperidine-1-carbonyl)-1,2,3,4-tetrahydro-[1,4]diazepino[6,7,1-hi]indol-9-yl)acetamide.
Embodiment 62. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-(difluoromethyl)-2-(piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 63. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(3,3-difluoropiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 64. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-((1R,4R)-2,5-diazabicyclo[2.2.1]heptane-2-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 65. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(8-oxa-3-azabicyclo[3.2.1]octane-3-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile.
Embodiment 66. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(3,3-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile,
Embodiment 67. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4,4-difluoropiperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile,
Embodiment 68. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoropiperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 69. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(8-oxa-3-azabicyclo[3.2.1]octane-3-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 70. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(aminomethyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 71. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(hydroxymethyl)piperidine-1-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 72. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4-(hydroxymethyl)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile,
Embodiment 73. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(3,3,4,4,5,5-hexafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 74. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(3,3,5,5-tetrafluoropiperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione
Embodiment 75. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(2,2,6,6-tetrafluoromorpholine-4-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 76. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4,4-difluoro-3-hydroxypiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 77. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(difluoro(hydroxy)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 78. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(6,6-difluoro-1,4-oxazepane-4-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 79. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-([1,2,4]triazolo[4,3-a]pyridin-3-yl)-4-(9-fluoro-2-(piperidine-1-carbonyl)-1,2,3,4-tetrahydro-[1,4]diazepino[6,7,1-hi]indol-7-yl)-1H-pyrrole-2,5-dione.
Embodiment 80. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl-d10)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 81. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(piperidine-1-carbonyl)-1,2,3,4-tetrahydro-[1,4]diazepino[6,7,1-hi]indol-7-yl-3,3,4,4-d4)-4-(imidazo[1,2-a]pyridin-3-yl)-1H-pyrrole-2,5-dione.
Embodiment 82. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(2,2,2-trifluoro-1-hydroxyethyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 83. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-((methylamino)methyl)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 84. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-((dimethylamino)methyl)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 85. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-aminopiperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 86. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(4-(methylamino)piperidine-1-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 87. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(4-(dimethylamino)piperidine-1-carbonyl)-9-fluoro-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 88. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-(piperidin-4-ylmethyl)-3,4-dihydro-[1,4]diazepino[6,7,1-hi]indole-2(1H)-carboxamide,
Embodiment 89. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-methyl-N-(piperidin-4-ylmethyl)-3,4-dihydro-[1,4]diazepino[6,7,1-hi]indole-2(1H)-carboxamide,
Embodiment 90. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-fluoro-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide,
Embodiment 91. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-((1R,4R)-5-methyl-2,5-diazabicyclo[2.2.1]heptane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 92. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(2-methyl-2,8-diazaspiro[4.5]decane-8-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 93. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(9-fluoro-2-(8-methyl-2,8-diazaspiro[4.5]decane-2-carbonyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 94. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(imidazo[1,2-a]pyridin-3-yl)-4-(2-(2,2,6,6-tetrafluoromorpholine-4-carbonyl)-9-(trifluoromethyl)-1,2,3,4-tetrahydro-[1,4]diazepino[6,7,1-hi]indol-7-yl)-1H-pyrrole-2,5-dione.
Embodiment 95. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 3-(2-(6,6-difluoro-1,4-oxazepane-4-carbonyl)-9-(trifluoromethyl)-1,2,3,4-tetrahydro-[1,4]diazepino[6,7,1-hi]indol-7-yl)-4-(imidazo[1,2-a]pyridin-3-yl)-1H-pyrrole-2,5-dione.
Embodiment 96. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 2-(4-(dimethylamino)piperidine-1-carbonyl)-7-(4-(imidazo[1,2-a]pyridin-3-yl)-2,5-dioxo-2,5-dihydro-1H-pyrrol-3-yl)-1,2,3,4-tetrahydro-[1,4]diazepino[6,7,1-hi]indole-9-carbonitrile,
Embodiment 97. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 9-cyano-7-(4-(imidazo[1,2-a]pyridin-3-yl)-2,5-dioxo-2,5-dihydro-1H-pyrrol-3-yl)-N-methyl-N-((1-methylpiperidin-4-yl)methyl)-3,4-dihydro-[1,4]diazepino[6,7,1-hi]indole-2(1H)-carboxamide,
Embodiment 98. The method of any of the above enumerated embodiments wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is 7-(4-(imidazo[1,2-a]pyridin-3-yl)-2,5-dioxo-2,5-dihydro-1H-pyrrol-3-yl)-2-(8-methyl-2,8-diazaspiro[4.5]decane-2-carbonyl)-1,2,3,4-tetrahydro-[1,4]diazepino[6,7,1-hi]indole-9-carbonitrile
Embodiment 99. The method of any of the above enumerated embodiments wherein the TAZ activator increases the stability or activity of TAZ by at least 5% relative to a control.
Embodiment 100. The method of any of the above enumerated embodiments wherein the TAZ activator increases the stability or activity of TAZ by at least 10% relative to a control.
Embodiment 101. The method of any of the above enumerated embodiments wherein the TAZ activator increases the stability or activity of TAZ by at least 20% relative to a control.
Embodiment 102. The method of any of the above enumerated embodiments wherein the TAZ activator increases the stability or activity of TAZ by at least 30% relative to a control.
Embodiment 103. The method of any of the above enumerated embodiments wherein the TAZ activator increases the stability or activity of TAZ by at least 40% relative to a control.
Embodiment 104. The method of any of the above enumerated embodiments wherein the TAZ activator increases the stability or activity of TAZ by at least 50% relative to a control.
Embodiment 105. The method of any of the above enumerated embodiments wherein the TAZ activator increases the stability or activity of TAZ by at least 60% relative to a control.
Embodiment 106. The method of any of the above enumerated embodiments wherein the TAZ activator increases the stability or activity of TAZ by at least 70% relative to a control.
Embodiment 107. The method of any of the above enumerated embodiments wherein the TAZ activator increases the stability or activity of TAZ by at least 80% relative to a control.
Embodiment 108. The method of any of the above enumerated embodiments wherein the TAZ activator increases the stability or activity of TAZ by at least 90% relative to a control.
Embodiment 109. The method of any of the above enumerated embodiments wherein the TAZ activator increases the stability or activity of TAZ by at least 100% relative to a control.
Embodiment 110. The method of any of the above enumerated embodiments wherein the TAZ activator decreases TAZ phosphorylation by at least about 1.1-fold or more relative to a control.
Embodiment 111. The method of any of the above enumerated embodiments wherein the TAZ activator decreases TAZ phosphorylation by at least about 1.2-fold or more relative to a control.
Embodiment 112. The method of any of the above enumerated embodiments wherein the TAZ activator decreases TAZ phosphorylation by at least about 1.3-fold or more relative to a control.
Embodiment 113. The method of any of the above enumerated embodiments wherein the KDM inhibitor decreases demethylation by at least about 1.4-fold or more relative to a control.
Embodiment 114. The method of any of the above enumerated embodiments wherein the TAZ activator decreases TAZ phosphorylation by at least about 1.5-fold or more relative to a control.
Embodiment 115. The method of any of the above enumerated embodiments wherein the TAZ activator decreases TAZ phosphorylation by at least about 1.6-fold or more relative to a control.
Embodiment 116. The method of any of the above enumerated embodiments wherein the TAZ activator decreases TAZ phosphorylation by at least about 1.7-fold or more relative to a control.
Embodiment 117. The method of any of the above enumerated embodiments wherein the TAZ activator decreases TAZ phosphorylation by at least about 1.8-fold or more relative to a control.
Embodiment 118. The method of any of the above enumerated embodiments wherein the TAZ activator decreases TAZ phosphorylation by at least about 1.9-fold or more relative to a control.
Embodiment 119. The method of any of the above enumerated embodiments wherein the TAZ activator decreases TAZ phosphorylation by at least about 2-fold or more relative to a control.
Embodiment 120. The method of any of the above enumerated embodiments wherein the TAZ activator decreases TAZ phosphorylation by at least about 3-fold or more relative to a control.
Embodiment 121. The method of any of the above enumerated embodiments wherein the TAZ activator decreases TAZ phosphorylation by at least about 4-fold or more relative to a control.
Embodiment 122. The method of any of the above enumerated embodiments wherein the TAZ activator decreases TAZ phosphorylation by at least about 5-fold or more relative to a control.
Embodiment 123. The method of any of the above enumerated embodiments wherein the TAZ activator decreases TAZ phosphorylation by at least about 6-fold or more relative to a control.
Embodiment 124. The method of any of the above enumerated embodiments wherein the TAZ activator decreases TAZ phosphorylation by at least about 7-fold or more relative to a control.
Embodiment 125. The method of any of the above enumerated embodiments wherein the TAZ activator decreases TAZ phosphorylation by at least about 8-fold or more relative to a control.
Embodiment 126. The method of any of the above enumerated embodiments wherein the TAZ activator decreases TAZ phosphorylation by at least about 9-fold or more relative to a control.
Embodiment 127. The method of any of the above enumerated embodiments wherein the TAZ activator decreases TAZ phosphorylation by at least about 10-fold or more relative to a control.
Embodiment 128. The method of any of the above enumerated embodiments wherein the TAZ activator decreases TAZ phosphorylation by at least about 15-fold or more relative to a control.
Embodiment 129. The method of any of the above enumerated embodiments wherein the TAZ activator decreases TAZ phosphorylation by at least about 20-fold or more relative to a control.
Embodiment 130. The method of any of the above enumerated embodiments wherein the TAZ activator decreases TAZ phosphorylation by at least about 30-fold or more relative to a control.
Embodiment 131. The method of any of the above enumerated embodiments wherein the TAZ activator decreases TAZ phosphorylation by at least about 40-fold or more relative to a control.
Embodiment 132. The method of any of the above enumerated embodiments wherein the TAZ activator decreases TAZ phosphorylation by at least about 50-fold or more relative to a control.
Embodiment 133. The method of any of the above enumerated embodiments wherein the TAZ activator decreases TAZ phosphorylation by at least about 60-fold or more relative to a control.
Embodiment 134. The method of any of the above enumerated embodiments wherein the TAZ activator decreases TAZ phosphorylation by at least about 70-fold or more relative to a control.
Embodiment 135. The method of any of the above enumerated embodiments wherein the TAZ activator decreases TAZ phosphorylation by at least about 80-fold or more relative to a control.
Embodiment 136. The method of any of the above enumerated embodiments wherein the TAZ activator decreases TAZ phosphorylation by at least about 90-fold or more relative to a control.
Embodiment 137. The method of any of the above enumerated embodiments wherein the TAZ activator decreases TAZ phosphorylation by at least about 100-fold or more relative to a control.
Embodiment 138. The method of any of the above enumerated embodiments wherein the TAZ activator decreases TAZ phosphorylation by at least about 200-fold or more relative to a control.
Embodiment 139. The method of any of the above enumerated embodiments wherein the TAZ activator decreases TAZ phosphorylation by at least about 500-fold or more relative to a control.
Embodiment 140. The method of any of the above enumerated embodiments wherein the TAZ activator decreases TAZ phosphorylation by at least about 1000-fold or more relative to a control.
Embodiment 141. The method of any of the above enumerated embodiments wherein the TAZ activator modulates expression or activity of a target gene by at least 5% relative to a control.
Embodiment 142. The method of any of the above enumerated embodiments wherein the TAZ activator modulates expression or activity of a target gene by at least 10% relative to a control.
Embodiment 143. The method of any of the above enumerated embodiments wherein the TAZ activator modulates expression or activity of a target gene by at least 20% relative to a control.
Embodiment 144. The method of any of the above enumerated embodiments wherein the TAZ activator modulates expression or activity of a target gene by at least 30% relative to a control.
Embodiment 145. The method of any of the above enumerated embodiments wherein the TAZ activator modulates expression or activity of a target gene by at least 40% relative to a control.
Embodiment 146. The method of any of the above enumerated embodiments wherein the TAZ activator modulates expression or activity of a target gene by at least 50% relative to a control.
Embodiment 147. The method of any of the above enumerated embodiments wherein the TAZ activator modulates expression or activity of a target gene by at least 60% relative to a control.
Embodiment 148. The method of any of the above enumerated embodiments wherein the TAZ activator modulates expression or activity of a target gene by at least 70% relative to a control.
Embodiment 149. The method of any of the above enumerated embodiments wherein the TAZ activator modulates expression or activity of a target gene by at least 80% relative to a control.
Embodiment 150. The method of any of the above enumerated embodiments wherein the TAZ activator modulates expression or activity of a target gene by at least 90% relative to a control.
Embodiment 151. The method of any of the above enumerated embodiments wherein the TAZ activator modulates expression or activity of a target gene by at least 1⁰⁰% relative to a control.
Embodiment 152. The method of any of the above enumerated embodiments wherein the TAZ activator increases expression or activity of a target gene relative to a control.
Embodiment 153. The method of any of the above enumerated embodiments wherein the TAZ activator decreases expression or activity of a target gene relative to a control.
Embodiment 154. The method of any of the above enumerated embodiments wherein the TAZ activator increases stability or activity of TAZ by at least about 1.1-fold or more relative to a control.
Embodiment 155. The method of any of the above enumerated embodiments wherein the TAZ activator increases stability or activity of TAZ by at least about 1.2-fold or more relative to a control.
Embodiment 156. The method of any of the above enumerated embodiments wherein the TAZ activator increases stability or activity of TAZ by at least about 1.3-fold or more relative to a control.
Embodiment 157. The method of any of the above enumerated embodiments wherein the TAZ activator increases stability or activity of TAZ by at least about 1.4-fold or more relative to a control.
Embodiment 158. The method of any of the above enumerated embodiments wherein the TAZ activator increases stability or activity of TAZ by at least about 1.5-fold or more relative to a control.
Embodiment 159. The method of any of the above enumerated embodiments wherein the TAZ activator increases stability or activity of TAZ by at least about 1.6-fold or more relative to a control.
Embodiment 160. The method of any of the above enumerated embodiments wherein the TAZ activator increases stability or activity of TAZ by at least about 1.7-fold or more relative to a control.
Embodiment 161. The method of any of the above enumerated embodiments wherein the TAZ activator increases stability or activity of TAZ by at least about 1.8-fold or more relative to a control.
Embodiment 162. The method of any of the above enumerated embodiments wherein the TAZ activator increases stability or activity of TAZ by at least about 1.9-fold or more relative to a control.
Embodiment 163. The method of any of the above enumerated embodiments wherein the TAZ activator increases stability or activity of TAZ by at least about 2-fold or more relative to a control.
Embodiment 164. The method of any of the above enumerated embodiments wherein the TAZ activator increases stability or activity of TAZ by at least about 3-fold or more relative to a control.
Embodiment 165. The method of any of the above enumerated embodiments wherein the TAZ activator increases stability or activity of TAZ by at least about 4-fold or more relative to a control.
Embodiment 166. The method of any of the above enumerated embodiments wherein the TAZ activator increases stability or activity of TAZ by at least about 5-fold or more relative to a control.
Embodiment 167. The method of any of the above enumerated embodiments wherein the TAZ activator increases stability or activity of TAZ by at least about 6-fold or more relative to a control.
Embodiment 168. The method of any of the above enumerated embodiments wherein the TAZ activator increases stability or activity of TAZ by at least about 7-fold or more relative to a control.
Embodiment 169. The method of any of the above enumerated embodiments wherein the TAZ activator increases stability or activity of TAZ by at least about 8-fold or more relative to a control.
Embodiment 170. The method of any of the above enumerated embodiments wherein the TAZ activator increases stability or activity of TAZ by at least about 9-fold or more relative to a control.
Embodiment 171. The method of any of the above enumerated embodiments wherein the TAZ activator increases stability or activity of TAZ by at least about 10-fold or more relative to a control.
Embodiment 172. The method of any of the above enumerated embodiments wherein the TAZ activator increases stability or activity of TAZ by at least about 15-fold or more relative to a control.
Embodiment 173. The method of any of the above enumerated embodiments wherein the TAZ activator increases stability or activity of TAZ by at least about 20-fold or more relative to a control.
Embodiment 174. The method of any of the above enumerated embodiments wherein the TAZ activator increases stability or activity of TAZ by at least about 30-fold or more relative to a control.
Embodiment 175. The method of any of the above enumerated embodiments wherein the TAZ activator increases stability or activity of TAZ by at least about 40-fold or more relative to a control.
Embodiment 176. The method of any of the above enumerated embodiments wherein the TAZ activator increases stability or activity of TAZ by at least about 50-fold or more relative to a control.
Embodiment 177. The method of any of the above enumerated embodiments wherein the TAZ activator increases stability or activity of TAZ by at least about 60-fold or more relative to a control.
Embodiment 178. The method of any of the above enumerated embodiments wherein the TAZ activator increases stability or activity of TAZ by at least about 70-fold or more relative to a control.
Embodiment 179. The method of any of the above enumerated embodiments wherein the TAZ activator increases stability or activity of TAZ by at least about 80-fold or more relative to a control.
Embodiment 180. The method of any of the above enumerated embodiments wherein the TAZ activator increases stability or activity of TAZ by at least about 90-fold or more relative to a control.
Embodiment 181. The method of any of the above enumerated embodiments wherein the TAZ activator increases stability or activity of TAZ by at least about 100-fold or more relative to a control.
Embodiment 182. The method of any of the above enumerated embodiments wherein the TAZ activator increases stability or activity of TAZ by at least about 200-fold or more relative to a control.
Embodiment 183. The method of any of the above enumerated embodiments wherein the TAZ activator increases stability or activity of TAZ by at least about 500-fold or more relative to a control.
Embodiment 184. The method of any of the above enumerated embodiments wherein the TAZ activator increases stability or activity of TAZ by at least about 1000-fold or more relative to a control.
Embodiment 185. The method of any of the above enumerated embodiments wherein the TAZ activator increases the expression of a target gene by at least about 1.1-fold or more relative to a control.
Embodiment 186. The method of any of the above enumerated embodiments wherein the TAZ activator increases the expression of a target gene by at least about 1.2-fold or more relative to a control.
Embodiment 187. The method of any of the above enumerated embodiments wherein the TAZ activator increases the expression of a target gene by at least about 1.3-fold or more relative to a control.
Embodiment 188. The method of any of the above enumerated embodiments wherein the TAZ activator increases the expression of a target gene by at least about 1.4-fold or more relative to a control.
Embodiment 189. The method of any of the above enumerated embodiments wherein the TAZ activator increases the expression of a target gene by at least about 1.5-fold or more relative to a control.
Embodiment 190. The method of any of the above enumerated embodiments wherein the TAZ activator increases the expression of a target gene by at least about 1.6-fold or more relative to a control.
Embodiment 191. The method of any of the above enumerated embodiments wherein the TAZ activator increases the expression of a target gene by at least about 1.7-fold or more relative to a control.
Embodiment 192. The method of any of the above enumerated embodiments wherein the TAZ activator increases the expression of a target gene by at least about 1.8-fold or more relative to a control.
Embodiment 193. The method of any of the above enumerated embodiments wherein the TAZ activator increases the expression of a target gene by at least about 1.9-fold or more relative to a control.
Embodiment 194. The method of any of the above enumerated embodiments wherein the TAZ activator increases the expression of a target gene by at least about 2-fold or more relative to a control.
Embodiment 195. The method of any of the above enumerated embodiments wherein the TAZ activator increases the expression of a target gene by at least about 3-fold or more relative to a control.
Embodiment 196. The method of any of the above enumerated embodiments wherein the TAZ activator increases the expression of a target gene by at least about 4-fold or more relative to a control.
Embodiment 197. The method of any of the above enumerated embodiments wherein the TAZ activator increases the expression of a target gene by at least about 5-fold or more relative to a control.
Embodiment 198. The method of any of the above enumerated embodiments wherein the TAZ activator increases the expression of a target gene by at least about 6-fold or more relative to a control.
Embodiment 199. The method of any of the above enumerated embodiments wherein the TAZ activator increases the expression of a target gene by at least about 7-fold or more relative to a control.
Embodiment 200. The method of any of the above enumerated embodiments wherein the TAZ activator increases the expression of a target gene by at least about 8-fold or more relative to a control.
Embodiment 201. The method of any of the above enumerated embodiments wherein the TAZ activator increases the expression of a target gene by at least about 9-fold or more relative to a control.
Embodiment 202. The method of any of the above enumerated embodiments wherein the TAZ activator increases the expression of a target gene by at least about 10-fold or more relative to a control.
Embodiment 203. The method of any of the above enumerated embodiments wherein the TAZ activator increases the expression of a target gene by at least about 15-fold or more relative to a control.
Embodiment 204. The method of any of the above enumerated embodiments wherein the TAZ activator increases the expression of a target gene by at least about 20-fold or more relative to a control.
Embodiment 205. The method of any of the above enumerated embodiments wherein the TAZ activator increases the expression of a target gene by at least about 30-fold or more relative to a control.
Embodiment 206. The method of any of the above enumerated embodiments wherein the TAZ activator increases the expression of a target gene by at least about 40-fold or more relative to a control.
Embodiment 207. The method of any of the above enumerated embodiments wherein the TAZ activator increases the expression of a target gene by at least about 50-fold or more relative to a control.
Embodiment 208. The method of any of the above enumerated embodiments wherein the TAZ activator increases the expression of a target gene by at least about 60-fold or more relative to a control.
Embodiment 209. The method of any of the above enumerated embodiments wherein the TAZ activator increases the expression of a target gene by at least about 70-fold or more relative to a control.
Embodiment 210. The method of any of the above enumerated embodiments wherein the TAZ activator increases the expression of a target gene by at least about 80-fold or more relative to a control.
Embodiment 211. The method of any of the above enumerated embodiments wherein the TAZ activator increases the expression of a target gene by at least about 90-fold or more relative to a control.
Embodiment 212. The method of any of the above enumerated embodiments wherein the TAZ activator increases the expression of a target gene by at least about 100-fold or more relative to a control.
Embodiment 213. The method of any of the above enumerated embodiments wherein the TAZ activator increases the expression of a target gene by at least about 200-fold or more relative to a control.
Embodiment 214. The method of any of the above enumerated embodiments wherein the TAZ activator increases the expression of a target gene by at least about 500-fold or more relative to a control.
Embodiment 215. The method of any of the above enumerated embodiments wherein the TAZ activator increases the expression of a target gene by at least about 1000-fold or more relative to a control.
Embodiment 216. The method of any of the above enumerated embodiments wherein the TAZ activator is reversible.
Embodiment 217. The method of any of the above enumerated embodiments wherein the TAZ activator is irreversible.
Embodiment 218. The method of any preceding embodiment, wherein the TAZ activator is selected from the group consisting of AS 8351 or TC-E 5002.
Embodiment 219. The method of any preceding embodiment, wherein the TAZ activator is IBS008738.
Embodiment 220. The method of any preceding embodiment, wherein the TAZ activator is TM-25659.
Embodiment 221. The method of any preceeding embodiment, wherein the TAZ activator is TT-10.
Embodiment 222. The method of embodiment 33, wherein IBS008738 is at a concentration of about between 1 μM to 30 μM.
Embodiment 223. The method of embodiment 34, wherein TM-25659 is at a concentration of about between 10 μM to 100 μM.
Embodiment 224. The method of embodiment 35, wherein TT-10 is at a concentration of about between 1 μM to 10 μM.
Embodiment 225. The method of any of the above enumerated embodiments wherein the TAZ activator is TAZ12 from JACC. Basic to translational science (2018), 3(5), 639-653.
Embodiment 226. The method of any of the above enumerated embodiments wherein the TAZ activator is TM-53 from British J Pharmacol 2014, 171(17), 4051-61 Novel TAZ modulators-TM-53, 54.
Embodiment 227. The method of any of the above enumerated embodiments wherein the TAZ activator is TM-54 from British J Pharmacol 2014, 171(17), 4051-61 Novel TAZ modulators-TM-53, 54.
Embodiment 228. The method of any of the above enumerated embodiments wherein the TAZ activator is ethacridine from Journal of Biochemistry (2015), 158(5), 413-423.
Embodiment 229. The method of any of the above enumerated embodiments wherein the TAZ activator is IBS003031 from Molecular Cancer Research (2018), 16(2), 197-211-YAP activator IBS003031.
Embodiment 230. The method of any of the above enumerated embodiments wherein the TAZ activator is KR 62980 is from Biochemical Pharmacology (2009), 78(10), 1323-1329-KR62980.
Embodiment 231. The method of any of the above enumerated embodiments wherein the TAZ activator is kaempferol from Bone 50 (2012) 364-372-kaempferol.
Embodiment 232. The method of any of the above enumerated embodiments wherein the TAZ activator is (−)epicatechin gallate from JBC 2014, 289(14), 9926-35-Epicatechin Gallate.
Embodiment 233. The method of any of the above enumerated embodiments wherein the TAZ activator is phorbaketal A is from FEBS Lett 2012, 586(8), 1086-Phorbaketal A
Embodiment 234. The method of any of the above enumerated embodiments wherein the agent having activity as a TAZ activator is an agent listed in Table 1.
Embodiment 235. The method of any of the above enumerated embodiments wherein the TAZ activator is administered in an amount sufficient to achieve a concentration of 0.001 μM to 100 mM in the perilymph fluid in the inner ear.
Embodiment 236. The method of any of the above enumerated embodiments wherein the TAZ activator is administered in an amount sufficient to achieve a concentration of about 0.001 μM to 10 mM in the perilymph fluid in the inner ear.
Embodiment 237. The method of any of the above enumerated embodiments wherein the TAZ activator is administered in an amount sufficient to achieve a concentration of about 0.01 μM to 10 mM in the perilymph fluid in the inner ear.
Embodiment 238. The method of any of the above enumerated embodiments wherein the TAZ activator is administered in an amount sufficient to achieve a concentration of about 0.1 μM to 1 mM in the perilymph fluid in the inner ear.
Embodiment 239. The method of any of the above enumerated embodiments wherein the TAZ activator is administered in an amount sufficient to achieve a concentration of about 0.01 μM to 1 mM in the perilymph fluid in the inner ear.
Embodiment 240. The method of any of the above enumerated embodiments wherein the TAZ activator is administered in an amount sufficient to achieve a concentration of about 0.1 μM to 100 μM in the perilymph fluid in the inner ear.
Embodiment 241. The method of any of the above enumerated embodiments wherein the TAZ activator is administered in an amount sufficient to achieve a concentration of about 1 μM to 100 μM in the perilymph fluid in the inner ear.
Embodiment 242. The method of any of the above enumerated embodiments wherein the TAZ activator is administered in an amount sufficient to achieve a concentration of about 0.01 nM to 1 μM in the perilymph fluid in the inner ear.
Embodiment 243. The method of any of the above enumerated embodiments wherein the TAZ activator is administered to in an amount sufficient to achieve a concentration of about 0.1 nM to 100 nM in the perilymph fluid in the inner ear.
Embodiment 244. The method of any of the above enumerated embodiments wherein the TAZ activator is administered to in an amount sufficient to achieve a concentration of about 0.001 nM to 0.01 nM in the perilymph fluid in the inner ear.
Embodiment 245. The method of any of the above enumerated embodiments wherein the TAZ activator is administered in an amount sufficient to achieve a concentration of about 0.01 nM to 0.1 nM in the perilymph fluid in the inner ear.
Embodiment 246. The method of any of the above enumerated embodiments wherein the TAZ activator is administered in an amount sufficient to achieve a concentration of about 0.1 nM to 1 nM in the perilymph fluid in the inner ear.
Embodiment 247. The method of any of the above enumerated embodiments wherein the TAZ activator is administered in an amount sufficient to achieve a concentration of about 1 nM to 10 nM in the perilymph fluid in the inner ear.
Embodiment 248. The method of any of the above enumerated embodiments wherein the TAZ activator is administered in an amount sufficient to achieve a concentration of about 10 nM to 100 nM in the perilymph fluid in the inner ear.
Embodiment 249. The method of any of the above enumerated embodiments wherein the TAZ activator is administered in an amount sufficient to achieve a concentration of about 100 nM to 1 μM in the perilymph fluid in the inner ear.
Embodiment 250. The method of any of the above enumerated embodiments wherein the TAZ activator is administered in amount sufficient to achieve a concentration of about 1 μM to 10 μM in the perilymph fluid in the inner ear.
Embodiment 251. The method of any of the above enumerated embodiments wherein the TAZ activator is administered in amount sufficient to achieve a concentration of about 10 μM to 100 μM in the perilymph fluid in the inner ear.
Embodiment 252. The method of any of the above enumerated embodiments wherein the TAZ activator is administered in amount sufficient to achieve a concentration of about 100 μM to 1 mM in the perilymph fluid in the inner ear.
Embodiment 253. The method of any of the above enumerated embodiments wherein the TAZ activator is administered in amount sufficient to achieve a concentration of about 1 mM to 10 mM in the perilymph fluid in the inner ear.
Embodiment 254. The method of any of the above enumerated embodiments wherein the TAZ activator is administered in an amount sufficient to achieve a concentration of about 0.1 nM in the perilymph fluid in the inner ear.
Embodiment 255. The method of any of the above enumerated embodiments wherein the TAZ activator is administered in an amount sufficient to achieve a concentration of about 0.2 nM in the perilymph fluid in the inner ear.
Embodiment 256. The method of any of the above enumerated embodiments wherein the TAZ activator is administered in an amount sufficient to achieve a concentration of about 0.3 nM in the perilymph fluid in the inner ear.
Embodiment 257. The method of any of the above enumerated embodiments wherein the TAZ activator is administered in an amount sufficient to achieve a concentration of about 0.4 nM in the perilymph fluid in the inner ear.
Embodiment 258. The method of any of the above enumerated embodiments wherein the TAZ activator is administered in an amount sufficient to achieve a concentration of about 0.5 nM in the perilymph fluid in the inner ear.
Embodiment 259. The method of any of the above enumerated embodiments wherein the TAZ activator is administered in an amount sufficient to achieve a concentration of about 0.6 nM in the perilymph fluid in the inner ear.
Embodiment 260. The method of any of the above enumerated embodiments wherein the TAZ activator is administered in an amount sufficient to achieve a concentration of about 0.7 nM in the perilymph fluid in the inner ear.
Embodiment 261. The method of any of the above enumerated embodiments wherein the TAZ activator is administered in an amount sufficient to achieve a concentration of about 0.8 nM in the perilymph fluid in the inner ear.
Embodiment 262. The method of any of the above enumerated embodiments wherein the TAZ activator is administered in an amount sufficient to achieve a concentration of about 0.9 nM in the perilymph fluid in the inner ear.
Embodiment 263. The method of any of the above enumerated embodiments wherein the TAZ activator is administered in an amount sufficient to achieve a concentration of about 1.0 nM in the perilymph fluid in the inner ear.
Embodiment 264. The method of any of the above enumerated embodiments wherein the TAZ activator is administered in an amount sufficient to achieve a concentration of about 2.0 nM in the perilymph fluid in the inner ear.
Embodiment 265. The method of any of the above enumerated embodiments wherein the TAZ activator is administered in an amount sufficient to achieve a concentration of about 3.0 nM in the perilymph fluid in the inner ear.
Embodiment 266. The method of any of the above enumerated embodiments wherein the TAZ activator is administered in an amount sufficient to achieve a concentration of about 4.0 nM in the perilymph fluid in the inner ear.
Embodiment 267. The method of any of the above enumerated embodiments wherein the TAZ activator is administered in an amount sufficient to achieve a concentration of about 5.0 nM in the perilymph fluid in the inner ear.
Embodiment 268. The method of any of the above enumerated embodiments wherein the TAZ activator is administered in an amount sufficient to achieve a concentration of about 6.0 nM in the perilymph fluid in the inner ear.
Embodiment 269. The method of any of the above enumerated embodiments wherein the TAZ activator is administered in an amount sufficient to achieve a concentration of about 7.0 nM in the perilymph fluid in the inner ear.
Embodiment 270. The method of any of the above enumerated embodiments wherein the TAZ activator is administered in an amount sufficient to achieve a concentration of about 8.0 nM in the perilymph fluid in the inner ear.
Embodiment 271. The method of any of the above enumerated embodiments wherein the TAZ activator is administered in an amount sufficient to achieve a concentration of about 9.0 nM in the perilymph fluid in the inner ear.
Embodiment 272. The method of any of the above enumerated embodiments wherein the TAZ activator is administered in an amount sufficient to achieve a concentration of about 10 nM in the perilymph fluid in the inner ear.
Embodiment 273. The method of any of the above enumerated embodiments wherein the TAZ activator is administered in an amount sufficient to achieve a concentration of about 20 nM in the perilymph fluid in the inner ear.
Embodiment 274. The method of any of the above enumerated embodiments wherein the TAZ activator is administered in an amount sufficient to achieve a concentration of about 30 nM in the perilymph fluid in the inner ear.
Embodiment 275. The method of any of the above enumerated embodiments wherein the TAZ activator is administered in an amount sufficient to achieve a concentration of about 40 nM in the perilymph fluid in the inner ear.
Embodiment 276. The method of any of the above enumerated embodiments wherein the TAZ activator is administered in an amount sufficient to achieve a concentration of about 50 nM in the perilymph fluid in the inner ear.
Embodiment 277. The method of any of the above enumerated embodiments wherein the TAZ activator is administered in an amount sufficient to achieve a concentration of about 60 nM in the perilymph fluid in the inner ear.
Embodiment 278. The method of any of the above enumerated embodiments wherein the TAZ activator is administered in an amount sufficient to achieve a concentration of about 70 nM in the perilymph fluid in the inner ear.
Embodiment 279. The method of any of the above enumerated embodiments wherein the TAZ activator is administered in an amount sufficient to achieve a concentration of about 80 nM in the perilymph fluid in the inner ear.
Embodiment 280. The method of any of the above enumerated embodiments wherein the TAZ activator is administered in an amount sufficient to achieve a concentration of about 90 nM in the perilymph fluid in the inner ear.
Embodiment 281. The method of any of the above enumerated embodiments wherein the TAZ activator is administered in an amount sufficient to achieve a concentration of about 100 nM in the perilymph fluid in the inner ear.
Embodiment 282. The method of any of the above enumerated embodiments wherein the TAZ activator is administered in an amount sufficient to achieve a concentration of about 200 nM in the perilymph fluid in the inner ear.
Embodiment 283. The method of any of the above enumerated embodiments wherein the TAZ activator is administered in an amount sufficient to achieve a concentration of about 300 nM in the perilymph fluid in the inner ear.
Embodiment 284. The method of any of the above enumerated embodiments wherein the TAZ activator is administered in an amount sufficient to achieve a concentration of about 400 nM in the perilymph fluid in the inner ear.
Embodiment 285. The method of any of the above enumerated embodiments wherein the TAZ activator is administered in an amount sufficient to achieve a concentration of about 500 nM in the perilymph fluid in the inner ear.
Embodiment 286. The method of any of the above enumerated embodiments wherein the TAZ activator is administered in an amount sufficient to achieve a concentration of about 0.6 μM in the perilymph fluid in the inner ear.
Embodiment 287. The method of any of the above enumerated embodiments wherein the TAZ activator is administered in an amount sufficient to achieve a concentration of about 0.7 μM in the perilymph fluid in the inner ear.
Embodiment 288. The method of any of the above enumerated embodiments wherein the TAZ activator is administered in an amount sufficient to achieve a concentration of about 0.8 μM in the perilymph fluid in the inner ear.
Embodiment 289. The method of any of the above enumerated embodiments the TAZ activator is administered in an amount sufficient to achieve a concentration of about 0.9 μM in the perilymph fluid in the inner ear.
Embodiment 290. The method of any of the above enumerated embodiments wherein the TAZ activator is administered in an amount sufficient to achieve a concentration of about 1 μM in the perilymph fluid in the inner ear.
Embodiment 291. The method of any of the above enumerated embodiments wherein the TAZ activator is administered in an amount sufficient to achieve a concentration of about 2 μM in the perilymph fluid in the inner ear.
Embodiment 292. The method of any of the above enumerated embodiments wherein the TAZ activator is administered in an amount sufficient to achieve a concentration of about 3 μM in the perilymph fluid in the inner ear.
Embodiment 293. The method of any of the above enumerated embodiments wherein the TAZ activator is administered in an amount sufficient to achieve a concentration of about 4 μM in the perilymph fluid in the inner ear.
Embodiment 294. The method of any of the above enumerated embodiments wherein the TAZ activator is administered in an amount sufficient to achieve a concentration of about 5 μM in the perilymph fluid in the inner ear.
Embodiment 295. The method of any of the above enumerated embodiments wherein the TAZ activator is administered in an amount sufficient to achieve a concentration of about 6 μM in the perilymph fluid in the inner ear.
Embodiment 296. The method of any of the above enumerated embodiments wherein the TAZ activator is administered in an amount sufficient to achieve a concentration of about 7 μM in the perilymph fluid in the inner ear.
Embodiment 297. The method of any of the above enumerated embodiments wherein the TAZ activator is administered in an amount sufficient to achieve a concentration of about 8 μM in the perilymph fluid in the inner ear.
Embodiment 298. The method of any of the above enumerated embodiments wherein the TAZ activator is administered in an amount sufficient to achieve a concentration of about 9 μM in the perilymph fluid in the inner ear.
Embodiment 299. The method of any of the above enumerated embodiments wherein the TAZ activator is administered in an amount sufficient to achieve a concentration of about 10 μM in the perilymph fluid in the inner ear.
Embodiment 300. The method of any of the above enumerated embodiments wherein the TAZ activator is administered in an amount sufficient to achieve a concentration of about 20 μM in the perilymph fluid in the inner ear.
Embodiment 301. The method of any of the above enumerated embodiments wherein the TAZ activator is administered in an amount sufficient to achieve a concentration of about 30 μM in the perilymph fluid in the inner ear.
Embodiment 302. The method of any of the above enumerated embodiments wherein the TAZ activator is administered in an amount sufficient to achieve a concentration of about 40 μM in the perilymph fluid in the inner ear.
Embodiment 303. The method of any of the above enumerated embodiments wherein the TAZ activator is administered in an amount sufficient to achieve a concentration of about 50 μM in the perilymph fluid in the inner ear.
Embodiment 304. The method of any of the above enumerated embodiments wherein the TAZ activator is administered in an amount sufficient to achieve a concentration of about 60 μM in the perilymph fluid in the inner ear.
Embodiment 305. The method of any of the above enumerated embodiments wherein the TAZ activator is administered in an amount sufficient to achieve a concentration of about 70 μM in the perilymph fluid in the inner ear.
Embodiment 306. The method of any of the above enumerated embodiments wherein the TAZ activator is administered in an amount sufficient to achieve a concentration of about 80 μM in the perilymph fluid in the inner ear.
Embodiment 307. The method of any of the above enumerated embodiments wherein the TAZ activator is administered in an amount sufficient to achieve a concentration of about 90 μM in the perilymph fluid in the inner ear.
Embodiment 308. The method of any of the above enumerated embodiments wherein the TAZ activator is administered in an amount sufficient to achieve a concentration of about 100 μM in the perilymph fluid in the inner ear.
Embodiment 309. The method of any of the above enumerated embodiments wherein the TAZ activator is administered in an amount sufficient to achieve a concentration of about 110 μM in the perilymph fluid in the inner ear.
Embodiment 310. The method of any of the above enumerated embodiments wherein the TAZ activator is administered in an amount sufficient to achieve a concentration of about 120 μM in the perilymph fluid in the inner ear.
Embodiment 311. The method of any of the above enumerated embodiments wherein the TAZ activator is administered in an amount sufficient to achieve a concentration of about 130 μM in the perilymph fluid in the inner ear.
Embodiment 312. The method of any of the above enumerated embodiments wherein the TAZ activator is administered in an amount sufficient to achieve a concentration of about 140 μM in the perilymph fluid in the inner ear.
Embodiment 313. The method of any of the above enumerated embodiments wherein the TAZ activator is administered in an amount sufficient to achieve a concentration of about 150 μM in the perilymph fluid in the inner ear.
Embodiment 314. The method of any of the above enumerated embodiments wherein the TAZ activator is administered in an amount sufficient to achieve a concentration of about 200 μM in the perilymph fluid in the inner ear.
Embodiment 315. The method of any of the above enumerated embodiments wherein the TAZ activator is administered in an amount sufficient to achieve a concentration of about 250 μM in the perilymph fluid in the inner ear.
Embodiment 316. The method of any of the above enumerated embodiments wherein the TAZ activator is administered in an amount sufficient to achieve a concentration of about 500 μM in the perilymph fluid in the inner ear.
Embodiment 317. The method of any of the above enumerated embodiments wherein the TAZ activator is administered to the middle ear at a concentration of about 0.01 μM to 100,000 μM.
Embodiment 318. The method of any of the above enumerated embodiments wherein the TAZ activator is administered to the middle ear at a concentration of about 0.1 μM to 10,000 μM.
Embodiment 319. The method of any of the above enumerated embodiments wherein the TAZ activator is administered to the middle ear at a concentration of about 1 μM to 1,000 μM.
Embodiment 320. The method of any of the above enumerated embodiments wherein the TAZ activator is administered to the middle ear at a concentration of about 1 μM to 10 μM.
Embodiment 321. The method of any of the above enumerated embodiments wherein the TAZ activator is administered to the middle ear at a concentration of about 10 μM to 100 μM.
Embodiment 322. The method of any of the above enumerated embodiments wherein the TAZ activator is administered to t the middle ear at a concentration of about 100 μM to 1 mM.
Embodiment 323. The method of any of the above enumerated embodiments wherein the TAZ activator is administered to the middle ear at a concentration of about 1 nM to 1000 μM
Embodiment 324. The method of any of the above enumerated embodiments wherein the TAZ activator is administered to the middle ear at a concentration of 0.001 μM to 10 mM.
Embodiment 325. The method of any of the above enumerated embodiments wherein the TAZ activator is administered to the middle ear at a concentration of 0.001 μM to 1,000 mM.
Embodiment 326. The method of any of the above enumerated embodiments wherein the TAZ activator is administered to the middle ear at a concentration of about 0.001 μM to 0.01 μM.
Embodiment 327. The method of any of the above enumerated embodiments wherein the TAZ activator is administered to the middle ear at a concentration of about 0.01 μM to 1 mM.
Embodiment 328. The method of any of the above enumerated embodiments wherein the TAZ activator is administered to the middle ear at a concentration of about 0.01 μM to 0.1 μM.
Embodiment 329. The method of any of the above enumerated embodiments wherein the TAZ activator is administered to the middle ear at a concentration of about 0.1 μM to 100 μM.
Embodiment 330. The method of any of the above enumerated embodiments wherein the TAZ activator is administered to the middle ear at a concentration of about 0.1 μM to 1 μM.
Embodiment 331. The method of any of the above enumerated embodiments wherein the TAZ activator is administered to the middle ear at a concentration of about 1 □M to 10 □M.
Embodiment 332. The method of any of the above enumerated embodiments wherein the TAZ activator is administered to the middle ear at a concentration of about 1 μM to 1000 μM.
Embodiment 333. The method of any of the above enumerated embodiments wherein the TAZ activator is administered to the middle ear at a concentration of about 10 μM to 100 μM.
Embodiment 334. The method of any of the above enumerated embodiments wherein the TAZ activator is administered to the middle ear at a concentration of about 100 μM to 1000 μM.
Embodiment 335. The method of any of the above enumerated embodiments wherein the TAZ activator is administered to the middle ear at a concentration of about 100 μM to 1 μM.
Embodiment 336. The method of any of the above enumerated embodiments wherein the TAZ activator is administered to the middle ear at a concentration of about 0.001 mM to 10,000 mM.
Embodiment 337. The method of any of the above enumerated embodiments wherein the TAZ activator is administered to the middle ear at a concentration of about 0.01 mM to 10,000 mM.
Embodiment 338. The method of any of the above enumerated embodiments wherein the TAZ activator is administered to the middle ear at a concentration of about 0.01 mM to 1,000 mM.
Embodiment 339. The method of any of the above enumerated embodiments wherein the TAZ activator is administered to the middle ear at a concentration of about 0.1 mM to 100,000 mM.
Embodiment 340. The method of any of the above enumerated embodiments wherein the TAZ activator is administered to the middle ear at a concentration of about 0.1 mM to 1,000 mM.
Embodiment 341. The method of any of the above enumerated embodiments wherein the TAZ activator is administered to the middle ear at a concentration of about 0.1 mM to 100 mM.
Embodiment 342. The method of any of the above enumerated embodiments wherein the TAZ activator is administered to the middle ear at a concentration of about 0.1 mM to 1 mM.
Embodiment 343. The method of any of the above enumerated embodiments wherein the TAZ activator is administered to the middle ear, at a concentration of about 1 mM to 100 mM.
Embodiment 344. The method of any of the above enumerated embodiments wherein the TAZ activator is administered to the middle ear at a concentration of about 1 mM to 10 mM.
Embodiment 345. The method of any of the above enumerated embodiments wherein the TAZ activator is administered to the middle ear at a concentration of about 10 mM to 100 mM.
Embodiment 346. The method of any of the above enumerated embodiments wherein the TAZ activator is administered to the middle ear at a concentration of about 100 mM to 1,000 mM.
Embodiment 347. The method of any of the above enumerated embodiments wherein the TAZ activator is administered to the middle ear at a concentration of about 1,000 mM to 10,000 mM.
Embodiment 348. The method of any of the above enumerated embodiments wherein the TAZ activator is administered to the middle ear at a concentration of about 0.1 μM.
Embodiment 349. The method of any of the above enumerated embodiments wherein the TAZ activator is administered to the middle ear at a concentration of about 0.2 μM.
Embodiment 350. The method of any of the above enumerated embodiments wherein the TAZ activator is administered to the middle ear at a concentration of about 0.3 μM.
Embodiment 351. The method of any of the above enumerated embodiments wherein the TAZ activator is administered to the middle ear at a concentration of about 0.4 μM.
Embodiment 352. The method of any of the above enumerated embodiments wherein the TAZ activator is administered to the middle ear at a concentration of about 0.5 μM.
Embodiment 353. The method of any of the above enumerated embodiments wherein the TAZ activator is administered to the middle ear at a concentration of about 0.6 μM.
Embodiment 354. The method of any of the above enumerated embodiments wherein the TAZ activator is administered to the middle ear at a concentration of about 0.7 μM.
Embodiment 355. The method of any of the above enumerated embodiments wherein the TAZ activator is administered to the middle ear at a concentration of about 0.8 μM.
Embodiment 356. The method of any of the above enumerated embodiments wherein the TAZ activator is administered to the middle ear at a concentration of about 0.9 μM.
Embodiment 357. The method of any of the above enumerated embodiments wherein the TAZ activator is administered to the middle ear at a concentration of about 1.0 μM.
Embodiment 358. The method of any of the above enumerated embodiments wherein the TAZ activator is administered to the middle ear at a concentration of about 2.0 μM.
Embodiment 359. The method of any of the above enumerated embodiments wherein the TAZ activator is administered to the middle ear at a concentration of about 3.0 μM.
Embodiment 360. The method of any of the above enumerated embodiments wherein the TAZ activator is administered to the middle ear at a concentration ofabout 4.0 μM.
Embodiment 361. The method of any of the above enumerated embodiments wherein the TAZ activator is administered to the middle ear at a concentration of about 5.0 μM.
Embodiment 362. The method of any of the above enumerated embodiments wherein the TAZ activator is administered to the middle ear at a concentration of about 6.0 μM.
Embodiment 363. The method of any of the above enumerated embodiments wherein the TAZ activator is administered to the middle ear at a concentration of about 7.0 μM.
Embodiment 364. The method of any of the above enumerated embodiments wherein the TAZ activator is administered to the middle ear at a concentration of about 8.0 μM
Embodiment 365. The method of any of the above enumerated embodiments wherein the TAZ activator is administered to the middle ear at a concentration of about 9.0 μM
Embodiment 366. The method of any of the above enumerated embodiments wherein the TAZ activator is administered to the middle ear at a concentration of about 10 μM.
Embodiment 367. The method of any of the above enumerated embodiments wherein the TAZ activator is administered to the middle ear at a concentration of about 20 μM.
Embodiment 368. The method of any of the above enumerated embodiments wherein the TAZ activator is administered to the middle ear at a concentration of about 30 μM.
Embodiment 369. The method of any of the above enumerated embodiments wherein the TAZ activator is administered to the middle ear at a concentration of about 40 μM.
Embodiment 370. The method of any of the above enumerated embodiments wherein the TAZ activator is administered to the middle ear at a concentration of about 50 μM.
Embodiment 371. The method of any of the above enumerated embodiments wherein the TAZ activator is administered to the middle ear at a concentration of about 60 μM.
Embodiment 372. The method of any of the above enumerated embodiments wherein the TAZ activator is administered to the middle ear at a concentration of about 70 μM.
Embodiment 373. The method of any of the above enumerated embodiments wherein the TAZ activator is administered to the middle ear at a concentration of about 80 μM.
Embodiment 374. The method of any of the above enumerated embodiments wherein the TAZ activator is administered to the middle ear at a concentration of about 90 μM.
Embodiment 375. The method of any of the above enumerated embodiments wherein the TAZ activator is administered to the middle ear at a concentration of about 0.1 mM.
Embodiment 376. The method of any of the above enumerated embodiments wherein the TAZ activator is administered to the middle ear at a concentration of about 1 mM.
Embodiment 377. The method of any of the above enumerated embodiments wherein the TAZ activator is administered to the middle ear at a concentration of about 2 mM.
Embodiment 378. The method of any of the above enumerated embodiments wherein the TAZ activator is administered to the middle ear at a concentration of about 3 mM.
Embodiment 379. The method of any of the above enumerated embodiments wherein the TAZ activator is administered to the middle ear at a concentration of about 4 mM.
Embodiment 380. The method of any of the above enumerated embodiments wherein the TAZ activator is administered to the middle ear at a concentration of about 5 mM.
Embodiment 381. The method of any of the above enumerated embodiments wherein the TAZ activator is administered to the middle ear at a concentration of about 6 mM.
Embodiment 382. The method of any of the above enumerated embodiments wherein the TAZ activator isinhibitor is administered to the middle ear at a concentration of about 7 mM.
Embodiment 383. The method of any of the above enumerated embodiments wherein the TAZ activator is administered to the middle ear at a concentration of about 8 mM.
Embodiment 384. The method of any of the above enumerated embodiments wherein the TAZ activator is administered to the middle ear at a concentration of about 9 mM.
Embodiment 385. The method of any of the above enumerated embodiments wherein the TAZ activator is administered to the middle ear at a concentration of about 10 mM.
Embodiment 386. The method of any of the above enumerated embodiments wherein the TAZ activator is administered to the middle ear at a concentration of about 20 mM.
Embodiment 387. The method of any of the above enumerated embodiments wherein the TAZ activator is administered to the middle ear at a concentration of about 30 mM.
Embodiment 388. The method of any of the above enumerated embodiments wherein the TAZ activator is administered to the middle ear at a concentration of about 40 mM.
Embodiment 389. The method of any of the above enumerated embodiments wherein the TAZ activator is administered to the middle ear at a concentration of about 50 mM.
Embodiment 390. The method of any of the above enumerated embodiments wherein the TAZ activator is administered to the middle ear at a concentration of about 60 mM.
Embodiment 391. The method of any of the above enumerated embodiments wherein the TAZ activator is administered to the middle ear at a concentration of about 70 mM.
Embodiment 392. The method of any of the above enumerated embodiments wherein the TAZ activator is administered to the middle ear at a concentration of about 80 mM.
Embodiment 393. The method of any of the above enumerated embodiments wherein the TAZ activator is administered to the middle ear at a concentration of about 90 mM.
Embodiment 394. The method of any of the above enumerated embodiments wherein the TAZ activator is administered to the middle ear at a concentration of about 100 mM.
Embodiment 395. The method of any of the above enumerated embodiments wherein the TAZ activator is administered to the middle ear at a concentration of about 150 mM.
Embodiment 396. The method of any of the above enumerated embodiments wherein the TAZ activator is administered to the middle ear at a concentration of about 200 mM.
Embodiment 397. The method of any of the above enumerated embodiments wherein the TAZ activator is administered to the middle ear at a concentration of about 500 mM.
Embodiment 398. The method of any of the above enumerated embodiments wherein the TAZ activator is administered to the middle ear at a concentration of about 1000 mM.
Embodiment 399. The method of any of the above enumerated embodiments wherein the TAZ activator is administered to a subject systemically at a daily dose of about 10 mg to 5000 mg/day.
Embodiment 400. The method of any of the above enumerated embodiments wherein the TAZ activator is administered to a subject systemically at a daily dose of about 50 mg to 5000 mg/day.
Embodiment 401. The method of any of the above enumerated embodiments wherein the TAZ activator is administered to a subject systemically at a daily dose of about 10 mg to 2500 mg/day.
Embodiment 402. The method of any of the above enumerated embodiments wherein the TAZ activator is administered to a subject systemically at a daily dose of about 15 mg to 1500 mg/day.
Embodiment 403. The method of any of the above enumerated embodiments wherein the TAZ activator is administered to a subject systemically at a daily dose of about 15 mg to 1000 mg/day.
Embodiment 404. The method of any of the above enumerated embodiments wherein the TAZ activator is administered to a subject systemically at a daily dose of about 100 mg to 200 mg/day.
Embodiment 405. The method of any of the above enumerated embodiments wherein the TAZ activator is administered to a subject systemically at a daily dose of about 200 mg to 300 mg/day.
Embodiment 406. The method of any of the above enumerated embodiments wherein the TAZ activator is administered to a subject systemically at a daily dose of about 300 mg to 400 mg/day.
Embodiment 407. The method of any of the above enumerated embodiments wherein the TAZ activator is administered to a subject systemically at a daily dose of about 400 mg to 500 mg/day.
Embodiment 408. The method of any of the above enumerated embodiments wherein the TAZ activator is administered to a subject systemically at a daily dose of about 500 mg to 600 mg/day.
Embodiment 409. The method of any of the above enumerated embodiments wherein the TAZ activator is administered to a subject systemically at a daily dose of about 600 mg to 700 mg/day.
Embodiment 410. The method of any of the above enumerated embodiments wherein the TAZ activator is administered to a subject systemically at a daily dose of about 700 mg to 800 mg/day.
Embodiment 411. The method of any of the above enumerated embodiments wherein the TAZ activator is administered to a subject systemically at a daily dose of about 900 mg to 1000 mg/day.
Embodiment 412. The method of any of the above enumerated embodiments wherein the TAZ activator is administered to a subject systemically at a daily dose of about 100 mg to 120 mg/day.
Embodiment 413. The method of any of the above enumerated embodiments wherein the TAZ activator is administered to a subject systemically at a daily dose of about 10 mg to 20 mg/day
Embodiment 414. The method of any of the above enumerated embodiments wherein the TAZ activator is administered to a subject systemically at a daily dose of about 0.01 mg to 500 mg/day.
Embodiment 415. The method of any of the above enumerated embodiments wherein the TAZ activator is administered to a subject systemically at a daily dose of about 0.1 mg to 100 mg/day.
Embodiment 416. The method of any of the above enumerated embodiments wherein the TAZ activator is administered to a subject systemically at a daily dose of about 1 mg to 50 mg/day.
Embodiment 417. The method of any of the above enumerated embodiments wherein the TAZ activator is administered to a subject systemically at a daily dose of about 1 mg to 25 mg/day.
Embodiment 418. The method of any of the above enumerated embodiments wherein the TAZ activator is administered to a subject systemically at a daily dose of about 1 mg to 10 mg/day.
Embodiment 419. The method of any of the above enumerated embodiments wherein the TAZ activator is administered to a subject systemically at a daily dose of about 1 mg to 5 mg/day.
Embodiment 420. The method of any of the above enumerated embodiments wherein the TAZ activator is administered to a subject systemically at a daily dose of about 0.01 mg to 0.1 mg/day.
Embodiment 421. The method of any of the above enumerated embodiments wherein the TAZ activator is administered to a subject systemically at a daily dose of about 0.1 mg to 1 mg/day.
Embodiment 422. The method of any of the above enumerated embodiments wherein the TAZ activator is administered to a subject systemically at a daily dose of about 1 mg to 10 mg/day.
Embodiment 423. The method of any of the above enumerated embodiments wherein the TAZ activator is administered to a subject systemically at a daily dose of about 10 mg to 100 mg/day.
Embodiment 424. The method of any of the above enumerated embodiments wherein the TAZ activator is administered to a subject systemically at a daily dose of about 100 mg to 500 mg/day.
Embodiment 425. The method of any of the above enumerated embodiments wherein the TAZ activator is administered to a subject systemically at a daily dose of about 0.5 mg to 1 mg/day.
Embodiment 426. The method of any of the above enumerated embodiments wherein the TAZ activator is administered to a subject systemically at a daily dose of about 10 mg to 25 mg/day.
Embodiment 427. The method of any of the above enumerated embodiments wherein the TAZ activator is administered to a subject systemically at a daily dose of about 25 mg to 50 mg/day.
Embodiment 428. The method of any of the above enumerated embodiments wherein the TAZ activator is administered to a subject systemically at a daily dose of about 50 mg to 75 mg/day.
Embodiment 429. The method of any of the above enumerated embodiments wherein the TAZ activator is administered to a subject systemically at a daily dose of about 75 mg to 100 mg/day.
Embodiment 430. The method of any of the above enumerated embodiments wherein the TAZ activator is administered to a subject systemically at a daily dose of about 100 to 200 mg/day.
Embodiment 431. The method of any of the above enumerated embodiments wherein the TAZ activator is administered to a subject systemically at a daily dose of about 200-300 mg/day.
Embodiment 432. The method of any of the above enumerated embodiments wherein the TAZ activator is administered to a subject systemically at a daily dose of about 300-400 mg/day.
Embodiment 433. The method of any of the above enumerated embodiments wherein the TAZ activator is administered to a subject systemically at a daily dose of about 400-500 mg/day.
Embodiment 434. The method of any of the above enumerated embodiments wherein the TAZ activator is administered to the subject at a concentration ratio of about 0.001 to 100 fold relative to an FDA approved concentration.
Embodiment 435. The method of any of the above enumerated embodiments wherein the TAZ activator is administered to the subject at a concentration ratio of about 0.01 to 50 fold relative to an FDA approved concentration.
Embodiment 436. The method of any of the above enumerated embodiments wherein the TAZ activator is administered to the subject at a concentration ratio of about 0.1 to 10 fold relative to an FDA approved concentration.
Embodiment 437. The method of any of the above enumerated embodiments wherein the TAZ activator is administered to the subject at a concentration ratio of about 0.1 to 5 fold relative to an FDA approved concentration.
Embodiment 438. The method of any of the above enumerated embodiments wherein the TAZ activator is administered to the subject at a concentration ratio of about 1 to 5 fold relative to an FDA approved concentration.
Embodiment 439. The method of any of the above enumerated embodiments wherein the TAZ activator is administered to the subject at about 0.01× relative to an FDA approved concentration.
Embodiment 440. The method of any of the above enumerated embodiments wherein the TAZ activator is administered to the subject at about 0.1× relative to an FDA approved concentration.
Embodiment 441. The method of any of the above enumerated embodiments wherein the TAZ activator is administered to the subject at about 2× relative to an FDA approved concentration.
Embodiment 442. The method of any of the above enumerated embodiments wherein the TAZ activator is administered to the subject at about 3× relative to an FDA approved concentration.
Embodiment 443. The method of any of the above enumerated embodiments wherein the TAZ activator is administered to the subject at about 4× relative to an FDA approved concentration.
Embodiment 444. The method of any of the above enumerated embodiments wherein the TAZ activator is administered to the subject at about 5× relative to an FDA approved concentration.
Embodiment 445. The method of any of the above enumerated embodiments wherein the TAZ activator is administered to the subject at about 10× relative to an FDA approved concentration.
Embodiment 446. The method of any of the above enumerated embodiments wherein the Wnt agonist is a GSK3-α inhibitor.
Embodiment 447. The method of any of the above enumerated embodiments wherein the Wnt agonist is a GSK3-R inhibitor.
Embodiment 448. The method of any of the above enumerated embodiments wherein the Wnt agonist increases Wnt signaling by about or at least about 1.1-fold or more relative to a control.
Embodiment 449. The method of any of the above enumerated embodiments wherein the Wnt agonist increases Wnt signaling about or at least about 1.2-fold or more relative to a control.
Embodiment 450. The method of any of the above enumerated embodiments wherein the Wnt agonist increases Wnt signaling by about or at least about 1.3-fold or more relative to a control.
Embodiment 451. The method of any of the above enumerated embodiments wherein the Wnt agonist increases Wnt signaling by about or at least about 1.4-fold or more relative to a control.
Embodiment 452. The method of any of the above enumerated embodiments wherein the Wnt agonist increases Wnt signaling by about or at least about 1.5-fold or more relative to a control.
Embodiment 453. The method of any of the above enumerated embodiments wherein the Wnt agonist increases Wnt signaling by about or at least about 1.6-fold or more relative to a control.
Embodiment 454. The method of any of the above enumerated embodiments wherein the Wnt agonist increases Wnt signaling by about or at least about 1.7-fold or more relative to a control.
Embodiment 455. The method of any of the above enumerated embodiments wherein the Wnt agonist increases Wnt signaling by about or at least about 1.8-fold or more relative to a control.
Embodiment 456. The method of any of the above enumerated embodiments wherein the Wnt agonist increases Wnt signaling by about or at least about 1.9-fold or more relative to a control.
Embodiment 457. The method of any of the above enumerated embodiments wherein the Wnt agonist increases Wnt signaling by about or at least about 2-fold or more relative to a control.
Embodiment 458. The method of any of the above enumerated embodiments wherein the Wnt agonist increases Wnt signaling by about or at least about 3-fold or more relative to a control.
Embodiment 459. The method of any of the above enumerated embodiments wherein the Wnt agonist increases Wnt signaling by about or at least about 4-fold or more relative to a control.
Embodiment 460. The method of any of the above enumerated embodiments wherein the Wnt agonist increases Wnt signaling by about or at least about 5-fold or more relative to a control.
Embodiment 461. The method of any of the above enumerated embodiments wherein the Wnt agonist increases Wnt signaling by about or at least about 6-fold or more relative to a control.
Embodiment 462. The method of any of the above enumerated embodiments wherein the Wnt agonist increases Wnt signaling by about or at least about 7-fold or more relative to a control.
Embodiment 463. The method of any of the above enumerated embodiments wherein the Wnt agonist increases Wnt signaling by about or at least about 8-fold or more relative to a control.
Embodiment 464. The method of any of the above enumerated embodiments wherein the Wnt agonist increases Wnt signaling by about or at least about 9-fold or more relative to a control.
Embodiment 465. The method of any of the above enumerated embodiments wherein the Wnt agonist increases Wnt signaling by about or at least about 10-fold or more relative to a control.
Embodiment 466. The method of any of the above enumerated embodiments wherein the Wnt agonist increases Wnt signaling by about or at least about 15-fold or more relative to a control.
Embodiment 467. The method of any of the above enumerated embodiments wherein the Wnt agonist increases Wnt signaling by about or at least about 20-fold or more relative to a control.
Embodiment 468. The method of any of the above enumerated embodiments wherein the Wnt agonist increases Wnt signaling by about or at least about 30-fold or more relative to a control.
Embodiment 469. The method of any of the above enumerated embodiments wherein the Wnt agonist increases Wnt signaling by about or at least about 40-fold or more relative to a control.
Embodiment 470. The method of any of the above enumerated embodiments wherein the Wnt agonist increases Wnt signaling by about or at least about 50-fold or more relative to a control.
Embodiment 471. The method of any of the above enumerated embodiments wherein the Wnt agonist increases Wnt signaling by about or at least about 60-fold or more relative to a control.
Embodiment 472. The method of any of the above enumerated embodiments wherein the Wnt agonist increases Wnt signaling by about or at least about 70-fold or more relative to a control.
Embodiment 473. The method of any of the above enumerated embodiments wherein the Wnt agonist increases Wnt signaling by about or at least about 80-fold or more relative to a control.
Embodiment 474. The method of any of the above enumerated embodiments wherein the Wnt agonist increases Wnt signaling by about or at least about 90-fold or more relative to a control.
Embodiment 475. The method of any of the above enumerated embodiments wherein the Wnt agonist increases Wnt signaling by about or at least about 100-fold or more relative to a control.
Embodiment 476. The method of any of the above enumerated embodiments wherein the Wnt agonist increases Wnt signaling by about or at least about 200-fold or more relative to a control.
Embodiment 477. The method of any of the above enumerated embodiments wherein the Wnt agonist increases Wnt signaling by about or at least about 500-fold or more relative to a control.
Embodiment 478. The method of any of the above enumerated embodiments wherein the Wnt agonist increases Wnt signaling by about or at least about 1000-fold or more relative to a control.
Embodiment 479. The method of any of the above enumerated embodiments wherein the Wnt agonist increases TCF/LEF-mediated transcription by about or at least about 1.1-fold or more relative to a control.
Embodiment 480. The method of any of the above enumerated embodiments wherein the Wnt agonist increases TCF/LEF-mediated transcription by about or at least about 1.2-fold or more relative to a control.
Embodiment 481. The method of any of the above enumerated embodiments wherein the Wnt agonist increases TCF/LEF-mediated transcription by about or at least about 1.3-fold or more relative to a control.
Embodiment 482. The method of any of the above enumerated embodiments wherein the Wnt agonist increases TCF/LEF-mediated transcription by about or at least about 1.4-fold or more relative to a control.
Embodiment 483. The method of any of the above enumerated embodiments wherein the Wnt agonist increases TCF/LEF-mediated transcription by about or at least about 1.5-fold or more relative to a control.
Embodiment 484. The method of any of the above enumerated embodiments wherein the Wnt agonist increases TCF/LEF-mediated transcription by about or at least about 1.6-fold or
Embodiment 485. The method of any of the above enumerated embodiments wherein the Wnt agonist increases TCF/LEF-mediated transcription by about or at least about 1.7-fold or more relative to a control.
Embodiment 486. The method of any of the above enumerated embodiments wherein the Wnt agonist increases TCF/LEF-mediated transcription by about or at least about 1.8-fold or more relative to a control.
Embodiment 487. The method of any of the above enumerated embodiments wherein the Wnt agonist increases TCF LEF-mediated transcription by about or at least about 1.9-fold or more relative to a control.
Embodiment 488. The method of any of the above enumerated embodiments wherein the Wnt agonist increases TCF/LEF-mediated transcription by about or at least about 2-fold or more relative to a control.
Embodiment 489. The method of any of the above enumerated embodiments wherein the Wnt agonist increases TCF/LEF-mediated transcription by about or at least about 3-fold or more relative to a control.
Embodiment 490. The method of any of the above enumerated embodiments wherein the Wnt agonist increases TCF/LEF-mediated transcription by about or at least about 4-fold or more relative to a control.
Embodiment 491. The method of any of the above enumerated embodiments wherein the Wnt agonist increases TCF/LEF-mediated transcription by about or at least about 5-fold or more relative to a control.
Embodiment 492. The method of any of the above enumerated embodiments wherein the Wnt agonist increases TCF/LEF-mediated transcription by about or at least about 6-fold or more relative to a control.
Embodiment 493. The method of any of the above enumerated embodiments wherein the Wnt agonist increases TCF/LEF-mediated transcription cell by about or at least about 7-fold or more relative to a control.
Embodiment 494. The method of any of the above enumerated embodiments wherein the Wnt agonist increases TCF/LEF-mediated transcription by about or at least about 8-fold or
Embodiment 495. The method of any of the above enumerated embodiments wherein the Wnt agonist increases TCF/LEF-mediated transcription by about or at least about 9-fold or more relative to a control.
Embodiment 496. The method of any of the above enumerated embodiments wherein the Wnt agonist increases TCF/LEF-mediated transcription by about or at least about 10-fold or more relative to a control.
Embodiment 497. The method of any of the above enumerated embodiments wherein the Wnt agonist increases TCF LEF-mediated transcription by about or at least about 15-fold or more relative to a control.
Embodiment 498. The method of any of the above enumerated embodiments wherein the Wnt agonist increases TCF/LEF-mediated transcription by about or at least about 20-fold or more relative to a control.
Embodiment 499. The method of any of the above enumerated embodiments wherein the Wnt agonist increases TCF/LEF-mediated transcription by about or at least about 30-fold or more relative to a control.
Embodiment 500. The method of any of the above enumerated embodiments wherein the Wnt agonist increases TCF/LEF-mediated transcription by about or at least about 40-fold or more relative to a control.
Embodiment 501. The method of any of the above enumerated embodiments wherein the Wnt agonist increases TCF/LEF-mediated transcription by about or at least about 50-fold or more relative to a control.
Embodiment 502. The method of any of the above enumerated embodiments wherein the Wnt agonist increases TCF/LEF-mediated transcription by about or at least about 60-fold or more relative to a control.
Embodiment 503. The method of any of the above enumerated embodiments wherein the Wnt agonist increases TCF/LEF-mediated transcription by about or at least about 70-fold or more relative to a control.
Embodiment 504. The method of any of the above enumerated embodiments wherein the Wnt agonist increases TCF/LEF-mediated transcription by about or at least about 80-fold or
Embodiment 505. The method of any of the above enumerated embodiments wherein the Wnt agonist increases TCF/LEF-mediated transcription by about or at least about 90-fold or more relative to a control.
Embodiment 506. The method of any of the above enumerated embodiments wherein the Wnt agonist increases TCF/LEF-mediated transcription by about or at least about 100-fold or more relative to a control.
Embodiment 507. The method of any of the above enumerated embodiments wherein the Wnt agonist increases TCF/LEF-mediated transcription by about or at least about 200-fold or more relative to a control.
Embodiment 508. The method of any of the above enumerated embodiments wherein the Wnt agonist increases TCF/LEF-mediated transcription by about or at least about 500-fold or more relative to a control.
Embodiment 509. The method of any of the above enumerated embodiments wherein the Wnt agonist increases TCF/LEF-mediated transcription by about or at least about 1000-fold or more relative to a control.
Embodiment 510. The method of any of the above enumerated embodiments wherein the Wnt agonist binds and activates a Frizzled receptor family member by about or at least about 1.1-fold or more relative to a control.
Embodiment 511. The method of any of the above enumerated embodiments wherein the Wnt agonist binds and activates a Frizzled receptor family member by about or at least about 1.2-fold or more relative to a control.
Embodiment 512. The method of any of the above enumerated embodiments wherein the Wnt agonist binds and activates a Frizzled receptor family member by about or at least about 1.3-fold or more relative to a control.
Embodiment 513. The method of any of the above enumerated embodiments wherein the Wnt agonist binds and activates a Frizzled receptor family member by about or at least about 1.4-fold or more relative to a control.
Embodiment 514. The method of any of the above enumerated embodiments wherein the Wnt agonist binds and activates a Frizzled receptor family member by about or at least about 1.5-fold or more relative to a control.
Embodiment 515. The method of any of the above enumerated embodiments wherein the Wnt agonist binds and activates a Frizzled receptor family member by about or at least about 1.6-fold or more relative to a control.
Embodiment 516. The method of any of the above enumerated embodiments wherein the Wnt agonist binds and activates a Frizzled receptor family member by about or at least about 1.7-fold or more relative to a control.
Embodiment 517. The method of any of the above enumerated embodiments wherein the Wnt agonist binds and activates a Frizzled receptor family member by about or at least about 1.8-fold or more relative to a control.
Embodiment 518. The method of any of the above enumerated embodiments wherein the Wnt agonist binds and activates a Frizzled receptor family member by about or at least about 1.9-fold or more relative to a control.
Embodiment 519. The method of any of the above enumerated embodiments wherein the Wnt agonist binds and activates a Frizzled receptor family member by about or at least about 2-fold or more relative to a control.
Embodiment 520. The method of any of the above enumerated embodiments wherein the Wnt agonist binds and activates a Frizzled receptor family member by about or at least about 3-fold or more relative to a control.
Embodiment 521. The method of any of the above enumerated embodiments wherein the Wnt agonist binds and activates a Frizzled receptor family member by about or at least about 4-fold or more relative to a control.
Embodiment 522. The method of any of the above enumerated embodiments wherein the Wnt agonist binds and activates a Frizzled receptor family member by about or at least about 5-fold or more relative to a control.
Embodiment 523. The method of any of the above enumerated embodiments wherein the Wnt agonist binds and activates a Frizzled receptor family member by about or at least about 6-fold or more relative to a control.
Embodiment 524. The method of any of the above enumerated embodiments wherein the Wnt agonist binds and activates a Frizzled receptor family member by about or at least about 7-fold or more relative to a control.
Embodiment 525. The method of any of the above enumerated embodiments wherein the Wnt agonist binds and activates a Frizzled receptor family member by about or at least about 8-fold or more relative to a control.
Embodiment 526. The method of any of the above enumerated embodiments wherein the Wnt agonist binds and activates a Frizzled receptor family member by about or at least about 9-fold or more relative to a control.
Embodiment 527. The method of any of the above enumerated embodiments wherein the Wnt agonist binds and activates a Frizzled receptor family member by about or at least about 10-fold or more relative to a control.
Embodiment 528. The method of any of the above enumerated embodiments wherein the Wnt agonist binds and activates a Frizzled receptor family member by about or at least about 15-fold or more relative to a control.
Embodiment 529. The method of any of the above enumerated embodiments wherein the Wnt agonist binds and activates a Frizzled receptor family member by about or at least about 20-fold or more relative to a control.
Embodiment 530. The method of any of the above enumerated embodiments wherein the Wnt agonist binds and activates a Frizzled receptor family member by about or at least about 30-fold or more relative to a control.
Embodiment 531. The method of any of the above enumerated embodiments wherein the Wnt agonist binds and activates a Frizzled receptor family member by about or at least about 40-fold or more relative to a control.
Embodiment 532. The method of any of the above enumerated embodiments wherein the Wnt agonist binds and activates a Frizzled receptor family member by about or at least about 50-fold or more relative to a control.
Embodiment 533. The method of any of the above enumerated embodiments wherein the Wnt agonist binds and activates a Frizzled receptor family member by about or at least about 60-fold or more relative to a control.
Embodiment 534. The method of any of the above enumerated embodiments wherein the Wnt agonist binds and activates a Frizzled receptor family member by about or at least about 70-fold or more relative to a control.
Embodiment 535. The method of any of the above enumerated embodiments wherein the Wnt agonist binds and activates a Frizzled receptor family member by about or at least about 80-fold or more relative to a control.
Embodiment 536. The method of any of the above enumerated embodiments wherein the Wnt agonist binds and activates a Frizzled receptor family member by about or at least about 90-fold or more relative to a control.
Embodiment 537. The method of any of the above enumerated embodiments wherein the Wnt agonist binds and activates a Frizzled receptor family member by about or at least about 100-fold or more relative to a control.
Embodiment 538. The method of any of the above enumerated embodiments wherein the Wnt agonist binds and activates a Frizzled receptor family member by about or at least about 200-fold or more relative to a control.
Embodiment 539. The method of any of the above enumerated embodiments wherein the Wnt agonist binds and activates a Frizzled receptor family member by about or at least about 500-fold or more relative to a control.
Embodiment 540. The method of any of the above enumerated embodiments wherein the Wnt agonist binds and activates a Frizzled receptor family member by about or at least about 1000-fold or more relative to a control.
Embodiment 541. The method of any of the above enumerated embodiments wherein the Wnt agonist inhibits GSK3 by about or at least about 1.1-fold or more relative to a control.
Embodiment 542. The method of any of the above enumerated embodiments wherein the Wnt agonist inhibits GSK3 by about or at least about 1.2-fold or more relative to a control.
Embodiment 543. The method of any of the above enumerated embodiments wherein the Wnt agonist inhibits GSK3 by about or at least about 1.3-fold or more relative to a control.
Embodiment 544. The method of any of the above enumerated embodiments wherein the Wnt agonist inhibits GSK3 by about or at least about 1.4-fold or more relative to a control.
Embodiment 545. The method of any of the above enumerated embodiments wherein the Wnt agonist inhibits GSK3 by about or at least about 1.5-fold or more relative to a control.
Embodiment 546. The method of any of the above enumerated embodiments wherein the Wnt agonist inhibits GSK3 by about or at least about 1.6-fold or more relative to a control.
Embodiment 547. The method of any of the above enumerated embodiments wherein the Wnt agonist inhibits GSK3 by about or at least about 1.7-fold or more relative to a control.
Embodiment 548. The method of any of the above enumerated embodiments wherein the Wnt agonist inhibits GSK3 by about or at least about 1.8-fold or more relative to a control.
Embodiment 549. The method of any of the above enumerated embodiments wherein the Wnt agonist inhibits GSK3 by about or at least about 1.9-fold or more relative to a control.
Embodiment 550. The method of any of the above enumerated embodiments wherein the Wnt agonist inhibits GSK3 by about or at least about 2-fold or more relative to a control.
Embodiment 551. The method of any of the above enumerated embodiments wherein the Wnt agonist inhibits GSK3 by about or at least about 3-fold or more relative to a control.
Embodiment 552. The method of any of the above enumerated embodiments wherein the Wnt agonist inhibits GSK3 by about or at least about 4-fold or more relative to a control.
Embodiment 553. The method of any of the above enumerated embodiments wherein the Wnt agonist inhibits GSK3 by about or at least about 5-fold or more relative to a control.
Embodiment 554. The method of any of the above enumerated embodiments wherein the Wnt agonist inhibits GSK3 by about or at least about 6-fold or more relative to a control.
Embodiment 555. The method of any of the above enumerated embodiments wherein the Wnt agonist inhibits GSK3 by about or at least about 7-fold or more relative to a control.
Embodiment 556. The method of any of the above enumerated embodiments wherein the Wnt agonist inhibits GSK3 by about or at least about 8-fold or more relative to a control.
Embodiment 557. The method of any of the above enumerated embodiments wherein the Wnt agonist inhibits GSK3 by about or at least about 9-fold or more relative to a control.
Embodiment 558. The method of any of the above enumerated embodiments wherein the Wnt agonist inhibits GSK3 by about or at least about 10-fold or more relative to a control.
Embodiment 559. The method of any of the above enumerated embodiments wherein the Wnt agonist inhibits GSK3 by about or at least about 15-fold or more relative to a control.
Embodiment 560. The method of any of the above enumerated embodiments wherein the Wnt agonist inhibits GSK3 by about or at least about 20-fold or more relative to a control.
Embodiment 561. The method of any of the above enumerated embodiments wherein the Wnt agonist inhibits GSK3 by about or at least about 30-fold or more relative to a control.
Embodiment 562. The method of any of the above enumerated embodiments wherein the Wnt agonist inhibits GSK3 by about or at least about 40-fold or more relative to a control.
Embodiment 563. The method of any of the above enumerated embodiments wherein the Wnt agonist inhibits GSK3 by about or at least about 50-fold or more relative to a control.
Embodiment 564. The method of any of the above enumerated embodiments wherein the Wnt agonist inhibits GSK3 by about or at least about 60-fold or more relative to a control.
Embodiment 565. The method of any of the above enumerated embodiments wherein the Wnt agonist inhibits GSK3 by about or at least about 70-fold or more relative to a control.
Embodiment 566. The method of any of the above enumerated embodiments wherein the Wnt agonist inhibits GSK3 by about or at least about 80-fold or more relative to a control.
Embodiment 567. The method of any of the above enumerated embodiments wherein the Wnt agonist inhibits GSK3 by about or at least about 90-fold or more relative to a control.
Embodiment 568. The method of any of the above enumerated embodiments wherein the Wnt agonist inhibits GSK3 by about or at least about 100-fold or more relative to a control.
Embodiment 569. The method of any of the above enumerated embodiments wherein the Wnt agonist inhibits GSK3 by about or at least about 200-fold or more relative to a control.
Embodiment 570. The method of any of the above enumerated embodiments wherein the Wnt agonist inhibits GSK3 by about or at least about 500-fold or more relative to a control.
Embodiment 571. The method of any of the above enumerated embodiments wherein the Wnt agonist inhibits GSK3 by about or at least about 1000-fold or more relative to a control.
Embodiment 572. The method of any of the above enumerated embodiments wherein the Wnt agonist preferentially upregulates Jag-1 more than the Wnt agonist upregulates Hes.
Embodiment 573. The method of any of the above enumerated embodiments wherein the Wnt agonist preferentially upregulates Deltex-1 more than the Wnt agonist upregulates Hes.
Embodiment 574. The method of any of the above enumerated embodiments wherein the Wnt agonist preferentially upregulates Hif-1 more than the Wnt agonist upregulates Hes.
Embodiment 575. The method of any of the above enumerated embodiments wherein the Wnt agonist preferentially upregulates Jag-1 more than the Wnt agonist upregulates Hey.
Embodiment 576. The method of any of the above enumerated embodiments wherein the Wnt agonist preferentially upregulates Deltex-1 more than the Wnt agonist upregulates Hey.
Embodiment 577. The method of any of the above enumerated embodiments wherein the Wnt agonist preferentially upregulates Hif-1 more than the Wnt agonist upregulates Hey.
Embodiment 578. The method of any of the above enumerated embodiments wherein the Wnt agonist increases the expression of Jag-1 by 10% or more than it increases the expression or activity of Hes.
Embodiment 579. The method of any of the above enumerated embodiments wherein the Wnt agonist increases the expression of Jag-1 by 25% or more than it increases the expression or activity of Hes.
Embodiment 580. The method of any of the above enumerated embodiments wherein the Wnt agonist increases the expression of Jag-1 by 50% or more than it increases the expression or activity of Hes.
Embodiment 581. The method of any of the above enumerated embodiments wherein the Wnt agonist increases the expression of Jag-1 by 75% or more than it increases the expression or activity of Hes.
Embodiment 582. The method of any of the above enumerated embodiments wherein the Wnt agonist increases the expression of Jag-1 by 100% or more than it increases the expression or activity of Hes.
Embodiment 583. The method of any of the above enumerated embodiments wherein the Wnt agonist increases the expression of Jag-1 by 125% or more than it increases the expression or activity of Hes.
Embodiment 584. The method of any of the above enumerated embodiments wherein the Wnt agonist increases the expression of Jag-1 by 150% or more than it increases the expression or activity of Hes.
Embodiment 585. The method of any of the above enumerated embodiments wherein the Wnt agonist increases the expression of Jag-1 by 175% or more than it increases the expression or activity of Hes.
Embodiment 586. The method of any of the above enumerated embodiments wherein the Wnt agonist increases the expression of Jag-1 by²⁰⁰% or more than it increases the expression or activity of Hes.
Embodiment 587. The method of any of the above enumerated embodiments wherein the Wnt agonist increases the expression of Jag-1 by 250% or more than it increases the expression or activity of Hes.
Embodiment 588. The method of any of the above enumerated embodiments wherein the Wnt agonist increases the expression of Deltex-1 by 10% or more than it increases the expression or activity of Hes.
Embodiment 589. The method of any of the above enumerated embodiments wherein the Wnt agonist increases the expression of Deltex-1 by 25% or more than it increases the expression or activity of Hes.
Embodiment 590. The method of any of the above enumerated embodiments wherein the Wnt agonist increases the expression of Deltex-1 by 50% or more than it increases the expression or activity of Hes.
Embodiment 591. The method of any of the above enumerated embodiments wherein the Wnt agonist increases the expression of Deltex-1 by 75% or more than it increases the expression or activity of Hes.
Embodiment 592. The method of any of the above enumerated embodiments wherein the Wnt agonist increases the expression of Deltex-1 by¹⁰⁰% or more than it increases the expression or activity of Hes.
Embodiment 593. The method of any of the above enumerated embodiments wherein the Wnt agonist increases the expression of Deltex-1 by 125% or more than it increases the expression or activity of Hes.
Embodiment 594. The method of any of the above enumerated embodiments wherein the Wnt agonist increases the expression of Deltex-1 by 1⁵⁰% or more than it increases the expression or activity of Hes.
Embodiment 595. The method of any of the above enumerated embodiments wherein the Wnt agonist increases the expression of Deltex-1 by 175% or more than it increases the expression or activity of Hes.
Embodiment 596. The method of any of the above enumerated embodiments wherein the Wnt agonist increases the expression of Deltex-1 by 200% or more than it increases the expression or activity of Hes.
Embodiment 597. The method of any of the above enumerated embodiments wherein the Wnt agonist increases the expression of Deltex-1 by 250% or more than it increases the expression or activity of Hes.
Embodiment 598. The method of any of the above enumerated embodiments wherein the Wnt agonist increases the expression of Hif-1 by 10% or more than it increases the expression or activity of Hes.
Embodiment 599. The method of any of the above enumerated embodiments wherein the Wnt agonist increases the expression of Hif-1 by 25% or more than it increases the expression or activity of Hes.
Embodiment 600. The method of any of the above enumerated embodiments wherein the Wnt agonist increases the expression of Hif-1 by 50% or more than it increases the expression or activity of Hes.
Embodiment 601. The method of any of the above enumerated embodiments wherein the Wnt agonist increases the expression of Hif-1 by 75% or more than it increases the expression or activity of Hes.
Embodiment 602. The method of any of the above enumerated embodiments wherein the Wnt agonist increases the expression of Hif-1 by¹⁰⁰% or more than it increases the expression or activity of Hes.
Embodiment 603. The method of any of the above enumerated embodiments wherein the Wnt agonist increases the expression of Hif-1 by 125% or more than it increases the expression or activity of Hes.
Embodiment 604. The method of any of the above enumerated embodiments wherein the Wnt agonist increases the expression of Hif-1 by¹⁵⁰% or more than it increases the expression or activity of Hes.
Embodiment 605. The method of any of the above enumerated embodiments wherein the Wnt agonist increases the expression of Hif-1 by 175% or more than it increases the expression or activity of Hes.
Embodiment 606. The method of any of the above enumerated embodiments wherein the Wnt agonist increases the expression of Hif-1 by 200% or more than it increases the expression or activity of Hes.
Embodiment 607. The method of any of the above enumerated embodiments wherein the Wnt agonist increases the expression of Hif-1 by 250% or more than it increases the expression or activity of Hes.
Embodiment 608. The method of any of the above enumerated embodiments wherein the Wnt agonist increases the expression of Jag-1 by 10% or more than it increases the expression or activity of Hey.
Embodiment 609. The method of any of the above enumerated embodiments wherein the Wnt agonist increases the expression of Jag-1 by 25% or more than it increases the expression or activity of Hey.
Embodiment 610. The method of any of the above enumerated embodiments wherein the Wnt agonist increases the expression of Jag-1 by 50% or more than it increases the expression or activity of Hey.
Embodiment 611. The method of any of the above enumerated embodiments wherein the Wnt agonist increases the expression of Jag-1 by 75% or more than it increases the expression or activity of Hey.
Embodiment 612. The method of any of the above enumerated embodiments wherein the Wnt agonist increases the expression of Jag-1 by 1⁰⁰% or more than it increases the expression or activity of Hey.
Embodiment 613. The method of any of the above enumerated embodiments wherein the Wnt agonist increases the expression of Jag-1 by 125% or more than it increases the expression or activity of Hey.
Embodiment 614. The method of any of the above enumerated embodiments wherein the Wnt agonist increases the expression of Jag-1 by 1⁵⁰% or more than it increases the expression or activity of Hey.
Embodiment 615. The method of any of the above enumerated embodiments wherein the Wnt agonist increases the expression of Jag-1 by 175% or more than it increases the expression or activity of Hey.
Embodiment 616. The method of any of the above enumerated embodiments wherein the Wnt agonist increases the expression of Jag-1 by 200% or more than it increases the expression or activity of Hey.
Embodiment 617. The method of any of the above enumerated embodiments wherein the Wnt agonist increases the expression of Jag-1 by 250% or more than it increases the expression or activity of Hey.
Embodiment 618. The method of any of the above enumerated embodiments wherein the Wnt agonist increases the expression of Deltex-1 by 10% or more than it increases the expression or activity of Hey.
Embodiment 619. The method of any of the above enumerated embodiments wherein the Wnt agonist increases the expression of Deltex-1 by 25% or more than it increases the expression or activity of Hey.
Embodiment 620. The method of any of the above enumerated embodiments wherein the Wnt agonist increases the expression of Deltex-1 by 50% or more than it increases the expression or activity of Hey.
Embodiment 621. The method of any of the above enumerated embodiments wherein the Wnt agonist increases the expression of Deltex-1 by 75% or more than it increases the expression or activity of Hey.
Embodiment 622. The method of any of the above enumerated embodiments wherein the Wnt agonist increases the expression of Deltex-1 by¹⁰⁰% or more than it increases the expression or activity of Hey.
Embodiment 623. The method of any of the above enumerated embodiments wherein the Wnt agonist increases the expression of Deltex-1 by 125% or more than it increases the expression or activity of Hey.
Embodiment 624. The method of any of the above enumerated embodiments wherein the Wnt agonist increases the expression of Deltex-1 by¹⁵⁰% or more than it increases the expression or activity of Hey.
Embodiment 625. The method of any of the above enumerated embodiments wherein the Wnt agonist increases the expression of Deltex-1 by 175% or more than it increases the expression or activity of Hey.
Embodiment 626. The method of any of the above enumerated embodiments wherein the Wnt agonist increases the expression of Deltex-1 by 200% or more than it increases the expression or activity of Hey.
Embodiment 627. The method of any of the above enumerated embodiments wherein the Wnt agonist increases the expression of Deltex-1 by 250% or more than it increases the expression or activity of Hey.
Embodiment 628. The method of any of the above enumerated embodiments wherein the Wnt agonist increases the expression of Hif-1 by 10% or more than it increases the expression or activity of Hey.
Embodiment 629. The method of any of the above enumerated embodiments wherein the Wnt agonist increases the expression of Hif-1 by 25% or more than it increases the expression or activity of Hey.
Embodiment 630. The method of any of the above enumerated embodiments wherein the Wnt agonist increases the expression of Hif-1 by 50% or more than it increases the expression or activity of Hey.
Embodiment 631. The method of any of the above enumerated embodiments wherein the Wnt agonist increases the expression of Hif-1 by 75% or more than it increases the expression or activity of Hey.
Embodiment 632. The method of any of the above enumerated embodiments wherein the Wnt agonist increases the expression of Hif-1 by¹⁰⁰% or more than it increases the expression or activity of Hey.
Embodiment 633. The method of any of the above enumerated embodiments wherein the Wnt agonist increases the expression of Hif-1 by 125% or more than it increases the expression or activity of Hey.
Embodiment 634. The method of any of the above enumerated embodiments wherein the Wnt agonist increases the expression of Hif-1 by¹⁵⁰% or more than it increases the expression or activity of Hey.
Embodiment 635. The method of any of the above enumerated embodiments wherein the Wnt agonist increases the expression of Hif-1 by 175% or more than it increases the expression or activity of Hey.
Embodiment 636. The method of any of the above enumerated embodiments wherein the Wnt agonist increases the expression of Hif-1 by 200% or more than it increases the expression or activity of Hey.
Embodiment 637. The method of any of the above enumerated embodiments wherein the Wnt agonist increases the expression of Hif-1 by 250% or more than it increases the expression or activity of Hey.
Embodiment 638. The method of any of the above enumerated embodiments wherein the agent having activity as a Wnt agonist is a Wnt agonist listed in Table 3.
Embodiment 639. The method of any of the above enumerated embodiments wherein the agent having activity as a Wnt agonist is AZD1080.
Embodiment 640. The method of any of the above enumerated embodiments wherein the agent having activity as a Wnt agonist is LY2090314.
Embodiment 641. The method of any of the above enumerated embodiments wherein the agent having activity as a Wnt agonist is GSK3 inhibitor XXII.
Embodiment 642. The method of any of the above enumerated embodiments wherein the agent having activity as a Wnt agonist is CHIR99021.
Embodiment 643. The method of any of the above enumerated embodiments wherein the Wnt agonist is an analogue of LY2090314 as known in the art.
Embodiment 644. The method of any of the above enumerated embodiments wherein the Wnt agonist is Wnt-1.
Embodiment 645. The method of any of the above enumerated embodiments wherein the Wnt agonist is Wnt-2/Irp.
Embodiment 646. The method of any of the above enumerated embodiments wherein the Wnt agonist is Wnt-2b/13.
Embodiment 647. The method of any of the above enumerated embodiments wherein the Wnt agonist is Wnt-3/Int-4.
Embodiment 648. The method of any of the above enumerated embodiments wherein the Wnt agonist is Wnt-3a.
Embodiment 649. The method of any of the above enumerated embodiments wherein the Wnt agonist is Wnt-4.
Embodiment 650. The method of any of the above enumerated embodiments wherein the Wnt agonist is Wnt-5a.
Embodiment 651. The method of any of the above enumerated embodiments wherein the Wnt agonist is Wnt-5b.
Embodiment 652. The method of any of the above enumerated embodiments wherein the Wnt agonist is Wnt-6.
Embodiment 653. The method of any of the above enumerated embodiments wherein the Wnt agonist is Wnt-7a.
Embodiment 654. The method of any of the above enumerated embodiments wherein the Wnt agonist is Wnt-7b.
Embodiment 655. The method of any of the above enumerated embodiments wherein the Wnt agonist is Wnt-8a/8d.
Embodiment 656. The method of any of the above enumerated embodiments wherein the Wnt agonist is Wnt-8b.
Embodiment 657. The method of any of the above enumerated embodiments wherein the Wnt agonist is Wnt-9a/14.
Embodiment 658. The method of any of the above enumerated embodiments wherein the Wnt agonist is Wnt-9b/14b/15.
Embodiment 659. The method of any of the above enumerated embodiments wherein the Wnt agonist is Wnt-IOa.
Embodiment 660. The method of any of the above enumerated embodiments wherein the Wnt agonist is Wnt-10b/12.
Embodiment 661. The method of any of the above enumerated embodiments wherein the Wnt agonist is Wnt-11.
Embodiment 662. The method of any of the above enumerated embodiments wherein the Wnt agonist is Wnt-16.
Embodiment 663. The method of any of the above enumerated embodiments wherein the Wnt agonist is R-Spondin 1/2/3/4.
Embodiment 664. The method of any of the above enumerated embodiments wherein the Wnt agonist is Norrin.
Embodiment 665. The method of any of the above enumerated embodiments wherein the Wnt agonist is BML-284.
Embodiment 666. The method of any of the above enumerated embodiments wherein the Wnt agonist is IQ 1.
Embodiment 667. The method of any of the above enumerated embodiments wherein the Wnt agonist is DCA.
Embodiment 668. The method of any of the above enumerated embodiments wherein the Wnt agonist is QS 11.
Embodiment 669. The method of any of the above enumerated embodiments wherein the Wnt agonist is WASP-1.
Embodiment 670. The method of any of the above enumerated embodiments wherein the Wnt agonist is WAY 316606.
Embodiment 671. The method of any of the above enumerated embodiments wherein the Wnt agonist is (Dimethylamino)propyl)-2-ethyl-5-(phenylsulfonyl)benzenesulfonamide.
Embodiment 672. The method of any of the above enumerated embodiments wherein the Wnt agonist is Cyclosporine A (CsA).
Embodiment 673. The method of any of the above enumerated embodiments wherein the Wnt agonist is PSC833 (Valspodar).
Embodiment 674. The method of any of the above enumerated embodiments wherein the Wnt agonist is a Cyclosporine analog.
Embodiment 675. The method of any of the above enumerated embodiments wherein the Wnt agonist is WAY-262611.
Embodiment 676. The method of any of the above enumerated embodiments wherein the Wnt agonist is HLY78.
Embodiment 677. The method of any of the above enumerated embodiments wherein the Wnt agonist is SKL2001.
Embodiment 678. The method of any of the above enumerated embodiments wherein the Wnt agonist is Cpdl.
Embodiment 679. The method of any of the above enumerated embodiments wherein the Wnt agonist is Cpd2.
Embodiment 680. The method of any of the above enumerated embodiments wherein the Wnt agonist is 1SX 9.
Embodiment 681. The method of any of the above enumerated embodiments wherein the Wnt agonist is Selumetinib.
Embodiment 682. The method of any of the above enumerated embodiments wherein the Wnt agonist is Radicicol.
Embodiment 683. The method of any of the above enumerated embodiments wherein the Wnt agonist is AT 7519.
Embodiment 684. The method of any of the above enumerated embodiments wherein the Wnt agonist is AZD1080.
Embodiment 685. The method of any of the above enumerated embodiments wherein the Wnt agonist is Tivantinib.
Embodiment 686. The method of any of the above enumerated embodiments wherein the Wnt agonist is 15.
Embodiment 687. The method of any of the above enumerated embodiments wherein the Wnt agonist is BRD4003 chiral.
Embodiment 688. The method of any of the above enumerated embodiments wherein the Wnt agonist is BRD1172.
Embodiment 689. The method of any of the above enumerated embodiments wherein the Wnt agonist is BRD1652.
Embodiment 690. The method of any of the above enumerated embodiments wherein the Wnt agonist is AR-A014418.
Embodiment 691. The method of any of the above enumerated embodiments wherein the Wnt agonist is Bikinin.
Embodiment 692. The method of any of the above enumerated embodiments wherein the Wnt agonist is Hymenialdisine.
Embodiment 693. The method of any of the above enumerated embodiments wherein the Wnt agonist is Aloisine A.
Embodiment 694. The method of any of the above enumerated embodiments wherein the Wnt agonist is Aloisine B.
Embodiment 695. The method of any of the above enumerated embodiments wherein the Wnt agonist is TWS119.
Embodiment 696. The method of any of the above enumerated embodiments wherein the Wnt agonist is CT20026.
Embodiment 697. The method of any of the above enumerated embodiments wherein the Wnt agonist is CHIR99021.
Embodiment 698. The method of any of the above enumerated embodiments wherein the Wnt agonist is CHIR98014.
Embodiment 699. The method of any of the above enumerated embodiments wherein the Wnt agonist is CHIR98023.
Embodiment 700. The method of any of the above enumerated embodiments wherein the Wnt agonist is CHIR98024.
Embodiment 701. The method of any of the above enumerated embodiments wherein the Wnt agonist is CGP60474.
Embodiment 702. The method of any of the above enumerated embodiments wherein the Wnt agonist is AZD2858 (AR28).
Embodiment 703. The method of any of the above enumerated embodiments wherein the Wnt agonist is CID 755673.
Embodiment 704. The method of any of the above enumerated embodiments wherein the Wnt agonist is TCS 2002.
Embodiment 705. The method of any of the above enumerated embodiments wherein the Wnt agonist is Dibromocantharelline.
Embodiment 706. The method of any of the above enumerated embodiments wherein the Wnt agonist is ML320.
Embodiment 707. The method of any of the above enumerated embodiments wherein the Wnt agonist is Flavopiridol.
Embodiment 708. The method of any of the above enumerated embodiments wherein the Wnt agonist is Hymenidin.
Embodiment 709. The method of any of the above enumerated embodiments wherein the Wnt agonist is 6-Bromoindirubin-3-acetoxime.
Embodiment 710. The method of any of the above enumerated embodiments wherein the Wnt agonist is lndirubin-3′-monoxime.
Embodiment 711. The method of any of the above enumerated embodiments wherein the Wnt agonist is 5-lodo-indirubin-3′-monoxime.
Embodiment 712. The method of any of the above enumerated embodiments wherein the Wnt agonist is Indirubin-5-sulfonic acid sodium salt.
Embodiment 713. The method of any of the above enumerated embodiments wherein the Wnt agonist is Indirubin.
Embodiment 714. The method of any of the above enumerated embodiments wherein the Wnt agonist is Lithium Chloride.
Embodiment 715. The method of any of the above enumerated embodiments wherein the Wnt agonist is Beryllium.
Embodiment 716. The method of any of the above enumerated embodiments wherein the Wnt agonist is Zinc.
Embodiment 717. The method of any of the above enumerated embodiments wherein the Wnt agonist is Tungstate.
Embodiment 718. The method of any of the above enumerated embodiments wherein the Wnt agonist is GF109203×.
Embodiment 719. The method of any of the above enumerated embodiments wherein the Wnt agonist is Ro318220.
Embodiment 720. The method of any of the above enumerated embodiments wherein the Wnt agonist is Bisindolylmaleimide X HCl.
Embodiment 721. The method of any of the above enumerated embodiments wherein the Wnt agonist is Enzastaurin.
Embodiment 722. The method of any of the above enumerated embodiments wherein the Wnt agonist is SB-216763.
Embodiment 723. The method of any of the above enumerated embodiments wherein the Wnt agonist is SB-415286.
Embodiment 724. The method of any of the above enumerated embodiments wherein the Wnt agonist is 3F8.
Embodiment 725. The method of any of the above enumerated embodiments wherein the Wnt agonist is TCS 21311.
Embodiment 726. The method of any of the above enumerated embodiments wherein the Wnt agonist is LY2090314.
Embodiment 727. The method of any of the above enumerated embodiments wherein the Wnt agonist is IM-12.
Embodiment 728. The method of any of the above enumerated embodiments wherein the Wnt agonist is KT 5720.
Embodiment 729. The method of any of the above enumerated embodiments wherein the Wnt agonist is Isogranulatimide.
Embodiment 730. The method of any of the above enumerated embodiments wherein the Wnt agonist is BIP-135.
Embodiment 731. The method of any of the above enumerated embodiments wherein the Wnt agonist is CP21R7.
Embodiment 732. The method of any of the above enumerated embodiments wherein the Wnt agonist is HB12.
Embodiment 733. The method of any of the above enumerated embodiments wherein the Wnt agonist is DW12.
Embodiment 734. The method of any of the above enumerated embodiments wherein the Wnt agonist is NP309.
Embodiment 735. The method of any of the above enumerated embodiments wherein the Wnt agonist is (RRu)-HB1229.
Embodiment 736. The method of any of the above enumerated embodiments wherein the Wnt agonist is (RRu)-NP549.
Embodiment 737. The method of any of the above enumerated embodiments wherein the Wnt agonist is Staurosporine.
Embodiment 738. The method of any of the above enumerated embodiments wherein the Wnt agonist is Manzamine A.
Embodiment 739. The method of any of the above enumerated embodiments wherein the Wnt agonist is TC-G 24.
Embodiment 740. The method of any of the above enumerated embodiments wherein the Wnt agonist is SU9516.
Embodiment 741. The method of any of the above enumerated embodiments wherein the Wnt agonist is AZD1080.
Embodiment 742. The method of any of the above enumerated embodiments wherein the Wnt agonist is Kenpaullone.
Embodiment 743. The method of any of the above enumerated embodiments wherein the Wnt agonist is Cmpd 17b.
Embodiment 744. The method of any of the above enumerated embodiments wherein the Wnt agonist is Azakenpaullone.
Embodiment 745. The method of any of the above enumerated embodiments wherein the Wnt agonist is Alsterpaullone.
Embodiment 746. The method of any of the above enumerated embodiments wherein the Wnt agonist is Alsterpaullone CN Ethyl.
Embodiment 747. The method of any of the above enumerated embodiments wherein the Wnt agonist is Cazpaullone.
Embodiment 748. The method of any of the above enumerated embodiments wherein the Wnt agonist is FRATtide.
Embodiment 749. The method of any of the above enumerated embodiments wherein the Wnt agonist is L803.
Embodiment 750. The method of any of the above enumerated embodiments wherein the Wnt agonist is L803-mts.
Embodiment 751. The method of any of the above enumerated embodiments wherein the Wnt agonist is AT 7519.
Embodiment 752. The method of any of the above enumerated embodiments wherein the Wnt agonist is NSC 693868.
Embodiment 753. The method of any of the above enumerated embodiments wherein the Wnt agonist is VP0.7.
Embodiment 754. The method of any of the above enumerated embodiments wherein the Wnt agonist is Palinurin.
Embodiment 755. The method of any of the above enumerated embodiments wherein the Wnt agonist is Tricantin.
Embodiment 756. The method of any of the above enumerated embodiments wherein the Wnt agonist is NPO31115.
Embodiment 757. The method of any of the above enumerated embodiments wherein the Wnt agonist is NPO31112 (Tideglusib).
Embodiment 758. The method of any of the above enumerated embodiments wherein the Wnt agonist is AR-A014418.
Embodiment 759. The method of any of the above enumerated embodiments wherein the Wnt agonist is A-1070722.
Embodiment 760. The method of any of the above enumerated embodiments wherein the agent having activity as an epigenetic agent is an epigenetic agent listed in Table 7, Table 8, Table 9 or Table 10.
Embodiment 761. The method of any of the above enumerated embodiments wherein the agent having activity as an epigenetic agent is an HDAC inhibitor, an LSD1 inhibitor, an EZH2 inhibitor, a DOT1L inhibitor, or a KDM inhibitor.
Embodiment 762. The method of any of the above enumerated embodiments wherein the HDAC inhibitor is Valproic acid.
Embodiment 763. The method of any of the above enumerated embodiments wherein the HDAC inhibitor is Phenyl butyrate.
Embodiment 764. The method of any of the above enumerated embodiments wherein the HDAC inhibitor is Butyrate.
Embodiment 765. The method of any of the above enumerated embodiments wherein the HDAC inhibitor is AN-9
Embodiment 766. The method of any of the above enumerated embodiments wherein the HDAC inhibitor is Entinostat.
Embodiment 767. The method of any of the above enumerated embodiments wherein the HDAC inhibitor is Mocetinostat.
Embodiment 768. The method of any of the above enumerated embodiments wherein the HDAC inhibitor is Tacedinaline.
Embodiment 769. The method of any of the above enumerated embodiments wherein the HDAC inhibitor is BML-210.
Embodiment 770. The method of any of the above enumerated embodiments wherein the HDAC inhibitor is NKL 22.
Embodiment 771. The method of any of the above enumerated embodiments wherein the HDAC inhibitor is RGFP109.
Embodiment 772. The method of any of the above enumerated embodiments wherein the HDAC inhibitor is RGFP136.
Embodiment 773. The method of any of the above enumerated embodiments wherein the HDAC inhibitor is RGFP966.
Embodiment 774. The method of any of the above enumerated embodiments wherein the HDAC inhibitor is 4SC-202.
Embodiment 775. The method of any of the above enumerated embodiments wherein the HDAC inhibitor is Chidamide.
Embodiment 776. The method of any of the above enumerated embodiments wherein the HDAC inhibitor is TC-H 106.
Embodiment 777. The method of any of the above enumerated embodiments wherein the HDAC inhibitor is Romidepsin.
Embodiment 778. The method of any of the above enumerated embodiments wherein the HDAC inhibitor is Trapoxin A.
Embodiment 779. The method of any of the above enumerated embodiments wherein the HDAC inhibitor is HC Toxin.
Embodiment 780. The method of any of the above enumerated embodiments wherein the HDAC inhibitor is Apicidin.
Embodiment 781. The method of any of the above enumerated embodiments wherein the HDAC inhibitor is Thailandepsin A.
Embodiment 782. The method of any of the above enumerated embodiments wherein the HDAC inhibitor is Dihydrochlamydocin.
Embodiment 783. The method of any of the above enumerated embodiments wherein the HDAC inhibitor is (−)-Depudecin.
Embodiment 784. The method of any of the above enumerated embodiments wherein the HDAC inhibitor is Parthenolide.
Embodiment 785. The method of any of the above enumerated embodiments wherein the HDAC inhibitor is Trichostatin A (TSA).
Embodiment 786. The method of any of the above enumerated embodiments wherein the HDAC inhibitor is SAHA.
Embodiment 787. The method of any of the above enumerated embodiments wherein the HDAC inhibitor is 4-iodo-SAHA.
Embodiment 788. The method of any of the above enumerated embodiments wherein the HDAC inhibitor is SBHA.
Embodiment 789. The method of any of the above enumerated embodiments wherein the HDAC inhibitor is CBHA.
Embodiment 790. The method of any of the above enumerated embodiments wherein the HDAC inhibitor is LAQ-824.
Embodiment 791. The method of any of the above enumerated embodiments wherein the HDAC inhibitor is PDX-101.
Embodiment 792. The method of any of the above enumerated embodiments wherein the HDAC inhibitor is LBH-589.
Embodiment 793. The method of any of the above enumerated embodiments wherein the HDAC inhibitor is ITF2357.
Embodiment 794. The method of any of the above enumerated embodiments wherein the HDAC inhibitor is PCI-34051.
Embodiment 795. The method of any of the above enumerated embodiments wherein the HDAC inhibitor is PCI-24781.
Embodiment 796. The method of any of the above enumerated embodiments wherein the HDAC inhibitor is Tubastatin A.
Embodiment 797. The method of any of the above enumerated embodiments wherein the HDAC inhibitor is CUDC-101.
Embodiment 798. The method of any of the above enumerated embodiments wherein the HDAC inhibitor is Oxamflatin.
Embodiment 799. The method of any of the above enumerated embodiments wherein the HDAC inhibitor is 1TF2357.
Embodiment 800. The method of any of the above enumerated embodiments wherein the HDAC inhibitor is Bufexamac.
Embodiment 801. The method of any of the above enumerated embodiments wherein the HDAC inhibitor is APHA Compound 8.
Embodiment 802. The method of any of the above enumerated embodiments wherein the HDAC inhibitor is Tubacin.
Embodiment 803. The method of any of the above enumerated embodiments wherein the HDAC inhibitor is Butyrylhydroxamic acid.
Embodiment 804. The method of any of the above enumerated embodiments wherein the HDAC inhibitor is MC 1568.
Embodiment 805. The method of any of the above enumerated embodiments wherein the HDAC inhibitor is SB939.
Embodiment 806. The method of any of the above enumerated embodiments wherein the HDAC inhibitor is 4SC-201.
Embodiment 807. The method of any of the above enumerated embodiments wherein the HDAC inhibitor is Tefinostat.
Embodiment 808. The method of any of the above enumerated embodiments wherein the HDAC inhibitor is CHR-3996.
Embodiment 809. The method of any of the above enumerated embodiments wherein the HDAC inhibitor is NSC 57457.
Embodiment 810. The method of any of the above enumerated embodiments wherein the HDAC inhibitor is CG200745.
Embodiment 811. The method of any of the above enumerated embodiments wherein the HDAC inhibitor is ACY1215.
Embodiment 812. The method of any of the above enumerated embodiments wherein the HDAC inhibitor is Nexturastat A.
Embodiment 813. The method of any of the above enumerated embodiments wherein the HDAC inhibitor is Droxinostat.
Embodiment 814. The method of any of the above enumerated embodiments wherein the HDAC inhibitor is Scriptaid.
Embodiment 815. The method of any of the above enumerated embodiments wherein the HDAC inhibitor is BRD9757.
Embodiment 816. The method of any of the above enumerated embodiments wherein the HDAC inhibitor is HPOB.
Embodiment 817. The method of any of the above enumerated embodiments wherein the HDAC inhibitor is CAY10603.
Embodiment 818. The method of any of the above enumerated embodiments wherein the HDAC inhibitor is HDAC6 Inhibitor III.
Embodiment 819. The method of any of the above enumerated embodiments wherein the HDAC inhibitor is M 344.
Embodiment 820. The method of any of the above enumerated embodiments wherein the HDAC inhibitor is 4-(dimethylamino)-N-[6-(hydroxyamino)-6-oxohexyl]-benzamide.
Embodiment 821. The method of any of the above enumerated embodiments wherein the HDAC inhibitor is (S)-HDAC-42.
Embodiment 822. The method of any of the above enumerated embodiments wherein the HDAC inhibitor is HNHA.
Embodiment 823. The method of any of the above enumerated embodiments wherein the HDAC inhibitor is Pyroxamide.
Embodiment 824. The method of any of the above enumerated embodiments wherein the HDAC inhibitor is LMK235.
Embodiment 825. The method of any of the above enumerated embodiments wherein the HDAC inhibitor is HDAC-IN-1.
Embodiment 826. The method of any of the above enumerated embodiments wherein the HDAC inhibitor is VAHA.
Embodiment 827. The method of any of the above enumerated embodiments wherein the HDAC inhibitor is Ratjadone A.
Embodiment 828. The method of any of the above enumerated embodiments wherein the HDAC inhibitor is TCS HDAC6 20b.
Embodiment 829. The method of any of the above enumerated embodiments wherein the HDAC inhibitor is PTACH.
Embodiment 830. The method of any of the above enumerated embodiments wherein the HDAC inhibitor is KD 5170.
Embodiment 831. The method of any of the above enumerated embodiments wherein the HDAC inhibitor is SIRT1/2 Inhibitor VII.
Embodiment 832. The method of any of the above enumerated embodiments wherein the HDAC inhibitor is TMP269.
Embodiment 833. The method of any of the above enumerated embodiments wherein the HDAC inhibitor is Tasquinimod.
Embodiment 834. The method of any of the above enumerated embodiments wherein the agent having activity as an epigenetic agent is an EZH2 inhibitor.
Embodiment 835. The method of any of the above enumerated embodiments wherein the EZH-2 inhibitor is GSK126 from GSK Phase I (GSK2816126).
Embodiment 836. The method of any of the above enumerated embodiments wherein the EZH-2 inhibitor is SHR2554.
Embodiment 837. The method of any of the above enumerated embodiments wherein the EZH-2 inhibitor is MAK683.
Embodiment 838. The method of any of the above enumerated embodiments wherein the EZH-2 inhibitor is CPI-0169.
Embodiment 839. The method of any of the above enumerated embodiments wherein the EZH-2 inhibitor is EPZ-011989.
Embodiment 840. The method of any of the above enumerated embodiments wherein the EZH-2 inhibitor is EPZ-005687.
Embodiment 841. The method of any of the above enumerated embodiments wherein the EZH-2 inhibitor is CPI-360.
Embodiment 842. The method of any of the above enumerated embodiments wherein the EZH-2 inhibitor is SKLB1049.
Embodiment 843. The method of any of the above enumerated embodiments wherein the EZH-2 inhibitor is ZLD1039.
Embodiment 844. The method of any of the above enumerated embodiments wherein the EZH-2 inhibitor is ZLD1122.
Embodiment 845. The method of any of the above enumerated embodiments wherein the EZH-2 inhibitor is GSK503 from Arch Otolaryngol Head Neck Surg. 2001,127(4), 447-452.
Embodiment 846. The method of any of the above enumerated embodiments wherein the EZH-2 inhibitor is GSK926 from ACS Med. Chem. Lett. 2012, 3, 1091-1096.
Embodiment 847. The method of any of the above enumerated embodiments wherein the EZH-2 inhibitor is GSK343 from ACS Med. Chem. Lett. 2012, 3, 1091-1096.
Embodiment 848. The method of any of the above enumerated embodiments wherein the EZH-2 inhibitor is EBI-2511.
Embodiment 849. The method of any of the above enumerated embodiments wherein the EZH-2 inhibitor is (R)-OR-S1.
Embodiment 850. The method of any of the above enumerated embodiments wherein the EZH-2 inhibitor is A-395.
Embodiment 851. The method of any of the above enumerated embodiments wherein the EZH-2 inhibitor is astemizole.
Embodiment 852. The method of any of the above enumerated embodiments wherein the EZH-2 inhibitor is EED162.
Embodiment 853. The method of any of the above enumerated embodiments wherein the EZH-2 inhibitor is CPI-0209.
Embodiment 854. The method of any of the above enumerated embodiments wherein the EZH-2 inhibitor is EED226.
Embodiment 855. The method of any of the above enumerated embodiments wherein the EZH-2 inhibitor is DZNep.
Embodiment 856. The method of any of the above enumerated embodiments wherein the EZH-2 inhibitor is UNC1999.
Embodiment 857. The method of any of the above enumerated embodiments wherein the EZH-2 inhibitor is sinefungin.
Embodiment 858. The method of any of the above enumerated embodiments wherein the EZH-2 inhibitor is tanshindiol B.
Embodiment 859. The method of any of the above enumerated embodiments wherein the EZH-2 inhibitor is tanshindiol C.
Embodiment 860. The method of any of the above enumerated embodiments wherein the EZH-2 inhibitor is Cmpd 44 from ACS Med. Chem. Lett. 2014, 5, 378-383.
Embodiment 861. The method of any of the above enumerated embodiments wherein the EZH-2 inhibitor is MC1945 from Arch Otolaryngol Head Neck Surg. 2001,127(4), 447-452.
Embodiment 862. The method of any of the above enumerated embodiments wherein the EZH-2 inhibitor is MC1947 from Arch Otolaryngol Head Neck Surg. 2001,127(4), 447-452.
Embodiment 863. The method of any of the above enumerated embodiments wherein the EZH-2 inhibitor is MC1948 from Arch Otolaryngol Head Neck Surg. 2001,127(4), 447-452.
Embodiment 864. The method of any of the above enumerated embodiments wherein the EZH-2 inhibitor is curcumin as described in European Journal of Pharmacology 2010, 637, 16-21.
Embodiment 865. The method of any of the above enumerated embodiments wherein the EZH-2 inhibitor is (−)-Epigallocatechin-3-gallate (EGCG) from Carcinogenesis. 2011; 32: 1525-32.
Embodiment 866. The method of any of the above enumerated embodiments wherein the EZH-2 inhibitor is sulforaphane from Mol Pharmacol. 2011, 80, 870-8.
Embodiment 867. The method of any of the above enumerated embodiments wherein the EZH-2 inhibitor is SAH-EZH2 from Current Medicinal Chemistry (2016), 23(27), 3025-3043.
Embodiment 868. The method of any of the above enumerated embodiments wherein the agent having activity as an epigenetic agent is a DOT1L inhibitor.
Embodiment 869. The method of any of the above embodiments wherein the small molecule targeting DOT1L is SYC-687.
Embodiment 870. The method of any of the above embodiments wherein the small molecule targeting DOT1L is SYC-522.
Embodiment 871. The method of any of the above embodiments wherein the small molecule targeting DOT1L is EPZ002696.
Embodiment 872. The method of any of the above embodiments wherein the small molecule targeting DOT1L is EPZ004450.
Embodiment 873. The method of any of the above embodiments wherein the small molecule targeting DOT1L is CN SAH.
Embodiment 874. The method of any of the above embodiments wherein the small molecule targeting DOT1L is SAH.
Embodiment 875. The method of any of the above embodiments wherein the small molecule targeting DOT1L is bromo-deaza-SAH.
Embodiment 876. The method of any of the above embodiments wherein the small molecule targeting DOT1L is compound 21 from ACS Medicinal Chemistry Letters (2018), 9(9), 895-900-Peptides.
Embodiment 877. The method of any of the above embodiments wherein the small molecule targeting DOT1L is compound 28 from ACS Medicinal Chemistry Letters (2018), 9(9), 895-900-Peptides.
Embodiment 878. The method of any of the above embodiments wherein the small molecule targeting DOT1L is compound 8H from Bioorganic Chemistry (2018), 80, 649-654.
Embodiment 879. The method of any of the above enumerated embodiments wherein the LSD1 inhibitor is reversible.
Embodiment 880. The method of any of the above enumerated embodiments wherein the LSD1 inhibitor is irreversible.
Embodiment 881. The method of any of the above enumerated embodiments wherein the agent having activity as an LSD1 inhibitor is Tranylcypromine (TCP)
Embodiment 882. The method of any of the above enumerated embodiments wherein the agent having activity as an LSD1 inhibitor is GSK-2879552.
Embodiment 883. The method of any of the above enumerated embodiments wherein the agent having activity as an LSD1 inhibitor is GSK-LSD1.
Embodiment 884. The method of any of the above enumerated embodiments wherein the agent having activity as an LSD1 inhibitor is Phenelzine sulfate.
Embodiment 885. The method of any of the above enumerated embodiments wherein the agent having activity as an LSD1 inhibitor is ORY-2001 (Vafidemstat).
Embodiment 886. The method of any of the above enumerated embodiments wherein the agent having activity as an LSD1 inhibitor is SP-2577 (Seclidemstat).
Embodiment 887. The method of any of the above enumerated embodiments wherein the agent having activity as an LSD1 inhibitor is Osimertinib (AZD9291).
Embodiment 888. The method of any of the above enumerated embodiments wherein the agent having activity as an LSD1 inhibitor is GCG-11047 (PG-11047).
Embodiment 889. The method of any of the above enumerated embodiments wherein the agent having activity as an LSD1 inhibitor is ORY-1001 (RG6016, R07051790, Iadademstat).
Embodiment 890. The method of any of the above enumerated embodiments wherein the agent having activity as an LSD1 inhibitor is IMG-7289.
Embodiment 891. The method of any of the above enumerated embodiments wherein the agent having activity as an LSD1 inhibitor is CC-90011.
Embodiment 892. The method of any of the above enumerated embodiments wherein the agent having activity as an LSD1 inhibitor is INCB059872.
Embodiment 893. The method of any of the above enumerated embodiments wherein the agent having activity as an LSD1 inhibitor is an agent listed in Table 9.
Embodiment 894. The method of any of the above enumerated embodiments wherein the LSD-1 inhibitor is ORY-2001.
Embodiment 895. The method of any of the above enumerated embodiments wherein the LSD-1 inhibitor is SP-2577.
Embodiment 896. The method of any of the above enumerated embodiments wherein the LSD-1 inhibitor is osimertinib.
Embodiment 897. The method of any of the above enumerated embodiments wherein the LSD-1 inhibitor is GCG-11047.
Embodiment 898. The method of any of the above enumerated embodiments wherein the LSD-1 inhibitor is ORY-1001.
Embodiment 899. The method of any of the above enumerated embodiments wherein the LSD-1 inhibitor is IMG-7289.
Embodiment 900. The method of any of the above enumerated embodiments wherein the LSD-1 inhibitor is CC-90011.
Embodiment 901. The method of any of the above enumerated embodiments wherein the LSD-1 inhibitor is INCB059872.
Embodiment 902. The method of any of the above enumerated embodiments wherein the LSD-1 inhibitor is TCP trans chiral from J. American Chemical Society (2010), 132(19), 6827-6833.
Embodiment 903. The method of any of the above enumerated embodiments wherein the LSD-1 inhibitor is TCP cis from Bioorganic Medicinal Chemistry 2008, 16(15), 7148-7166.
Embodiment 904. The method of any of the above enumerated embodiments wherein the LSD-1 inhibitor is TCP cis chiral.
Embodiment 905. The method of any of the above enumerated embodiments wherein the LSD-1 inhibitor is RN-1 from Medicinal Research Reviews 2013, 33(4), 873-910.
Embodiment 906. The method of any of the above enumerated embodiments wherein the LSD-1 inhibitor is compound 1 from PLoS One (2017), 12(2), e0170301.
Embodiment 907. The method of any of the above enumerated embodiments wherein the LSD-1 inhibitor is compound 45 from Medicinal Research Reviews 2013, 33(4), 873-910.
Embodiment 908. The method of any of the above enumerated embodiments wherein the LSD-1 inhibitor is RN-7 from Epigenomics (2015), 7(8), 1379-1396.
Embodiment 909. The method of any of the above enumerated embodiments wherein the LSD-1 inhibitor is compound 5A from Future Med. Chem. (2017) 9(11), 1161-1174.
Embodiment 910. The method of any of the above enumerated embodiments wherein the LSD-1 inhibitor is compound 2 from Medicinal Research Reviews 2015, 35(3), 586-618.
Embodiment 911. The method of any of the above enumerated embodiments wherein the LSD-1 inhibitor is compound 12f from Med. Chem. Commun., 2015, 6, 665-670.
Embodiment 912. The method of any of the above enumerated embodiments wherein the LSD-1 inhibitor is T-3775440 from Medicinal Research Reviews 2013, 33(4), 873-910.
Embodiment 913. The method of any of the above enumerated embodiments wherein the LSD-1 inhibitor is OG-L002.
Embodiment 914. The method of any of the above enumerated embodiments wherein the LSD-1 inhibitor is S2101.
Embodiment 915. The method of any of the above enumerated embodiments wherein the LSD-1 inhibitor is NCL-1.
Embodiment 916. The method of any of the above enumerated embodiments wherein the LSD-1 inhibitor is compound 9A from Bioorganic Medicinal Chemistry Letters 27 (2017) 2099-2101.
Embodiment 917. The method of any of the above enumerated embodiments wherein the LSD-1 inhibitor is compound 191 from Chem. Pharm. Bull. 63, 882-889 (2015).
Embodiment 918. The method of any of the above enumerated embodiments wherein the LSD-1 inhibitor is NCD-25.
Embodiment 919. The method of any of the above enumerated embodiments wherein the LSD-1 inhibitor is NCD-38.
Embodiment 920. The method of any of the above enumerated embodiments wherein the LSD-1 inhibitor is compound 14A from RSC Advances (2018), 8(3), 1666-1676.
Embodiment 921. The method of any of the above enumerated embodiments wherein the LSD-1 inhibitor is compound 15A from RSC Advances (2018), 8(3), 1666-1676.
Embodiment 922. The method of any of the above enumerated embodiments wherein the LSD-1 inhibitor is compound 15B from RSC Advances (2018), 8(3), 1666-1676.
Embodiment 923. The method of any of the above enumerated embodiments wherein the LSD-1 inhibitor is compound 4 from Bioorganic Medicinal Chemistry Letters 28 (2018) 1001-1004.
Embodiment 924. The method of any of the above enumerated embodiments wherein the LSD-1 inhibitor is pargyline.
Embodiment 925. The method of any of the above enumerated embodiments wherein the LSD-1 inhibitor the peptide disclosed in Nature Structural & Molecular Biology, 2007, 14(6), 535.
Embodiment 926. The method of any of the above enumerated embodiments wherein the LSD-1 inhibitor is bizine.
Embodiment 927. The method of any of the above enumerated embodiments wherein the LSD-1 inhibitor is compound 5a from Bioorganic Medicinal Chemistry Letters 26 (2016) 4552-4557.
Embodiment 928. The method of any of the above enumerated embodiments wherein the LSD-1 inhibitor is compound 5n from Bioorganic Medicinal Chemistry Letters 26 (2016) 4552-4557.
Embodiment 929. The method of any of the above enumerated embodiments wherein the LSD-1 inhibitor is SP-2509.
Embodiment 930. The method of any of the above enumerated embodiments wherein the LSD-1 inhibitor is LSD1-1N-32 from J. Med. Chem. 2017, 60, 7984-7999.
Embodiment 931. The method of any of the above enumerated embodiments wherein the LSD-1 inhibitor is LSD1-IN-1 Ip from Bioorganic Medicinal Chemistry Letters 27 (2017) 3190-3195.
Embodiment 932. The method of any of the above enumerated embodiments wherein the LSD-1 inhibitor is resveratrol.
Embodiment 933. The method of any of the above enumerated embodiments wherein the LSD-1 inhibitor is hydroxylamine.
Embodiment 934. The method of any of the above enumerated embodiments wherein the LSD-1 inhibitor is compound 8c from Bioorganic Medicinal Chemistry 2018, 26, 6000.
Embodiment 935. The method of any of the above enumerated embodiments wherein the LSD-1 inhibitor is CBB-1007.
Embodiment 936. The method of any of the above enumerated embodiments wherein the LSD-1 inhibitor is namoline from Int. J. Cancer 2012, 131, 2704-2709.
Embodiment 937. The method of any of the above enumerated embodiments wherein the LSD-1 inhibitor is GSK-354 from Future Medicinal Chemistry (2017), 9(11), 1227-1242.
Embodiment 938. The method of any of the above enumerated embodiments wherein the LSD-1 inhibitor is GSK-690 from Future Medicinal Chemistry (2017), 9(11), 1227-1242.
Embodiment 939. The method of any of the above enumerated embodiments wherein the LSD-1 inhibitor is E11.
Embodiment 940. The method of any of the above enumerated embodiments wherein the LSD-1 inhibitor is MC2694.
Embodiment 941. The method of any of the above enumerated embodiments wherein the LSD-1 inhibitor is alpha-mangostin.
Embodiment 942. The method of any of the above enumerated embodiments wherein the LSD-1 inhibitor is compound 4 from European Journal of Medicinal Chemistry (2019), 162, 555-567.
Embodiment 943. The method of any of the above enumerated embodiments wherein the LSD-1 inhibitor is compound 10d from Bioorganic Chemistry 2018, 78, 7-16.
Embodiment 944. The method of any of the above enumerated embodiments wherein the LSD-1 inhibitor is compound 90 from J. Med. Chem. 2017, 60, 1673-1692.
Embodiment 945. The method of any of the above enumerated embodiments wherein the LSD-1 inhibitor is compound 46 J. Med. Chem. 2017, 60, 1693-1715.
Embodiment 946. The method of any of the above enumerated embodiments wherein the LSD-1 inhibitor is compound 49 J. Med. Chem. 2017, 60, 1693-1715.
Embodiment 947. The method of any of the above enumerated embodiments wherein the LSD-1 inhibitor is compound 50 J. Med. Chem. 2017, 60, 1693-1715.
Embodiment 948. The method of any of the above enumerated embodiments wherein the LSD-1 inhibitor is polymyxin B from Future Medicinal Chemistry (2017), 9(11), 1227-1242.
Embodiment 949. The method of any of the above enumerated embodiments wherein the LSD-1 inhibitor is polymyxin E from Future Medicinal Chemistry (2017), 9(11), 1227-1242.
Embodiment 950. The method of any of the above enumerated embodiments wherein the LSD-1 inhibitor is baicalin.
Embodiment 951. The method of any of the above enumerated embodiments wherein the LSD-1 inhibitor is compound 16Q from Med. Chem. Commun., 2013, 4, 1513.
Embodiment 952. The method of any of the above enumerated embodiments wherein the LSD-1 inhibitor is LSD1 inhibitor 24.
Embodiment 953. The method of any of the above enumerated embodiments wherein the LSD-1 inhibitor is geranylgeranoic acid from Biochemical and Biophysical Research Communications 444 (2014) 24-29.
Embodiment 954. The method of any of the above enumerated embodiments wherein the LSD-1 inhibitor is geranylgeraniol.
Embodiment 955. The method of any of the above enumerated embodiments wherein the LSD-1 inhibitor is thiocarbamate.
Embodiment 956. The method of any of the above enumerated embodiments wherein the LSD-1 inhibitor is thiourea.
Embodiment 957. The method of any of the above enumerated embodiments wherein the LSD-1 inhibitor is thienopyrrole.
Embodiment 958. The method of any of the above enumerated embodiments wherein the LSD-1 inhibitor is 4SC-202.
Embodiment 959. The method of any of the above enumerated embodiments wherein the LSD-1 inhibitor is ORY-3001.
Embodiment 960. The method of any of the above enumerated embodiments wherein the LSD-1 inhibitor is JL1037 from Oncotarget 2017, 8(19), 31901-31914.
Embodiment 961. The method of any of the above enumerated embodiments wherein the LSD-1 inhibitor is FLI-06.
Embodiment 962. The method of any of the above enumerated embodiments wherein the LSD-1 inhibitor is rhodium complex 1 from J. Med. Chem. 2017, 60, 2597-2603.
Embodiment 963. The method of any of the above enumerated embodiments wherein the KDM inhibitor is TC-E 5002 from Journal of Medicinal Chemistry (2013), 56(18), 7222-7231-TC-E 5002.
Embodiment 964. The method of any of the above enumerated embodiments wherein the KDM inhibitor is AS 8351 from Science (2016), 352(6290), 1216-1220.
Embodiment 965. The method of any of the above enumerated embodiments wherein the KDM inhibitor is EPT-103182 from Clinical Epigenetics (2016) 8, 57-histone methyltransferases and demethylase review.
Embodiment 966. The method of any of the above enumerated embodiments wherein the KDM inhibitor is Compound 54j from Journal of Medicinal Chemistry (2016), 59(4), 1388-1409-Inhibitors of the KDM4 and KDM5.
Embodiment 967. The method of any of the above enumerated embodiments wherein the KDM inhibitor is Compound 54k from Journal of Medicinal Chemistry (2016), 59(4), 1388-1409-Inhibitors of the KDM4 and KDM5.
Embodiment 968. The method of any of the above enumerated embodiments wherein the KDM inhibitor is Compound 1 from Journal of Medicinal Chemistry (2014), 57(1), 42-55-LSD1 plus KDM inhibition.
Embodiment 969. The method of any of the above enumerated embodiments wherein the KDM inhibitor is Compound 34 from Journal of Medicinal Chemistry (2016), 59(4), 1357-1369-Inhibitors of the KDM4 and KDM5.
Embodiment 970. The method of any of the above enumerated embodiments wherein the KDM inhibitor is Compound 41 from Journal of Medicinal Chemistry (2016), 59(4), 1357-1369-Inhibitors of the KDM4 and KDM5.
Embodiment 971. The method of any of the above enumerated embodiments wherein the KDM inhibitor is Compound 48 from Bioorganic & Medicinal Chemistry Letters (2016), 26(16), 4036-4041-pyrazolopyrimidin KDM5.
Embodiment 972. The method of any of the above enumerated embodiments wherein the KDM inhibitor is CPI-4203.
Embodiment 973. The method of any of the above enumerated embodiments wherein the KDM inhibitor is CPI-455.
Embodiment 974. The method of any of the above enumerated embodiments wherein the KDM inhibitor is E67-2.
Embodiment 975. The method of any of the above enumerated embodiments wherein the KDM inhibitor is KDOAM25.
Embodiment 976. The method of any of the above enumerated embodiments wherein the KDM inhibitor is Compound 33 from Bioorganic & Medicinal Chemistry Letters (2018), 28(9), 1490-1494-potent and selective KDM5.
Embodiment 977. The method of any of the above enumerated embodiments wherein the KDM inhibitor is N11 from Cell Chemical Biology (2016), 23(7), 749-751.
Embodiment 978. The method of any of the above enumerated embodiments wherein the KDM inhibitor is GSK-467 from Pharmacological Research (2019), 141, 1-20-Histone demethylase KDM5B-based targeting.
Embodiment 979. The method of any of the above enumerated embodiments wherein the KDM inhibitor is GSK-J1 from Pharmacological Research (2019), 141, 1-20-Histone demethylase KDM5B-based targeting.
Embodiment 980. The method of any of the above enumerated embodiments wherein the KDM inhibitor is GSK-J4 from Pharmacological Research (2019), 141, 1-20-Histone demethylase KDM5B-based targeting.
Embodiment 981. The method of any of the above enumerated embodiments wherein the KDM inhibitor is KDM5-C49 from Pharmacological Research (2019), 141, 1-20-Histone demethylase KDM5B-based targeting.
Embodiment 982. The method of any of the above enumerated embodiments wherein the KDM inhibitor is KDM5-C50 from Pharmacological Research (2019), 141, 1-20-Histone demethylase KDM5B-based targeting.
Embodiment 983. The method of any of the above enumerated embodiments wherein the KDM inhibitor is Compound 48 from Bioorganic & Medicinal Chemistry Letters (2017), 27(13), 2974-2981-Oral KDM5 Inhib.
Embodiment 984. The method of any of the above enumerated embodiments wherein the KDM inhibitor is Compound 49 from Bioorganic & Medicinal Chemistry Letters (2017), 27(13), 2974-2981-Oral KDM5 Inhib.
Embodiment 985. The method of any of the above enumerated embodiments wherein the KDM inhibitor is Compound 50 from Bioorganic & Medicinal Chemistry Letters (2017), 27(13), 2974-2981-Oral KDM5 Inhib.
Embodiment 986. The method of any of the above enumerated embodiments wherein the KDM inhibitor is Compound R-35 from MedChemComm (2014), 5(12), 1879-1886-Optimisation of a triazolopyridine.
Embodiment 987. The method of any of the above enumerated embodiments wherein the KDM inhibitor is Compound 1 from Angewandte Chemie, International Edition (2018), 57(40), 13091-13095-Rhodium(III) Complex.
Embodiment 988. The method of any of the above enumerated embodiments wherein the KDM inhibitor is Daminozide.
Embodiment 989. The method of any of the above enumerated embodiments wherein the KDM inhibitor is JIB-04.
Embodiment 990. The method of any of the above enumerated embodiments wherein the KDM inhibitor is Methylstat.
Embodiment 991. The method of any of the above enumerated embodiments wherein the KDM inhibitor is NSC 6369819.
Embodiment 992. The method of any of the above enumerated embodiments wherein the KDM inhibitor is Compound 10r from Bioorganic & Medicinal Chemistry Letters (2017), 27(14), 3201-3204-histone lysine demethylase 4D.
Embodiment 993. The method of any of the above enumerated embodiments wherein the KDM inhibitor is N71 from Journal of Medicinal Chemistry (2018), 61(23), 10588-10601-Irreversible Inhibitors.
Embodiment 994. The method of any of the above enumerated embodiments wherein the KDM inhibitor is TC-E 5002.
Embodiment 995. The method of any of the above enumerated embodiments wherein the KDM inhibitor is AS 8351.
Embodiment 996. The method of any of the above enumerated embodiments wherein the HDAC inhibitor results in a decrease in histone deacetylation of a target gene in a cell.
Embodiment 997. The method of any of the above enumerated embodiments wherein the HDAC inhibitor decreases expression or enzymatic activity of HDAC by at least about 1.1-fold or more relative to a control.
Embodiment 998. The method of any of the above enumerated embodiments wherein the HDAC inhibitor decreases expression or enzymatic activity of HDAC by at least about 1.2-fold or more relative to a control.
Embodiment 999. The method of any of the above enumerated embodiments wherein the HDAC inhibitor decreases expression or enzymatic activity of HDAC by at least about 1.3-fold or more relative to a control.
Embodiment 1000. The method of any of the above enumerated embodiments wherein the HDAC inhibitor decreases expression or enzymatic activity of HDAC by at least about 1.4-fold or more relative to a control.
Embodiment 1001. The method of any of the above enumerated embodiments wherein the HDAC inhibitor decreases expression or enzymatic activity of HDAC by at least about 1.5-fold or more relative to a control.
Embodiment 1002. The method of any of the above enumerated embodiments wherein the HDAC inhibitor decreases expression or enzymatic activity of HDAC by at least about 1.6-fold or more relative to a control.
Embodiment 1003. The method of any of the above enumerated embodiments wherein the HDAC inhibitor decreases expression or enzymatic activity of HDAC by at least about 1.7-fold or more relative to a control.
Embodiment 1004. The method of any of the above enumerated embodiments wherein the HDAC inhibitor decreases expression or enzymatic activity of HDAC by at least about 1.8-fold or more relative to a control.
Embodiment 1005. The method of any of the above enumerated embodiments wherein the HDAC inhibitor decreases expression or enzymatic activity of HDAC by at least about 1.9-fold or more relative to a control.
Embodiment 1006. The method of any of the above enumerated embodiments wherein the HDAC inhibitor decreases expression or enzymatic activity of HDAC by at least about 2-fold or more relative to a control.
Embodiment 1007. The method of any of the above enumerated embodiments wherein the HDAC inhibitor decreases expression or enzymatic activity of HDAC by at least about 3-fold or more relative to a control.
Embodiment 1008. The method of any of the above enumerated embodiments wherein the HDAC inhibitor decreases expression or enzymatic activity of HDAC by at least about 4-fold or more relative to a control.
Embodiment 1009. The method of any of the above enumerated embodiments wherein the HDAC inhibitor decreases expression or enzymatic activity of HDAC by at least about 5-fold or more relative to a control.
Embodiment 1010. The method of any of the above enumerated embodiments wherein the HDAC inhibitor decreases expression or enzymatic activity of HDAC by at least about 6-fold or more relative to a control.
Embodiment 1011. The method of any of the above enumerated embodiments wherein the HDAC inhibitor decreases expression or enzymatic activity of HDAC by at least about 7-fold or more relative to a control.
Embodiment 1012. The method of any of the above enumerated embodiments wherein the HDAC inhibitor decreases expression or enzymatic activity of HDAC by at least about 8-fold or more relative to a control.
Embodiment 1013. The method of any of the above enumerated embodiments wherein the HDAC inhibitor decreases expression or enzymatic activity of HDAC by at least about 9-fold or more relative to a control.
Embodiment 1014. The method of any of the above enumerated embodiments wherein the HDAC inhibitor decreases expression or enzymatic activity of HDAC by at least about 10-fold or more relative to a control.
Embodiment 1015. The method of any of the above enumerated embodiments wherein the HDAC inhibitor decreases expression or enzymatic activity of HDAC by at least about 15-fold or more relative to a control.
Embodiment 1016. The method of any of the above enumerated embodiments wherein the HDAC inhibitor decreases expression or enzymatic activity of HDAC by at least about 20-fold or more relative to a control.
Embodiment 1017. The method of any of the above enumerated embodiments wherein the HDAC inhibitor decreases expression or enzymatic activity of HDAC by at least about 30-fold or more relative to a control.
Embodiment 1018. The method of any of the above enumerated embodiments wherein the HDAC inhibitor decreases expression or enzymatic activity of HDAC by at least about 40-fold or more relative to a control.
Embodiment 1019. The method of any of the above enumerated embodiments wherein the HDAC inhibitor decreases expression or enzymatic activity of HDAC by at least about 50-fold or more relative to a control.
Embodiment 1020. The method of any of the above enumerated embodiments wherein the HDAC inhibitor decreases expression or enzymatic activity of HDAC by at least about 60-fold or more relative to a control.
Embodiment 1021. The method of any of the above enumerated embodiments wherein the HDAC inhibitor decreases expression or enzymatic activity of HDAC by at least about 70-fold or more relative to a control.
Embodiment 1022. The method of any of the above enumerated embodiments wherein the HDAC inhibitor decreases expression or enzymatic activity of HDAC by at least about 80-fold or more relative to a control.
Embodiment 1023. The method of any of the above enumerated embodiments wherein the HDAC inhibitor decreases expression or enzymatic activity of HDAC by at least about 90-fold or more relative to a control.
Embodiment 1024. The method of any of the above enumerated embodiments wherein the HDAC inhibitor decreases expression or enzymatic activity of HDAC by at least about 100-fold or more relative to a control.
Embodiment 1025. The method of any of the above enumerated embodiments wherein the HDAC inhibitor decreases expression or enzymatic activity of HDAC by at least about 200-fold or more relative to a control.
Embodiment 1026. The method of any of the above enumerated embodiments wherein the HDAC inhibitor decreases expression or enzymatic activity of HDAC by at least about 500-fold or more relative to a control.
Embodiment 1027. The method of any of the above enumerated embodiments wherein the HDAC inhibitor decreases expression or enzymatic activity of HDAC by at least about 1000-fold or more relative to a control.
Embodiment 1028. The method of any of the above enumerated embodiments wherein the HDAC inhibitor decreases histone deacetylation of a target gene by at least about 1.1-fold or more relative to a control.
Embodiment 1029. The method of any of the above enumerated embodiments wherein the HDAC inhibitor decreases histone deacetylation of a target gene by at least about 1.2-fold or more relative to a control.
Embodiment 1030. The method of any of the above enumerated embodiments wherein the HDAC inhibitor decreases histone deacetylation of a target gene by at least about 1.3-fold or more relative to a control.
Embodiment 1031. The method of any of the above enumerated embodiments wherein the HDAC inhibitor decreases histone deacetylation of a target gene by at least about 1.4-fold or more relative to a control.
Embodiment 1032. The method of any of the above enumerated embodiments wherein the HDAC inhibitor decreases histone deacetylation of a target gene by at least about 1.5-fold or more relative to a control.
Embodiment 1033. The method of any of the above enumerated embodiments wherein the HDAC inhibitor decreases histone deacetylation of a target gene by at least about 1.6-fold or more relative to a control.
Embodiment 1034. The method of any of the above enumerated embodiments wherein the HDAC inhibitor decreases histone deacetylation of a target gene by at least about 1.7-fold or more relative to a control.
Embodiment 1035. The method of any of the above enumerated embodiments wherein the HDAC inhibitor decreases histone deacetylation of a target gene by at least about 1.8-fold or
Embodiment 1036. The method of any of the above enumerated embodiments wherein the HDAC inhibitor decreases histone deacetylation of a target gene by at least about 1.9-fold or more relative to a control.
Embodiment 1037. The method of any of the above enumerated embodiments wherein the HDAC inhibitor decreases histone deacetylation of a target gene by at least about 2-fold or more relative to a control.
Embodiment 1038. The method of any of the above enumerated embodiments wherein the HDAC inhibitor decreases histone deacetylation of a target gene by at least about 3-fold or more relative to a control.
Embodiment 1039. The method of any of the above enumerated embodiments wherein the HDAC inhibitor decreases histone deacetylation of a target gene by at least about 4-fold or more relative to a control.
Embodiment 1040. The method of any of the above enumerated embodiments wherein the HDAC inhibitor decreases histone deacetylation of a target gene by at least about 5-fold or more relative to a control.
Embodiment 1041. The method of any of the above enumerated embodiments wherein the HDAC inhibitor decreases histone deacetylation of a target gene by at least about 6-fold or more relative to a control.
Embodiment 1042. The method of any of the above enumerated embodiments wherein the HDAC inhibitor decreases histone deacetylation of a target gene by at least about 7-fold or more relative to a control.
Embodiment 1043. The method of any of the above enumerated embodiments wherein the HDAC inhibitor decreases histone deacetylation of a target gene by at least about 8-fold or more relative to a control.
Embodiment 1044. The method of any of the above enumerated embodiments wherein the HDAC inhibitor decreases histone deacetylation of a target gene by at least about 9-fold or more relative to a control.
Embodiment 1045. The method of any of the above enumerated embodiments wherein the HDAC inhibitor decreases histone deacetylation of a target gene by at least about 10-fold or
Embodiment 1046. The method of any of the above enumerated embodiments wherein the HDAC inhibitor decreases histone deacetylation of a target gene by at least about 15-fold or more relative to a control.
Embodiment 1047. The method of any of the above enumerated embodiments wherein the HDAC inhibitor decreases histone deacetylation of a target gene by at least about 20-fold or more relative to a control.
Embodiment 1048. The method of any of the above enumerated embodiments wherein the HDAC inhibitor decreases histone deacetylation of a target gene by at least about 30-fold or more relative to a control.
Embodiment 1049. The method of any of the above enumerated embodiments wherein the HDAC inhibitor decreases histone deacetylation of a target gene by at least about 40-fold or more relative to a control.
Embodiment 1050. The method of any of the above enumerated embodiments wherein the HDAC inhibitor decreases histone deacetylation of a target gene by at least about 50-fold or more relative to a control.
Embodiment 1051. The method of any of the above enumerated embodiments wherein the HDAC inhibitor decreases histone deacetylation of a target gene by at least about 60-fold or more relative to a control.
Embodiment 1052. The method of any of the above enumerated embodiments wherein the HDAC inhibitor decreases histone deacetylation of a target gene by at least about 70-fold or more relative to a control.
Embodiment 1053. The method of any of the above enumerated embodiments wherein the HDAC inhibitor decreases histone deacetylation of a target gene by at least about 80-fold or more relative to a control.
Embodiment 1054. The method of any of the above enumerated embodiments wherein the HDAC inhibitor decreases histone deacetylation of a target gene by at least about 90-fold or more relative to a control.
Embodiment 1055. The method of any of the above enumerated embodiments wherein the HDAC inhibitor decreases histone deacetylation of a target gene by at least about 100-fold or
Embodiment 1056. The method of any of the above enumerated embodiments wherein the HDAC inhibitor decreases histone deacetylation of a target gene by at least about 200-fold or more relative to a control.
Embodiment 1057. The method of any of the above enumerated embodiments wherein the HDAC inhibitor decreases histone deacetylation of a target gene by at least about 500-fold or more relative to a control.
Embodiment 1058. The method of any of the above enumerated embodiments wherein the HDAC inhibitor decreases histone deacetylation of a target gene by at least about 1000-fold or more relative to a control.
Embodiment 1059. The method of any of the above enumerated embodiments wherein the HDAC inhibitor increases expression or activity of a target gene by at least about 1.1-fold or more relative to a control.
Embodiment 1060. The method of any of the above enumerated embodiments wherein the HDAC inhibitor increases expression or activity of a target gene by at least about 1.2-fold or more relative to a control.
Embodiment 1061. The method of any of the above enumerated embodiments wherein the HDAC inhibitor increases expression or activity of a target gene by at least about 1.3-fold or more relative to a control.
Embodiment 1062. The method of any of the above enumerated embodiments wherein the HDAC inhibitor increases expression or activity of a target gene by at least about 1.4-fold or more relative to a control.
Embodiment 1063. The method of any of the above enumerated embodiments wherein the HDAC inhibitor increases expression or activity of a target gene by at least about 1.5-fold or more relative to a control.
Embodiment 1064. The method of any of the above enumerated embodiments wherein the HDAC inhibitor increases expression or activity of a target gene by at least about 1.6-fold or more relative to a control.
Embodiment 1065. The method of any of the above enumerated embodiments wherein the HDAC inhibitor increases expression or activity of a target gene by at least about 1.7-fold or
Embodiment 1066. The method of any of the above enumerated embodiments wherein the HDAC inhibitor increases expression or activity of a target gene by at least about 1.8-fold or more relative to a control.
Embodiment 1067. The method of any of the above enumerated embodiments wherein the HDAC inhibitor increases expression or activity of a target gene by at least about 1.9-fold or more relative to a control.
Embodiment 1068. The method of any of the above enumerated embodiments wherein the HDAC inhibitor increases expression or activity of a target gene by at least about 2-fold or more relative to a control.
Embodiment 1069. The method of any of the above enumerated embodiments wherein the HDAC inhibitor increases expression or activity of a target gene by at least about 3-fold or more relative to a control.
Embodiment 1070. The method of any of the above enumerated embodiments wherein the HDAC inhibitor increases expression or activity of a target gene by at least about 4-fold or more relative to a control.
Embodiment 1071. The method of any of the above enumerated embodiments wherein the HDAC inhibitor increases expression or activity of a target gene by at least about 5-fold or more relative to a control.
Embodiment 1072. The method of any of the above enumerated embodiments wherein the HDAC inhibitor increases expression or activity of a target gene by at least about 6-fold or more relative to a control.
Embodiment 1073. The method of any of the above enumerated embodiments wherein the HDAC inhibitor increases expression or activity of a target gene by at least about 7-fold or more relative to a control.
Embodiment 1074. The method of any of the above enumerated embodiments wherein the HDAC inhibitor increases expression or activity of a target gene by at least about 8-fold or more relative to a control.
Embodiment 1075. The method of any of the above enumerated embodiments wherein the HDAC inhibitor increases expression or activity of a target gene by at least about 9-fold or
Embodiment 1076. The method of any of the above enumerated embodiments wherein the HDAC inhibitor increases expression or activity of a target gene by at least about 10-fold or more relative to a control.
Embodiment 1077. The method of any of the above enumerated embodiments wherein the HDAC inhibitor increases expression or activity of a target gene by at least about 15-fold or more relative to a control.
Embodiment 1078. The method of any of the above enumerated embodiments wherein the HDAC inhibitor increases expression or activity of a target gene by at least about 20-fold or more relative to a control.
Embodiment 1079. The method of any of the above enumerated embodiments wherein the HDAC inhibitor increases expression or activity of a target gene by at least about 30-fold or more relative to a control.
Embodiment 1080. The method of any of the above enumerated embodiments wherein the HDAC inhibitor increases expression or activity of a target gene by at least about 40-fold or more relative to a control.
Embodiment 1081. The method of any of the above enumerated embodiments wherein the HDAC inhibitor increases expression or activity of a target gene by at least about 50-fold or more relative to a control.
Embodiment 1082. The method of any of the above enumerated embodiments wherein the HDAC inhibitor increases expression or activity of a target gene by at least about 60-fold or more relative to a control.
Embodiment 1083. The method of any of the above enumerated embodiments wherein the HDAC inhibitor increases expression or activity of a target gene by at least about 70-fold or more relative to a control.
Embodiment 1084. The method of any of the above enumerated embodiments wherein the HDAC inhibitor increases expression or activity of a target gene by at least about 80-fold or more relative to a control.
Embodiment 1085. The method of any of the above enumerated embodiments wherein the HDAC inhibitor increases expression or activity of a target gene by at least about 90-fold or
Embodiment 1086. The method of any of the above enumerated embodiments wherein the HDAC inhibitor increases expression or activity of a target gene by at least about 100-fold or more relative to a control.
Embodiment 1087. The method of any of the above enumerated embodiments wherein the HDAC inhibitor increases expression or activity of a target gene by at least about 200-fold or more relative to a control.
Embodiment 1088. The method of any of the above enumerated embodiments wherein the HDAC inhibitor increases expression or activity of a target gene by at least about 500-fold or more relative to a control.
Embodiment 1089. The method of any of the above enumerated embodiments wherein the HDAC inhibitor increases expression or activity of a target gene by at least about 1000-fold or more relative to a control.
Embodiment 1090. The method of any of the above enumerated embodiments wherein the HDAC inhibitor decreases expression or activity of a target gene by at least about 1.1-fold or more relative to a control.
Embodiment 1091. The method of any of the above enumerated embodiments wherein the HDAC inhibitor decreases expression or activity of a target gene by at least about 1.2-fold or more relative to a control.
Embodiment 1092. The method of any of the above enumerated embodiments wherein the HDAC inhibitor decreases expression or activity of a target gene by at least about 1.3-fold or more relative to a control.
Embodiment 1093. The method of any of the above enumerated embodiments wherein the HDAC inhibitor decreases expression or activity of a target gene by at least about 1.4-fold or more relative to a control.
Embodiment 1094. The method of any of the above enumerated embodiments wherein the HDAC inhibitor decreases expression or activity of a target gene by at least about 1.5-fold or more relative to a control.
Embodiment 1095. The method of any of the above enumerated embodiments wherein the HDAC inhibitor decreases expression or activity of a target gene by at least about 1.6-fold or
Embodiment 1096. The method of any of the above enumerated embodiments wherein the HDAC inhibitor decreases expression or activity of a target gene by at least about 1.7-fold or more relative to a control.
Embodiment 1097. The method of any of the above enumerated embodiments wherein the HDAC inhibitor decreases expression or activity of a target gene by at least about 1.8-fold or more relative to a control.
Embodiment 1098. The method of any of the above enumerated embodiments wherein the HDAC inhibitor decreases expression or activity of a target gene by at least about 1.9-fold or more relative to a control.
Embodiment 1099. The method of any of the above enumerated embodiments wherein the HDAC inhibitor decreases expression or activity of a target gene by at least about 2-fold or more relative to a control.
Embodiment 1100. The method of any of the above enumerated embodiments wherein the HDAC inhibitor decreases expression or activity of a target gene by at least about 3-fold or more relative to a control.
Embodiment 1101. The method of any of the above enumerated embodiments wherein the HDAC inhibitor decreases expression or activity of a target gene by at least about 4-fold or more relative to a control.
Embodiment 1102. The method of any of the above enumerated embodiments wherein the HDAC inhibitor decreases expression or activity of a target gene by at least about 5-fold or more relative to a control.
Embodiment 1103. The method of any of the above enumerated embodiments wherein the HDAC inhibitor decreases expression or activity of a target gene by at least about 6-fold or more relative to a control.
Embodiment 1104. The method of any of the above enumerated embodiments wherein the HDAC inhibitor decreases expression or activity of a target gene by at least about 7-fold or more relative to a control.
Embodiment 1105. The method of any of the above enumerated embodiments wherein the HDAC inhibitor decreases expression or activity of a target gene by at least about 8-fold or
Embodiment 1106. The method of any of the above enumerated embodiments wherein the HDAC inhibitor decreases expression or activity of a target gene by at least about 9-fold or more relative to a control.
Embodiment 1107. The method of any of the above enumerated embodiments wherein the HDAC inhibitor decreases expression or activity of a target gene by at least about 10-fold or more relative to a control.
Embodiment 1108. The method of any of the above enumerated embodiments wherein the HDAC inhibitor decreases expression or activity of a target gene by at least about 15-fold or more relative to a control.
Embodiment 1109. The method of any of the above enumerated embodiments wherein the HDAC inhibitor decreases expression or activity of a target gene by at least about 20-fold or more relative to a control.
Embodiment 1110. The method of any of the above enumerated embodiments wherein the HDAC inhibitor decreases expression or activity of a target gene by at least about 30-fold or more relative to a control.
Embodiment 1111. The method of any of the above enumerated embodiments wherein the HDAC inhibitor decreases expression or activity of a target gene by at least about 40-fold or more relative to a control.
Embodiment 1112. The method of any of the above enumerated embodiments wherein the HDAC inhibitor decreases expression or activity of a target gene by at least about 50-fold or more relative to a control.
Embodiment 1113. The method of any of the above enumerated embodiments wherein the HDAC inhibitor decreases expression or activity of a target gene by at least about 60-fold or more relative to a control.
Embodiment 1114. The method of any of the above enumerated embodiments wherein the HDAC inhibitor decreases expression or activity of a target gene by at least about 70-fold or more relative to a control.
Embodiment 1115. The method of any of the above enumerated embodiments wherein the HDAC inhibitor decreases expression or activity of a target gene by at least about 80-fold or
Embodiment 1116. The method of any of the above enumerated embodiments wherein the HDAC inhibitor decreases expression or activity of a target gene by at least about 90-fold or more relative to a control.
Embodiment 1117. The method of any of the above enumerated embodiments wherein the HDAC inhibitor decreases expression or activity of a target gene by at least about 100-fold or more relative to a control.
Embodiment 1118. The method of any of the above enumerated embodiments wherein the HDAC inhibitor decreases expression or activity of a target gene by at least about 200-fold or more relative to a control.
Embodiment 1119. The method of any of the above enumerated embodiments wherein the HDAC inhibitor decreases expression or activity of a target gene by at least about 500-fold or more relative to a control.
Embodiment 1120. The method of any of the above enumerated embodiments wherein the HDAC inhibitor decreases expression or activity of a target gene by at least about 1000-fold or more relative to a control.
Embodiment 1121. The method of any of the above enumerated embodiments wherein the HDAC inhibitor is an HDAC inhibitor listed in Table 5.
Embodiment 1122. The method of any of the above enumerated embodiments wherein the HDAC inhibitor is an HDAC inhibitor listed in Table 6.
Embodiment 1123. The method of any of the above enumerated embodiments wherein the HDAC inhibitor is a class I HDAC inhibitor.
Embodiment 1124. The method of any of the above enumerated embodiments wherein the class I HDAC inhibitor is a short chain carboxylic acid.
Embodiment 1125. The method of any of the above enumerated embodiments wherein the class I HDAC inhibitor is valproic acid (VPA).
Embodiment 1126. The method of any of the above enumerated embodiments wherein the method induces growth of inner ear tissue, particularly inner ear supporting cells and hair cells.
Embodiment 1127. The method of any of the above enumerated embodiments wherein the method promotes growth of inner ear tissue, particularly inner ear supporting cells and hair cells.
Embodiment 1128. The method of any of the above enumerated embodiments wherein the method enhances growth of inner ear tissue, particularly inner ear supporting cells and hair cells.
Embodiment 1129. The method of any of the above enumerated embodiments wherein the method induces proliferation of inner ear tissue, particularly inner ear supporting cells and hair cells.
Embodiment 1130. The method of any of the above enumerated embodiments wherein the method promotes proliferation of inner ear tissue, particularly inner ear supporting cells and hair cells.
Embodiment 1131. The method of any of the above enumerated embodiments wherein the method enhances proliferation of inner ear tissue, particularly inner ear supporting cells and hair cells.
Embodiment 1132. The method of any of the above enumerated embodiments wherein the method induces regeneration of inner ear tissue, particularly inner ear supporting cells and hair cells.
Embodiment 1133. The method of any of the above enumerated embodiments wherein the method promotes regeneration of inner ear tissue, particularly inner ear supporting cells and hair cells.
Embodiment 1134. The method of any of the above enumerated embodiments wherein the method enhances regeneration of inner ear tissue, particularly inner ear supporting cells and hair cells.
Embodiment 1135. The method of any of the above enumerated embodiments wherein the method controls proliferation of stem cells comprising an initial phase of inducing stemness while inhibiting differentiation and a subsequent phase of differentiation of the stem cells into tissue cells.
Embodiment 1136. The method of any of the above enumerated embodiments wherein the method induces the supporting cells to produce daughter stem cells that can divide for many generations and maintain the ability to have a high proportion of the resulting cells differentiate into hair cells.
Embodiment 1137. The method of any of the above enumerated embodiments wherein the treated supporting cells exhibit stem-like behavior in that the treated supporting cells have the capacity to proliferate and differentiate into cochlear or vestibular hair cells.
Embodiment 1138. The method of any of the above enumerated embodiments wherein the proliferating stem cells express the stem cell marker Lgr5.
Embodiment 1139. The method of any of the above enumerated embodiments wherein the proliferating stem cells express the stem cell marker Sox2.
Embodiment 1140. The method of any of the above enumerated embodiments wherein the proliferating stem cells express the stem cell marker Opem1.
Embodiment 1141. The method of any of the above enumerated embodiments wherein the proliferating stem cells express the stem cell marker Phex.
Embodiment 1142. The method of any of the above enumerated embodiments wherein the proliferating stem cells express the stem cell marker lin28.
Embodiment 1143. The method of any of the above enumerated embodiments wherein the proliferating stem cells express the stem cell marker Lgr6.
Embodiment 1144. The method of any of the above enumerated embodiments wherein the proliferating stem cells express the stem cell marker cyclin D1.
Embodiment 1145. The method of any of the above enumerated embodiments wherein the proliferating stem cells express the stem cell marker Myb.
Embodiment 1146. The method of any of the above enumerated embodiments wherein the proliferating stem cells express the stem cell marker Kit.
Embodiment 1147. The method of any of the above enumerated embodiments wherein the proliferating stem cells express the stem cell marker Gdnf3.
Embodiment 1148. The method of any of the above enumerated embodiments wherein the proliferating stem cells express the stem cell marker Zic3.
Embodiment 1149. The method of any of the above enumerated embodiments wherein the proliferating stem cells express the stem cell marker Dppa3.
Embodiment 1150. The method of any of the above enumerated embodiments wherein the proliferating stem cells express the stem cell marker Dppa4.
Embodiment 1151. The method of any of the above enumerated embodiments wherein the proliferating stem cells express the stem cell marker Dppa5.
Embodiment 1152. The method of any of the above enumerated embodiments wherein the proliferating stem cells express the stem cell marker Nanog.
Embodiment 1153. The method of any of the above enumerated embodiments wherein the proliferating stem cells express the stem cell marker Esrrb.
Embodiment 1154. The method of any of the above enumerated embodiments wherein the proliferating stem cells express the stem cell marker Rex1.
Embodiment 1155. The method of any of the above enumerated embodiments wherein the proliferating stem cells express the stem cell marker Dnmt3a.
Embodiment 1156. The method of any of the above enumerated embodiments wherein the proliferating stem cells express the stem cell marker Dnmt3b.
Embodiment 1157. The method of any of the above enumerated embodiments wherein the proliferating stem cells express the stem cell marker Dnmt3l.
Embodiment 1158. The method of any of the above enumerated embodiments wherein the proliferating stem cells express the stem cell marker Utf1.
Embodiment 1159. The method of any of the above enumerated embodiments wherein the proliferating stem cells express the stem cell marker Tcl1.
Embodiment 1160. The method of any of the above enumerated embodiments wherein the proliferating stem cells express the stem cell marker Oct4.
Embodiment 1161. The method of any of the above enumerated embodiments wherein the proliferating stem cells express the stem cell marker Klf4.
Embodiment 1162. The method of any of the above enumerated embodiments wherein the proliferating stem cells express the stem cell marker Pax6.
Embodiment 1163. The method of any of the above enumerated embodiments wherein the proliferating stem cells express the stem cell marker Six2.
Embodiment 1164. The method of any of the above enumerated embodiments wherein the proliferating stem cells express the stem cell marker Zic1.
Embodiment 1165. The method of any of the above enumerated embodiments wherein the proliferating stem cells express the stem cell marker Zic2.
Embodiment 1166. The method of any of the above enumerated embodiments wherein the proliferating stem cells express the stem cell marker Otx2.
Embodiment 1167. The method of any of the above enumerated embodiments wherein the proliferating stem cells express the stem cell marker Bmi1.
Embodiment 1168. The method of any of the above enumerated embodiments wherein the proliferating stem cells express the stem cell marker CDX2.
Embodiment 1169. The method of any of the above enumerated embodiments wherein the proliferating stem cells express the stem cell marker STAT3.
Embodiment 1170. The method of any of the above enumerated embodiments wherein the proliferating stem cells express the stem cell marker Smad1.
Embodiment 1171. The method of any of the above enumerated embodiments wherein the proliferating stem cells express the stem cell marker Smad2.
Embodiment 1172. The method of any of the above enumerated embodiments wherein the proliferating stem cells express the stem cell marker Smad2/3.
Embodiment 1173. The method of any of the above enumerated embodiments wherein the proliferating stem cells express the stem cell marker Smad4.
Embodiment 1174. The method of any of the above enumerated embodiments wherein the proliferating stem cells express the stem cell marker Smad5.
Embodiment 1175. The method of any of the above enumerated embodiments wherein the proliferating stem cells express the stem cell marker Smad7.
Embodiment 1176. The method of any of the above enumerated embodiments wherein the method is used to maintain, or even transiently increase stemnes of a pre-existing supporting cell population prior to significant hair cell formation.
Embodiment 1177. The method of any of the above enumerated embodiments wherein the pre-existing supporting cell population comprises inner pillar cells.
Embodiment 1178. The method of any of the above enumerated embodiments wherein the pre-existing supporting cell population comprises outer pillar cells.
Embodiment 1179. The method of any of the above enumerated embodiments wherein the pre-existing supporting cell population comprises inner phalangeal cells.
Embodiment 1180. The method of any of the above enumerated embodiments wherein the pre-existing supporting cell population comprises Deiter cells.
Embodiment 1181. The method of any of the above enumerated embodiments wherein the pre-existing supporting cell population comprises Hensen cells.
Embodiment 1182. The method of any of the above enumerated embodiments wherein the pre-existing supporting cell population comprises Boettcher cells.
Embodiment 1183. The method of any of the above enumerated embodiments wherein the pre-existing supporting cell population comprises Claudius cells.
Embodiment 1184. The method of any of the above enumerated embodiments wherein the pre-existing supporting cell population comprises Lgr5+ cells.
Embodiment 1185. The method of any of the above enumerated embodiments wherein the expansion of the pre-existing supporting cell population is confirmed by morphological analyses with immunostaining.
Embodiment 1186. The method of any of the above enumerated embodiments wherein the expansion of the pre-existing supporting cell population is confirmed by lineage tracing across a Representative Microscopy Samples.
Embodiment 1187. The method of any of the above enumerated embodiments wherein the upregulation of Lgr5 amongst the pre-existing supporting cell population is confirmed by morphological analyses with immunostaining.
Embodiment 1188. The method of any of the above enumerated embodiments wherein the upregulation of Lgr5 amongst the pre-existing supporting cell population is confirmed by morphological analyses with qPCR hybridization.
Embodiment 1189. The method of any of the above enumerated embodiments wherein the upregulation of Lgr5 amongst the pre-existing supporting cell population is confirmed by morphological analyses with RNA hybridization.
Embodiment 1190. The method of any of the above enumerated embodiments wherein the therapy involves the administration of a small molecule.
Embodiment 1191. The method of any of the above enumerated embodiments wherein the therapy involves the administration of a peptide.
Embodiment 1192. The method of any of the above enumerated embodiments wherein the therapy involves the administration of an antibody.
Embodiment 1193. The method of any of the above enumerated embodiments wherein the therapy involves the administration of a nucleic acid delivery vector unaccompanied by gene therapy.
Embodiment 1194. The method of any of the above enumerated embodiments wherein the therapy involves the administration of a small organic molecule.
Embodiment 1195. The method of any of the above enumerated embodiments wherein hearing protection or restoration is achieved through the use of a non-genetic therapeutic composition that is injected in the middle ear and diffuses into the cochlea.
Embodiment 1196. The method of any of the above enumerated embodiments wherein the cell density of hair cells in a cochlear cell population is expanded in a manner that maintains the rosette pattern characteristic of cochlear epithelia or vestibular epithelia.
Embodiment 1197. The method of any of the above enumerated embodiments wherein the cell density of hair cells in a cochlear cell population is expanded in a manner that establishes the rosette pattern characteristic of cochlear epithelia or vestibular epithelia.
Embodiment 1198. The method of any of the above enumerated embodiments wherein the cell density of hair cells is increased in a population of cochlear cells comprising both hair cells and supporting cells.
Embodiment 1199. The method of any of the above enumerated embodiments wherein the cell density of hair cells is increased in a population of vestibular cells comprising both hair cells and supporting cells.
Embodiment 1200. The method of any of the above enumerated embodiments wherein the cochlear cell population is an in vimn population.
Embodiment 1201. The method of any of the above enumerated embodiments wherein the cochlear cell population is an in vitro population.
Embodiment 1202. The method of any of the above enumerated embodiments wherein the increase in cell density is determined by reference to a Representative Microscopy Sample of the population taken prior and subsequent to any treatment.
Embodiment 1203. The method of any of the above enumerated embodiments wherein the increase in cell density is determined indirectly by determining an effect upon the hearing of the subject with an increase in hair cell density correlating to an improvement in hearing.
Embodiment 1204. The method of any of the above enumerated embodiments wherein the supporting cells placed in a Stem Cell Proliferation Assay in the absence of neuronal cells form ribbon synapses.
Embodiment 1205. The method of any of the above enumerated embodiments wherein the proliferation of supporting cells in a cochlear cell population is expanded in a manner that the basilar membrane of the cochlear epithelia is maintained.
Embodiment 1206. The method of any of the above enumerated embodiments wherein the number of supporting cells in an initial cochlear cell population is selectively expanded by treating the initial cochlear cell population with a composition of the present disclosure to form an intermediate cochlear cell population.
Embodiment 1207. The method of any of the above enumerated embodiments wherein the ratio of supporting cells to hair cells in the intermediate cochlear cell population exceeds the ratio of supporting cells to hair cells in the initial cochlear cell population.
Embodiment 1208. The method of any of the above enumerated embodiments wherein the ratio of supporting cells to hair cells in the intermediate cochlear cell population exceeds the ratio of supporting cells to hair cells in the initial cochlear cell population by a factor of 1.1 or more.
Embodiment 1209. The method of any of the above enumerated embodiments wherein the ratio of supporting cells to hair cells in the intermediate cochlear cell population exceeds the ratio of supporting cells to hair cells in the initial cochlear cell population by a factor of 1.5 or more.
Embodiment 1210. The method of any of the above enumerated embodiments wherein the ratio of supporting cells to hair cells in the intermediate cochlear cell population exceeds the ratio of supporting cells to hair cells in the initial cochlear cell population by a factor of 2 or more.
Embodiment 1211. The method of any of the above enumerated embodiments wherein the ratio of supporting cells to hair cells in the intermediate cochlear cell population exceeds the ratio of supporting cells to hair cells in the initial cochlear cell population by a factor of 3 or more.
Embodiment 1212. The method of any of the above enumerated embodiments wherein the ratio of supporting cells to hair cells in the intermediate cochlear cell population exceeds the ratio of supporting cells to hair cells in the initial cochlear cell population by a factor of 4 or more.
Embodiment 1213. The method of any of the above enumerated embodiments wherein the ratio of supporting cells to hair cells in the intermediate cochlear cell population exceeds the ratio of supporting cells to hair cells in the initial cochlear cell population by a factor of 5 or more.
Embodiment 1214. The method of any of the above enumerated embodiments wherein the capacity of a composition to expand a cochlear cell population is be determined by means of a Stem Cell Proliferation Assay.
Embodiment 1215. The method of any of the above enumerated embodiments wherein the number of stem cells in a cochlear cell population is expanded to form an intermediate cochlear cell population by treating a cochlear cell population with a composition of the present disclosure
Embodiment 1216. The method of any of the above enumerated embodiments wherein the cell density of stem cells in the intermediate cochlear cell population exceeds the cell density of stem cells in the initial cochlear cell population.
Embodiment 1217. The method of any of the above enumerated embodiments wherein the expanded cochlear cell population is an in vivo population.
Embodiment 1218. The method of any of the above enumerated embodiments wherein the expanded cochlear cell population is an in vitro population.
Embodiment 1219. The method of any of the above enumerated embodiments wherein the cell density of stem cells in the treated cochlear cell population exceeds the cell density of stem cells in the initial cochlear cell population by a factor of at least 1.1 or more.
Embodiment 1220. The method of any of the above enumerated embodiments wherein the cell density of stem cells in the treated cochlear cell population exceeds the cell density of stem cells in the initial cochlear cell population by a factor of at least 1.25 or more.
Embodiment 1221. The method of any of the above enumerated embodiments wherein the cell density of stem cells in the treated cochlear cell population exceeds the cell density of stem cells in the initial cochlear cell population by a factor of at least 1.5 or more.
Embodiment 1222. The method of any of the above enumerated embodiments wherein the cell density of stem cells in the treated cochlear cell population exceeds the cell density of stem cells in the initial cochlear cell population by a factor of at least 2 or more.
Embodiment 1223. The method of any of the above enumerated embodiments wherein the cell density of stem cells in the treated cochlear cell population exceeds the cell density of stem cells in the initial cochlear cell population by a factor of at least 3 or more.
Embodiment 1224. The method of any of the above enumerated embodiments wherein the cell density of stem cells in the treated cochlear cell population exceeds the cell density of stem cells in the initial cochlear cell population by a factor of at least 4 or more.
Embodiment 1225. The method of any of the above enumerated embodiments wherein the cell density of stem cells in the treated cochlear cell population exceeds the cell density of stem cells in the initial cochlear cell population by a factor of at least 5 or more.
Embodiment 1226. The method of any of the above enumerated embodiments wherein the cell density of stem cells in an expanded in vitro population of stem cells is at least 4 times greater than the cell density of the stem cells in the initial cochlear cell population.
Embodiment 1227. The method of any of the above enumerated embodiments wherein the cell density of stem cells in an expanded in vitro population of stem cells is at least 5 times greater than the cell density of the stem cells in the initial cochlear cell population.
Embodiment 1228. The method of any of the above enumerated embodiments wherein the cell density of stem cells in an expanded in vitro population of stem cells is at least 6 times greater than the cell density of the stem cells in the initial cochlear cell population.
Embodiment 1229. The method of any of the above enumerated embodiments wherein the cell density of stem cells in an expanded in vitro population of stem cells is at least 7 times greater than the cell density of the stem cells in the initial cochlear cell population.
Embodiment 1230. The method of any of the above enumerated embodiments wherein the cell density of stem cells in an expanded in vitro population of stem cells is at least 8 times greater than the cell density of the stem cells in the initial cochlear cell population.
Embodiment 1231. The method of any of the above enumerated embodiments wherein the cell density of stem cells in an expanded in vitro population of stem cells is at least 9 times greater than the cell density of the stem cells in the initial cochlear cell population.
Embodiment 1232. The method of any of the above enumerated embodiments wherein the cell density of stem cells in an expanded in vitro population of stem cells is at least 10 times greater than the cell density of the stem cells in the initial cochlear cell population.
Embodiment 1233. The method of any of the above enumerated embodiments wherein the cell density of stem cells in an expanded in vitro population of stem cells is at least 15 times greater than the cell density of the stem cells in the initial cochlear cell population.
Embodiment 1234. The method of any of the above enumerated embodiments wherein the cell density of stem cells in an expanded in vitro population of stem cells is at least 20 times greater than the cell density of the stem cells in the initial cochlear cell population.
Embodiment 1235. The method of any of the above enumerated embodiments wherein the cell density of stem cells in an expanded in vitro population of stem cells is at least 25 times greater than the cell density of the stem cells in the initial cochlear cell population.
Embodiment 1236. The method of any of the above enumerated embodiments wherein the cell density of stem cells in an expanded in vitro population of stem cells is at least 30 times greater than the cell density of the stem cells in the initial cochlear cell population.
Embodiment 1237. The method of any of the above enumerated embodiments wherein the cell density of stem cells in an expanded in vitro population of stem cells is at least 35 times greater than the cell density of the stem cells in the initial cochlear cell population.
Embodiment 1238. The method of any of the above enumerated embodiments wherein the cell density of stem cells in an expanded in vitro population of stem cells is at least 40 times greater than the cell density of the stem cells in the initial cochlear cell population.
Embodiment 1239. The method of any of the above enumerated embodiments wherein the cell density of stem cells in an expanded in vitro population of stem cells is at least 45 times greater than the cell density of the stem cells in the initial cochlear cell population.
Embodiment 1240. The method of any of the above enumerated embodiments wherein the cell density of stem cells in an expanded in vitro population of stem cells is at least 50 times greater than the cell density of the stem cells in the initial cochlear cell population.
Embodiment 1241. The method of any of the above enumerated embodiments wherein the cell density of stem cells in an expanded in vitro population of stem cells is at least 75 times greater than the cell density of the stem cells in the initial cochlear cell population.
Embodiment 1242. The method of any of the above enumerated embodiments wherein the cell density of stem cells in an expanded in vitro population of stem cells is at least 100 times greater than the cell density of the stem cells in the initial cochlear cell population.
Embodiment 1243. The method of any of the above enumerated embodiments wherein the cell density of stem cells in an expanded in vitro population of stem cells is at least 200 times greater than the cell density of the stem cells in the initial cochlear cell population.
Embodiment 1244. The method of any of the above enumerated embodiments wherein the cell density of stem cells in an expanded in vitro population of stem cells is at least 200 times greater than the cell density of the stem cells in the initial cochlear cell population.
Embodiment 1245. The method of any of the above enumerated embodiments wherein the cell density of stem cells in an expanded in vitro population of stem cells is at least 300 times greater than the cell density of the stem cells in the initial cochlear cell population.
Embodiment 1246. The method of any of the above enumerated embodiments wherein the cell density of stem cells in an expanded in vitro population of stem cells is at least 400 times greater than the cell density of the stem cells in the initial cochlear cell population.
Embodiment 1247. The method of any of the above enumerated embodiments wherein the cell density of stem cells in an expanded in vitro population of stem cells is at least 500 times greater than the cell density of the stem cells in the initial cochlear cell population.
Embodiment 1248. The method of any of the above enumerated embodiments wherein the cell density of stem cells in an expanded in vitro population of stem cells is at least 600 times greater than the cell density of the stem cells in the initial cochlear cell population.
Embodiment 1249. The method of any of the above enumerated embodiments wherein the cell density of stem cells in an expanded in vitro population of stem cells is at least 700 times greater than the cell density of the stem cells in the initial cochlear cell population.
Embodiment 1250. The method of any of the above enumerated embodiments wherein the cell density of stem cells in an expanded in vitro population of stem cells is at least 800 times greater than the cell density of the stem cells in the initial cochlear cell population.
Embodiment 1251. The method of any of the above enumerated embodiments wherein the cell density of stem cells in an expanded in vitro population of stem cells is at least 900 times greater than the cell density of the stem cells in the initial cochlear cell population.
Embodiment 1252. The method of any of the above enumerated embodiments wherein the cell density of stem cells in an expanded in vitro population of stem cells is at least 1000 times greater than the cell density of the stem cells in the initial cochlear cell population.
Embodiment 1253. The method of any of the above enumerated embodiments wherein the cochlea supporting cell population is treated with a composition of the present disclosure to increase the Lgr5 activity of the population.
Embodiment 1254. The method of any of the above enumerated embodiments wherein the TAZ activator and the Wnt agonist have the capacity to increase and maintain the Lgr5 activity of an in vitro population of cochlea supporting cells by factor of at least 1.2 or more.
Embodiment 1255. The method of any of the above enumerated embodiments wherein the TAZ activator and the Wnt agonist have the capacity to increase and maintain the Lgr5 activity of an in vitro population of cochlea supporting cells by factor of at least 1.5 or more.
Embodiment 1256. The method of any of the above enumerated embodiments wherein the TAZ activator and the Wnt agonist have the capacity to increase and maintain the Lgr5 activity of an in vitro population of cochlea supporting cells by factor of at least 2 or more.
Embodiment 1257. The method of any of the above enumerated embodiments wherein the TAZ activator and the Wnt agonist have the capacity to increase and maintain the Lgr5 activity of an in vitro population of cochlea supporting cells by factor of at least 3 or more.
Embodiment 1258. The method of any of the above enumerated embodiments wherein the TAZ activator and the Wnt agonist have the capacity to increase and maintain the Lgr5 activity of an in vitro population of cochlea supporting cells by factor of at least 4 or more.
Embodiment 1259. The method of any of the above enumerated embodiments wherein the TAZ activator and the Wnt agonist have the capacity to increase and maintain the Lgr5 activity of an in vitro population of cochlea supporting cells by factor of at least 5 or more.
Embodiment 1260. The method of any of the above enumerated embodiments wherein the TAZ activator and the Wnt agonist have the capacity to increase and maintain the Lgr5 activity of an in vitro population of cochlea supporting cells by factor of at least 10 or more.
Embodiment 1261. The method of any of the above enumerated embodiments wherein the TAZ activator and the Wnt agonist have the capacity to increase and maintain the Lgr5 activity of an in vitro population of cochlea supporting cells by factor of at least 100 or more.
Embodiment 1262. The method of any of the above enumerated embodiments wherein the TAZ activator and the Wnt agonist have the capacity to increase and maintain the Lgr5 activity of an in vitro population of cochlea supporting cells by factor of at least 500 or more.
Embodiment 1263. The method of any of the above enumerated embodiments wherein the TAZ activator and the Wnt agonist have the capacity to increase and maintain the Lgr5 activity of an in vitro population of cochlea supporting cells by factor of at least 1000 or more.
Embodiment 1264. The method of any of the above enumerated embodiments wherein the TAZ activator and the Wnt agonist have the capacity to increase and maintain the Lgr5 activity of an in vitro population of cochlea supporting cells by factor of at least 2000 or more.
Embodiment 1265. The method of any of the above enumerated embodiments wherein the TAZ activator and the Wnt agonist have the capacity to increase and maintain the Lgr5 activity of an in vitro population of cochlea supporting cells by factor of at least 3000 or more.
Embodiment 1266. The method of any of the above enumerated embodiments wherein the TAZ activator and the Wnt agonist inhibitor have the capacity to increase the Lgr5 activity of an in vivo population of cochlea supporting cells by about or at least about 5% or more.
Embodiment 1267. The method of any of the above enumerated embodiments wherein the TAZ activator and the Wnt agonist inhibitor have the capacity to increase the Lgr5 activity of an in vivo population of cochlea supporting cells by about or at least about 10% or more.
Embodiment 1268. The method of any of the above enumerated embodiments wherein the TAZ activator and the Wnt agonist inhibitor have the capacity to increase the Lgr5 activity of an in vivo population of cochlea supporting cells by about or at least about 20% or more.
Embodiment 1269. The method of any of the above enumerated embodiments wherein the TAZ activator and the Wnt agonist inhibitor have the capacity to increase the Lgr5 activity of an in vivo population of cochlea supporting cells by about or at least about 30% or more.
Embodiment 1270. The method of any of the above enumerated embodiments wherein the capacity of the TAZ activator and the Wnt agonist to increase Lgr5 activity is demonstrated in an In vitro Lgr5+ Activity Assay as measured by isolating the organ and performing morphological analyses using immunostaining, endogenous fluorescent protein expression of Lgr5, and qPCR for Lgr5.
Embodiment 1271. The method of any of the above enumerated embodiments wherein the capacity of the TAZ activator and the Wnt agonist to increase Lgr5 activity is demonstrated in an In vivo Lgr5+ Activity Assay as measured by isolating the organ and performing morphological analyses using immunostaining, endogenous fluorescent protein expression of Lgr5, and qPCR for Lgr5.
Embodiment 1272. The method of any of the above enumerated embodiments wherein the TAZ activator and the Wnt agonist has the capacity to increase Lgr5 Activity of an in vitro population of cochlea supporting cells by a factor of 10% compared to a Wnt agonist alone as measured in an In vitro Lgr5+ Activity Assay.
Embodiment 1273. The method of any of the above enumerated embodiments wherein the TAZ activator and the Wnt agonist has the capacity to increase Lgr5 Activity of an in vitro population of cochlea supporting cells by a factor of 20% compared to a Wnt agonist alone as measured in an In vitro Lgr5+ Activity Assay.
Embodiment 1274. The method of any of the above enumerated embodiments wherein the TAZ activator and the Wnt agonist has the capacity to increase Lgr5 Activity of an in vitro population of cochlea supporting cells by a factor of 30% compared to a Wnt agonist alone as measured in an In vitro Lgr5+ Activity Assay.
Embodiment 1275. The method of any of the above enumerated embodiments wherein the TAZ activator and the Wnt agonist has the capacity to increase Lgr5 Activity of an in vitro population of cochlea supporting cells by a factor of 40% compared to a Wnt agonist alone as measured in an In vitro Lgr5+ Activity Assay.
Embodiment 1276. The method of any of the above enumerated embodiments wherein the TAZ activator and the Wnt agonist has the capacity to increase Lgr5 Activity of an in vitro population of cochlea supporting cells by a factor of 50% compared to a Wnt agonist alone as measured in an In vitro Lgr5+ Activity Assay.
Embodiment 1277. The method of any of the above enumerated embodiments wherein the TAZ activator and the Wnt agonist has the capacity to increase Lgr5 Activity of an in vitro population of cochlea supporting cells by a factor of 75% compared to a Wnt agonist alone as measured in an In vitro Lgr5+ Activity Assay.
Embodiment 1278. The method of any of the above enumerated embodiments wherein the TAZ activator and the Wnt agonist has the capacity to increase Lgr5 Activity of an in vitro population of cochlea supporting cells by a factor of 100% compared to a Wnt agonist alone as measured in an In vitro Lgr5+ Activity Assay.
Embodiment 1279. The method of any of the above enumerated embodiments wherein the TAZ activator and the Wnt agonist has the capacity to increase Lgr5 Activity of an in vitro population of cochlea supporting cells by a factor of 2⁰⁰% compared to a Wnt agonist alone as measured in an In vitro Lgr5+ Activity Assay.
Embodiment 1280. The method of any of the above enumerated embodiments wherein the TAZ activator and the Wnt agonist has the capacity to increase the Lgr5 proliferation of an in vitro population of cochlea supporting cells by a factor of 10% compared to a Wnt agonist alone as measured in a Stem Cell Proliferation Assay.
Embodiment 1281. The method of any of the above enumerated embodiments wherein the TAZ activator and the Wnt agonist has the capacity to increase the Lgr5 proliferation of an in vitro population of cochlea supporting cells by a factor of 20% compared to a Wnt agonist alone as measured in a Stem Cell Proliferation Assay.
Embodiment 1282. The method of any of the above enumerated embodiments wherein the TAZ activator and the Wnt agonist has the capacity to increase the Lgr5 proliferation of an in vitro population of cochlea supporting cells by a factor of 30% compared to a Wnt agonist alone as measured in a Stem Cell Proliferation Assay.
Embodiment 1283. The method of any of the above enumerated embodiments wherein the TAZ activator and the Wnt agonist has the capacity to increase the Lgr5 proliferation of an in vitro population of cochlea supporting cells by a factor of 40% compared to a Wnt agonist alone as measured in a Stem Cell Proliferation Assay.
Embodiment 1284. The method of any of the above enumerated embodiments wherein the TAZ activator and the Wnt agonist has the capacity to increase the Lgr5 proliferation of an in vitro population of cochlea supporting cells by a factor of 50% compared to a Wnt agonist alone as measured in a Stem Cell Proliferation Assay.
Embodiment 1285. The method of any of the above enumerated embodiments wherein the TAZ activator and the Wnt agonist has the capacity to increase the Lgr5 proliferation of an in vitro population of cochlea supporting cells by a factor of 75% compared to a Wnt agonist alone as measured in a Stem Cell Proliferation Assay.
Embodiment 1286. The method of any of the above enumerated embodiments wherein the TAZ activator and the Wnt agonist has the capacity to increase the Lgr5 proliferation of an in vitro population of cochlea supporting cells by a factor of 100% compared to a Wnt agonist alone as measured in a Stem Cell Proliferation Assay.
Embodiment 1287. The method of any of the above enumerated embodiments wherein the TAZ activator and the Wnt agonist has the capacity to increase the Lgr5 proliferation of an in vitro population of cochlea supporting cells by a factor of 200% compared to a Wnt agonist alone as measured in a Stem Cell Proliferation Assay.
Embodiment 1288. The method of any of the above enumerated embodiments wherein the TAZ activator and the Wnt agonist has the capacity to increase the Lgr5 proliferation of an in vitro population of cochlea supporting cells by a factor of 10% compared to a Wnt agonist in combination with VPA, as measured in an In vitro Lgr5+ Activity Assay.
Embodiment 1289. The method of any of the above enumerated embodiments wherein the TAZ activator and the Wnt agonist has the capacity to increase the Lgr5 proliferation of an in vitro population of cochlea supporting cells by a factor of 20% compared to a Wnt agonist in combination with VPA, as measured in an In vitro Lgr5+ Activity Assay.
Embodiment 1290. The method of any of the above enumerated embodiments wherein the TAZ activator and the Wnt agonist has the capacity to increase the Lgr5 proliferation of an in vitro population of cochlea supporting cells by a factor of 30% compared to a Wnt agonist in combination with VPA, as measured in an In vitro Lgr5+ Activity Assay.
Embodiment 1291. The method of any of the above enumerated embodiments wherein the TAZ activator and the Wnt agonist has the capacity to increase the Lgr5 proliferation of an in vitro population of cochlea supporting cells by a factor of 40% compared to a Wnt agonist in combination with VPA, as measured in an In vitro Lgr5+ Activity Assay.
Embodiment 1292. The method of any of the above enumerated embodiments wherein the TAZ activator and the Wnt agonist has the capacity to increase the Lgr5 proliferation of an in vitro population of cochlea supporting cells by a factor of 50% compared to a Wnt agonist in combination with VPA, as measured in an In vitro Lgr5+ Activity Assay.
Embodiment 1293. The method of any of the above enumerated embodiments wherein the TAZ activator and the Wnt agonist has the capacity to increase the Lgr5 proliferation of an in vitro population of cochlea supporting cells by a factor of 75% compared to a Wnt agonist in combination with VPA, as measured in an In vitro Lgr5+ Activity Assay.
Embodiment 1294. The method of any of the above enumerated embodiments wherein the TAZ activator and the Wnt agonist has the capacity to increase the Lgr5 proliferation of an in vitro population of cochlea supporting cells by a factor of 100% compared to a Wnt agonist in combination with VPA, as measured in an In vitro Lgr5+ Activity Assay.
Embodiment 1295. The method of any of the above enumerated embodiments wherein the TAZ activator and the Wnt agonist has the capacity to increase the Lgr5 proliferation of an in vitro population of cochlea supporting cells by a factor of 2⁰⁰% compared to a Wnt agonist in combination with VPA, as measured in an In vitro Lgr5+ Activity Assay.
Embodiment 1296. The method of any of the above enumerated embodiments wherein no daughter Lgr5+ cells are generated by cell division, but pre-existing Lgr5+ supporting cells are induced to differentiate into hair cells.
Embodiment 1297. The method of any of the above enumerated embodiments wherein no daughter cells are generated by cell division, but Lgr5-supporting cells are activated to a greater level of Lgr5 activity and the activated supporting cells are then able to differentiate into hair cells.
Embodiment 1298. The method of any of the above enumerated embodiments wherein newly generated Lgr5+ supporting cells have increased stem cell propensity.
Embodiment 1299. The method of any of the above enumerated embodiments wherein a composition of the present disclosure has the capacity to increase the cell density of Lgr5+ supporting cells in an in vitro isolated cell population of cochlea supporting cells by factor of at least 5.
Embodiment 1300. The method of any of the above enumerated embodiments wherein a composition of the present disclosure has the capacity to increase the cell density of Lgr5+ supporting cells in an in vitro isolated cell population of cochlea supporting cells by factor of at least 10.
Embodiment 1301. The method of any of the above enumerated embodiments wherein a composition of the present disclosure has the capacity to increase the cell density of Lgr5+ supporting cells in an in vitro isolated cell population of cochlea supporting cells by factor of at least 50.
Embodiment 1302. The method of any of the above enumerated embodiments wherein a composition of the present disclosure has the capacity to increase the cell density of Lgr5+ supporting cells in an in vitro isolated cell population of cochlea supporting cells by factor of at least 100.
Embodiment 1303. The method of any of the above enumerated embodiments wherein a composition of the present disclosure has the capacity to increase the cell density of Lgr5+ supporting cells in an in vitro isolated cell population of cochlea supporting cells by factor of at least 500.
Embodiment 1304. The method of any of the above enumerated embodiments wherein a composition of the present disclosure has the capacity to increase the cell density of Lgr5+ supporting cells in an in vitro isolated cell population of cochlea supporting cells by factor of at least 1000.
Embodiment 1305. The method of any of the above enumerated embodiments wherein a composition of the present disclosure has the capacity to increase the cell density of Lgr5+ supporting cells in an in vitro isolated cell population of cochlea supporting cells by factor of at least 2000.
Embodiment 1306. The method of any of the above enumerated embodiments wherein a composition of the present disclosure has the capacity to increase the cell density of Lgr5+ supporting cells in an in vivo population of cochlear supporting cells by about or at least about 5%.
Embodiment 1307. The method of any of the above enumerated embodiments wherein a composition of the present disclosure has the capacity to increase the cell density of Lgr5+ supporting cells in an in vivo population of cochlear supporting cells by about or at least about 10% or more.
Embodiment 1308. The method of any of the above enumerated embodiments wherein a composition of the present disclosure has the capacity to increase the cell density of Lgr5+ supporting cells in an in vivo population of cochlear supporting cells by about or at least about 20% or more.
Embodiment 1309. The method of any of the above enumerated embodiments wherein a composition of the present disclosure has the capacity to increase the cell density of Lgr5+ supporting cells in an in vivo population of cochlear supporting cells by about or at least about 30% or more.
Embodiment 1310. The method of any of the above enumerated embodiments wherein a composition of the present disclosure has the capacity to increase the number of Lgr5+ cells in the cochlea by inducing expression of Lgr5 in cells with absent or low detection levels of the protein, while maintaining Native Morphology.
Embodiment 1311. The method of any of the above enumerated embodiments wherein a composition of the present disclosure has the capacity to increase the number of Lgr5+ cells in the cochlea by inducing expression of Lgr5 in cells with absent or low detection levels of the protein, while maintaining Native Morphology and without producing Cell Aggregates.
Embodiment 1312. The method of any of the above enumerated embodiments wherein proliferation of a Lgr5+ cochlear cell is increased with the TAZ activator and the Wnt agonist.
Embodiment 1313. The method of any of the above enumerated embodiments wherein the cell is further contacted with an epigenetic agent such as an HDAC inhibitor.
Embodiment 1314. The method of any of the above enumerated embodiments wherein the HDAC inhibitor is VPA.
Embodiment 1315. The method of any of the above enumerated embodiments wherein the Lgr5+ cochlear cell proliferation is increased compared to a vehicle control.
Embodiment 1316. The method of any of the above enumerated embodiments wherein the TAZ activator and the Wnt agonist increases Lgr5+ cochlear cell proliferation by at least about 1.1-fold or more, relative to a vehicle control.
Embodiment 1317. The method of any of the above enumerated embodiments wherein the TAZ activator and the Wnt agonist increases Lgr5+ cochlear cell proliferation by at least about 1.2-fold or more, relative to a vehicle control.
Embodiment 1318. The method of any of the above enumerated embodiments wherein the TAZ activator and the Wnt agonist increases Lgr5+ cochlear cell proliferation by at least about 1.2-fold or more, relative to a vehicle control.
Embodiment 1319. The method of any of the above enumerated embodiments wherein the TAZ activator and the Wnt agonist increases Lgr5+ cochlear cell proliferation by at least about 1.3-fold or more, relative to a vehicle control.
Embodiment 1320. The method of any of the above enumerated embodiments wherein the TAZ activator and the Wnt agonist increases Lgr5+ cochlear cell proliferation by at least about 1.4-fold or more, relative to a vehicle control.
Embodiment 1321. The method of any of the above enumerated embodiments wherein the TAZ activator and the Wnt agonist increases Lgr5+ cochlear cell proliferation by at least about 1.5-fold or more, relative to a vehicle control.
Embodiment 1322. The method of any of the above enumerated embodiments wherein the TAZ activator and the Wnt agonist increases Lgr5+ cochlear cell proliferation by at least about 1.6-fold or more, relative to a vehicle control.
Embodiment 1323. The method of any of the above enumerated embodiments wherein the TAZ activator and the Wnt agonist increases Lgr5+ cochlear cell proliferation by at least about 1.7-fold or more, relative to a vehicle control.
Embodiment 1324. The method of any of the above enumerated embodiments wherein the TAZ activator and the Wnt agonist increases Lgr5+ cochlear cell proliferation by at least about 1.8-fold or more, relative to a vehicle control.
Embodiment 1325. The method of any of the above enumerated embodiments wherein the TAZ activator and the Wnt agonist increases Lgr5+ cochlear cell proliferation by at least about 1.9-fold or more, relative to a vehicle control.
Embodiment 1326. The method of any of the above enumerated embodiments wherein the TAZ activator and the Wnt agonist increases Lgr5+ cochlear cell proliferation by at least about 2-fold or more, relative to a vehicle control.
Embodiment 1327. The method of any of the above enumerated embodiments wherein the TAZ activator and the Wnt agonist increases Lgr5+ cochlear cell proliferation by at least about 3-fold or more, relative to a vehicle control.
Embodiment 1328. The method of any of the above enumerated embodiments wherein the TAZ activator and the Wnt agonist increases Lgr5+ cochlear cell proliferation by at least about 4-fold or more, relative to a vehicle control.
Embodiment 1329. The method of any of the above enumerated embodiments wherein the TAZ activator and the Wnt agonist increases Lgr5+ cochlear cell proliferation by at least about 5-fold or more, relative to a vehicle control.
Embodiment 1330. The method of any of the above enumerated embodiments wherein the TAZ activator and the Wnt agonist increases Lgr5+ cochlear cell proliferation by at least about 6-fold or more, relative to a vehicle control.
Embodiment 1331. The method of any of the above enumerated embodiments wherein the TAZ activator and the Wnt agonist increases Lgr5+ cochlear cell proliferation by at least about 7-fold or more, relative to a vehicle control.
Embodiment 1332. The method of any of the above enumerated embodiments wherein the TAZ activator and the Wnt agonist increases Lgr5+ cochlear cell proliferation by at least about 8-fold or more, relative to a vehicle control.
Embodiment 1333. The method of any of the above enumerated embodiments wherein the TAZ activator and the Wnt agonist increases Lgr5+ cochlear cell proliferation by at least about 9-fold or more, relative to a vehicle control.
Embodiment 1334. The method of any of the above enumerated embodiments wherein the TAZ activator and the Wnt agonist increases Lgr5+ cochlear cell proliferation by at least about 10-fold or more, relative to a vehicle control.
Embodiment 1335. The method of any of the above enumerated embodiments wherein the TAZ activator and the Wnt agonist increases Lgr5+ cochlear cell proliferation by at least about 15-fold or more, relative to a vehicle control.
Embodiment 1336. The method of any of the above enumerated embodiments wherein the TAZ activator and the Wnt agonist increases Lgr5+ cochlear cell proliferation by at least about 20-fold or more, relative to a vehicle control.
Embodiment 1337. The method of any of the above enumerated embodiments wherein the TAZ activator and the Wnt agonist increases Lgr5+ cochlear cell proliferation by at least about 30-fold or more, relative to a vehicle control.
Embodiment 1338. The method of any of the above enumerated embodiments wherein the TAZ activator and the Wnt agonist increases Lgr5+ cochlear cell proliferation by at least about 40-fold or more, relative to a vehicle control.
Embodiment 1339. The method of any of the above enumerated embodiments wherein the TAZ activator and the Wnt agonist increases Lgr5+ cochlear cell proliferation by at least about 50-fold or more, relative to a vehicle control.
Embodiment 1340. The method of any of the above enumerated embodiments wherein the TAZ activator and the Wnt agonist increases Lgr5+ cochlear cell proliferation by at least about 60-fold or more, relative to a vehicle control.
Embodiment 1341. The method of any of the above enumerated embodiments wherein the TAZ activator and the Wnt agonist increases Lgr5+ cochlear cell proliferation by at least about 70-fold or more, relative to a vehicle control.
Embodiment 1342. The method of any of the above enumerated embodiments wherein the TAZ activator and the Wnt agonist increases Lgr5+ cochlear cell proliferation by at least about 80-fold or more, relative to a vehicle control.
Embodiment 1343. The method of any of the above enumerated embodiments wherein the TAZ activator and the Wnt agonist increases Lgr5+ cochlear cell proliferation by at least about 90-fold or more, relative to a vehicle control.
Embodiment 1344. The method of any of the above enumerated embodiments wherein the TAZ activator and the Wnt agonist increases Lgr5+ cochlear cell proliferation by at least about 100-fold or more, relative to a vehicle control.
Embodiment 1345. The method of any of the above enumerated embodiments wherein the TAZ activator and the Wnt agonist increases Lgr5+ cochlear cell proliferation by at least about 200-fold or more, relative to a vehicle control.
Embodiment 1346. The method of any of the above enumerated embodiments wherein the TAZ activator and the Wnt agonist increases Lgr5+ cochlear cell proliferation by at least about 500-fold or more, relative to a vehicle control.
Embodiment 1347. The method of any of the above enumerated embodiments wherein the TAZ activator and the Wnt agonist increases Lgr5+ cochlear cell proliferation by at least about 1000-fold or more, relative to a vehicle control.
Embodiment 1348. The method of any of the above enumerated embodiments wherein the TAZ activator and the Wnt agonist in combination with an epigenetic agent increases Lgr5+ cochlear cell proliferation by at least about 1.1-fold or more than the TAZ activator and the Wnt agonist in a Stem Cell Proliferation Assay.
Embodiment 1349. The method of any of the above enumerated embodiments wherein the TAZ activator and the Wnt agonist in combination with an epigenetic agent increases Lgr5+ cochlear cell proliferation by at least about 1.2-fold or more than the TAZ activator and the Wnt agonist in a Stem Cell Proliferation Assay.
Embodiment 1350. The method of any of the above enumerated embodiments wherein the TAZ activator and the Wnt agonist in combination with an epigenetic agent increases Lgr5+ cochlear cell proliferation by at least about 1.2-fold or more than the TAZ activator and the Wnt agonist in a Stem Cell Proliferation Assay.
Embodiment 1351. The method of any of the above enumerated embodiments wherein the TAZ activator and the Wnt agonist in combination with an epigenetic agent increases Lgr5+ cochlear cell proliferation by at least about 1.3-fold or more than the TAZ activator and the Wnt agonist in a Stem Cell Proliferation Assay.
Embodiment 1352. The method of any of the above enumerated embodiments wherein the TAZ activator and the Wnt agonist in combination with an epigenetic agent increases Lgr5+ cochlear cell proliferation by at least about 1.4-fold or more than the TAZ activator and the Wnt agonist in a Stem Cell Proliferation Assay.
Embodiment 1353. The method of any of the above enumerated embodiments wherein the TAZ activator and the Wnt agonist in combination with an epigenetic agent increases Lgr5+ cochlear cell proliferation by at least about 1.5-fold or more than the TAZ activator and the Wnt agonist in a Stem Cell Proliferation Assay.
Embodiment 1354. The method of any of the above enumerated embodiments wherein the TAZ activator and the Wnt agonist in combination with an epigenetic agent increases Lgr5+ cochlear cell proliferation by at least about 1.6-fold or more than the TAZ activator and the Wnt agonist in a Stem Cell Proliferation Assay.
Embodiment 1355. The method of any of the above enumerated embodiments wherein the TAZ activator and the Wnt agonist in combination with an epigenetic agent increases Lgr5+ cochlear cell proliferation by at least about 1.7-fold or more than the TAZ activator and the Wnt agonist in a Stem Cell Proliferation Assay.
Embodiment 1356. The method of any of the above enumerated embodiments wherein the TAZ activator and the Wnt agonist in combination with an epigenetic agent increases Lgr5+ cochlear cell proliferation by at least about 1.8-fold or more than the TAZ activator and the Wnt agonist in a Stem Cell Proliferation Assay.
Embodiment 1357. The method of any of the above enumerated embodiments wherein the TAZ activator and the Wnt agonist in combination with an epigenetic agent increases Lgr5+ cochlear cell proliferation by at least about 1.9-fold or more than the TAZ activator and the Wnt agonist in a Stem Cell Proliferation Assay.
Embodiment 1358. The method of any of the above enumerated embodiments wherein the TAZ activator and the Wnt agonist in combination with an epigenetic agent increases Lgr5+ cochlear cell proliferation by at least about 2-fold or more than the TAZ activator and the Wnt agonist in a Stem Cell Proliferation Assay.
Embodiment 1359. The method of any of the above enumerated embodiments wherein the TAZ activator and the Wnt agonist in combination with an epigenetic agent increases Lgr5+ cochlear cell proliferation by at least about 3-fold or more than the TAZ activator and the Wnt agonist in a Stem Cell Proliferation Assay.
Embodiment 1360. The method of any of the above enumerated embodiments wherein the TAZ activator and the Wnt agonist in combination with an epigenetic agent increases Lgr5+ cochlear cell proliferation by at least about 4-fold or more than the TAZ activator and the Wnt agonist in a Stem Cell Proliferation Assay.
Embodiment 1361. The method of any of the above enumerated embodiments wherein the TAZ activator and the Wnt agonist in combination with an epigenetic agent increases Lgr5+ cochlear cell proliferation by at least about 5-fold or more than the TAZ activator and the Wnt agonist in a Stem Cell Proliferation Assay.
Embodiment 1362. The method of any of the above enumerated embodiments wherein the TAZ activator and the Wnt agonist in combination with an epigenetic agent increases Lgr5+ cochlear cell proliferation by at least about 6-fold or more than the TAZ activator and the Wnt agonist in a Stem Cell Proliferation Assay.
Embodiment 1363. The method of any of the above enumerated embodiments wherein the TAZ activator and the Wnt agonist in combination with an epigenetic agent increases Lgr5+ cochlear cell proliferation by at least about 7-fold or more than the TAZ activator and the Wnt agonist in a Stem Cell Proliferation Assay.
Embodiment 1364. The method of any of the above enumerated embodiments wherein the TAZ activator and the Wnt agonist in combination with an epigenetic agent increases Lgr5+ cochlear cell proliferation by at least about 8-fold or more than the TAZ activator and the Wnt agonist in a Stem Cell Proliferation Assay.
Embodiment 1365. The method of any of the above enumerated embodiments wherein the TAZ activator and the Wnt agonist in combination with an epigenetic agent increases Lgr5+ cochlear cell proliferation by at least about 9-fold or more than the TAZ activator and the Wnt agonist in a Stem Cell Proliferation Assay.
Embodiment 1366. The method of any of the above enumerated embodiments wherein the TAZ activator and the Wnt agonist in combination with an epigenetic agent increases Lgr5+ cochlear cell proliferation by at least about 10-fold or more than the TAZ activator and the Wnt agonist in a Stem Cell Proliferation Assay.
Embodiment 1367. The method of any of the above enumerated embodiments wherein the TAZ activator and the Wnt agonist in combination with an epigenetic agent increases Lgr5+ cochlear cell proliferation by at least about 15-fold or more than the TAZ activator and the Wnt agonist in a Stem Cell Proliferation Assay.
Embodiment 1368. The method of any of the above enumerated embodiments wherein the TAZ activator and the Wnt agonist in combination with an epigenetic agent increases Lgr5+ cochlear cell proliferation by at least about 20-fold or more than the TAZ activator and the Wnt agonist in a Stem Cell Proliferation Assay.
Embodiment 1369. The method of any of the above enumerated embodiments wherein the TAZ activator and the Wnt agonist in combination with an epigenetic agent increases Lgr5+ cochlear cell proliferation by at least about 30-fold or more than the TAZ activator and the Wnt agonist in a Stem Cell Proliferation Assay.
Embodiment 1370. The method of any of the above enumerated embodiments wherein the TAZ activator and the Wnt agonist in combination with an epigenetic agent increases Lgr5+ cochlear cell proliferation by at least about 40-fold or more than the TAZ activator and the Wnt agonist in a Stem Cell Proliferation Assay.
Embodiment 1371. The method of any of the above enumerated embodiments wherein the TAZ activator and the Wnt agonist in combination with an epigenetic agent increases Lgr5+ cochlear cell proliferation by at least about 50-fold or more than the TAZ activator and the Wnt agonist in a Stem Cell Proliferation Assay.
Embodiment 1372. The method of any of the above enumerated embodiments wherein the TAZ activator and the Wnt agonist in combination with an epigenetic agent increases Lgr5+ cochlear cell proliferation by at least about 60-fold or more than the TAZ activator and the Wnt agonist in a Stem Cell Proliferation Assay.
Embodiment 1373. The method of any of the above enumerated embodiments wherein the TAZ activator and the Wnt agonist in combination with an epigenetic agent increases Lgr5+ cochlear cell proliferation by at least about 70-fold or more than the TAZ activator and the Wnt agonist in a Stem Cell Proliferation Assay.
Embodiment 1374. The method of any of the above enumerated embodiments wherein the TAZ activator and the Wnt agonist in combination with an epigenetic agent increases Lgr5+ cochlear cell proliferation by at least about 80-fold or more than the TAZ activator and the Wnt agonist in a Stem Cell Proliferation Assay.
Embodiment 1375. The method of any of the above enumerated embodiments wherein the TAZ activator and the Wnt agonist in combination with an epigenetic agent increases Lgr5+ cochlear cell proliferation by at least about 90-fold or more than the TAZ activator and the Wnt agonist in a Stem Cell Proliferation Assay.
Embodiment 1376. The method of any of the above enumerated embodiments wherein the TAZ activator and the Wnt agonist in combination with an epigenetic agent increases Lgr5+ cochlear cell proliferation by at least about 100-fold or more than the TAZ activator and the Wnt agonist in a Stem Cell Proliferation Assay.
Embodiment 1377. The method of any of the above enumerated embodiments wherein the TAZ activator and the Wnt agonist in combination with an epigenetic agent increases Lgr5+ cochlear cell proliferation by at least about 200-fold or more than the TAZ activator and the Wnt agonist in a Stem Cell Proliferation Assay.
Embodiment 1378. The method of any of the above enumerated embodiments wherein the TAZ activator and the Wnt agonist in combination with an epigenetic agent increases Lgr5+ cochlear cell proliferation by at least about 500-fold or more than the TAZ activator and the Wnt agonist in a Stem Cell Proliferation Assay.
Embodiment 1379. The method of any of the above enumerated embodiments wherein the TAZ activator and the Wnt agonist in combination with an epigenetic agent increases Lgr5+ cochlear cell proliferation by at least about 1000-fold or more than the TAZ activator and the Wnt agonist in a Stem Cell Proliferation Assay.
Embodiment 1380. The method of any of the above enumerated embodiments wherein the TAZ activator and the Wnt agonist in combination with an epigenetic agent increases Lgr5+ cochlear cell proliferation by at least about 1.1-fold relative to a Wnt agonist in combination with VPA.
Embodiment 1381. The method of any of the above enumerated embodiments wherein the TAZ activator and the Wnt agonist in combination with an epigenetic agent increases Lgr5+ cochlear cell proliferation by at least about 1.2-fold relative to a Wnt agonist in combination with VPA.
Embodiment 1382. The method of any of the above enumerated embodiments wherein the TAZ activator and the Wnt agonist in combination with an epigenetic agent increases Lgr5+ cochlear cell proliferation by at least about 1.3-fold relative to a Wnt agonist in combination with VPA.
Embodiment 1383. The method of any of the above enumerated embodiments wherein the TAZ activator and the Wnt agonist in combination with an epigenetic agent increases Lgr5+ cochlear cell proliferation by at least about 1.4-fold relative to a Wnt agonist in combination with VPA.
Embodiment 1384. The method of any of the above enumerated embodiments wherein the TAZ activator and the Wnt agonist in combination with an epigenetic agent increases Lgr5+ cochlear cell proliferation by at least about 1.5-fold relative to a Wnt agonist in combination with VPA.
Embodiment 1385. The method of any of the above enumerated embodiments wherein the TAZ activator and the Wnt agonist in combination with an epigenetic agent increases Lgr5+ cochlear cell proliferation by at least about 1.6-fold relative to a Wnt agonist in combination with VPA.
Embodiment 1386. The method of any of the above enumerated embodiments wherein the TAZ activator and the Wnt agonist in combination with an epigenetic agent increases Lgr5+ cochlear cell proliferation by at least about 1.7-fold relative to a Wnt agonist in combination with VPA.
Embodiment 1387. The method of any of the above enumerated embodiments wherein the TAZ activator and the Wnt agonist in combination with an epigenetic agent increases Lgr5+ cochlear cell proliferation by at least about 1.8-fold relative to a Wnt agonist in combination with VPA.
Embodiment 1388. The method of any of the above enumerated embodiments wherein the TAZ activator and the Wnt agonist in combination with an epigenetic agent increases Lgr5+ cochlear cell proliferation by at least about 1.9-fold relative to a Wnt agonist in combination with VPA.
Embodiment 1389. The method of any of the above enumerated embodiments wherein the TAZ activator and the Wnt agonist in combination with an epigenetic agent increases Lgr5+ cochlear cell proliferation by at least about 2-fold relative to a Wnt agonist in combination with VPA.
Embodiment 1390. The method of any of the above enumerated embodiments wherein the TAZ activator and the Wnt agonist in combination with an epigenetic agent increases Lgr5+ cochlear cell proliferation by at least about 3-fold relative to a Wnt agonist in combination with VPA.
Embodiment 1391. The method of any of the above enumerated embodiments wherein the TAZ activator and the Wnt agonist in combination with an epigenetic agent increases Lgr5+ cochlear cell proliferation by at least about 4-fold relative to a Wnt agonist in combination with VPA.
Embodiment 1392. The method of any of the above enumerated embodiments wherein the TAZ activator and the Wnt agonist in combination with an epigenetic agent increases Lgr5+ cochlear cell proliferation by at least about 5-fold relative to a Wnt agonist in combination with VPA.
Embodiment 1393. The method of any of the above enumerated embodiments wherein the TAZ activator and the Wnt agonist in combination with an epigenetic agent increases Lgr5+ cochlear cell proliferation by at least about 6-fold relative to a Wnt agonist in combination with VPA.
Embodiment 1394. The method of any of the above enumerated embodiments wherein the TAZ activator and the Wnt agonist in combination with an epigenetic agent increases Lgr5+ cochlear cell proliferation by at least about 7-fold relative to a Wnt agonist in combination with VPA.
Embodiment 1395. The method of any of the above enumerated embodiments wherein the TAZ activator and the Wnt agonist in combination with an epigenetic agent increases Lgr5+ cochlear cell proliferation by at least about 8-fold relative to a Wnt agonist in combination with VPA.
Embodiment 1396. The method of any of the above enumerated embodiments wherein the TAZ activator and the Wnt agonist in combination with an epigenetic agent increases Lgr5+ cochlear cell proliferation by at least about 9-fold relative to a Wnt agonist in combination with VPA.
Embodiment 1397. The method of any of the above enumerated embodiments wherein the TAZ activator and the Wnt agonist in combination with an epigenetic agent increases Lgr5+ cochlear cell proliferation by at least about 10-fold relative to a Wnt agonist in combination with VPA.
Embodiment 1398. The method of any of the above enumerated embodiments wherein the TAZ activator and the Wnt agonist in combination with an epigenetic agent increases Lgr5+ cochlear cell proliferation by at least about 15-fold relative to a Wnt agonist in combination with VPA.
Embodiment 1399. The method of any of the above enumerated embodiments wherein the TAZ activator and the Wnt agonist in combination with an epigenetic agent increases Lgr5+ cochlear cell proliferation by at least about 20-fold relative to a Wnt agonist in combination with VPA.
Embodiment 1400. The method of any of the above enumerated embodiments wherein the TAZ activator and the Wnt agonist in combination with an epigenetic agent increases Lgr5+ cochlear cell proliferation by at least about 30-fold relative to a Wnt agonist in combination with VPA.
Embodiment 1401. The method of any of the above enumerated embodiments wherein the TAZ activator and the Wnt agonist in combination with an epigenetic agent increases Lgr5+ cochlear cell proliferation by at least about 40-fold relative to a Wnt agonist in combination with VPA.
Embodiment 1402. The method of any of the above enumerated embodiments wherein the TAZ activator and the Wnt agonist in combination with an epigenetic agent increases Lgr5+ cochlear cell proliferation by at least about 50-fold relative to a Wnt agonist in combination with VPA.
Embodiment 1403. The method of any of the above enumerated embodiments wherein the TAZ activator and the Wnt agonist in combination with an epigenetic agent increases Lgr5+ cochlear cell proliferation by at least about 60-fold relative to a Wnt agonist in combination with VPA.
Embodiment 1404. The method of any of the above enumerated embodiments wherein the TAZ activator and the Wnt agonist in combination with an epigenetic agent increases Lgr5+ cochlear cell proliferation by at least about 70-fold relative to a Wnt agonist in combination with VPA.
Embodiment 1405. The method of any of the above enumerated embodiments wherein the TAZ activator and the Wnt agonist in combination with an epigenetic agent increases Lgr5+ cochlear cell proliferation by at least about 80-fold relative to a Wnt agonist in combination with VPA.
Embodiment 1406. The method of any of the above enumerated embodiments wherein the TAZ activator and the Wnt agonist in combination with an epigenetic agent increases Lgr5+ cochlear cell proliferation by at least about 90-fold relative to a Wnt agonist in combination with VPA.
Embodiment 1407. The method of any of the above enumerated embodiments wherein the TAZ activator and the Wnt agonist in combination with an epigenetic agent increases Lgr5+ cochlear cell proliferation by at least about 100-fold relative to a Wnt agonist in combination with VPA.
Embodiment 1408. The method of any of the above enumerated embodiments wherein the TAZ activator and the Wnt agonist in combination with an epigenetic agent increases Lgr5+ cochlear cell proliferation by at least about 200-fold relative to a Wnt agonist in combination with VPA.
Embodiment 1409. The method of any of the above enumerated embodiments wherein the TAZ activator and the Wnt agonist in combination with an epigenetic agent increases Lgr5+ cochlear cell proliferation by at least about 500-fold relative to a Wnt agonist in combination with VPA.
Embodiment 1410. The method of any of the above enumerated embodiments wherein the TAZ activator and the Wnt agonist in combination with an epigenetic agent increases Lgr5+ cochlear cell proliferation by at least about 1000-fold relative to a Wnt agonist in combination with VPA.
Embodiment 1411. The method of any of the above enumerated embodiments wherein the TAZ activator and the Wnt agonist increases Lgr5+ cochlear cell proliferation by at least about 1.1-fold or more relative to a Wnt agonist alone, as measured against a Stem Cell Proliferation Assay.
Embodiment 1412. The method of any of the above enumerated embodiments wherein the TAZ activator and the Wnt agonist increases Lgr5+ cochlear cell proliferation by at least about 1.2-fold or more relative to a Wnt agonist alone, as measured against a Stem Cell Proliferation Assay.
Embodiment 1413. The method of any of the above enumerated embodiments wherein the TAZ activator and the Wnt agonist increases Lgr5+ cochlear cell proliferation by at least about 1.2-fold or more relative to a Wnt agonist alone, as measured against a Stem Cell Proliferation Assay.
Embodiment 1414. The method of any of the above enumerated embodiments wherein the TAZ activator and the Wnt agonist increases Lgr5+ cochlear cell proliferation by at least about 1.3-fold or more relative to a Wnt agonist alone, as measured against a Stem Cell Proliferation Assay.
Embodiment 1415. The method of any of the above enumerated embodiments wherein the TAZ activator and the Wnt agonist increases Lgr5+ cochlear cell proliferation by at least about 1.4-fold or more relative to a Wnt agonist alone, as measured against a Stem Cell Proliferation Assay.
Embodiment 1416. The method of any of the above enumerated embodiments wherein the TAZ activator and the Wnt agonist increases Lgr5+ cochlear cell proliferation by at least about 1.5-fold or more relative to a Wnt agonist alone, as measured against a Stem Cell Proliferation Assay.
Embodiment 1417. The method of any of the above enumerated embodiments wherein the TAZ activator and the Wnt agonist increases Lgr5+ cochlear cell proliferation by at least about 1.6-fold or more relative to a Wnt agonist alone, as measured against a Stem Cell Proliferation Assay.
Embodiment 1418. The method of any of the above enumerated embodiments wherein the TAZ activator and the Wnt agonist increases Lgr5+ cochlear cell proliferation by at least about 1.7-fold or more relative to a Wnt agonist alone, as measured against a Stem Cell Proliferation Assay.
Embodiment 1419. The method of any of the above enumerated embodiments wherein the TAZ activator and the Wnt agonist increases Lgr5+ cochlear cell proliferation by at least about 1.8-fold or more relative to a Wnt agonist alone, as measured against a Stem Cell Proliferation Assay.
Embodiment 1420. The method of any of the above enumerated embodiments wherein the TAZ activator and the Wnt agonist increases Lgr5+ cochlear cell proliferation by at least about 1.9-fold or more relative to a Wnt agonist alone, as measured against a Stem Cell Proliferation Assay.
Embodiment 1421. The method of any of the above enumerated embodiments wherein the TAZ activator and the Wnt agonist increases Lgr5+ cochlear cell proliferation by at least about 2-fold or more relative to a Wnt agonist alone, as measured against a Stem Cell Proliferation Assay.
Embodiment 1422. The method of any of the above enumerated embodiments wherein the TAZ activator and the Wnt agonist increases Lgr5+ cochlear cell proliferation by at least about 3-fold or more relative to a Wnt agonist alone, as measured against a Stem Cell Proliferation Assay.
Embodiment 1423. The method of any of the above enumerated embodiments wherein the TAZ activator and the Wnt agonist increases Lgr5+ cochlear cell proliferation by at least about 4-fold or more relative to a Wnt agonist alone, as measured against a Stem Cell Proliferation Assay.
Embodiment 1424. The method of any of the above enumerated embodiments wherein the TAZ activator and the Wnt agonist increases Lgr5+ cochlear cell proliferation by at least about 5-fold or more relative to a Wnt agonist alone, as measured against a Stem Cell Proliferation Assay.
Embodiment 1425. The method of any of the above enumerated embodiments wherein the TAZ activator and the Wnt agonist increases Lgr5+ cochlear cell proliferation by at least about 6-fold or more relative to a Wnt agonist alone, as measured against a Stem Cell Proliferation Assay.
Embodiment 1426. The method of any of the above enumerated embodiments wherein the TAZ activator and the Wnt agonist increases Lgr5+ cochlear cell proliferation by at least about 7-fold or more relative to a Wnt agonist alone, as measured against a Stem Cell Proliferation Assay.
Embodiment 1427. The method of any of the above enumerated embodiments wherein the TAZ activator and the Wnt agonist increases Lgr5+ cochlear cell proliferation by at least about 8-fold or more relative to a Wnt agonist alone, as measured against a Stem Cell Proliferation Assay.
Embodiment 1428. The method of any of the above enumerated embodiments wherein the TAZ activator and the Wnt agonist increases Lgr5+ cochlear cell proliferation by at least about 9-fold or more relative to a Wnt agonist alone, as measured against a Stem Cell Proliferation Assay.
Embodiment 1429. The method of any of the above enumerated embodiments wherein the TAZ activator and the Wnt agonist increases Lgr5+ cochlear cell proliferation by at least about 10-fold or more relative to a Wnt agonist alone, as measured against a Stem Cell Proliferation Assay.
Embodiment 1430. The method of any of the above enumerated embodiments wherein the TAZ activator and the Wnt agonist increases Lgr5+ cochlear cell proliferation by at least about 15-fold or more relative to a Wnt agonist alone, as measured against a Stem Cell Proliferation Assay.
Embodiment 1431. The method of any of the above enumerated embodiments wherein the TAZ activator and the Wnt agonist increases Lgr5+ cochlear cell proliferation by at least about 20-fold or more relative to a Wnt agonist alone, as measured against a Stem Cell Proliferation Assay.
Embodiment 1432. The method of any of the above enumerated embodiments wherein the TAZ activator and the Wnt agonist increases Lgr5+ cochlear cell proliferation by at least about 30-fold or more relative to a Wnt agonist alone, as measured against a Stem Cell Proliferation Assay.
Embodiment 1433. The method of any of the above enumerated embodiments wherein the TAZ activator and the Wnt agonist increases Lgr5+ cochlear cell proliferation by at least about 40-fold or more relative to a Wnt agonist alone, as measured against a Stem Cell Proliferation Assay.
Embodiment 1434. The method of any of the above enumerated embodiments wherein the TAZ activator and the Wnt agonist increases Lgr5+ cochlear cell proliferation by at least about 50-fold or more relative to a Wnt agonist alone, as measured against a Stem Cell Proliferation Assay.
Embodiment 1435. The method of any of the above enumerated embodiments wherein the TAZ activator and the Wnt agonist increases Lgr5+ cochlear cell proliferation by at least about 60-fold or more relative to a Wnt agonist alone, as measured against a Stem Cell Proliferation Assay.
Embodiment 1436. The method of any of the above enumerated embodiments wherein the TAZ activator and the Wnt agonist increases Lgr5+ cochlear cell proliferation by at least about 70-fold or more relative to a Wnt agonist alone, as measured against a Stem Cell Proliferation Assay.
Embodiment 1437. The method of any of the above enumerated embodiments wherein the TAZ activator and the Wnt agonist increases Lgr5+ cochlear cell proliferation by at least about 80-fold or more relative to a Wnt agonist alone, as measured against a Stem Cell Proliferation Assay.
Embodiment 1438. The method of any of the above enumerated embodiments wherein the TAZ activator and the Wnt agonist increases Lgr5+ cochlear cell proliferation by at least about 90-fold or more relative to a Wnt agonist alone, as measured against a Stem Cell Proliferation Assay.
Embodiment 1439. The method of any of the above enumerated embodiments wherein the TAZ activator and the Wnt agonist increases Lgr5+ cochlear cell proliferation by at least about 100-fold or more relative to a Wnt agonist alone, as measured against a Stem Cell Proliferation Assay.
Embodiment 1440. The method of any of the above enumerated embodiments wherein the TAZ activator and the Wnt agonist increases Lgr5+ cochlear cell proliferation by at least about 200-fold or more relative to a Wnt agonist alone, as measured against a Stem Cell Proliferation Assay.
Embodiment 1441. The method of any of the above enumerated embodiments wherein the TAZ activator and the Wnt agonist increases Lgr5+ cochlear cell proliferation by at least about 500-fold or more relative to a Wnt agonist alone, as measured against a Stem Cell Proliferation Assay.
Embodiment 1442. The method of any of the above enumerated embodiments wherein the TAZ activator and the Wnt agonist increases Lgr5+ cochlear cell proliferation by at least about 1000-fold or more relative to a Wnt agonist alone, as measured against a Stem Cell Proliferation Assay.
Embodiment 1443. The method of any of the above enumerated embodiments wherein the TAZ activator and the Wnt agonist increases Lgr5+ cochlear cell proliferation by at least about 1.1-fold or more relative to a Wnt agonist in combination with VPA, as measured against a Stem Cell Proliferation Assay.
Embodiment 1444. The method of any of the above enumerated embodiments wherein the TAZ activator and the Wnt agonist increases Lgr5+ cochlear cell proliferation by at least about 1.2-fold or more relative to a Wnt agonist in combination with VPA, as measured against a Stem Cell Proliferation Assay.
Embodiment 1445. The method of any of the above enumerated embodiments wherein the TAZ activator and the Wnt agonist increases Lgr5+ cochlear cell proliferation by at least about 1.2-fold or more relative to a Wnt agonist in combination with VPA, as measured against a Stem Cell Proliferation Assay.
Embodiment 1446. The method of any of the above enumerated embodiments wherein the TAZ activator and the Wnt agonist increases Lgr5+ cochlear cell proliferation by at least about 1.3-fold or more relative to a Wnt agonist in combination with VPA, as measured against a Stem Cell Proliferation Assay.
Embodiment 1447. The method of any of the above enumerated embodiments wherein the TAZ activator and the Wnt agonist increases Lgr5+ cochlear cell proliferation by at least about 1.4-fold or more relative to a Wnt agonist in combination with VPA, as measured against a Stem Cell Proliferation Assay.
Embodiment 1448. The method of any of the above enumerated embodiments wherein the TAZ activator and the Wnt agonist increases Lgr5+ cochlear cell proliferation by at least about 1.5-fold or more relative to a Wnt agonist in combination with VPA, as measured against a Stem Cell Proliferation Assay.
Embodiment 1449. The method of any of the above enumerated embodiments wherein the TAZ activator and the Wnt agonist increases Lgr5+ cochlear cell proliferation by at least about 1.6-fold or more relative to a Wnt agonist in combination with VPA, as measured against a Stem Cell Proliferation Assay.
Embodiment 1450. The method of any of the above enumerated embodiments wherein the TAZ activator and the Wnt agonist increases Lgr5+ cochlear cell proliferation by at least about 1.7-fold or more relative to a Wnt agonist in combination with VPA, as measured against a Stem Cell Proliferation Assay.
Embodiment 1451. The method of any of the above enumerated embodiments wherein the TAZ activator and the Wnt agonist increases Lgr5+ cochlear cell proliferation by at least about 1.8-fold or more relative to a Wnt agonist in combination with VPA, as measured against a Stem Cell Proliferation Assay.
Embodiment 1452. The method of any of the above enumerated embodiments wherein the TAZ activator and the Wnt agonist increases Lgr5+ cochlear cell proliferation by at least about 1.9-fold or more relative to a Wnt agonist in combination with VPA, as measured against a Stem Cell Proliferation Assay.
Embodiment 1453. The method of any of the above enumerated embodiments wherein the TAZ activator and the Wnt agonist increases Lgr5+ cochlear cell proliferation by at least about 2-fold or more relative to a Wnt agonist in combination with VPA, as measured against a Stem Cell Proliferation Assay.
Embodiment 1454. The method of any of the above enumerated embodiments wherein the TAZ activator and the Wnt agonist increases Lgr5+ cochlear cell proliferation by at least about 3-fold or more relative to a Wnt agonist in combination with VPA, as measured against a Stem Cell Proliferation Assay.
Embodiment 1455. The method of any of the above enumerated embodiments wherein the TAZ activator and the Wnt agonist increases Lgr5+ cochlear cell proliferation by at least about 4-fold or more relative to a Wnt agonist in combination with VPA, as measured against a Stem Cell Proliferation Assay.
Embodiment 1456. The method of any of the above enumerated embodiments wherein the TAZ activator and the Wnt agonist increases Lgr5+ cochlear cell proliferation by at least about 5-fold or more relative to a Wnt agonist in combination with VPA, as measured against a Stem Cell Proliferation Assay.
Embodiment 1457. The method of any of the above enumerated embodiments wherein the TAZ activator and the Wnt agonist increases Lgr5+ cochlear cell proliferation by at least about 6-fold or more relative to a Wnt agonist in combination with VPA, as measured against a Stem Cell Proliferation Assay.
Embodiment 1458. The method of any of the above enumerated embodiments wherein the TAZ activator and the Wnt agonist increases Lgr5+ cochlear cell proliferation by at least about 7-fold or more relative to a Wnt agonist in combination with VPA, as measured against a Stem Cell Proliferation Assay.
Embodiment 1459. The method of any of the above enumerated embodiments wherein the TAZ activator and the Wnt agonist increases Lgr5+ cochlear cell proliferation by at least about 8-fold or more relative to a Wnt agonist in combination with VPA, as measured against a Stem Cell Proliferation Assay.
Embodiment 1460. The method of any of the above enumerated embodiments wherein the TAZ activator and the Wnt agonist increases Lgr5+ cochlear cell proliferation by at least about 9-fold or more relative to a Wnt agonist in combination with VPA, as measured against a Stem Cell Proliferation Assay.
Embodiment 1461. The method of any of the above enumerated embodiments wherein the TAZ activator and the Wnt agonist increases Lgr5+ cochlear cell proliferation by at least about 10-fold or more relative to a Wnt agonist in combination with VPA, as measured against a Stem Cell Proliferation Assay.
Embodiment 1462. The method of any of the above enumerated embodiments wherein the TAZ activator and the Wnt agonist increases Lgr5+ cochlear cell proliferation by at least about 15-fold or more relative to a Wnt agonist in combination with VPA, as measured against a Stem Cell Proliferation Assay.
Embodiment 1463. The method of any of the above enumerated embodiments wherein the TAZ activator and the Wnt agonist increases Lgr5+ cochlear cell proliferation by at least about 20-fold or more relative to a Wnt agonist in combination with VPA, as measured against a Stem Cell Proliferation Assay.
Embodiment 1464. The method of any of the above enumerated embodiments wherein the TAZ activator and the Wnt agonist increases Lgr5+ cochlear cell proliferation by at least about 30-fold or more relative to a Wnt agonist in combination with VPA, as measured against a Stem Cell Proliferation Assay.
Embodiment 1465. The method of any of the above enumerated embodiments wherein the TAZ activator and the Wnt agonist increases Lgr5+ cochlear cell proliferation by at least about 40-fold or more relative to a Wnt agonist in combination with VPA, as measured against a Stem Cell Proliferation Assay.
Embodiment 1466. The method of any of the above enumerated embodiments wherein the TAZ activator and the Wnt agonist increases Lgr5+ cochlear cell proliferation by at least about 50-fold or more relative to a Wnt agonist in combination with VPA, as measured against a Stem Cell Proliferation Assay.
Embodiment 1467. The method of any of the above enumerated embodiments wherein the TAZ activator and the Wnt agonist increases Lgr5+ cochlear cell proliferation by at least about 60-fold or more relative to a Wnt agonist in combination with VPA, as measured against a Stem Cell Proliferation Assay.
Embodiment 1468. The method of any of the above enumerated embodiments wherein the TAZ activator and the Wnt agonist increases Lgr5+ cochlear cell proliferation by at least about 70-fold or more relative to a Wnt agonist in combination with VPA, as measured against a Stem Cell Proliferation Assay.
Embodiment 1469. The method of any of the above enumerated embodiments wherein the TAZ activator and the Wnt agonist increases Lgr5+ cochlear cell proliferation by at least about 80-fold or more relative to a Wnt agonist in combination with VPA, as measured against a Stem Cell Proliferation Assay.
Embodiment 1470. The method of any of the above enumerated embodiments wherein the TAZ activator and the Wnt agonist increases Lgr5+ cochlear cell proliferation by at least about 90-fold or more relative to a Wnt agonist in combination with VPA, as measured against a Stem Cell Proliferation Assay.
Embodiment 1471. The method of any of the above enumerated embodiments wherein the TAZ activator and the Wnt agonist increases Lgr5+ cochlear cell proliferation by at least about 100-fold or more relative to a Wnt agonist in combination with VPA, as measured against a Stem Cell Proliferation Assay.
Embodiment 1472. The method of any of the above enumerated embodiments wherein the TAZ activator and the Wnt agonist increases Lgr5+ cochlear cell proliferation by at least about 200-fold or more relative to a Wnt agonist in combination with VPA, as measured against a Stem Cell Proliferation Assay.
Embodiment 1473. The method of any of the above enumerated embodiments wherein the TAZ activator and the Wnt agonist increases Lgr5+ cochlear cell proliferation by at least about 500-fold or more relative to a Wnt agonist in combination with VPA, as measured against a Stem Cell Proliferation Assay.
Embodiment 1474. The method of any of the above enumerated embodiments wherein the TAZ activator and the Wnt agonist increases Lgr5+ cochlear cell proliferation by at least about 1000-fold or more relative to a Wnt agonist in combination with VPA, as measured against a Stem Cell Proliferation Assay.
Embodiment 1475. The method of any of the above enumerated embodiments wherein a population of cochlear cells in a cochlear tissue comprising a parent population of cells is expanded by contacting the cochlear tissue with an TAZ activator and a Wnt agonist to form an expanded population of cells in the cochlear tissue.
Embodiment 1476. The method of any of the above enumerated embodiments wherein a population of Lgr5+ cochlear cells is expanded by contacting the cell population with the TAZ activator and the Wnt agonist to form an expanded population of cells in the cochlear tissue.
Embodiment 1477. The method of any of the above enumerated embodiments wherein the expanded population is capable of differentiating into hair cells as measured in a stem cell differentiation assay.
Embodiment 1478. The method of any of the above enumerated embodiments wherein the cochlear cell is a cochlear tissue.
Embodiment 1479. The method of any of the above enumerated embodiments wherein the cochlear cell is in a cochlear tissue in a subject.
Embodiment 1480. The method of any of the above enumerated embodiments wherein the method is for treating a subject who has hearing loss.
Embodiment 1481. The method of any of the above enumerated embodiments wherein the method is for treating a subject who has reduced auditory function.
Embodiment 1482. The method of any of the above enumerated embodiments wherein the method is for treating a subject who is at risk of developing hearing loss.
Embodiment 1483. The method of any of the above enumerated embodiments wherein the method is for treating a subject who is at risk of developing reduced auditory function.
Embodiment 1484. The method of any of the above enumerated embodiments wherein the method is for treating acute ear disease and hearing loss.
Embodiment 1485. The method of any of the above enumerated embodiments wherein the method is for treating chronic ear disease and hearing loss.
Embodiment 1486. The method of any of the above enumerated embodiments wherein the method is for treating dizziness and balance problems especially of sudden hearing loss.
Embodiment 1487. The method of any of the above enumerated embodiments wherein the method is for treating acoustic trauma.
Embodiment 1488. The method of any of the above enumerated embodiments wherein the method is for treating hearing loss due to chronic noise exposure.
Embodiment 1489. The method of any of the above enumerated embodiments wherein the method is for treating presbycusis.
Embodiment 1490. The method of any of the above enumerated embodiments wherein the method is for treating trauma during implantation of the inner ear prosthesis (insertion trauma).
Embodiment 1491. The method of any of the above enumerated embodiments wherein the method is for treating dizziness due to diseases of the inner ear area.
Embodiment 1492. The method of any of the above enumerated embodiments wherein the method is for treating dizziness related and/or as a symptom of Meniere's disease.
Embodiment 1493. The method of any of the above enumerated embodiments wherein the method is for treating vertigo related and/or as a symptom of Meniere's disease.
Embodiment 1494. The method of any of the above enumerated embodiments wherein the method is for treating tinnitus.
Embodiment 1495. The method of any of the above enumerated embodiments wherein the method is for treating hearing loss due to antibiotics.
Embodiment 1496. The method of any of the above enumerated embodiments wherein the method is for treating hearing loss due to cytostatics.
Embodiment 1497. The method of any of the above enumerated embodiments wherein the method is for treating hearing loss due to other drugs.
Embodiment 1498. The method of any of the above enumerated embodiments wherein the method is used to prevent, reduce or treat the incidence of inner ear disorders involving inner ear hair cells and their progenitors.
Embodiment 1499. The method of any of the above enumerated embodiments wherein the method is used to prevent, reduce or treat the severity of inner ear disorders involving inner ear hair cells and their progenitors.
Embodiment 1500. The method of any of the above enumerated embodiments wherein the method is used to prevent, reduce or treat the incidence of hearing impairments involving inner ear hair cells and their progenitors.
Embodiment 1501. The method of any of the above enumerated embodiments wherein the method is used to prevent, reduce or treat the severity of hearing impairments involving inner ear hair cells and their progenitors.
Embodiment 1502. The method of any of the above enumerated embodiments wherein the condition being treated is arising as an unwanted side-effect of ototoxic therapeutic drugs including cisplatin and its analogs.
Embodiment 1503. The method of any of the above enumerated embodiments wherein the condition being treated is arising as an unwanted side-effect of ototoxic therapeutic drugs including aminoglycoside antibiotics.
Embodiment 1504. The method of any of the above enumerated embodiments wherein the condition being treated is arising as an unwanted side-effect of ototoxic therapeutic drugs including salicylate and its analogs.
Embodiment 1505. The method of any of the above enumerated embodiments wherein the condition being treated is arising as an unwanted side-effect of ototoxic therapeutic drugs including loop diuretics.
Embodiment 1506. The method of any of the above enumerated embodiments wherein the subject experiences an improvement in hearing as measured by behavior audiometry.
Embodiment 1507. The method of any of the above enumerated embodiments wherein the subject experiences an improvement in hearing as measured by auditory brainstem response (ABR) testing.
Embodiment 1508. The method of any of the above enumerated embodiments wherein hearing loss is treated by contacting a Lgr5+ cochlear cell with an TAZ activator and a Wnt agonist to form an expanded population of cells in the cochlear tissue.
Embodiment 1509. The method of any of the above enumerated embodiments wherein reduced auditory function is treated by contacting a Lgr5+ cochlear cell with an TAZ activator and a Wnt agonist to form an expanded population of cells in the cochlear tissue.
Embodiment 1510. The method of any of the above enumerated embodiments wherein hearing loss is prevented by contacting a Lgr5+ cochlear cell with an TAZ activator and a Wnt agonist to form an expanded population of cells in the cochlear tissue.
Embodiment 1511. The method of any of the above enumerated embodiments wherein reduced auditory function is prevented by contacting a Lgr5+ cochlear cell with an TAZ activator and a Wnt agonist to form an expanded population of cells in the cochlear tissue.
Embodiment 1512. The method of any of the above enumerated embodiments wherein hearing loss is treated by administering to the subject an TAZ activator and a Wnt agonist to form an expanded population of cells in the cochlear tissue.
Embodiment 1513. The method of any of the above enumerated embodiments wherein reduced auditory function is treated by administering to the subject an TAZ activator and a Wnt agonist to form an expanded population of cells in the cochlear tissue.
Embodiment 1514. The method of any of the above enumerated embodiments wherein hearing loss is prevented by administering to the subject an TAZ activator and a Wnt agonist to form an expanded population of cells in the cochlear tissue.
Embodiment 1515. The method of any of the above enumerated embodiments wherein reduced auditory function is prevented by administering to the subject an TAZ activator and a Wnt agonist to form an expanded population of cells in the cochlear tissue.
Embodiment 1516. The method of any of the above enumerated embodiments wherein the TAZ activator and Wnt agonist are administered to the subject systemically.
Embodiment 1517. The method of any of the above enumerated embodiments wherein the TAZ activator and Wnt agonist are administered to the subject locally.
Embodiment 1518. The method of any of the above enumerated embodiments wherein the the TAZ activator is administered locally and the Wnt agonist is administered systemically.
Embodiment 1519. The method of any of the above enumerated embodiments wherein the TAZ activator is administered systemically and the Wnt agonist is administered locally.
Embodiment 1520. The method of any of the above enumerated embodiments wherein the TAZ activator and Wnt agonist and the epigenetic agent are administered to the subject systemically.
Embodiment 1521. The method of any of the above enumerated embodiments wherein the TAZ activator and Wnt agonist and the epigenetic agent are administered to the subject locally.
Embodiment 1522. The method of any of the above enumerated embodiments wherein the TAZ activator and Wnt agonist are administered to the subject systemically.
Embodiment 1523. The method of any of the above enumerated embodiments wherein the TAZ activator and Wnt agonist are administered to the subject locally.
Embodiment 1524. The method of any of the above enumerated embodiments wherein the TAZ activator and Wnt agonist and one or more epigenetic agents are administered to the subject systemically.
Embodiment 1525. The method of any of the above enumerated embodiments wherein the TAZ activator and Wnt agonist and one or more epigenetic agents are administered to the subject locally.
Embodiment 1526. The method of any of the above enumerated embodiments wherein the composition is administered orally,
Embodiment 1527. The method of any of the above enumerated embodiments wherein the composition is administered parenterally.
Embodiment 1528. The method of any of the above enumerated embodiments wherein the parenteral administration route is intramuscular (IM).
Embodiment 1529. The method of any of the above enumerated embodiments wherein the parenteral administration route is subcutaneous (SC).
Embodiment 1530. The method of any of the above enumerated embodiments wherein the parenteral administration route is intravenous (IV).
Embodiment 1531. The method of any of the above enumerated embodiments wherein the local administration route is intratympanic.
Embodiment 1532. The method of any of the above enumerated embodiments wherein the local administration route is intracochlear.
Embodiment 1533. The method of any of the above enumerated embodiments wherein the TAZ activator and Wnt agonist are administered at the same time.
Embodiment 1534. The method of any of the above enumerated embodiments wherein the TAZ activator and Wnt agonist are administered at different times.
Embodiment 1535. The method of any of the above enumerated embodiments wherein the TAZ activator is administered a period of time before the Wnt agonist.
Embodiment 1536. The method of any of the above enumerated embodiments wherein the TAZ activator is administered at a period of time after the Wnt agonist.
Embodiment 1537. The method of any of the above enumerated embodiments wherein the TAZ activator is administered 1 hour or more before the Wnt agonist.
Embodiment 1538. The method of any of the above enumerated embodiments wherein the TAZ activator is administered 2 hours or more before the Wnt agonist.
Embodiment 1539. The method of any of the above enumerated embodiments wherein the TAZ activator is administered 3 hours or more before the Wnt agonist.
Embodiment 1540. The method of any of the above enumerated embodiments wherein the TAZ activator is administered 4 hours or more before the Wnt agonist.
Embodiment 1541. The method of any of the above enumerated embodiments wherein the TAZ activator is administered 5 hours or more before the Wnt agonist.
Embodiment 1542. The method of any of the above enumerated embodiments wherein the TAZ activator is administered 6 hours or more before the Wnt agonist.
Embodiment 1543. The method of any of the above enumerated embodiments wherein the TAZ activator is administered 7 hours or more before the Wnt agonist.
Embodiment 1544. The method of any of the above enumerated embodiments wherein the TAZ activator is administered 8 hours or more before the Wnt agonist.
Embodiment 1545. The method of any of the above enumerated embodiments wherein the TAZ activator is administered 9 hours or more before the Wnt agonist.
Embodiment 1546. The method of any of the above enumerated embodiments wherein the TAZ activator is administered 10 hours or more before the Wnt agonist.
Embodiment 1547. The method of any of the above enumerated embodiments wherein the TAZ activator is administered 11 hours or more before the Wnt agonist.
Embodiment 1548. The method of any of the above enumerated embodiments wherein the TAZ activator is administered 12 hours or more before the Wnt agonist.
Embodiment 1549. The method of any of the above enumerated embodiments wherein the TAZ activator is administered 13 hours or more before the Wnt agonist.
Embodiment 1550. The method of any of the above enumerated embodiments wherein the TAZ activator is administered 14 hours or more before the Wnt agonist.
Embodiment 1551. The method of any of the above enumerated embodiments wherein the TAZ activator is administered 15 hours or more before the Wnt agonist.
Embodiment 1552. The method of any of the above enumerated embodiments wherein the TAZ activator is administered 16 hours or more before the Wnt agonist.
Embodiment 1553. The method of any of the above enumerated embodiments wherein the TAZ activator is administered 17 hours or more before the Wnt agonist.
Embodiment 1554. The method of any of the above enumerated embodiments wherein the TAZ activator is administered 18 hours or more before the Wnt agonist.
Embodiment 1555. The method of any of the above enumerated embodiments wherein the TAZ activator is administered 19 hours or more before the Wnt agonist.
Embodiment 1556. The method of any of the above enumerated embodiments wherein the TAZ activator is administered 20 hours or more before the Wnt agonist.
Embodiment 1557. The method of any of the above enumerated embodiments wherein the TAZ activator is administered 21 hours or more before the Wnt agonist.
Embodiment 1558. The method of any of the above enumerated embodiments wherein the TAZ activator is administered 22 hours or more before the Wnt agonist.
Embodiment 1559. The method of any of the above enumerated embodiments wherein the TAZ activator is administered 23 hours or more before the Wnt agonist.
Embodiment 1560. The method of any of the above enumerated embodiments wherein the TAZ activator is administered 24 hours or more before the Wnt agonist.
Embodiment 1561. The method of any of the above enumerated embodiments wherein the TAZ activator is administered 1 day or more before the Wnt agonist.
Embodiment 1562. The method of any of the above enumerated embodiments wherein the TAZ activator is administered 2 days or more before the Wnt agonist.
Embodiment 1563. The method of any of the above enumerated embodiments wherein the TAZ activator is administered 3 days or more before the Wnt agonist.
Embodiment 1564. The method of any of the above enumerated embodiments wherein the TAZ activator is administered 4 days or more before the Wnt agonist.
Embodiment 1565. The method of any of the above enumerated embodiments wherein the TAZ activator is administered 5 days or more before the Wnt agonist.
Embodiment 1566. The method of any of the above enumerated embodiments wherein the TAZ activator is administered 6 days or more before the Wnt agonist.
Embodiment 1567. The method of any of the above enumerated embodiments wherein the TAZ activator is administered 7 days or more before the Wnt agonist.
Embodiment 1568. The method of any of the above enumerated embodiments wherein the Wnt agonist is administered 1 hour or more before the TAZ activator.
Embodiment 1569. The method of any of the above enumerated embodiments wherein the Wnt agonist is administered 2 hours or more before the TAZ activator.
Embodiment 1570. The method of any of the above enumerated embodiments wherein the Wnt agonist is administered 3 hours or more before the TAZ activator.
Embodiment 1571. The method of any of the above enumerated embodiments wherein the Wnt agonist is administered 4 hours or more before the TAZ activator.
Embodiment 1572. The method of any of the above enumerated embodiments wherein the Wnt agonist is administered 5 hours or more before the TAZ activator.
Embodiment 1573. The method of any of the above enumerated embodiments wherein the Wnt agonist is administered 6 hours or more before the TAZ activator.
Embodiment 1574. The method of any of the above enumerated embodiments wherein the Wnt agonist is administered 7 hours or more before the TAZ activator.
Embodiment 1575. The method of any of the above enumerated embodiments wherein the Wnt agonist is administered 8 hours or more before the TAZ activator.
Embodiment 1576. The method of any of the above enumerated embodiments wherein the Wnt agonist is administered 9 hours or more before the TAZ activator.
Embodiment 1577. The method of any of the above enumerated embodiments wherein the Wnt agonist is administered 10 hours or more before the TAZ activator.
Embodiment 1578. The method of any of the above enumerated embodiments wherein the Wnt agonist is administered 11 hours or more before the TAZ activator.
Embodiment 1579. The method of any of the above enumerated embodiments wherein the Wnt agonist is administered 12 hours or more before the TAZ activator.
Embodiment 1580. The method of any of the above enumerated embodiments wherein the Wnt agonist is administered 13 hours or more before the TAZ activator.
Embodiment 1581. The method of any of the above enumerated embodiments wherein the Wnt agonist is administered 14 hours or more before the TAZ activator.
Embodiment 1582. The method of any of the above enumerated embodiments wherein the Wnt agonist is administered 15 hours or more before the TAZ activator.
Embodiment 1583. The method of any of the above enumerated embodiments wherein the Wnt agonist is administered 16 hours or more before the TAZ activator.
Embodiment 1584. The method of any of the above enumerated embodiments wherein the Wnt agonist is administered 17 hours or more before the TAZ activator.
Embodiment 1585. The method of any of the above enumerated embodiments wherein the Wnt agonist is administered 18 hours or more before the TAZ activator.
Embodiment 1586. The method of any of the above enumerated embodiments wherein the Wnt agonist is administered 19 hours or more before the TAZ activator.
Embodiment 1587. The method of any of the above enumerated embodiments wherein the Wnt agonist is administered 20 hours or more before the TAZ activator.
Embodiment 1588. The method of any of the above enumerated embodiments wherein the Wnt agonist is administered 21 hours or more before the TAZ activator.
Embodiment 1589. The method of any of the above enumerated embodiments wherein the Wnt agonist is administered 22 hours or more before the TAZ activator.
Embodiment 1590. The method of any of the above enumerated embodiments wherein the Wnt agonist is administered 23 hours or more before the TAZ activator.
Embodiment 1591. The method of any of the above enumerated embodiments wherein the Wnt agonist is administered 24 hours or more before the TAZ activator.
Embodiment 1592. The method of any of the above enumerated embodiments wherein the Wnt agonist is administered 1 day or more before the TAZ activator.
Embodiment 1593. The method of any of the above enumerated embodiments wherein the Wnt agonist is administered 2 days or more before the TAZ activator.
Embodiment 1594. The method of any of the above enumerated embodiments wherein the Wnt agonist is administered 3 days or more before the TAZ activator.
Embodiment 1595. The method of any of the above enumerated embodiments wherein the Wnt agonist is administered 4 days or more before the TAZ activator.
Embodiment 1596. The method of any of the above enumerated embodiments wherein the Wnt agonist is administered 5 days or more before the TAZ activator.
Embodiment 1597. The method of any of the above enumerated embodiments wherein the Wnt agonist is administered 6 days or more before the TAZ activator.
Embodiment 1598. The method of any of the above enumerated embodiments wherein the Wnt agonist is administered 7 days or more before the TAZ activator.
Embodiment 1599. The method of any of the above enumerated embodiments wherein a cochlear cell is contacted with a TAZ activator and a Wnt agonist at a “cell effective concentration” to form an expanded population of cells in the cochlear tissue.
Embodiment 1600. The method of any of the above enumerated embodiments wherein the cell effective concentration is the minimum concentration of the compound that induces at least a 1.1-fold or more increase in gene expression.
Embodiment 1601. The method of any of the above enumerated embodiments wherein the cell effective concentration is the minimum concentration of the compound that induces at least a 1.2-fold or more increase in gene expression.
Embodiment 1602. The method of any of the above enumerated embodiments wherein the cell effective concentration is the minimum concentration of the compound that induces at least a 1.3-fold or more increase in gene expression.
Embodiment 1603. The method of any of the above enumerated embodiments wherein the cell effective concentration is the minimum concentration of the compound that induces at least a 1.4-fold or more increase in gene expression.
Embodiment 1604. The method of any of the above enumerated embodiments wherein the cell effective concentration is the minimum concentration of the compound that induces at least a 1.5-fold or more increase in gene expression.
Embodiment 1605. The method of any of the above enumerated embodiments wherein the cell effective concentration is the minimum concentration of the compound that induces at least a 1.6-fold or more increase in gene expression.
Embodiment 1606. The method of any of the above enumerated embodiments wherein the cell effective concentration is the minimum concentration of the compound that induces at least a 1.7-fold or more increase in gene expression.
Embodiment 1607. The method of any of the above enumerated embodiments wherein the cell effective concentration is the minimum concentration of the compound that induces at least a 1.8-fold or more increase in gene expression.
Embodiment 1608. The method of any of the above enumerated embodiments wherein the cell effective concentration is the minimum concentration of the compound that induces at least a 1.9-fold or more increase in gene expression.
Embodiment 1609. The method of any of the above enumerated embodiments wherein the cell effective concentration is the minimum concentration of the compound that induces at least a 2-fold or more increase in gene expression.
Embodiment 1610. The method of any of the above enumerated embodiments wherein the cell effective concentration is the minimum concentration of the compound that induces at least a 3-fold or more increase in gene expression.
Embodiment 1611. The method of any of the above enumerated embodiments wherein the cell effective concentration is the minimum concentration of the compound that induces at least a 4-fold or more increase in gene expression.
Embodiment 1612. The method of any of the above enumerated embodiments wherein the cell effective concentration is the minimum concentration of the compound that induces at least a 5-fold or more increase in gene expression.
Embodiment 1613. The method of any of the above enumerated embodiments wherein the cell effective concentration is the minimum concentration of the compound that induces at least a 6-fold or more increase in gene expression.
Embodiment 1614. The method of any of the above enumerated embodiments wherein the cell effective concentration is the minimum concentration of the compound that induces at least a 7-fold or more increase in gene expression.
Embodiment 1615. The method of any of the above enumerated embodiments wherein the cell effective concentration is the minimum concentration of the compound that induces at least a 8-fold or more increase in gene expression.
Embodiment 1616. The method of any of the above enumerated embodiments wherein the cell effective concentration is the minimum concentration of the compound that induces at least a 9-fold or more increase in gene expression.
Embodiment 1617. The method of any of the above enumerated embodiments wherein the cell effective concentration is the minimum concentration of the compound that induces at least a 10-fold or more increase in gene expression.
Embodiment 1618. The method of any of the above enumerated embodiments wherein the cell effective concentration is the minimum concentration of the compound that induces at least a 15-fold or more increase in gene expression.
Embodiment 1619. The method of any of the above enumerated embodiments wherein the cell effective concentration is the minimum concentration of the compound that induces at least a 20-fold or more increase in gene expression.
Embodiment 1620. The method of any of the above enumerated embodiments wherein the cell effective concentration is the minimum concentration of the compound that induces at least a 30-fold or more increase in gene expression.
Embodiment 1621. The method of any of the above enumerated embodiments wherein the cell effective concentration is the minimum concentration of the compound that induces at least a 40-fold or more increase in gene expression.
Embodiment 1622. The method of any of the above enumerated embodiments wherein the cell effective concentration is the minimum concentration of the compound that induces at least a 50-fold or more increase in gene expression.
Embodiment 1623. The method of any of the above enumerated embodiments wherein the cell effective concentration is the minimum concentration of the compound that induces at least a 60-fold or more increase in gene expression.
Embodiment 1624. The method of any of the above enumerated embodiments wherein the cell effective concentration is the minimum concentration of the compound that induces at least a 70-fold or more increase in gene expression.
Embodiment 1625. The method of any of the above enumerated embodiments wherein the cell effective concentration is the minimum concentration of the compound that induces at least a 80-fold or more increase in gene expression.
Embodiment 1626. The method of any of the above enumerated embodiments wherein the cell effective concentration is the minimum concentration of the compound that induces at least a 90-fold or more increase in gene expression.
Embodiment 1627. The method of any of the above enumerated embodiments wherein the cell effective concentration is the minimum concentration of the compound that induces at least a 100-fold or more increase in gene expression.
Embodiment 1628. The method of any of the above enumerated embodiments wherein the cell effective concentration is the minimum concentration of the compound that induces at least a 200-fold or more increase in gene expression.
Embodiment 1629. The method of any of the above enumerated embodiments wherein the cell effective concentration is the minimum concentration of the compound that induces at least a 300-fold or more increase in gene expression.
Embodiment 1630. The method of any of the above enumerated embodiments wherein the cell effective concentration is the minimum concentration of the compound that induces at least a 400-fold or more increase in gene expression.
Embodiment 1631. The method of any of the above enumerated embodiments wherein the cell effective concentration is the minimum concentration of the compound that induces at least a 500-fold or more increase in gene expression.
Embodiment 1632. The method of any of the above enumerated embodiments wherein the cell effective concentration is the minimum concentration of the compound that induces at least a 1000-fold or more increase in gene expression.
Embodiment 1633. The method of any of the above enumerated embodiments wherein the cell effective concentration is the minimum concentration of the compound that induces about a 1.5-fold increase in number of Lgr5+ cells in a Stem Cell Proliferation Assay compared to a vehicle control.
Embodiment 1634. The method of any of the above enumerated embodiments wherein the Lgr5+ cochlear cell(s) are contacted in vitro with the compound(s) at the “cell effective concentration” in a cell culture.
Embodiment 1635. The method of any of the above enumerated embodiments wherein the Lgr5+ cochlear cell(s) are contacted with the compound(s) at the “cell effective concentration” in situ.
Embodiment 1636. The method of any of the above enumerated embodiments wherein sufficient compound is delivered to achieve the “cell effective concentration” throughout the speech region of the human cochlea.
Embodiment 1637. The method of any of the above enumerated embodiments wherein the compound is administered in a concentration higher than the “cell effective concentration” in the cochlea and diffuses throughout the speech region.
Embodiment 1638. The method of any of the above enumerated embodiments wherein the concentration of compound contacted with the Lgr5+ cochlear cells is 2-fold more than the “cell effective concentration”, in situ.
Embodiment 1639. The method of any of the above enumerated embodiments wherein the concentration of compound contacted with the Lgr5+ cochlear cells is 3-fold more than the “cell effective concentration”, in situ.
Embodiment 1640. The method of any of the above enumerated embodiments wherein the concentration of compound contacted with the Lgr5+ cochlear cells is 4-fold more than the “cell effective concentration”, in sins.
Embodiment 1641. The method of any of the above enumerated embodiments wherein the concentration of compound contacted with the Lgr5+ cochlear cells is 10-fold more than the “cell effective concentration”, in situ.
Embodiment 1642. The method of any of the above enumerated embodiments wherein the concentration of compound contacted with the Lgr5+ cochlear cells is 20-fold more than the “cell effective concentration”, in situ.
Embodiment 1643. The method of any of the above enumerated embodiments wherein the concentration of compound contacted with the Lgr5+ cochlear cells is 50-fold more than the “cell effective concentration”, in situ.
Embodiment 1644. The method of any of the above enumerated embodiments wherein the concentration of compound contacted with the Lgr5+ cochlear cells is 100-fold more than the “cell effective concentration”, in situ.
Embodiment 1645. The method of any of the above enumerated embodiments wherein the concentration of compound contacted with the Lgr5+ cochlear cells is 200-fold more than the “cell effective concentration”, in situ.
Embodiment 1646. The method of any of the above enumerated embodiments wherein the concentration of compound contacted with the Lgr5+ cochlear cells is 300-fold more than the “cell effective concentration”, in situ.
Embodiment 1647. The method of any of the above enumerated embodiments wherein the concentration of compound contacted with the Lgr5+ cochlear cells is 400-fold more than the “cell effective concentration”, in situ.
Embodiment 1648. The method of any of the above enumerated embodiments wherein the concentration of compound contacted with the Lgr5+ cochlear cells is 500-fold more than the “cell effective concentration”, in situ.
Embodiment 1649. The method of any of the above enumerated embodiments wherein the concentration of compound contacted with the Lgr5+ cochlear cells is 600-fold more than the “cell effective concentration”, in situ.
Embodiment 1650. The method of any of the above enumerated embodiments wherein the concentration of compound contacted with the Lgr5+ cochlear cells is 700-fold more than the “cell effective concentration”, in situ.
Embodiment 1651. The method of any of the above enumerated embodiments wherein the concentration of compound contacted with the Lgr5+ cochlear cells is 800-fold more than the “cell effective concentration”, in situ.
Embodiment 1652. The method of any of the above enumerated embodiments wherein the concentration of compound contacted with the Lgr5+ cochlear cells is 900-fold more than the “cell effective concentration”, in situ.
Embodiment 1653. The method of any of the above enumerated embodiments wherein the concentration of compound contacted with the Lgr5+ cochlear cells is 1000-fold more than the “cell effective concentration”, in situ.
Embodiment 1654. The method of any of the above enumerated embodiments wherein the hearing loss is treated by administering the compound(s) at the “formulation effective concentration”.
Embodiment 1655. The method of any of the above enumerated embodiments wherein the reduced auditory function is treated by administering the compound(s) at the “formulation effective concentration”.
Embodiment 1656. The method of any of the above enumerated embodiments wherein the “formulation effective concentration” is at least about 100 to 5000 fold higher than the “cell effective concentration”.
Embodiment 1657. The method of any of the above enumerated embodiments wherein the “formulation effective concentration” is at about 20-fold higher than the “cell effective concentration”.
Embodiment 1658. The method of any of the above enumerated embodiments wherein the “formulation effective concentration” is at about 100-fold higher than the “cell effective concentration”.
Embodiment 1659. The method of any of the above enumerated embodiments wherein the “formulation effective concentration” is at about 200-fold higher than the “cell effective concentration”.
Embodiment 1660. The method of any of the above enumerated embodiments wherein the “formulation effective concentration” is at about 250-fold higher than the “cell effective concentration”.
Embodiment 1661. The method of any of the above enumerated embodiments wherein the “formulation effective concentration” is at about 300-fold higher than the “cell effective concentration”.
Embodiment 1662. The method of any of the above enumerated embodiments wherein the “formulation effective concentration” is at about 400-fold higher than the “cell effective concentration”.
Embodiment 1663. The method of any of the above enumerated embodiments wherein the “formulation effective concentration” is at about 500-fold higher than the “cell effective concentration”.
Embodiment 1664. The method of any of the above enumerated embodiments wherein the “formulation effective concentration” is at about 600-fold higher than the “cell effective concentration”.
Embodiment 1665. The method of any of the above enumerated embodiments wherein the “formulation effective concentration” is at about 700-fold higher than the “cell effective concentration”.
Embodiment 1666. The method of any of the above enumerated embodiments wherein the “formulation effective concentration” is at about 750-fold higher than the “cell effective concentration”.
Embodiment 1667. The method of any of the above enumerated embodiments wherein the “formulation effective concentration” is at about 800-fold higher than the “cell effective concentration”.
Embodiment 1668. The method of any of the above enumerated embodiments wherein the “formulation effective concentration” is at about 900-fold higher than the “cell effective concentration”.
Embodiment 1669. The method of any of the above enumerated embodiments wherein the “formulation effective concentration” is at about 1000-fold higher than the “cell effective concentration”.
Embodiment 1670. The method of any of the above enumerated embodiments wherein the “formulation effective concentration” is at about 1250-fold higher than the “cell effective concentration”.
Embodiment 1671. The method of any of the above enumerated embodiments wherein the “formulation effective concentration” is at about 1500-fold higher than the “cell effective concentration”.
Embodiment 1672. The method of any of the above enumerated embodiments wherein the “formulation effective concentration” is at about 1750-fold higher than the “cell effective concentration”.
Embodiment 1673. The method of any of the above enumerated embodiments wherein the “formulation effective concentration” is at about 2000-fold higher than the “cell effective concentration”.
Embodiment 1674. The method of any of the above enumerated embodiments wherein the “formulation effective concentration” is at least about 100 to 1000 fold higher than the “cell effective concentration”.
Embodiment 1675. The method of any of the above enumerated embodiments wherein the “formulation effective concentration” is at least about 1000 fold higher than the “cell effective concentration”.
Embodiment 1676. The method of any of the above enumerated embodiments wherein the hearing loss is treated by administering the compound(s) at a set daily dose.
Embodiment 1677. The method of any of the above enumerated embodiments wherein the reduced auditory function is treated by administering the compound(s) at a set daily dose.
Embodiment 1678. The method of any of the above enumerated embodiments wherein the compounds are formulated at the “cell effective concentration”.
Embodiment 1679. The method of any of the above enumerated embodiments wherein the compounds are formulated at the “formulation effective concentration”.
Embodiment 1680. The method of any of the above enumerated embodiments wherein the “cell effective concentration” of the compound(s) is about 0.01 μM to 1000 nM.
Embodiment 1681. The method of any of the above enumerated embodiments wherein the “cell effective concentration” of the compound(s) is about 1 μM to 100 nM.
Embodiment 1682. The method of any of the above enumerated embodiments wherein the “cell effective concentration” of the compound(s) is about 10 μM to 10 nM.
Embodiment 1683. The method of any of the above enumerated embodiments wherein the “cell effective concentration” of the compound(s) is about 1 μM to 10 μM.
Embodiment 1684. The method of any of the above enumerated embodiments wherein the “cell effective concentration” of the compound(s) is about 10 nM to 100 nM.
Embodiment 1685. The method of any of the above enumerated embodiments wherein the “cell effective concentration” of the compound(s) is about 100 nM to 1000 nM.
Embodiment 1686. The method of any of the above enumerated embodiments wherein the “cell effective concentration” of the compound(s) is about 1 nM to 10 nM.
Embodiment 1687. The method of any of the above enumerated embodiments wherein the “cell effective concentration” of the compound(s) is about 0.01 μM to 1000 μM.
Embodiment 1688. The method of any of the above enumerated embodiments wherein the “cell effective concentration” of the compound(s) is about 1 μM to 100 μM.
Embodiment 1689. The method of any of the above enumerated embodiments wherein the “cell effective concentration” of the compound(s) is about 10 μM to 10 μM.
Embodiment 1690. The method of any of the above enumerated embodiments wherein the “cell effective concentration” of the compound(s) is about 1 □□ to 1 mM.
Embodiment 1691. The method of any of the above enumerated embodiments wherein the “cell effective concentration” of the compound(s) is about 10 mM to 100 mM,
Embodiment 1692. The method of any of the above enumerated embodiments wherein the “formulation effective concentration” is about 0.01 nM to 1000 μM.
Embodiment 1693. The method of any of the above enumerated embodiments wherein the “formulation effective concentration” is about 1 nM to 100 μM.
Embodiment 1694. The method of any of the above enumerated embodiments wherein the “formulation effective concentration” is about 10 nM to 10 μM.
Embodiment 1695. The method of any of the above enumerated embodiments wherein the “formulation effective concentration” is about 1 nM to 10 μM.
Embodiment 1696. The method of any of the above enumerated embodiments wherein the “formulation effective concentration” is about 10 μM to 100 μM.
Embodiment 1697. The method of any of the above enumerated embodiments wherein the “formulation effective concentration” is about 100 μM to 1000 μM.
Embodiment 1698. The method of any of the above enumerated embodiments wherein the “formulation effective concentration” is about 1 μM to 10 μM.
Embodiment 1699. The method of any of the above enumerated embodiments wherein the “formulation effective concentration” is about 0.01 mM to 1000 mM.
Embodiment 1700. The method of any of the above enumerated embodiments wherein the “formulation effective concentration” is about 1 mM to 100 mM.
Embodiment 1701. The method of any of the above enumerated embodiments wherein the “formulation effective concentration” is about 10 mM to 100 mM.
Embodiment 1702. The method of any of the above enumerated embodiments wherein the compound is administered to the subject systemically at a daily dose of about 0.01 mg to 1000 mg/day.
Embodiment 1703. The method of any of the above enumerated embodiments wherein the compound is administered to the subject systemically at a daily dose of about 0.01 mg to 500 mg/day.
Embodiment 1704. The method of any of the above enumerated embodiments wherein the compound is administered to the subject systemically at a daily dose of about 0.01 mg to 250 mg/day.
Embodiment 1705. The method of any of the above enumerated embodiments wherein the compound is administered to the subject systemically at a daily dose of about 0.01 mg to 100 mg/day.
Embodiment 1706. The method of any of the above enumerated embodiments wherein the compound is administered to the subject systemically at a daily dose of about 0.01 mg to 50 mg/day.
Embodiment 1707. The method of any of the above enumerated embodiments wherein the compound is administered to the subject systemically at a daily dose of about 0.01 mg to 25 mg/day.
Embodiment 1708. The method of any of the above enumerated embodiments wherein the compound is administered to the subject systemically at a daily dose of about 0.01 mg to 10 mg/day.
Embodiment 1709. The method of any of the above enumerated embodiments wherein the compound is administered to the subject systemically at a daily dose of about 0.01 mg to 5 mg/day.
Embodiment 1710. The method of any of the above enumerated embodiments wherein the compound is administered to the subject systemically at a daily dose of about 0.1 mg to 100 mg/day.
Embodiment 1711. The method of any of the above enumerated embodiments wherein the compound is administered to the subject systemically at a daily dose of about 0.1 mg to 50 mg/day.
Embodiment 1712. The method of any of the above enumerated embodiments wherein the compound is administered to the subject systemically at a daily dose of about 0.01 mg to 25 mg/day.
Embodiment 1713. The method of any of the above enumerated embodiments wherein the compound is administered to the subject systemically at a daily dose of about 0.01 mg to 10 mg/day.
Embodiment 1714. The method of any of the above enumerated embodiments wherein the compound is administered to the subject systemically at a daily dose of about 0.01 mg to 5 mg/day.
Embodiment 1715. The method of any of the above enumerated embodiments wherein the compound is administered to the subject systemically at a daily dose of about 0.01 mg to 2.5 mg/day.
Embodiment 1716. The method of any of the above enumerated embodiments wherein the compound is administered to the subject systemically at a daily dose of about 00.1 mg to 10 mg/day.
Embodiment 1717. The method of any of the above enumerated embodiments wherein the compound is administered to the subject systemically at a daily dose of about 0.1 mg to 5 mg/day.
Embodiment 1718. The method of any of the above enumerated embodiments wherein the compound is administered to the subject systemically at a daily dose of about 0.1 mg to 4 mg/day.
Embodiment 1719. The method of any of the above enumerated embodiments wherein the compound is administered to the subject systemically at a daily dose of about 0.01 mg to 3 mg/day.
Embodiment 1720. The method of any of the above enumerated embodiments wherein the compound is administered to the subject systemically at a daily dose of about 0.01 mg to 2 mg/day.
Embodiment 1721. The method of any of the above enumerated embodiments wherein the compound is administered to the subject systemically at a daily dose of about 1 mg to 5 mg/day.
Embodiment 1722. The method of any of the above enumerated embodiments wherein the compound is administered to the subject at a concentration ratio of about 0.001 to 10 fold relative to an FDA approved concentration.
Embodiment 1723. The method of any of the above enumerated embodiments wherein the compound is administered to the subject at a concentration ratio of about 0.1 to 50 fold relative to an FDA approved concentration.
Embodiment 1724. The method of any of the above enumerated embodiments wherein the compound is administered to the subject at a concentration ratio of about 0.1 to 5 fold relative to an FDA approved concentration.
Embodiment 1725. The method of any of the above enumerated embodiments wherein the compound is administered to the subject at a concentration ratio of about 1 to 5 fold relative to an FDA approved concentration.
Embodiment 1726. The method of any of the above enumerated embodiments wherein the compound is administered to a subject at about 0.01×, relative to an FDA approved concentration.
Embodiment 1727. The method of any of the above enumerated embodiments wherein the compound is administered to a subject at about 0.1× relative to an FDA approved concentration.
Embodiment 1728. The method of any of the above enumerated embodiments wherein the compound is administered to a subject at about 2×, relative to an FDA approved concentration.
Embodiment 1729. The method of any of the above enumerated embodiments wherein the compound is administered to a subject at about 3×, relative to an FDA approved concentration.
Embodiment 1730. The method of any of the above enumerated embodiments wherein the compound is administered to a subject at about 5×, relative to an FDA approved concentration.
Embodiment 1731. The method of any of the above enumerated embodiments wherein the compound is administered to a subject at about 10×, relative to an FDA approved concentration.
Embodiment 1732. The method of any of the above enumerated embodiments wherein the TAZ activator “cell effective concentration” is about 0.01 μM to 1000 μM.
Embodiment 1733. The method of any of the above enumerated embodiments wherein the TAZ activator “cell effective concentration” is about 0.1 μM to 100 μM.
Embodiment 1734. The method of any of the above enumerated embodiments wherein the TAZ activator “cell effective concentration” is about 1 μM to 10 μM.
Embodiment 1735. The method of any of the above enumerated embodiments wherein the TAZ activator “cell effective concentration” is about 0.01 μM to 100 μM.
Embodiment 1736. The method of any of the above enumerated embodiments wherein the TAZ activator “cell effective concentration” is about 0.1 μM to 100 μM.
Embodiment 1737. The method of any of the above enumerated embodiments wherein the TAZ activator “cell effective concentration” is about 1 μM to 10 μM.
Embodiment 1738. The method of any of the above enumerated embodiments wherein the TAZ activator “cell effective concentration” is about 10 μM to 100 μM.
Embodiment 1739. The method of any of the above enumerated embodiments wherein the TAZ activator “cell effective concentration” is about 100 μM to 1000 mM.
Embodiment 1740. The method of any of the above enumerated embodiments wherein the TAZ activator “formulation effective concentration” is about 0.01 nM to 10000 μM.
Embodiment 1741. The method of any of the above enumerated embodiments wherein the TAZ activator “formulation effective concentration” is about 0.1 nM to 1000 mM.
Embodiment 1742. The method of any of the above enumerated embodiments wherein the TAZ activator “formulation effective concentration” is about 1 nM to 10 mM.
Embodiment 1743. The method of any of the above enumerated embodiments wherein the TAZ activator “formulation effective concentration” is about 0.01 μM to 100 mM.
Embodiment 1744. The method of any of the above enumerated embodiments wherein the TAZ activator “formulation effective concentration” is about 0.1 μM to 100 mM.
Embodiment 1745. The method of any of the above enumerated embodiments wherein the TAZ activator “formulation effective concentration” is about 1 nM to 10 mM.
Embodiment 1746. The method of any of the above enumerated embodiments wherein the TAZ activator “formulation effective concentration” is about 10 nM to 100 mM.
Embodiment 1747. The method of any of the above enumerated embodiments wherein the TAZ activator “formulation effective concentration” is about 100 nM to 1000 mM.
Embodiment 1748. The method of any of the above enumerated embodiments wherein the TAZ activator is administered to a subject systemically at a daily dose of about 0.01 mg to 1000 mg/day.
Embodiment 1749. The method of any of the above enumerated embodiments wherein the TAZ activator is administered to a subject systemically at a daily dose of about 0.01 mg to 5000 mg/day.
Embodiment 1750. The method of any of the above enumerated embodiments wherein the TAZ activator is administered to a subject systemically at a daily dose of about 0.01 mg to 2500 mg/day.
Embodiment 1751. The method of any of the above enumerated embodiments wherein the TAZ activator is administered to a subject systemically at a daily dose of about 0.01 mg to 1000 mg/day.
Embodiment 1752. The method of any of the above enumerated embodiments wherein the TAZ activator is administered to a subject systemically at a daily dose of about 0.01 mg to 500 mg/day.
Embodiment 1753. The method of any of the above enumerated embodiments wherein the TAZ activator is administered to a subject systemically at a daily dose of about 0.01 mg to 250 mg/day.
Embodiment 1754. The method of any of the above enumerated embodiments wherein the TAZ activator is administered to a subject systemically at a daily dose of about 0.01 mg to 100 mg/day.
Embodiment 1755. The method of any of the above enumerated embodiments wherein the TAZ activator is administered to a subject systemically at a daily dose of about 0.01 mg to 50 mg/day.
Embodiment 1756. The method of any of the above enumerated embodiments wherein the TAZ activator is administered to a subject systemically at a daily dose of about 0.1 mg to 1000 mg/day.
Embodiment 1757. The method of any of the above enumerated embodiments wherein the TAZ activator is administered to a subject systemically at a daily dose of about 0.1 mg to 500 mg/day.
Embodiment 1758. The method of any of the above enumerated embodiments wherein the TAZ activator is administered to a subject systemically at a daily dose of about 0.01 mg to 250 mg/day.
Embodiment 1759. The method of any of the above enumerated embodiments wherein the TAZ activator is administered to a subject systemically at a daily dose of about 0.01 mg to 100 mg/day.
Embodiment 1760. The method of any of the above enumerated embodiments wherein the TAZ activator is administered to a subject systemically at a daily dose of about 0.01 mg to 50 mg/day.
Embodiment 1761. The method of any of the above enumerated embodiments wherein the TAZ activator is administered to a subject systemically at a daily dose of about 0.01 mg to 2.50 mg/day.
Embodiment 1762. The method of any of the above enumerated embodiments wherein the TAZ activator is administered to a subject systemically at a daily dose of about 0.1 mg to 100 mg/day.
Embodiment 1763. The method of any of the above enumerated embodiments wherein the TAZ activator is administered to a subject systemically at a daily dose of about 0.1 mg to 50 mg/day.
Embodiment 1764. The method of any of the above enumerated embodiments wherein the TAZ activator is administered to a subject systemically at a daily dose of about 0.1 mg to 400 mg/day.
Embodiment 1765. The method of any of the above enumerated embodiments wherein the TAZ activator is administered to a subject systemically at a daily dose of about 0.1 mg to 30 mg/day.
Embodiment 1766. The method of any of the above enumerated embodiments wherein the TAZ activator is administered to a subject systemically at a daily dose of about 0.1 mg to 20 mg/day.
Embodiment 1767. The method of any of the above enumerated embodiments wherein the TAZ activator is administered to a subject systemically at a daily dose of about 1 mg to 50 mg/day.
Embodiment 1768. The method of any of the above enumerated embodiments wherein the TAZ activator is administered in an amount sufficient to achieve a concentration of about 1 μM to 1000 μM in the perilymph fluid in the inner ear.
Embodiment 1769. The method of any of the above enumerated embodiments wherein the TAZ activator is administered in an amount sufficient to achieve a concentration of about 10 μM to 1000 μM in the perilymph fluid in the inner ear.
Embodiment 1770. The method of any of the above enumerated embodiments wherein the TAZ activator is administered in an amount sufficient to achieve a concentration of about 1 nM to 1000 μM in the perilymph fluid in the inner ear.
Embodiment 1771. The method of any of the above enumerated embodiments wherein the TAZ activator is administered in an amount sufficient to achieve a concentration of about 10 nM to 100 μM in the perilymph fluid in the inner ear.
Embodiment 1772. The method of any of the above enumerated embodiments wherein the TAZ activator is administered in an amount sufficient to achieve a concentration of about 10 nM to 10 μM in the perilymph fluid in the inner ear.
Embodiment 1773. The method of any of the above enumerated embodiments wherein the TAZ activator is administered in an amount sufficient to achieve a concentration of about 100 nM to 1000 μM in the perilymph fluid in the inner ear.
Embodiment 1774. The method of any of the above enumerated embodiments wherein the TAZ activator is administered in an amount sufficient to achieve a concentration of about 100 nM to 100 μM in the perilymph fluid in the inner ear.
Embodiment 1775. The method of any of the above enumerated embodiments wherein the TAZ activator is administered in an amount sufficient to achieve a concentration of about 100 nM to 1 μM in the perilymph fluid in the inner ear.
Embodiment 1776. The method of any of the above enumerated embodiments wherein the TAZ activator is administered in an amount sufficient to achieve a concentration of about 0.1 mM to 1000 mM in the perilymph fluid in the inner ear.
Embodiment 1777. The method of any of the above enumerated embodiments wherein the TAZ activator is administered in an amount sufficient to achieve a concentration of about 1 mM to 100 mM in the perilymph fluid in the inner ear.
Embodiment 1778. The method of any of the above enumerated embodiments wherein the TAZ activator is administered in an amount sufficient to achieve a concentration of about 0.001 nM to 1 mM in the perilymph fluid in the inner ear.
Embodiment 1779. The method of any of the above enumerated embodiments wherein the TAZ activator is administered in an amount sufficient to achieve a concentration of about 0.01 nM to 100 μM in the perilymph fluid in the inner ear.
Embodiment 1780. The method of any of the above enumerated embodiments wherein the TAZ activator is administered in an amount sufficient to achieve a concentration of about 0.1 nM to 10 μM in the perilymph fluid in the inner ear.
Embodiment 1781. The method of any of the above enumerated embodiments wherein the TAZ activator is administered in an amount sufficient to achieve a concentration of about 1 nM to 1 μM in the perilymph fluid in the inner ear.
Embodiment 1782. The method of any of the above enumerated embodiments wherein the TAZ activator is administered in an amount sufficient to achieve a concentration of about 1 nM to 10 nM in the perilymph fluid in the inner ear.
Embodiment 1783. The method of any of the above enumerated embodiments wherein the TAZ activator is administered in an amount sufficient to achieve a concentration of about 10 nM to 100 nM in the perilymph fluid in the inner ear.
Embodiment 1784. The method of any of the above enumerated embodiments wherein the TAZ activator is administered in an amount sufficient to achieve a concentration of about 100 nM to 1 μM in the perilymph fluid in the inner ear.
Embodiment 1785. The method of any of the above enumerated embodiments wherein the TAZ activator is administered in an amount sufficient to achieve a concentration of about 1 μM to 10 μM in the perilymph fluid in the inner ear.
Embodiment 1786. The method of any of the above enumerated embodiments wherein the TAZ activator is administered to a subject at about 0.1 μM.
Embodiment 1787. The method of any of the above enumerated embodiments wherein the TAZ activator is administered to a subject at about 0.2 μM.
Embodiment 1788. The method of any of the above enumerated embodiments wherein the TAZ activator is administered to a subject at about 0.3 μM.
Embodiment 1789. The method of any of the above enumerated embodiments wherein the TAZ activator is administered to a subject at about 0.4 μM.
Embodiment 1790. The method of any of the above enumerated embodiments wherein the TAZ activator is administered to a subject at about 0.5 μM.
Embodiment 1791. The method of any of the above enumerated embodiments wherein the TAZ activator is administered to a subject at about 0.6 μM.
Embodiment 1792. The method of any of the above enumerated embodiments wherein the TAZ activator is administered to a subject at about 0.7 μM.
Embodiment 1793. The method of any of the above enumerated embodiments wherein the TAZ activator is administered to a subject at about 0.8 μM.
Embodiment 1794. The method of any of the above enumerated embodiments wherein the TAZ activator is administered to a subject at about 0.9 μM.
Embodiment 1795. The method of any of the above enumerated embodiments wherein the TAZ activator is administered to a subject at about 1.0 μM.
Embodiment 1796. The method of any of the above enumerated embodiments wherein the TAZ activator is administered to a subject at about 2.0 μM.
Embodiment 1797. The method of any of the above enumerated embodiments wherein the TAZ activator is administered to a subject at about 3.0 μM.
Embodiment 1798. The method of any of the above enumerated embodiments wherein the TAZ activator is administered to a subject at about 4.0 μM.
Embodiment 1799. The method of any of the above enumerated embodiments wherein the TAZ activator is administered to a subject at about 5.0 μM.
Embodiment 1800. The method of any of the above enumerated embodiments wherein the TAZ activator is administered to a subject at about 6.0 μM.
Embodiment 1801. The method of any of the above enumerated embodiments wherein the TAZ activator is administered to a subject at about 7.0 μM.
Embodiment 1802. The method of any of the above enumerated embodiments wherein the TAZ activator is administered to a subject at about 8.0 μM.
Embodiment 1803. The method of any of the above enumerated embodiments wherein the TAZ activator is administered to a subject at about 9.0 μM.
Embodiment 1804. The method of any of the above enumerated embodiments wherein the TAZ activator is administered to a subject at about 10 μM.
Embodiment 1805. The method of any of the above enumerated embodiments wherein the TAZ activator is administered to a subject at about 20 μM.
Embodiment 1806. The method of any of the above enumerated embodiments wherein the TAZ activator is administered to a subject at about 30 μM.
Embodiment 1807. The method of any of the above enumerated embodiments wherein the TAZ activator is administered to a subject at about 40 μM.
Embodiment 1808. The method of any of the above enumerated embodiments wherein the TAZ activator is administered to a subject at about 50 μM.
Embodiment 1809. The method of any of the above enumerated embodiments wherein the TAZ activator is administered to a subject at about 60 μM.
Embodiment 1810. The method of any of the above enumerated embodiments wherein the TAZ activator is administered to a subject at about 70 μM.
Embodiment 1811. The method of any of the above enumerated embodiments wherein the TAZ activator is administered to a subject at about 80 μM.
Embodiment 1812. The method of any of the above enumerated embodiments wherein the TAZ activator is administered to a subject at about 90 μM.
Embodiment 1813. The method of any of the above enumerated embodiments wherein the TAZ activator is administered to a subject at about 100 μM.
Embodiment 1814. The method of any of the above enumerated embodiments wherein the TAZ activator is administered to a subject at about 200 μM.
Embodiment 1815. The method of any of the above enumerated embodiments wherein the TAZ activator is administered to a subject at about 300 μM.
Embodiment 1816. The method of any of the above enumerated embodiments wherein the TAZ activator is administered to a subject at about 400 μM.
Embodiment 1817. The method of any of the above enumerated embodiments wherein the TAZ activator is administered to a subject at about 500 μM.
Embodiment 1818. The method of any of the above enumerated embodiments wherein the TAZ activator is administered to a subject at about 600 μM.
Embodiment 1819. The method of any of the above enumerated embodiments wherein the TAZ activator is administered to a subject at about 700 μM.
Embodiment 1820. The method of any of the above enumerated embodiments wherein the TAZ activator is administered to a subject at about 800 μM.
Embodiment 1821. The method of any of the above enumerated embodiments wherein the TAZ activator is administered to a subject at about 900 μM.
Embodiment 1822. The method of any of the above enumerated embodiments wherein the TAZ activator is administered to a subject at about 1000 μM.
Embodiment 1823. The method of any of the above enumerated embodiments wherein the TAZ activator is administered to a subject at about 1.0 mM.
Embodiment 1824. The method of any of the above enumerated embodiments wherein the TAZ activator is administered to a subject at about 2.0 mM.
Embodiment 1825. The method of any of the above enumerated embodiments wherein the TAZ activator is administered to a subject at about 3.0 mM.
Embodiment 1826. The method of any of the above enumerated embodiments wherein the TAZ activator is administered to a subject at about 4.0 mM.
Embodiment 1827. The method of any of the above enumerated embodiments wherein the TAZ activator is administered to a subject at about 5.0 mM.
Embodiment 1828. The method of any of the above enumerated embodiments wherein the TAZ activator is administered to a subject at about 6.0 mM.
Embodiment 1829. The method of any of the above enumerated embodiments wherein the TAZ activator is administered to a subject at about 7.0 mM.
Embodiment 1830. The method of any of the above enumerated embodiments wherein the TAZ activator is administered to a subject at about 8.0 mM.
Embodiment 1831. The method of any of the above enumerated embodiments wherein the TAZ activator is administered to a subject at about 9.0 mM.
Embodiment 1832. The method of any of the above enumerated embodiments wherein the TAZ activator is administered to a subject at about 10 mM.
Embodiment 1833. The method of any of the above enumerated embodiments wherein the TAZ activator is administered to a subject at about 20 mM.
Embodiment 1834. The method of any of the above enumerated embodiments wherein the TAZ activator is administered to a subject at about 30 mM.
Embodiment 1835. The method of any of the above enumerated embodiments wherein the TAZ activator is administered to a subject at about 40 mM.
Embodiment 1836. The method of any of the above enumerated embodiments wherein the TAZ activator is administered to a subject at about 50 mM.
Embodiment 1837. The method of any of the above enumerated embodiments wherein the TAZ activator is administered to a subject at about 60 mM.
Embodiment 1838. The method of any of the above enumerated embodiments wherein the TAZ activator is administered to a subject at about 70 mM.
Embodiment 1839. The method of any of the above enumerated embodiments wherein the TAZ activator is administered to a subject at about 80 mM.
Embodiment 1840. The method of any of the above enumerated embodiments wherein the TAZ activator is administered to a subject at about 90 mM.
Embodiment 1841. The method of any of the above enumerated embodiments wherein the TAZ activator is administered to a subject at about 100 mM.
Embodiment 1842. The method of any of the above enumerated embodiments wherein the TAZ activator is administered to a subject at about 200 mM.
Embodiment 1843. The method of any of the above enumerated embodiments wherein the TAZ activator is administered to a subject at about 300 mM.
Embodiment 1844. The method of any of the above enumerated embodiments wherein the TAZ activator is administered to a subject at about 400 mM
Embodiment 1845. The method of any of the above enumerated embodiments wherein the TAZ activator is administered to a subject at about 500 mM
Embodiment 1846. The method of any of the above enumerated embodiments wherein the TAZ activator is administered to a subject at about 600 mM
Embodiment 1847. The method of any of the above enumerated embodiments wherein the TAZ activator is administered to a subject at about 700 mM.
Embodiment 1848. The method of any of the above enumerated embodiments wherein the TAZ activator is administered to a subject at about 800 mM.
Embodiment 1849. The method of any of the above enumerated embodiments wherein the TAZ activator is administered to a subject at about 900 mM.
Embodiment 1850. The method of any of the above enumerated embodiments wherein the TAZ activator is administered to a subject at about 1000 mM.
Embodiment 1851. The method of any of the above enumerated embodiments wherein the “cell effective formulation” of a Wnt agonist is about 0.01 μM to 1000 mM.
Embodiment 1852. The method of any of the above enumerated embodiments wherein the “cell effective formulation” of a Wnt agonist is about 1 μM to 100 mM.
Embodiment 1853. The method of any of the above enumerated embodiments wherein the “cell effective formulation” of a Wnt agonist is about 10 μM to 10 mM.
Embodiment 1854. The method of any of the above enumerated embodiments wherein the “cell effective formulation” of a Wnt agonist is about 1 μM to 10 μM.
Embodiment 1855. The method of any of the above enumerated embodiments wherein the “cell effective formulation” of a Wnt agonist is about 10 μM to 100 μM.
Embodiment 1856. The method of any of the above enumerated embodiments wherein the “cell effective formulation” of a Wnt agonist is about 100 μM to 1000 μM.
Embodiment 1857. The method of any of the above enumerated embodiments wherein the “cell effective formulation” of a Wnt agonist is about 1 mM to 10 mM.
Embodiment 1858. The method of any of the above enumerated embodiments wherein the “cell effective formulation” of a Wnt agonist is about 10 mM to 100 mM.
Embodiment 1859. The method of any of the above enumerated embodiments wherein the “formulation effective concentration” of a Wnt agonist is about 1000 μM to 100,000 mM.
Embodiment 1860. The method of any of the above enumerated embodiments wherein the “formulation effective concentration” of a Wnt agonist is about 10,000 μM to 10,000 mM
Embodiment 1861. The method of any of the above enumerated embodiments wherein the “formulation effective concentration” of a Wnt agonist is about 1000 μM to 10,000 μM.
Embodiment 1862. The method of any of the above enumerated embodiments wherein the “formulation effective concentration” of a Wnt agonist is about 10,000 μM to 100,000 μM.
Embodiment 1863. The method of any of the above enumerated embodiments wherein the “formulation effective concentration” of a Wnt agonist is about 100,000 μM to 1,000,000 μM.
Embodiment 1864. The method of any of the above enumerated embodiments wherein the “formulation effective concentration” of a Wnt agonist is about 1,000 mM to 10,000 mM.
Embodiment 1865. The method of any of the above enumerated embodiments wherein the “formulation effective concentration” of a Wnt agonist is about 10,000 mM to 100,000 mM.
Embodiment 1866. The method of any of the above enumerated embodiments wherein the Wnt agonist is a GSK3 inhibitor and the “cell effective formulation” of the GSK3 Inhibitor is about 0.01 μM to 1000 mM.
Embodiment 1867. The method of any of the above enumerated embodiments wherein the Wnt agonist is a GSK3 inhibitor and the “cell effective formulation” of the GSK3 Inhibitor is about 1 μM to 100 mM.
Embodiment 1868. The method of any of the above enumerated embodiments wherein the Wnt agonist is a GSK3 inhibitor and the “cell effective formulation” of the GSK3 Inhibitor is about 10 μM to 10 mM.
Embodiment 1869. The method of any of the above enumerated embodiments wherein the Wnt agonist is a GSK3 inhibitor and the “cell effective formulation” of the GSK3 Inhibitor is about 1 μM to 10 μM.
Embodiment 1870. The method of any of the above enumerated embodiments wherein the Wnt agonist is a GSK3 inhibitor and the “cell effective formulation” of the GSK3 inhibitor is about 10 μM to 100 μM.
Embodiment 1871. The method of any of the above enumerated embodiments wherein the Wnt agonist is a GSK3 inhibitor and the “cell effective formulation” of the GSK3 Inhibitor is about 100 μM to 1000 μM.
Embodiment 1872. The method of any of the above enumerated embodiments wherein the Wnt agonist is a GSK3 inhibitor and the “cell effective formulation” of the GSK3 Inhibitor is about 1 mM to 10 mM.
Embodiment 1873. The method of any of the above enumerated embodiments wherein the Wnt agonist is a GSK3 inhibitor and the “cell effective formulation” of the GSK3 Inhibitor is about 10 mM to 100 mM.
Embodiment 1874. The method of any of the above enumerated embodiments wherein the Wnt agonist is a GSK3 inhibitor.
Embodiment 1875. The method of any of the above enumerated embodiments wherein the Wnt agonist is a GSK3 inhibitor and the “formulation effective concentration” of the GSK3 inhibitor is about 10 μM to 1,000,000 mM.
Embodiment 1876. The method of any of the above enumerated embodiments wherein the Wnt agonist is a GSK3 inhibitor and the “formulation effective concentration” of the GSK3 inhibitor is about 1000 μM to 100,000 mM.
Embodiment 1877. The method of any of the above enumerated embodiments wherein the Wnt agonist is a GSK3 inhibitor and the “formulation effective concentration” of the GSK3 inhibitor is about 10,000 μM to 10,000 mM.
Embodiment 1878. The method of any of the above enumerated embodiments wherein the Wnt agonist is a GSK3 inhibitor and the “formulation effective concentration” of the GSK3 inhibitor is about 1000 μM to 10,000 μM.
Embodiment 1879. The method of any of the above enumerated embodiments wherein the Wnt agonist is a GSK3 inhibitor and the “formulation effective concentration” of the GSK3 inhibitor is about 10,000 μM to 100,000 μM.
Embodiment 1880. The method of any of the above enumerated embodiments wherein the Wnt agonist is a GSK3 inhibitor and the “formulation effective concentration” of the GSK3 inhibitor is about 100,000 μM to 1,000,000 μM.
Embodiment 1881. The method of any of the above enumerated embodiments wherein the Wnt agonist is a GSK3 inhibitor and the “formulation effective concentration” of the GSK3 inhibitor is about 1,000 mM to 10,000 mM
Embodiment 1882. The method of any of the above enumerated embodiments wherein the Wnt agonist is a GSK3 inhibitor and the “formulation effective concentration” of the GSK3 inhibitor is about 10,000 mM to 100,000 mM.
Embodiment 1883. The method of any of the above enumerated embodiments wherein the Wnt agonist is AZD1080.
Embodiment 1884. The method of any of the above enumerated embodiments wherein the Wnt agonist is GSK3-inhibitor XXII.
Embodiment 1885. The method of any of the above enumerated embodiments wherein the Wnt agonist is CHIR99021.
Embodiment 1886. The method of any of the above enumerated embodiments wherein the Wnt agonist is LY2090314.
Embodiment 1887. The method of any of the above enumerated embodiments wherein the Wnt agonist is an LY2090314 analogue.
Embodiment 1888. The method of any of the above enumerated embodiments wherein the Wnt agonist is administered to a cochlear cell.
Embodiment 1889. The method of any of the above enumerated embodiments wherein the Wnt agonist is administered at a concentration of about 0.01 uM to 1000 mM.
Embodiment 1890. The method of any of the above enumerated embodiments wherein the Wnt agonist is administered at a concentration of about 0.1 μM to 1000 mM.
Embodiment 1891. The method of any of the above enumerated embodiments wherein the Wnt agonist is administered at a concentration of about 1 μM to 100 mM.
Embodiment 1892. The method of any of the above enumerated embodiments wherein the Wnt agonist is administered at a concentration of about 10 μM to 10 mM.
Embodiment 1893. The method of any of the above enumerated embodiments wherein the Wnt agonist is administered at a concentration of about 1 μM to 10 μM
Embodiment 1894. The method of any of the above enumerated embodiments wherein the Wnt agonist is administered at a concentration of about 10 μM to 100 μM.
Embodiment 1895. The method of any of the above enumerated embodiments wherein the Wnt agonist is administered at a concentration of about 100 μM to 1000 μM.
Embodiment 1896. The method of any of the above enumerated embodiments wherein the Wnt agonist is administered at a concentration of about 1 mM to 10 mM.
Embodiment 1897. The method of any of the above enumerated embodiments wherein the Wnt agonist is administered at a concentration of about 10 mM to 100 mM.
Embodiment 1898. The method of any of the above enumerated embodiments wherein the Wnt agonist is administered at a concentration of about 0.1 pIM.
Embodiment 1899. The method of any of the above enumerated embodiments wherein the Wnt agonist is administered at a concentration of about 0.2 μM.
Embodiment 1900. The method of any of the above enumerated embodiments wherein the Wnt agonist is administered at a concentration of about 0.3 μM.
Embodiment 1901. The method of any of the above enumerated embodiments wherein the Wnt agonist is administered at a concentration of about 0.4 μM.
Embodiment 1902. The method of any of the above enumerated embodiments wherein the Wnt agonist is administered at a concentration of about 0.5 μM.
Embodiment 1903. The method of any of the above enumerated embodiments wherein the Wnt agonist is administered at a concentration of about 0.6 stM.
Embodiment 1904. The method of any of the above enumerated embodiments wherein the Wnt agonist is administered at a concentration of about 0.7 stM.
Embodiment 1905. The method of any of the above enumerated embodiments wherein the Wnt agonist is administered at a concentration of about 0.8 μM.
Embodiment 1906. The method of any of the above enumerated embodiments wherein the Wnt agonist is administered at a concentration of about 0.9 μM.
Embodiment 1907. The method of any of the above enumerated embodiments wherein the Wnt agonist is administered at a concentration of about 1 μM.
Embodiment 1908. The method of any of the above enumerated embodiments wherein the Wnt agonist is administered at a concentration of about 2 μM.
Embodiment 1909. The method of any of the above enumerated embodiments wherein the Wnt agonist is administered at a concentration of about 3 μM.
Embodiment 1910. The method of any of the above enumerated embodiments wherein the Wnt agonist is administered at a concentration of about 4 μM.
Embodiment 1911. The method of any of the above enumerated embodiments wherein the Wnt agonist is administered at a concentration of about 5 μM
Embodiment 1912. The method of any of the above enumerated embodiments wherein the Wnt agonist is administered at a concentration of about 6 μM.
Embodiment 1913. The method of any of the above enumerated embodiments wherein the Wnt agonist is administered at a concentration of about 7 μM.
Embodiment 1914. The method of any of the above enumerated embodiments wherein the Wnt agonist is administered at a concentration of about 8 μM
Embodiment 1915. The method of any of the above enumerated embodiments wherein the Wnt agonist is administered at a concentration of about 9 μM.
Embodiment 1916. The method of any of the above enumerated embodiments wherein the Wnt agonist is administered at a concentration of about 10 μM.
Embodiment 1917. The method of any of the above enumerated embodiments wherein the Wnt agonist is administered at a concentration of about 15 μM.
Embodiment 1918. The method of any of the above enumerated embodiments wherein the Wnt agonist is administered at a concentration of about 20 μM.
Embodiment 1919. The method of any of the above enumerated embodiments wherein the Wnt agonist is administered at a concentration of about 30 μM.
Embodiment 1920. The method of any of the above enumerated embodiments wherein the Wnt agonist is administered at a concentration of about 50 μM.
Embodiment 1921. The method of any of the above enumerated embodiments wherein the Wnt agonist is administered at a concentration of about 100 μM.
Embodiment 1922. The method of any of the above enumerated embodiments wherein the Wnt agonist is administered at a concentration of about 250 μM.
Embodiment 1923. The method of any of the above enumerated embodiments wherein the Wnt agonist is administered at a concentration of about 500 μM.
Embodiment 1924. The method of any of the above enumerated embodiments wherein the Wnt agonist is administered at a concentration of about 100 μM.
Embodiment 1925. The method of any of the above enumerated embodiments wherein the Wnt agonist is administered at a concentration of about 1500 μM.
Embodiment 1926. The method of any of the above enumerated embodiments wherein the Wnt agonist is administered at a concentration of about 10 μM to 1,000,000 mM.
Embodiment 1927. The method of any of the above enumerated embodiments wherein the Wnt agonist is administered at a concentration of about 1000 μM to 100,000 mM.
Embodiment 1928. The method of any of the above enumerated embodiments wherein the Wnt agonist is administered at a concentration of about 10,000 μM to 10,000 mM.
Embodiment 1929. The method of any of the above enumerated embodiments wherein the Wnt agonist is administered at a concentration of about 1000 μM to 10,000 μM.
Embodiment 1930. The method of any of the above enumerated embodiments wherein the Wnt agonist is administered at a concentration of about 10,000 μM to 100,000 μM.
Embodiment 1931. The method of any of the above enumerated embodiments wherein the Wnt agonist is administered at a concentration of about 100,000 μM to 1,000,000 μM.
Embodiment 1932. The method of any of the above enumerated embodiments wherein the Wnt agonist is administered at a concentration of about 1,000 mM to 10,000 mM.
Embodiment 1933. The method of any of the above enumerated embodiments wherein the Wnt agonist is administered at a concentration of about 10,000 mM to 100,000 mM.
Embodiment 1934. The method of any of the above enumerated embodiments wherein the Wnt agonist is administered at a concentration of about 0.1 mM.
Embodiment 1935. The method of any of the above enumerated embodiments wherein the Wnt agonist is administered at a concentration of about 0.2 mM.
Embodiment 1936. The method of any of the above enumerated embodiments wherein the Wnt agonist is administered at a concentration of about 0.3 mM.
Embodiment 1937. The method of any of the above enumerated embodiments wherein the Wnt agonist is administered at a concentration of about 0.4 mM.
Embodiment 1938. The method of any of the above enumerated embodiments wherein the Wnt agonist is administered at a concentration of about 0.5 mM.
Embodiment 1939. The method of any of the above enumerated embodiments wherein the Wnt agonist is administered at a concentration of about 0.6 mM.
Embodiment 1940. The method of any of the above enumerated embodiments wherein the Wnt agonist is administered at a concentration of about 0.7 mM.
Embodiment 1941. The method of any of the above enumerated embodiments wherein the Wnt agonist is administered at a concentration of about 0.8 mM.
Embodiment 1942. The method of any of the above enumerated embodiments wherein the Wnt agonist is administered at a concentration of about 0.9 mM.
Embodiment 1943. The method of any of the above enumerated embodiments wherein the Wnt agonist is administered at a concentration of about 1 mM.
Embodiment 1944. The method of any of the above enumerated embodiments wherein the Wnt agonist is administered at a concentration of about 2 mM.
Embodiment 1945. The method of any of the above enumerated embodiments wherein the Wnt agonist is administered at a concentration of about 3 mM.
Embodiment 1946. The method of any of the above enumerated embodiments wherein the Wnt agonist is administered at a concentration of about 4 mM.
Embodiment 1947. The method of any of the above enumerated embodiments wherein the Wnt agonist is administered at a concentration of about 5 mM.
Embodiment 1948. The method of any of the above enumerated embodiments wherein the Wnt agonist is administered at a concentration of about 6 mM.
Embodiment 1949. The method of any of the above enumerated embodiments wherein the Wnt agonist is administered at a concentration of about 7 mM.
Embodiment 1950. The method of any of the above enumerated embodiments wherein the Wnt agonist is administered at a concentration of about 8 mM.
Embodiment 1951. The method of any of the above enumerated embodiments wherein the Wnt agonist is administered at a concentration of about 9 mM.
Embodiment 1952. The method of any of the above enumerated embodiments wherein the Wnt agonist is administered at a concentration of about 10 mM.
Embodiment 1953. The method of any of the above enumerated embodiments wherein the Wnt agonist is administered at a concentration of about 15 nM.
Embodiment 1954. The method of any of the above enumerated embodiments wherein the Wnt agonist is administered at a concentration of about 20 nM.
Embodiment 1955. The method of any of the above enumerated embodiments wherein the Wnt agonist is administered at a concentration of about 0.01 μM to 1000 mM.
Embodiment 1956. The method of any of the above enumerated embodiments wherein the Wnt agonist is administered at a concentration of about 0.1 μM to 1000 mM.
Embodiment 1957. The method of any of the above enumerated embodiments wherein the Wnt agonist is administered at a concentration of about 1 μM to 100 mM.
Embodiment 1958. The method of any of the above enumerated embodiments wherein the Wnt agonist is administered at a concentration of about 10 μM to 10 mM.
Embodiment 1959. The method of any of the above enumerated embodiments wherein the Wnt agonist is administered at a concentration of about 1 μM to 10 μM.
Embodiment 1960. The method of any of the above enumerated embodiments wherein the Wnt agonist is administered at a concentration of about 10 μM to 100 μM.
Embodiment 1961. The method of any of the above enumerated embodiments wherein the Wnt agonist is administered at a concentration of about 100 μM to 1000 μM.
Embodiment 1962. The method of any of the above enumerated embodiments wherein the Wnt agonist is administered at a concentration of about 1 mM to 10 mM.
Embodiment 1963. The method of any of the above enumerated embodiments wherein the Wnt agonist is administered at a concentration of about 10 mM to 100 mM.
Embodiment 1964. The method of any of the above enumerated embodiments wherein the Wnt agonist is administered at a concentration of about 1 nM.
Embodiment 1965. The method of any of the above enumerated embodiments wherein the Wnt agonist is administered at a concentration of about 5 nM.
Embodiment 1966. The method of any of the above enumerated embodiments wherein the Wnt agonist is administered at a concentration of about 10 nM.
Embodiment 1967. The method of any of the above enumerated embodiments wherein the Wnt agonist is administered at a concentration of about 15 nM.
Embodiment 1968. The method of any of the above enumerated embodiments wherein the Wnt agonist is administered at a concentration of about 20 nM.
Embodiment 1969. The method of any of the above enumerated embodiments further comprising administering one or more additional epigenetic agents as described herein.
Embodiment 1970. The method of any of the above enumerated embodiments wherein the epigenetic agent is an HDAC inhibitor.
Embodiment 1971. The method of any of the above enumerated embodiments wherein the epigenetic agent is an HDAC inhibitor and “cell effective formulation” of a HDAC inhibitor is about 0.01 μM to 1000 mM.
Embodiment 1972. The method of any of the above enumerated embodiments wherein the epigenetic agent is an HDAC inhibitor and “cell effective formulation” of a HDAC inhibitor is about 1 μM to 100 mM.
Embodiment 1973. The method of any of the above enumerated embodiments wherein the epigenetic agent is an HDAC inhibitor and “cell effective formulation” of a HDAC inhibitor is about 10 μM to 10 mM.
Embodiment 1974. The method of any of the above enumerated embodiments wherein the epigenetic agent is an HDAC inhibitor and “cell effective formulation” of a HDAC inhibitor is about 1 μM to 10 μM.
Embodiment 1975. The method of any of the above enumerated embodiments wherein the epigenetic agent is an HDAC inhibitor and “cell effective formulation” of a HDAC inhibitor is about 10 μM to 100 μM.
Embodiment 1976. The method of any of the above enumerated embodiments wherein the epigenetic agent is an HDAC inhibitor and “cell effective formulation” of a HDAC inhibitor is about 100 μM to 1000 μM.
Embodiment 1977. The method of any of the above enumerated embodiments wherein the epigenetic agent is an HDAC inhibitor and “cell effective formulation” of a HDAC inhibitor is about 1 mM to 10 mM.
Embodiment 1978. The method of any of the above enumerated embodiments wherein the epigenetic agent is an HDAC inhibitor and “cell effective formulation” of a HDAC inhibitor is about 10 mM to 100 mM.
Embodiment 1979. The method of any of the above enumerated embodiments wherein the epigenetic agent is an HDAC inhibitor.
Embodiment 1980. The method of any of the above enumerated embodiments wherein the epigenetic agent is an HDAC inhibitor and the “formulation effective concentration” of an HDAC inhibitor is about 10 μM to 1,000,000 mM.
Embodiment 1981. The method of any of the above enumerated embodiments wherein the epigenetic agent is an HDAC inhibitor and the “formulation effective concentration” of an HDAC inhibitor is about 1000 μM to 100,000 mM.
Embodiment 1982. The method of any of the above enumerated embodiments wherein the epigenetic agent is an HDAC inhibitor and the “formulation effective concentration” of an HDAC inhibitor is about 10,000 μM to 10,000 mM.
Embodiment 1983. The method of any of the above enumerated embodiments wherein the epigenetic agent is an HDAC inhibitor and the “formulation effective concentration” of an HDAC inhibitor is about 1000 μM to 10,000 μM.
Embodiment 1984. The method of any of the above enumerated embodiments wherein the epigenetic agent is an HDAC inhibitor and the “formulation effective concentration” of an HDAC inhibitor is about 10,000 μM to 100,000 μM.
Embodiment 1985. The method of any of the above enumerated embodiments wherein the epigenetic agent is an HDAC inhibitor and the “formulation effective concentration” of an HDAC inhibitor is about 100,000 μM to 1,000,000 μM.
Embodiment 1986. The method of any of the above enumerated embodiments wherein the epigenetic agent is an HDAC inhibitor and the “formulation effective concentration” of an HDAC inhibitor is about 1,000 mM to 10,000 mM.
Embodiment 1987. The method of any of the above enumerated embodiments wherein the epigenetic agent is an HDAC inhibitor and the “formulation effective concentration” of an HDAC inhibitor is about 10,000 mM to 100,000 mM.
Embodiment 1988. The method of any of the above enumerated embodiments wherein the HDAC inhibitor is VPA.
Embodiment 1989. The method of any of the above enumerated embodiments wherein the HDAC inhibitor is administered at a concentration of about 10 μM to 10,000 μM.
Embodiment 1990. The method of any of the above enumerated embodiments wherein the HDAC inhibitor is administered at a concentration of about 10 mM to 10,000 mM.
Embodiment 1991. The method of any of the above enumerated embodiments wherein the HDAC inhibitor is an oral dosage form.
Embodiment 1992. The method of any of the above enumerated embodiments wherein the HDAC inhibitor is an oral dosage form with about 50 mg.
Embodiment 1993. The method of any of the above enumerated embodiments wherein the HDAC inhibitor is an oral dosage form with about 100 mg.
Embodiment 1994. The method of any of the above enumerated embodiments wherein the HDAC inhibitor is an oral dosage form with about 125 mg.
Embodiment 1995. The method of any of the above enumerated embodiments wherein the HDAC inhibitor is an oral dosage form with about 250 mg.
Embodiment 1996. The method of any of the above enumerated embodiments wherein the HDAC inhibitor is an oral dosage form with about 500 mg.
Embodiment 1997. The method of any of the above enumerated embodiments wherein the HDAC inhibitor is an oral dosage form with about 1000 mg.
Embodiment 1998. The method of any of the above enumerated embodiments wherein the HDAC inhibitor is an oral dosage form with about 2000 mg.
Embodiment 1999. The method of any of the above enumerated embodiments wherein the HDAC inhibitor is an oral dosage form with about 3000 mg.
Embodiment 2000. The method of any of the above enumerated embodiments wherein the HDAC inhibitor is an oral dosage form with about 4000 mg.
Embodiment 2001. The method of any of the above enumerated embodiments wherein the HDAC inhibitor is an oral dosage form with about 5000 mg.
Embodiment 2002. The method of any of the above enumerated embodiments comprising administering the (i) TAZ activator and (ii) Wnt agonist together in the same pharmaceutical composition.
Embodiment 2003. The method of any of the above enumerated embodiments comprising administering the (i) TAZ activator and (ii) Wnt agonist separately in separate pharmaceutical compositions.
Embodiment 2004. The method of any of the above enumerated embodiments comprising administering the (i) TAZ activator, (ii) Wnt agonist, and (iii) the additional epigenetic agent(s) together in the same pharmaceutical composition.
Embodiment 2005. The method of any of the above enumerated embodiments comprising administering the (i) TAZ activator, (ii) Wnt agonist, and (iii) the additional epigenetic agent(s) separately in separate pharmaceutical compositions.
Embodiment 2006. The method of any of the above enumerated embodiments comprising administering the (i) TAZ activator, (ii) Wnt agonist together in the same pharmaceutical composition and the (iii) epigenetic agent in a separate pharmaceutical composition.
Embodiment 2007. The method of any of the above enumerated embodiments comprising a pharmaceutically-acceptable carrier.
Embodiment 2008. The method of any of the above enumerated embodiments comprising a pharmaceutically-acceptable salt.
Embodiment 2009. The method of any of the above enumerated embodiments comprising an TAZ activator at a concentration of about 10 μM to 1,000,000 mM.
Embodiment 2010. The method of any of the above enumerated embodiments comprising TAZ activator at a concentration of about 100 μM to 1,000,000 mM.
Embodiment 2011. The method of any of the above enumerated embodiments comprising an TAZ activator at a concentration of about 1000 μM to 100,000 mM.
Embodiment 2012. The method of any of the above enumerated embodiments comprising an TAZ activator at a concentration of about 10,000 μM to 10,000 mM.
Embodiment 2013. The method of any of the above enumerated embodiments comprising an TAZ activator at a concentration of about 1000 μM to 10,000 mM.
Embodiment 2014. The method of any of the above enumerated embodiments comprising an TAZ activator at a concentration of about 10,000 μM to 100,000 μM.
Embodiment 2015. The method of any of the above enumerated embodiments comprising an TAZ activator at a concentration of about 100,000 μM to 1,000,000 μM.
Embodiment 2016. The method of any of the above enumerated embodiments comprising an TAZ activator at a concentration of about 1000 mM to 10,000 mM.
Embodiment 2017. The method of any of the above enumerated embodiments comprising an TAZ activator at a concentration of about 10,000 mM to 100,000 mM.
Embodiment 2018. The method of any of the above enumerated embodiments comprising an TAZ activator at a concentration of about 0.1 μM.
Embodiment 2019. The method of any of the above enumerated embodiments comprising an TAZ activator at a concentration of about 0.2 μM.
Embodiment 2020. The method of any of the above enumerated embodiments comprising an TAZ activator at a concentration of about 0.3 μM.
Embodiment 2021. The method of any of the above enumerated embodiments comprising an TAZ activator at a concentration of about 0.4 μM.
Embodiment 2022. The method of any of the above enumerated embodiments comprising an TAZ activator at a concentration of about 0.5 μM.
Embodiment 2023. The method of any of the above enumerated embodiments comprising an TAZ activator at a concentration of about 0.6 μM.
Embodiment 2024. The method of any of the above enumerated embodiments comprising an TAZ activator at a concentration of about 0.7 μM.
Embodiment 2025. The method of any of the above enumerated embodiments comprising an TAZ activator at a concentration of about 0.8 μM.
Embodiment 2026. The method of any of the above enumerated embodiments comprising an TAZ activator at a concentration of about 0.9 μM.
Embodiment 2027. The method of any of the above enumerated embodiments comprising an TAZ activator at a concentration of about 1.0 μM.
Embodiment 2028. The method of any of the above enumerated embodiments comprising an TAZ activator at a concentration of about 2.0 μM.
Embodiment 2029. The method of any of the above enumerated embodiments comprising an TAZ activator at a concentration of about 3.0 μM.
Embodiment 2030. The method of any of the above enumerated embodiments comprising an TAZ activator at a concentration of about 4.0 μM.
Embodiment 2031. The method of any of the above enumerated embodiments comprising an TAZ activator at a concentration of about 5.0 μM.
Embodiment 2032. The method of any of the above enumerated embodiments comprising an TAZ activator at a concentration of about 6.0 μM.
Embodiment 2033. The method of any of the above enumerated embodiments comprising an TAZ activator at a concentration of about 7.0 μM.
Embodiment 2034. The method of any of the above enumerated embodiments comprising an TAZ activator at a concentration of about 8.0 μM.
Embodiment 2035. The method of any of the above enumerated embodiments comprising an TAZ activator at a concentration of about 9.0 μM.
Embodiment 2036. The method of any of the above enumerated embodiments comprising an TAZ activator at a concentration of about 10 μM.
Embodiment 2037. The method of any of the above enumerated embodiments comprising an TAZ activator at a concentration of about 20 μM.
Embodiment 2038. The method of any of the above enumerated embodiments comprising an TAZ activator at a concentration of about 30 μM.
Embodiment 2039. The method of any of the above enumerated embodiments comprising an TAZ activator at a concentration of about 40 μM.
Embodiment 2040. The method of any of the above enumerated embodiments comprising an TAZ activator at a concentration of about 50 μM.
Embodiment 2041. The method of any of the above enumerated embodiments comprising an TAZ activator at a concentration of about 60 μM.
Embodiment 2042. The method of any of the above enumerated embodiments comprising an TAZ activator at a concentration of about 70 μM.
Embodiment 2043. The method of any of the above enumerated embodiments comprising an TAZ activator at a concentration of about 80 μM.
Embodiment 2044. The method of any of the above enumerated embodiments comprising an TAZ activator at a concentration of about 90 μM.
Embodiment 2045. The method of any of the above enumerated embodiments comprising an TAZ activator at a concentration of about 100 μM.
Embodiment 2046. The method of any of the above enumerated embodiments comprising an TAZ activator at a concentration of about 200 μM.
Embodiment 2047. The method of any of the above enumerated embodiments comprising an TAZ activator at a concentration of about 300 μM.
Embodiment 2048. The method of any of the above enumerated embodiments comprising an TAZ activator at a concentration of about 400 μM.
Embodiment 2049. The method of any of the above enumerated embodiments comprising an TAZ activator at a concentration of about 500 μM.
Embodiment 2050. The method of any of the above enumerated embodiments comprising an TAZ activator at a concentration of about 600 μM.
Embodiment 2051. The method of any of the above enumerated embodiments comprising an TAZ activator at a concentration of about 700 μM.
Embodiment 2052. The method of any of the above enumerated embodiments comprising an TAZ activator at a concentration of about 800 μM.
Embodiment 2053. The method of any of the above enumerated embodiments comprising an TAZ activator at a concentration of about 900 μM.
Embodiment 2054. The method of any of the above enumerated embodiments comprising an TAZ activator at a concentration of about 1,000 μM.
Embodiment 2055. The method of any of the above enumerated embodiments wherein the TAZ activator is adapted for administration to the inner ear.
Embodiment 2056. The method of any of the above enumerated embodiments wherein the TAZ activator is adapted for administration to the middle ear.
Embodiment 2057. The method of any of the above enumerated embodiments wherein the TAZ activator is adapted for local administration to the round window membrane.
Embodiment 2058. The method of any of the above enumerated embodiments wherein the TAZ activator is adapted for intratympanic administration.
Embodiment 2059. The method of any of the above enumerated embodiments wherein the TAZ activator is adapted for transtympanic administration.
Embodiment 2060. The method of any of the above enumerated embodiments wherein the TAZ activator is adapted for administration to the cochlear tissue.
Embodiment 2061. The method of any of the above enumerated embodiments wherein the TAZ activator is adapted for administration systematically.
Embodiment 2062. The method of any of the above enumerated embodiments wherein the TAZ activator is adapted for systemic oral administration.
Embodiment 2063. The method of any of the above enumerated embodiments wherein the TAZ activator is adapted for systemic parenteral administration.
Embodiment 2064. The method of any of the above enumerated embodiments wherein the Wnt agonist is adapted for administration to the inner ear.
Embodiment 2065. The method of any of the above enumerated embodiments wherein the Wnt agonist is adapted for administration to the middle ear.
Embodiment 2066. The method of any of the above enumerated embodiments wherein the Wnt agonist is adapted for local administration to the round window membrane.
Embodiment 2067. The method of any of the above enumerated embodiments wherein the Wnt agonist is adapted for intratympanic administration.
Embodiment 2068. The method of any of the above enumerated embodiments wherein the Wnt agonist is adapted for transtympanic administration.
Embodiment 2069. The method of any of the above enumerated embodiments wherein the Wnt agonist is adapted for administration to the cochlear tissue.
Embodiment 2070. The method of any of the above enumerated embodiments wherein the Wnt agonist is adapted for administration systematically.
Embodiment 2071. The method of any of the above enumerated embodiments wherein the Wnt agonist is adapted for systemic oral administration.
Embodiment 2072. The method of any of the above enumerated embodiments wherein Wnt agonist is adapted for systemic parenteral administration.
Embodiment 2073. The method of any of the above enumerated embodiments wherein the agent(s) are administered at a unit dose of about 25 μl to 500 μl.
Embodiment 2074. The method of any of the above enumerated embodiments wherein the agent(s) are administered at a unit dose of about 50 μl to 200 μl.
Embodiment 2075. The method of any of the above enumerated embodiments wherein the dose is administered to the inner ear.
Embodiment 2076. The method of any of the above enumerated embodiments wherein the dose is administered to the middle ear.
Embodiment 2077. The method of any of the above enumerated embodiments wherein the pharmaceutically-acceptable carrier is a sugar.
Embodiment 2078. The method of any of the above enumerated embodiments wherein the pharmaceutically-acceptable carrier is starch.
Embodiment 2079. The method of any of the above enumerated embodiments wherein the pharmaceutically-acceptable carrier is cellulose.
Embodiment 2080. The method of any of the above enumerated embodiments wherein the pharmaceutically-acceptable carrier is tragacanth.
Embodiment 2081. The method of any of the above enumerated embodiments wherein the pharmaceutically-acceptable carrier is malt.
Embodiment 2082. The method of any of the above enumerated embodiments wherein the pharmaceutically-acceptable carrier is gelatin.
Embodiment 2083. The method of any of the above enumerated embodiments wherein the pharmaceutically-acceptable carrier is talc.
Embodiment 2084. The method of any of the above enumerated embodiments wherein the pharmaceutically-acceptable carrier is cocoa butter.
Embodiment 2085. The method of any of the above enumerated embodiments wherein the pharmaceutically-acceptable carrier is a wax.
Embodiment 2086. The method of any of the above enumerated embodiments wherein the pharmaceutically-acceptable carrier is an oill.
Embodiment 2087. The method of any of the above enumerated embodiments wherein the pharmaceutically-acceptable carrier is a glycol.
Embodiment 2088. The method of any of the above enumerated embodiments wherein the pharmaceutically-acceptable carrier is a polyol.
Embodiment 2089. The method of any of the above enumerated embodiments wherein the pharmaceutically-acceptable carrier is an ester.
Embodiment 2090. The method of any of the above enumerated embodiments wherein the pharmaceutically-acceptable carrier is agar.
Embodiment 2091. The method of any of the above enumerated embodiments wherein the pharmaceutically-acceptable carrier is a buffering agent.
Embodiment 2092. The method of any of the above enumerated embodiments wherein the pharmaceutically-acceptable carrier is alginic acid.
Embodiment 2093. The method of any of the above enumerated embodiments wherein the pharmaceutically-acceptable carrier is pyrogen-free water.
Embodiment 2094. The method of any of the above enumerated embodiments wherein the pharmaceutically-acceptable carrier is isotonic saline.
Embodiment 2095. The method of any of the above enumerated embodiments wherein the pharmaceutically-acceptable carrier is Ringer's solution.
Embodiment 2096. The method of any of the above enumerated embodiments wherein the pharmaceutically-acceptable carrier is ethyl alcohol.
Embodiment 2097. The method of any of the above enumerated embodiments wherein the pharmaceutically-acceptable carrier is a phosphate buffer solution.
Embodiment 2098. The method of any of the above enumerated embodiments wherein the pharmaceutically-acceptable carrier is any other compatible substance employed in pharmaceutical formulations.
Embodiment 2099. The method of any one of the above enumerated embodiments wherein the composition comprises at least one biocompatible matrix.
Embodiment 2100. The method of any one of the above enumerated embodiments wherein the biocompatible matrix is a biocompatible gel.
Embodiment 2101. The method of any one of the above enumerated embodiments wherein the biocompatible matrix is a biocompatible foam.
Embodiment 2102. The method of any one of the above enumerated embodiments wherein the biocompatible matrix is a biocompatible fiber.
Embodiment 2103. The method of any one of the above enumerated embodiments wherein the biocompatible matrix is a biocompatible film.
Embodiment 2104. The method of any one of the above enumerated embodiments wherein the biocompatible matrix is a biocompatible mat.
Embodiment 2105. The method of any one of the above enumerated embodiments wherein the biocompatible matrix is derived from silk.
Embodiment 2106. The method of any of the above enumerated embodiments wherein the polymer used for formulating the biologically active composition is a polyamide.
Embodiment 2107. The method of any of the above enumerated embodiments wherein the polymer used for formulating the biologically active composition is a polycarbonate.
Embodiment 2108. The method of any of the above enumerated embodiments wherein the polymer used for formulating the biologically active composition is a polyalkene (polyethylene glycol (PEG)).
Embodiment 2109. The method of any of the above enumerated embodiments wherein the polymer used for formulating the biologically active composition is a polymer of acrylic esters.
Embodiment 2110. The method of any of the above enumerated embodiments wherein the polymer used for formulating the biologically active composition is a polymer of methacrylic esters.
Embodiment 2111. The method of any of the above enumerated embodiments wherein the polymer used for formulating the biologically active composition is a polyvinyl polymer.
Embodiment 2112. The method of any of the above enumerated embodiments wherein the polymer used for formulating the biologically active composition is a polyglycolide.
Embodiment 2113. The method of any of the above enumerated embodiments wherein the polymer used for formulating the biologically active composition is a polysiloxane.
Embodiment 2114. The method of any of the above enumerated embodiments wherein the polymer used for formulating the biologically active composition is a polyurethane.
Embodiment 2115. The method of any of the above enumerated embodiments wherein the polymer used for formulating the biologically active composition is a polyurethane and co-polymers thereof.
Embodiment 2116. The method of any of the above enumerated embodiments wherein the polymer used for formulating the biologically active composition is celluloses.
Embodiment 2117. The method of any of the above enumerated embodiments wherein the polymer used for formulating the biologically active composition is polypropylene.
Embodiment 2118. The method of any of the above enumerated embodiments wherein the polymers used for formulating the biologically active composition are polyethylenes.
Embodiment 2119. The method of any of the above enumerated embodiments wherein the polymer used for formulating the biologically active composition is polystyrene.
Embodiment 2120. The method of any of the above enumerated embodiments wherein the polymers used for formulating the biologically active composition are polymers of lactic acid and glycolic acid.
Embodiment 2121. The method of any of the above enumerated embodiments wherein the polymers used for formulating the biologically active composition are polyanhydrides.
Embodiment 2122. The method of any of the above enumerated embodiments wherein the polymers used for formulating the biologically active composition are poly(ortho)esters.
Embodiment 2123. The method of any of the above enumerated embodiments wherein the polymer used for formulating the biologically active composition is poly(butic acid).
Embodiment 2124. The method of any of the above enumerated embodiments wherein the polymer used for formulating the biologically active composition is poly(valeric acid).
Embodiment 2125. The method of any of the above enumerated embodiments wherein the polymer used for formulating the biologically active composition is poly(lactide-co-capralactone).
Embodiment 2126. The method of any of the above enumerated embodiments wherein the polymers used for formulating the biologically active composition are polysaccharides.
Embodiment 2127. The method of any of the above enumerated embodiments wherein the polymer used for formulating the biologically active composition is a protein.
Embodiment 2128. The method of any of the above enumerated embodiments wherein the polymers used for formulating the biologically active composition are polyhyauronic acids.
Embodiment 2129. The method of any of the above enumerated embodiments wherein the polymers used for formulating the biologically active composition are polycyanoacrylates.
Embodiment 2130. The method of any of the above enumerated embodiments wherein the polymer used for formulating the biologically active composition is a blend of polymers.
Embodiment 2131. The method of any of the above enumerated embodiments wherein the polymer used for formulating the biologically active composition is a mixture of polymers.
Embodiment 2132. The method of any of the above enumerated embodiments wherein the polymer used for formulating the biologically active composition is a copolymer.
Embodiment 2133. The method of any of the above enumerated embodiments wherein the polymer is in a concentration of about 5 wt % and about 25 wt % relative to the composition.
Embodiment 2134. The method of any of the above enumerated embodiments wherein the polymer is in a concentration of about 5 wt % relative to the composition.
Embodiment 2135. The method of any of the above enumerated embodiments wherein the polymer is in a concentration of about 6 wt % relative to the composition.
Embodiment 2136. The method of any of the above enumerated embodiments wherein the polymer is in a concentration of about 7 wt % relative to the composition.
Embodiment 2137. The method of any of the above enumerated embodiments wherein the polymer is in a concentration of about 8 wt % relative to the composition.
Embodiment 2138. The method of any of the above enumerated embodiments wherein the polymer is in a concentration of about 9 wt % relative to the composition.
Embodiment 2139. The method of any of the above enumerated embodiments wherein the polymer is in a concentration of about 10 wt % relative to the composition.
Embodiment 2140. The method of any of the above enumerated embodiments wherein the polymer is in a concentration of about 11 wt % relative to the composition.
Embodiment 2141. The method of any of the above enumerated embodiments wherein the polymer is in a concentration of about 12 wt % relative to the composition.
Embodiment 2142. The method of any of the above enumerated embodiments wherein the polymer is in a concentration of about 13 wt % relative to the composition.
Embodiment 2143. The method of any of the above enumerated embodiments wherein the polymer is in a concentration of about 14 wt % relative to the composition.
Embodiment 2144. The method of any of the above enumerated embodiments wherein the polymer is in a concentration of about 15 wt % relative to the composition.
Embodiment 2145. The method of any of the above enumerated embodiments wherein the polymer is in a concentration of about 16 wt % relative to the composition.
Embodiment 2146. The method of any of the above enumerated embodiments wherein the polymer is in a concentration of about 17 wt % relative to the composition.
Embodiment 2147. The method of any of the above enumerated embodiments wherein the polymer is in a concentration of about 18 wt % relative to the composition.
Embodiment 2148. The method of any of the above enumerated embodiments wherein the polymer is in a concentration of about 19 wt % relative to the composition.
Embodiment 2149. The method of any of the above enumerated embodiments wherein the polymer is in a concentration of about 20 wt % relative to the composition.
Embodiment 2150. The method of any of the above enumerated embodiments wherein the polymer is in a concentration of about 21 wt % relative to the composition.
Embodiment 2151. The method of any of the above enumerated embodiments wherein the polymer is in a concentration of about 22 wt % relative to the composition.
Embodiment 2152. The method of any of the above enumerated embodiments wherein the polymer is in a concentration of about 23 wt % relative to the composition.
Embodiment 2153. The method of any of the above enumerated embodiments wherein the polymer is in a concentration of about 24 wt % relative to the composition.
Embodiment 2154. The method of any of the above enumerated embodiments wherein the polymer is in a concentration of about 25 wt % relative to the composition.
Embodiment 2155. The method of any of the above enumerated embodiments wherein the polymer is in a concentration of about 10 wt % to about 23 wt % relative to the composition.
Embodiment 2156. The method of any of the above enumerated embodiments wherein the polymer is in a concentration of about 15 wt % to about 20 wt % relative to the composition.
Embodiment 2157. The method of any of the above enumerated embodiments wherein the polymer is in a concentration of approximately 17 wt % relative to the composition.
Embodiment 2158. The method of any of the above enumerated embodiments wherein the copolymer is in a concentration between about 5 wt % and about 25 wt % relative to the composition.
Embodiment 2159. The method of any of the above enumerated embodiments wherein the copolymer is in a concentration of about 5 wt % relative to the composition.
Embodiment 2160. The method of any of the above enumerated embodiments wherein the copolymer is in a concentration of about 6 wt % relative to the composition.
Embodiment 2161. The method of any of the above enumerated embodiments wherein the copolymer is in a concentration of about 7 wt % relative to the composition.
Embodiment 2162. The method of any of the above enumerated embodiments wherein the copolymer is in a concentration of about 8 wt % relative to the composition.
Embodiment 2163. The method of any of the above enumerated embodiments wherein the copolymer is in a concentration of about 9 wt % relative to the composition.
Embodiment 2164. The method of any of the above enumerated embodiments wherein the copolymer is in a concentration of about 10 wt % relative to the composition.
Embodiment 2165. The method of any of the above enumerated embodiments wherein the copolymer is in a concentration of about 11 wt % relative to the composition.
Embodiment 2166. The method of any of the above enumerated embodiments wherein the copolymer is in a concentration of about 12 wt % relative to the composition.
Embodiment 2167. The method of any of the above enumerated embodiments wherein the copolymer is in a concentration of about 13 wt % relative to the composition.
Embodiment 2168. The method of any of the above enumerated embodiments wherein the copolymer is in a concentration of about 14 wt % relative to the composition.
Embodiment 2169. The method of any of the above enumerated embodiments wherein the copolymer is in a concentration of about 15 wt % relative to the composition.
Embodiment 2170. The method of any of the above enumerated embodiments wherein the copolymer is in a concentration of about 16 wt % relative to the composition.
Embodiment 2171. The method of any of the above enumerated embodiments wherein the copolymer is in a concentration of about 17 wt % relative to the composition.
Embodiment 2172. The method of any of the above enumerated embodiments wherein the copolymer is in a concentration of about 18 wt % relative to the composition.
Embodiment 2173. The method of any of the above enumerated embodiments wherein the copolymer is in a concentration of about 19 wt % relative to the composition.
Embodiment 2174. The method of any of the above enumerated embodiments wherein the copolymer is in a concentration of about 20 wt % relative to the composition.
Embodiment 2175. The method of any of the above enumerated embodiments wherein the copolymer is in a concentration of about 21 wt % relative to the composition.
Embodiment 2176. The method of any of the above enumerated embodiments wherein the copolymer is in a concentration of about 22 wt %
Embodiment 2177. The method of any of the above enumerated embodiments wherein the copolymer is in a concentration of about 23 wt % relative to the composition.
Embodiment 2178. The method of any of the above enumerated embodiments wherein the copolymer is in a concentration of about 24 wt % relative to the composition.
Embodiment 2179. The method of any of the above enumerated embodiments wherein the copolymer is in a concentration of about 25 wt % relative to the composition.
Embodiment 2180. The method of any of the above enumerated embodiments wherein the copolymer is in a concentration between about 10 wt % to about 23 wt % relative to the composition.
Embodiment 2181. The method of any of the above enumerated embodiments wherein the copolymer is in a concentration between about 15 wt % to about 20 wt % relative to the composition.
Embodiment 2182. The method of any of the above enumerated embodiments wherein the copolymer is in a concentration of approximately 17 wt % relative to the composition.
Embodiment 2183. The method of any of the above embodiments wherein the compositions comprise at least one poloxamer.
Embodiment 2184. The method of any of the above enumerated embodiments wherein the polaxamer comprises Polaxamer 124.
Embodiment 2185. The method of any of the above enumerated embodiments wherein the polaxamer comprises Polaxamer 188.
Embodiment 2186. The method of any of the above enumerated embodiments wherein the polaxamer comprises Polaxamer 237.
Embodiment 2187. The method of any of the above enumerated embodiments wherein the polaxamer comprises Polaxamer 338.
Embodiment 2188. The method of any of the above enumerated embodiments wherein the polaxamer comprises Polaxamer 407.
Embodiment 2189. The method of any of the above enumerated embodiments wherein the polaxamer comprises Polaxamer 407 and Poloxamer 124.
Embodiment 2190. The method of any of the above enumerated embodiments wherein the polaxamer comprises at least one of Polaxamer 188 and Poloxamer 407.
Embodiment 2191. The method of any of the above enumerated embodiments wherein the polaxamer is in a concentration between about 5 wt % and about 25 wt % relative to the composition.
Embodiment 2192. The method of any of the above enumerated embodiments wherein the polaxamer is in a concentration of about 5 wt % relative to the composition.
Embodiment 2193. The method of any of the above enumerated embodiments wherein the polaxamer is in a concentration of about 6 wt % relative to the composition.
Embodiment 2194. The method of any of the above enumerated embodiments wherein the polaxamer is in a concentration of about 7 wt % relative to the composition.
Embodiment 2195. The method of any of the above enumerated embodiments wherein the polaxamer is in a concentration of about 8 wt % relative to the composition.
Embodiment 2196. The method of any of the above enumerated embodiments wherein the polaxamer is in a concentration of about 9 wt % relative to the composition.
Embodiment 2197. The method of any of the above enumerated embodiments wherein the polaxamer is in a concentration of about 10 wt % relative to the composition.
Embodiment 2198. The method of any of the above enumerated embodiments wherein the polaxamer is in a concentration of about 11 wt % relative to the composition.
Embodiment 2199. The method of any of the above enumerated embodiments wherein the polaxamer is in a concentration of about 12 wt % relative to the composition.
Embodiment 2200. The method of any of the above enumerated embodiments wherein the polaxamer is in a concentration of about 13 wt % relative to the composition.
Embodiment 2201. The method of any of the above enumerated embodiments wherein the polaxamer is in a concentration of about 14 wt % relative to the composition.
Embodiment 2202. The method of any of the above enumerated embodiments wherein the polaxamer is in a concentration of about 15 wt % relative to the composition.
Embodiment 2203. The method of any of the above enumerated embodiments wherein the polaxamer is in a concentration of about 16 wt % relative to the composition.
Embodiment 2204. The method of any of the above enumerated embodiments wherein the polaxamer is in a concentration of about 17 wt % relative to the composition.
Embodiment 2205. The method of any of the above enumerated embodiments wherein the polaxamer is in a concentration of about 18 wt % relative to the composition.
Embodiment 2206. The method of any of the above enumerated embodiments wherein the polaxamer is in a concentration of about 19 wt % relative to the composition.
Embodiment 2207. The method of any of the above enumerated embodiments wherein the polaxamer is in a concentration of about 20 wt % relative to the composition.
Embodiment 2208. The method of any of the above enumerated embodiments wherein the polaxamer is in a concentration of about 21 wt % relative to the composition.
Embodiment 2209. The method of any of the above enumerated embodiments wherein the polaxamer is in a concentration of about 22 wt % relative to the composition.
Embodiment 2210. The method of any of the above enumerated embodiments wherein the polaxamer is in a concentration of about 23 wt % relative to the composition.
Embodiment 2211. The method of any of the above enumerated embodiments wherein the polaxamer is in a concentration of about 24 wt % relative to the composition.
Embodiment 2212. The method of any of the above enumerated embodiments wherein the polaxamer is in a concentration of about 25 wt % relative to the composition.
Embodiment 2213. The method of any of the above enumerated embodiments wherein the polaxamer is in a concentration between about 10 wt % to about 23 wt % relative to the composition.
Embodiment 2214. The method of any of the above enumerated embodiments wherein the polaxamer is in a concentration between about 15 wt % to about 20 wt % relative to the composition.
Embodiment 2215. The method of any of the above enumerated embodiments wherein the polaxamer concentration is approximately 17 wt % relative to the composition.
Embodiment 2216. The method of any of the above enumerated embodiments comprising a wetting agent.
Embodiment 2217. The method of any of the above enumerated embodiments comprising an emulsifier.
Embodiment 2218. The method of any of the above enumerated embodiments comprising a lubricant.
Embodiment 2219. The method of any of the above enumerated embodiments wherein the lubricant is sodium lauryl sulfate.
Embodiment 2220. The method of any of the above enumerated embodiments wherein the lubricant is magnesium stearate.
Embodiment 2221. The method of any of the above enumerated embodiments comprising a coloring agent.
Embodiment 2222. The method of any of the above enumerated embodiments comprising a release agent.
Embodiment 2223. The method of any of the above enumerated embodiments comprising a coating agent.
Embodiment 2224. The method of any of the above enumerated embodiments comprising a sweetening agent.
Embodiment 2225. The method of any of the above enumerated embodiments comprising a flavoring agent.
Embodiment 2226. The method of any of the above enumerated embodiments comprising a perfuming agent.
Embodiment 2227. The method of any of the above enumerated embodiments comprising a preservative.
Embodiment 2228. The method of any of the above enumerated embodiments comprising an antioxidant.
Embodiment 2229. The method of any of the above enumerated embodiments comprising at least one antioxidant.
Embodiment 2230. The method of any of the above enumerated embodiments comprising water soluble antioxidants.
Embodiment 2231. The method of any of the above enumerated embodiments comprising oil-soluble antioxidants, such as ascorbyl palmitate, butylated hydroxyanisole (BHA), butylated hydroxytoluene (BHT), lecithin, propyl gallate, and alpha-tocopherol.
Embodiment 2232. The method of any of the above enumerated embodiments comprising metal chelating agents, such as citric acid, ethylenediamine tetraacetic acid (EDTA), sorbitol, tartaric acid, and phosphoric acid.
Embodiment 2233. The method of any of the above enumerated embodiments wherein the viscosity of the composition at about body temperature is surprisingly lesser than the viscosity of the composition at room temperature.
Embodiment 2234. The method of any of the above enumerated embodiments wherein the viscosity of the composition at about body temperature is surprisingly greater than the viscosity of the composition at room temperature.
Embodiment 2235. The method of any of the above enumerated embodiments comprising a buffer.
Embodiment 2236. The method of any of the above enumerated embodiments comprising a physiological saline buffer.
Embodiment 2237. The method of any of the above enumerated embodiments comprising a phosphate-buffered saline (PBS) buffer.
Embodiment 2238. The method of any of the above enumerated embodiments wherein the composition is at or near physiological pH.
Embodiment 2239. The method of any of the above enumerated embodiments wherein the composition has a pH of between about 6 and about 8, including all integers, decimals, and ranges in between.
Embodiment 2240. The method of any of the above enumerated embodiments wherein the composition has a pH of about 7.4 (f0.2).
Embodiment 2241. The method of any of the above enumerated embodiments wherein the compounds or compositions described herein can be formulated in any manner suitable for a desired delivery route.
Embodiment 2242. The method of any of the above enumerated embodiments wherein the compounds or compositions described herein can be formulated in any manner suitable for transtympanic injection.
Embodiment 2243. The method of any of the above enumerated embodiments wherein the compounds or compositions described herein can be formulated in any manner suitable for transtympanic wicks.
Embodiment 2244. The method of any of the above enumerated embodiments wherein the compounds or compositions described herein can be formulated in any manner suitable for catheters.
Embodiment 2245. The method of any of the above enumerated embodiments wherein the compounds or compositions described herein can be formulated in any manner suitable for cochlear implants.
Embodiment 2246. The method of any of the above enumerated embodiments wherein the compounds or compositions described herein can be formulated in any manner suitable for injectable depots.
Embodiment 2247. The method of any of the above enumerated embodiments wherein the compositions or formulations include one or more physiologically-acceptable components.
Embodiment 2248. The method of any of the above enumerated embodiments wherein the compositions or formulations include derivatives.
Embodiment 2249. The method of any of the above enumerated embodiments wherein the compositions or formulations include prodrugs.
Embodiment 2250. The method of any of the above enumerated embodiments wherein the compositions or formulations include solvates.
Embodiment 2251. The method of any of the above enumerated embodiments wherein the compositions or formulations include stereoisomers.
Embodiment 2252. The method of any of the above enumerated embodiments wherein the compositions or formulations include racemates.
Embodiment 2253. The method of any of the above enumerated embodiments wherein the compositions or formulations include tautomers.
Embodiment 2254. The method of any of the above enumerated embodiments wherein the compositions or formulations include physiologically acceptable carriers.
Embodiment 2255. The method of any of the above enumerated embodiments wherein the compositions or formulations include diluents.
Embodiment 2256. The method of any of the above enumerated embodiments wherein the compositions or formulations include excipients.
Embodiment 2257. The method of any of the above enumerated embodiments wherein the compositions are adapted for administration to the middle ear.
Embodiment 2258. The method of any of the above enumerated embodiments wherein the compositions are adapted for administration to the inner ear.
Embodiment 2259. The method of any of the above enumerated embodiments wherein the compositions are adapted for local administration to the round window membrane.
Embodiment 2260. The method of any of the above enumerated embodiments wherein the drug can operatively be placed locally to the round window membrane and can then penetrate through the round window membrane.
Embodiment 2261. The method of any of the above enumerated embodiments wherein the drug can operatively be placed locally to the round window membrane by injection through the tympanic membrane.
Embodiment 2262. The method of any of the above enumerated embodiments wherein the compositions or formulations may also contain a membrane penetration enhancer.
Embodiment 2263. The method of any of the above enumerated embodiments wherein liquid formulations are used.
Embodiment 2264. The method of any of the above enumerated embodiments wherein gel formulations are used.
Embodiment 2265. The method of any of the above enumerated embodiments wherein foam formulations are used.
Embodiment 2266. The method of any of the above enumerated embodiments wherein the active ingredient is applied orally.
Embodiment 2267. The method of any of the above enumerated embodiments wherein the active ingredient is applied by employing a combination of delivery approaches.
Embodiment 2268. The method of any of the above enumerated embodiments wherein the compositions are adapted intratympanic administration.
Embodiment 2269. The method of any of the above enumerated embodiments wherein the compositions are adapted transtympanic administration.
Embodiment 2270. The method of any of the above enumerated embodiments wherein the injection approach is by osmotic pump.
Embodiment 2271. The method of any of the above enumerated embodiments wherein the injection approach is by combination with implanted biomaterial.
Embodiment 2272. The method of any of the above enumerated embodiments wherein the injection approach is by injection.
Embodiment 2273. The method of any of the above enumerated embodiments wherein the injection approach is by infusion.
Embodiment 2274. The method of any of the above enumerated embodiments wherein the biomaterial can aid in controlling release kinetics.
Embodiment 2275. The method of any of the above enumerated embodiments wherein the biomaterial can aid in distribution of drug.
Embodiment 2276. The method of any of the above enumerated embodiments wherein the biomaterial is a hydrogel material.
Embodiment 2277. The method of any of the above enumerated embodiments wherein the biomaterial is a degradable material.
Embodiment 2278. The method of any of the above enumerated embodiments wherein the biomaterial is an in situ gelling material.
Embodiment 2279. The method of any of the above enumerated embodiments wherein the biomaterial is collagen.
Embodiment 2280. The method of any of the above enumerated embodiments wherein the biomaterial is fibrin.
Embodiment 2281. The method of any of the above enumerated embodiments wherein the biomaterial is gelatin.
Embodiment 2282. The method of any of the above enumerated embodiments wherein the biomaterial is decellularized tissue.
Embodiment 2283. The method of any of the above enumerated embodiments wherein the biomaterial is gelfoam.
Embodiment 2284. The method of any of the above enumerated embodiments wherein delivery is enhanced via alternate means.
Embodiment 2285. The method of any of the above enumerated embodiments wherein delivery is enhanced by adding agents to the delivered composition.
Embodiment 2286. The method of any of the above enumerated embodiments wherein delivery is enhanced by adding a penetration enhancer to the delivered composition.
Embodiment 2287. The method of any of the above enumerated embodiments wherein delivery is enhanced through devices.
Embodiment 2288. The method of any of the above enumerated embodiments wherein delivery is enhanced through devices via ultrasound.
Embodiment 2289. The method of any of the above enumerated embodiments wherein delivery is enhanced through devices via electroporation.
Embodiment 2290. The method of any of the above enumerated embodiments wherein delivery is enhanced through devices via high-speed jet.
Embodiment 2291. The method of any of the above enumerated embodiments wherein the agents described herein are administered in a therapeutically effective amount to a subject in need of treatment.
Embodiment 2292. The method of any of the above enumerated embodiments wherein administration of the compositions described herein can be via any of suitable route of administration.
Embodiment 2293. The method of any of the above enumerated embodiments wherein administration of the compositions described herein can be by intratympanic administration.
Embodiment 2294. The method of any of the above enumerated embodiments wherein administration of the compositions described herein can be by ingestion.
Embodiment 2295. The method of any of the above enumerated embodiments wherein administration of the compositions described herein can be by parental administration.
Embodiment 2296. The method of any of the above enumerated embodiments wherein administration of the compositions described herein is intravenous.
Embodiment 2297. The method of any of the above enumerated embodiments wherein administration of the compositions described herein is intra-arterial.
Embodiment 2298. The method of any of the above enumerated embodiments wherein administration of the compositions described herein is intraperitoneal.
Embodiment 2299. The method of any of the above enumerated embodiments wherein administration of the compositions described herein is intrathecal.
Embodiment 2300. The method of any of the above enumerated embodiments wherein administration of the compositions described herein is intraventricular.
Embodiment 2301. The method of any of the above enumerated embodiments wherein administration of the compositions described herein is intraurethral.
Embodiment 2302. The method of any of the above enumerated embodiments wherein administration of the compositions described herein is intrasternal.
Embodiment 2303. The method of any of the above enumerated embodiments wherein administration of the compositions described herein is intracranial.
Embodiment 2304. The method of any of the above enumerated embodiments wherein administration of the compositions described herein is intramuscular.
Embodiment 2305. The method of any of the above enumerated embodiments wherein administration of the compositions described herein is intranasal.
Embodiment 2306. The method of any of the above enumerated embodiments wherein administration of the compositions described herein is subcutaneous.
Embodiment 2307. The method of any of the above enumerated embodiments wherein administration of the compositions described herein is sublingual.
Embodiment 2308. The method of any of the above enumerated embodiments wherein administration of the compositions described herein is transdermal.
Embodiment 2309. The method of any of the above enumerated embodiments wherein administration of the compositions described herein is by inhalation.
Embodiment 2310. The method of any of the above enumerated embodiments wherein administration of the compositions described herein is by insufflations.
Embodiment 2311. The method of any of the above enumerated embodiments wherein administration of the compositions described herein is topical by ear instillation for absorption through the skin of the ear canal and membranes of the eardrum.
Embodiment 2312. The method of any of the above enumerated embodiments wherein administration of the compositions described herein is by single oral dose.
Embodiment 2313. The method of any of the above enumerated embodiments wherein administration of the compositions described herein is by multiple oral dose.
Embodiment 2314. The method of any of the above enumerated embodiments wherein administration of the compositions described herein is by a defined number of ear drops.
Embodiment 2315. The method of any of the above enumerated embodiments wherein administration of the compositions described herein is by a bolus injection.
Embodiment 2316. The method of any of the above enumerated embodiments wherein administration of the compositions described herein is by multiple injections.
Embodiment 2317. The method of any of the above enumerated embodiments wherein administration of the compositions described herein is by short duration infusion.
Embodiment 2318. The method of any of the above enumerated embodiments wherein administration of the compositions described herein is by long duration infusion.
Embodiment 2319. The method of any of the above enumerated embodiments wherein administration of the compositions described herein is by an implantable device that employs periodic parenteral delivery over time of equivalent dosages of the particular formulation.
Embodiment 2320. The method of any of the above enumerated embodiments wherein administration of the compositions described herein is by an implantable device that employs periodic parenteral delivery over time of varying dosages of the particular formulation.
Embodiment 2321. The method of any of the above enumerated embodiments wherein administration of the compositions described herein is by an implantable infusion.
Embodiment 2322. The method of any of the above enumerated embodiments wherein for such parenteral administration, the compounds are formulated as a sterile solution in water.
Embodiment 2323. The method of any of the above enumerated embodiments wherein for such parenteral administration, the compounds are formulated as a sterile solution in a suitable solvent.
Embodiment 2324. The method of any of the above enumerated embodiments wherein for such parenteral administration, the compounds are formulated as a sterile solution in a suitable mixture of solvents.
Embodiment 2325. The method of any of the above enumerated embodiments wherein for such parenteral administration, the solution may contain other substances.
Embodiment 2326. The method of any of the above enumerated embodiments wherein for such parenteral administration, the solution may contain salts.
Embodiment 2327. The method of any of the above enumerated embodiments wherein for such parenteral administration, the solution may contain sugars.
Embodiment 2328. The method of any of the above enumerated embodiments wherein for such parenteral administration, the solution may contain glucose.
Embodiment 2329. The method of any of the above enumerated embodiments wherein for such parenteral administration, the solution may contain mannitol.
Embodiment 2330. The method of any of the above enumerated embodiments wherein for such parenteral administration, the solution may contain buffering agents to make the solution isotonic with blood, such as acetic, citric, and/or phosphoric acids and their sodium salts.
Embodiment 2331. The method of any of the above enumerated embodiments wherein for such parenteral administration, the solution may contain preservatives.
Embodiment 2332. The method of any of the above enumerated embodiments wherein the compositions described herein can be administered by several methods sufficient to deliver the composition to the inner ear.
Embodiment 2333. The method of any of the above enumerated embodiments wherein the compositions described herein can be administered by direct injection through the round window to the inner ear.
Embodiment 2334. The method of any of the above enumerated embodiments wherein the compositions described herein can be administered by auricular administration.
Embodiment 2335. The method of any of the above enumerated embodiments wherein the compositions described herein can be administered by transtympanic wicks.
Embodiment 2336. The method of any of the above enumerated embodiments wherein the compositions described herein can be administered by catheters.
Embodiment 2337. The method of any of the above enumerated embodiments wherein the compositions described herein can be administered by parental administration.
Embodiment 2338. The method of any of the above enumerated embodiments wherein the compositions described herein can be administered by intraauricular injection.
Embodiment 2339. The method of any of the above enumerated embodiments wherein the compositions described herein can be administered by transtympanic injection.
Embodiment 2340. The method of any of the above enumerated embodiments wherein the compositions described herein can be administered intracochlear injection.
Embodiment 2341. The method of any of the above enumerated embodiments wherein the compounds, compositions and formulations of the disclosure are locally administered.
Embodiment 2342. The method of any of the above enumerated embodiments wherein the compounds, compositions and formulations of the disclosure are not administered systemically.
Embodiment 2343. The method of any of the above enumerated embodiments wherein a syringe and needle apparatus is used to administer compounds or compositions to a subject using auricular administration.
Embodiment 2344. The method of any of the above enumerated embodiments wherein a device is inserted such that it is in contact with the round window.
Embodiment 2345. The method of any of the above enumerated embodiments wherein a device is inserted immediately adjacent to the round window.
Embodiment 2346. The method of any of the above enumerated embodiments wherein the device used for auricular administration is a transtympanic wick.
Embodiment 2347. The method of any of the above enumerated embodiments wherein the device used for auricular administration is a transtympanic catheter.
Embodiment 2348. The method of any of the above enumerated embodiments wherein the device used for auricular administration is a round window microcatheter.
Embodiment 2349. The method of any of the above enumerated embodiments wherein the device used for auricular administration is a Silverstein Microwick™.
Embodiment 2350. The method of any of the above enumerated embodiments wherein a syringe and needle apparatus is used to administer compounds or compositions to a subject using transtympanic injection, injection behind the tympanic membrane into the middle and/or inner ear.
Embodiment 2351. The method of any of the above enumerated embodiments wherein the formulation is administered directly onto the round window membrane via transtympanic injection.
Embodiment 2352. The method of any of the above enumerated embodiments wherein the formulation is administered directly to the cochlea via intracochlear injection.
Embodiment 2353. The method of any of the above enumerated embodiments wherein the formulation is administered directly to the vestibular organs via intravestibular injection.
Embodiment 2354. The method of any of the above enumerated embodiments wherein the delivery device is an apparatus designed for administration of compounds or compositions to the middle ear.
Embodiment 2355. The method of any of the above enumerated embodiments wherein the delivery device is an apparatus designed for administration of compounds or compositions to the inner ear.
Embodiment 2356. The method of any of the above enumerated embodiments wherein a compound or composition disclosed herein is administered to a subject in need thereof once.
Embodiment 2357. The method of any of the above enumerated embodiments wherein a compound or composition disclosed herein is administered to a subject in need thereof more than once.
Embodiment 2358. The method of any of the above enumerated embodiments wherein a first administration of a compound or composition disclosed herein is followed by a second, third, fourth or fifth administration of a compound or composition disclosed herein.
Embodiment 2359. The method of any of the above enumerated embodiments wherein the compound or composition disclosed herein is administered once to a subject in need thereof with a mild acute condition.
Embodiment 2360. The method of any of the above enumerated embodiments wherein a compound or composition disclosed herein is administered more than once to a subject in need thereof with a moderate or severe acute condition.
Embodiment 2361. The method of any of the above enumerated embodiments wherein a subject's condition does not improve, upon the doctor's discretion the compound or composition is administered chronically, that is, for an extended period of time.
Embodiment 2362. The method of any of the above enumerated embodiments wherein the compound or composition is administered chronically in order to ameliorate the symptoms of a subject's disease or condition.
Embodiment 2363. The method of any of the above enumerated embodiments wherein the compound or composition is administered chronically in order to control the symptoms of a subject's disease or condition.
Embodiment 2364. The method of any of the above enumerated embodiments wherein the compound or composition is administered chronically in order to limit the symptoms of a subject's disease or condition.
Embodiment 2365. The method of any of the above enumerated embodiments wherein if the subject's status does improve, upon the doctor's discretion the compound, or composition, may administered continuously.
Embodiment 2366. The method of any of the above enumerated embodiments wherein if the subject's status does improve, upon the doctor's discretion the dose of the drug being administered my temporarily be reduced.
Embodiment 2367. The method of any of the above enumerated embodiments wherein if the subject's status does improve, upon the doctor's discretion the dose of the drug being administered is temporarily suspended for a certain length of time (i.e. a “drug holiday”).
Embodiment 2368. The method of any of the above enumerated embodiments wherein the drug holiday varies between 2 days and 1 year.
Embodiment 2369. The method of any of the above enumerated embodiments wherein the drug holiday is 2 days.
Embodiment 2370. The method of any of the above enumerated embodiments wherein the drug holiday is 3 days.
Embodiment 2371. The method of any of the above enumerated embodiments wherein the drug holiday is 4 days.
Embodiment 2372. The method of any of the above enumerated embodiments wherein the drug holiday is 5 days.
Embodiment 2373. The method of any of the above enumerated embodiments wherein the drug holiday is 6 days.
Embodiment 2374. The method of any of the above enumerated embodiments wherein the drug holiday is 7 days.
Embodiment 2375. The method of any of the above enumerated embodiments wherein the drug holiday is 10 days.
Embodiment 2376. The method of any of the above enumerated embodiments wherein the drug holiday is 12 days.
Embodiment 2377. The method of any of the above enumerated embodiments wherein the drug holiday is 15 days.
Embodiment 2378. The method of any of the above enumerated embodiments wherein the drug holiday is 20 days.
Embodiment 2379. The method of any of the above enumerated embodiments wherein the drug holiday is 28 days.
Embodiment 2380. The method of any of the above enumerated embodiments wherein the drug holiday is 35 days.
Embodiment 2381. The method of any of the above enumerated embodiments wherein the drug holiday is 50 days.
Embodiment 2382. The method of any of the above enumerated embodiments wherein the drug holiday is 70 days.
Embodiment 2383. The method of any of the above enumerated embodiments wherein the drug holiday is 100 days.
Embodiment 2384. The method of any of the above enumerated embodiments wherein the drug holiday is 120 days.
Embodiment 2385. The method of any of the above enumerated embodiments wherein the drug holiday is 150 days.
Embodiment 2386. The method of any of the above enumerated embodiments wherein the drug holiday is 180 days.
Embodiment 2387. The method of any of the above enumerated embodiments wherein the drug holiday is 200 days.
Embodiment 2388. The method of any of the above enumerated embodiments wherein the drug holiday is 250 days.
Embodiment 2389. The method of any of the above enumerated embodiments wherein the drug holiday is 280 days.
Embodiment 2390. The method of any of the above enumerated embodiments wherein the drug holiday is 300 days.
Embodiment 2391. The method of any of the above enumerated embodiments wherein the drug holiday is 320 days.
Embodiment 2392. The method of any of the above enumerated embodiments wherein the drug holiday is 350 days.
Embodiment 2393. The method of any of the above enumerated embodiments wherein the drug holiday is 365 days.
Embodiment 2394. The method of any of the above enumerated embodiments wherein the dose reduction during a drug holiday is from 10%-100%.
Embodiment 2395. The method of any of the above enumerated embodiments wherein the dose reduction during a drug holiday is 10%.
Embodiment 2396. The method of any of the above enumerated embodiments wherein the dose reduction during a drug holiday is 15%.
Embodiment 2397. The method of any of the above enumerated embodiments wherein the dose reduction during a drug holiday is 20%.
Embodiment 2398. The method of any of the above enumerated embodiments wherein the dose reduction during a drug holiday is 25%.
Embodiment 2399. The method of any of the above enumerated embodiments wherein the dose reduction during a drug holiday is 30%.
Embodiment 2400. The method of any of the above enumerated embodiments wherein the dose reduction during a drug holiday is 35%.
Embodiment 2401. The method of any of the above enumerated embodiments wherein the dose reduction during a drug holiday is 40%.
Embodiment 2402. The method of any of the above enumerated embodiments wherein the dose reduction during a drug holiday is 45%.
Embodiment 2403. The method of any of the above enumerated embodiments wherein the dose reduction during a drug holiday is 50%.
Embodiment 2404. The method of any of the above enumerated embodiments wherein the dose reduction during a drug holiday is 55%.
Embodiment 2405. The method of any of the above enumerated embodiments wherein the dose reduction during a drug holiday is 60%.
Embodiment 2406. The method of any of the above enumerated embodiments wherein the dose reduction during a drug holiday is 65%.
Embodiment 2407. The method of any of the above enumerated embodiments wherein the dose reduction during a drug holiday is 70%.
Embodiment 2408. The method of any of the above enumerated embodiments wherein the dose reduction during a drug holiday is 75%.
Embodiment 2409. The method of any of the above enumerated embodiments wherein the dose reduction during a drug holiday is 80%.
Embodiment 2410. The method of any of the above enumerated embodiments wherein the dose reduction during a drug holiday is 85%.
Embodiment 2411. The method of any of the above enumerated embodiments wherein the dose reduction during a drug holiday is 90°/c.
Embodiment 2412. The method of any of the above enumerated embodiments wherein the dose reduction during a drug holiday is 95%.
Embodiment 2413. The method of any of the above enumerated embodiments wherein the dose reduction during a drug holiday is 100%.
Embodiment 2414. The method of any of the above enumerated embodiments wherein once the subject's hearing has improved, a maintenance dose can be administered, if necessary.
Embodiment 2415. The method of any of the above enumerated embodiments wherein once the subject's balance has improved, a maintenance dose can be administered, if necessary.
Embodiment 2416. The method of any of the above enumerated embodiments wherein once the subject's hearing and balance has improved, a maintenance dose can be administered, if necessary.
Embodiment 2417. The method of any of the above enumerated embodiments wherein once the subject's hearing and balance has improved, a maintenance dose can be administered, if necessary.
Embodiment 2418. The method of any of the above enumerated embodiments wherein the dosage of administration is optionally reduced as a function of the symptoms, to a level at which the improved disease is maintained.
Embodiment 2419. The method of any of the above enumerated embodiments wherein the frequency of administration is optionally reduced as a function of the symptoms, to a level at which the improved disease is maintained.
Embodiment 2420. The method of any of the above enumerated embodiments wherein the dosage of administration is optionally reduced as a function of the symptoms, to a level at which the improved disorder is maintained.
Embodiment 2421. The method of any of the above enumerated embodiments wherein the frequency of administration is optionally reduced as a function of the symptoms, to a level at which the improved disorder is maintained.
Embodiment 2422. The method of any of the above enumerated embodiments wherein the dosage of administration is optionally reduced as a function of the symptoms, to a level at which the improved condition is maintained.
Embodiment 2423. The method of any of the above enumerated embodiments wherein the frequency of administration is optionally reduced as a function of the symptoms, to a level at which the improved condition is maintained.
Embodiment 2424. The method of any of the above enumerated embodiments wherein the subjects require intermittent treatment on a long-term basis upon any recurrence of symptoms.
Embodiment 2425. A pharmaceutical composition comprising a first agent that is a TAZ activator, a Wnt agonist and a pharmaceutically acceptable carrier.
Embodiment 2426. The pharmaceutical composition of any of the above enumerated embodiments, wherein the TAZ activator is IBS008738, TM-25659 or TT10.
Embodiment 2427. The pharmaceutical composition of any of the above enumerated embodiments, wherein the TAZ activator is IBS008738.
Embodiment 2428. The pharmaceutical composition of any of the above enumerated embodiments, wherein the TAZ activator is TM-25659.
Embodiment 2429. The pharmaceutical composition of any of the above enumerated embodiments, wherein the TAZ activator is TT10
Embodiment 2430. The pharmaceutical composition of any of the above enumerated embodiments, wherein the 1BS008738 is at a concentration of about between 1 mM to 30 mM.
Embodiment 2431. The pharmaceutical composition of any of the above enumerated embodiments, wherein the TM-25659 is at a concentration of about between 1 mM to 10 mM.
Embodiment 2432. The pharmaceutical composition of any of the above enumerated embodiments, wherein the TT10 is at a concentration of about between 10 mM to 100 mM.
Embodiment 2433. The pharmaceutical composition of any of the above enumerated embodiments, wherein the Wnt agonist is a GSK3 inhibitor.
Embodiment 2434. The pharmaceutical composition of any of the above enumerated embodiments, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.
Embodiment 2435. The pharmaceutical composition of any of the above enumerated embodiments wherein the GSK3 inhibitor is AZD1080.
Embodiment 2436. The pharmaceutical composition of any of the above enumerated embodiments wherein the GSK3 inhibitor is LY2090314.
Embodiment 2437. The pharmaceutical composition of any of the above enumerated embodiments, wherein the GSK3 inhibitor is a substituted 3-Imidazo[1,2-a]pyridin-3-yl-4-(1,2,3,4-tetrahydro-[1,4]diazepino-[6,7,1-hi]indol-7-yl)pyrrole-2,5-dione.
Embodiment 2438. The pharmaceutical composition of any preceding claim, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is as defined in Formula A.
Embodiment 2439. The pharmaceutical composition of any preceding claim, wherein the substituted 3-Imidazo[1,2-a]pyridin-3-yl-4-(1,2,3,4-tetrahydro-[1,4]diazepino-[6,7,1-hi]indol-7-yl)pyrrole-2,5-dione is selected from those disclosed in Table 4.
Embodiment 2440. The pharmaceutical composition of any of the above enumerated embodiments, wherein the GSK3 inhibitor is GSK3 inhibitor XXII.
Embodiment 2441. The pharmaceutical composition of any of the above enumerated embodiments, wherein the GSK3 inhibitor is CHIR99021.
Embodiment 2442. The pharmaceutical composition of any of the above enumerated embodiments, wherein AZD1080 is at a concentration of about between 1 mM to 5 mM.
Embodiment 2443. The pharmaceutical composition of any of the above enumerated embodiments, wherein LY2090314 is at a concentration of about between 5 mM to 20 mM.
Embodiment 2444. The pharmaceutical composition of any of the above enumerated embodiments a substituted 3-Imidazo[1,2-a]pyridin-3-yl-4-(1,2,3,4-tetrahydro-[1,4]diazepino-[6,7,1-hi]indol-7-yl)pyrrole-2,5-dione is at a concentration of about between 5 μM to 500 μM.
Embodiment 2445. The pharmaceutical composition of any of the above enumerated embodiments, wherein GSK3 inhibitor XXH at a concentration of about between 0.2 mM to 1 mM.
Embodiment 2446. The pharmaceutical composition of any of the above enumerated embodiments, wherein CHIR99021 is at a concentration of about between 2 mM to 6 mM.
Embodiment 2447. The pharmaceutical composition of any of the above enumerated embodiments, further comprising further comprising an epigenetic agent.
Embodiment 2448. The pharmaceutical composition of any of the above enumerated embodiments, wherein the epigenetic agent is an HDAC inhibitor, an EZH2 inhibitor, a DOT1L inhibitor an LSD1 inhibitor or a KDM inhibitor.
Embodiment 2449. The pharmaceutical composition of any of the above enumerated embodiments, wherein the HDAC inhibitor is Valproic Acid (VPA)
Embodiment 2450. The pharmaceutical composition of any of the above enumerated embodiments, wherein the VPA is at a concentration of about between 100 mM to 4,000 mM.
Embodiment 2451. The pharmaceutical composition of any of the above enumerated embodiments, wherein the EZH2 inhibitor an enzymatic inhibitor.
Embodiment 2452. The pharmaceutical composition of any of the above enumerated embodiments, wherein the EZH2 inhibitor is selected from the group consisting of: CPI-1205, CPI-169, E11, PF-06821497, tazemetostat, valemetostat, CPI-360, EPZ011989, UNC 2399, and PF 06726304.
Embodiment 2453. The pharmaceutical composition of any of the above enumerated embodiments, wherein the EZH2 inhibitor is CPI-1205.
Embodiment 2454. The pharmaceutical composition of any of the above enumerated embodiments, wherein the EZH2 inhibitor is E11.
Embodiment 2455. The pharmaceutical composition of any of the above enumerated embodiments, wherein the EZH2 inhibitor is PF-06821497.
Embodiment 2456. The pharmaceutical composition of any of the above enumerated embodiments, wherein the EZH2 inhibitor is tazemetostat.
Embodiment 2457. The pharmaceutical composition of any of the above enumerated embodiments, wherein the EZH2 inhibitor is valemetostat.
Embodiment 2458. The pharmaceutical composition of any of the above enumerated embodiments, wherein the CPI-1205 is at a concentration of about between 10 mM to 1000 mM.
Embodiment 2459. The pharmaceutical composition of any of the above enumerated embodiments, wherein the E11 is at a concentration of about between 10 mM to 1000 mM.
Embodiment 2460. The pharmaceutical composition of any of the above enumerated embodiments, wherein the PF-06821497 is at a concentration of about between 1 mM to 10 mM.
Embodiment 2461. The pharmaceutical composition of any of the above enumerated embodiments, wherein the tazemetostat is at a concentration of about between 0.1 mM to 1.5 mM.
Embodiment 2462. The pharmaceutical composition of any of the above enumerated embodiments, wherein the valemetostat is at a concentration of about between 10 mM to 1000 mM.
Embodiment 2463. The pharmaceutical composition of any of the above enumerated embodiments, wherein the DOT1L inhibitor and S-adenosyl methionine (SAM) competitive inhibitor.
Embodiment 2464. The pharmaceutical composition of any of the above enumerated embodiments, wherein the DOT1L inhibitor is selected from the group consisting of EPZ004777, pinometostat and SGC0946.
Embodiment 2465. The pharmaceutical composition of any of the above enumerated embodiments, wherein the DOT1L inhibitor is EPZ004777.
Embodiment 2466. The pharmaceutical composition of any of the above enumerated embodiments, wherein the DOT1L inhibitor is pinometostat.
Embodiment 2467. The pharmaceutical composition of any of the above enumerated embodiments, wherein the DOT1L inhibitor is SGC0946.
Embodiment 2468. The pharmaceutical composition of any of the above enumerated embodiments, wherein the DOT1L EPZ004777 is at a concentration of about between 0.5 mM to 45 mM.
Embodiment 2469. The pharmaceutical composition of any of the above enumerated embodiments, wherein the pinometostat is at a concentration of about between 0.1 mM to 10 mM.
Embodiment 2470. The pharmaceutical composition of any of the above enumerated embodiments, wherein the SGC0946 is at a concentration of about between 0.5 mM to 5 mM.
Embodiment 2471. The pharmaceutical composition of any of the above enumerated embodiments, wherein the KDM inhibitor is AS 835129 or TC-E 5002.
Embodiment 2472. The pharmaceutical composition of any of the above enumerated embodiments, wherein the KDM inhibitor is AS 8351.
Embodiment 2473. The pharmaceutical composition of any of the above enumerated embodiments, wherein the KDM inhibitor is TC-E 5002.
Embodiment 2474. The pharmaceutical composition of any of the above enumerated embodiments, wherein the AS 8351 is at a concentration of about between 0.1 mM to 10 mM.
Embodiment 2475. The pharmaceutical composition of any of the above enumerated embodiments, wherein the TC-E 5002 is at a concentration of about between 1 mM to 3 mM.
Embodiment 2476. The pharmaceutical composition of any of the above enumerated embodiments, wherein the pharmaceutical composition is in a biocompatible matrix.
Embodiment 2477. The pharmaceutical composition of any of the above enumerated embodiments, wherein the biocompatible matrix comprises hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.
Embodiment 2478. The pharmaceutical compostion of any of the above enumerated embodiments wherein the LSD1 inhibitor is Tranylcypromine (TCP)
Embodiment 2479. The pharmaceutical composition of any of the above enumerated embodiments wherein the LSD1 inhibitor is GSK-2879552.
Embodiment 2480. The pharmaceutical composition of any of the above enumerated embodiments wherein the LSD1 inhibitor is GSK-LSD1.
Embodiment 2481. The pharmaceutical composition of any of the above enumerated embodiments wherein the LSD1 inhibitor is Phenelzine sulfate.
Embodiment 2482. The pharmaceutical composition of any of the above enumerated embodiments wherein the LSD1 inhibitor is ORY-2001 (Vafidemstat).
Embodiment 2483. The pharmaceutical composition of any of the above enumerated embodiments wherein the LSD1 inhibitor is SP-2577 (Seclidemstat).
Embodiment 2484. The pharmaceutical composition of any of the above enumerated embodiments wherein the LSD1 inhibitor is Osimertinib (AZD9291).
Embodiment 2485. The pharmaceutical composition of any of the above enumerated embodiments wherein the LSD1 inhibitor is GCG-11047 (PG-11047).
Embodiment 2486. The pharmaceutical composition of any of the above enumerated embodiments wherein the LSD1 inhibitor is ORY-1001 (RG6016, R07051790, Iadademstat).
Embodiment 2487. The pharmaceutical composition of any of the above enumerated embodiments wherein the LSD1 inhibitor is IMG-7289.
Embodiment 2488. The pharmaceutical composition of any of the above enumerated embodiments wherein the LSD1 inhibitor is CC-90011.
Embodiment 2489. The pharmaceutical composition of any of the above enumerated embodiments wherein the LSD1 inhibitor is INCB059872.
Embodiment 2490. The pharmaceutical composition of any of the above enumerated embodiments wherein the TAZ activator is at a concentration of about 0.001 nM to 1,000 mM.
Embodiment 2491. The pharmaceutical composition of any of the above enumerated embodiments wherein the TAZ activator is at a concentration of about 0.01 nM to 100,000 μM.
Embodiment 2492. The pharmaceutical composition of any of the above enumerated embodiments wherein the TAZ activator is at a concentration of about 0.1 nM to 10,000 μM.
Embodiment 2493. The pharmaceutical composition of any of the above enumerated embodiments wherein the TAZ activator is at a concentration of about 1 nM to 1,000 μM.
Embodiment 2494. The pharmaceutical composition of any of the above enumerated embodiments wherein the TAZ activator is at a concentration of about 1 nM to 10 μM.
Embodiment 2495. The pharmaceutical composition of any of the above enumerated embodiments wherein the TAZ activator is at a concentration of about 10 nM to 100 μM.
Embodiment 2496. The pharmaceutical composition of any of the above enumerated embodiments wherein the TAZ activator is at a concentration of about 100 nM to 1 mM.
Embodiment 2497. The pharmaceutical composition of any of the above enumerated embodiments wherein the TAZ activator is at a concentration of about 1 nM to 10 mM.
Embodiment 2498. The pharmaceutical composition of any of the above enumerated embodiments wherein the TAZ activator is at a concentration of about 0.1 nM.
Embodiment 2499. The pharmaceutical composition of any of the above enumerated embodiments wherein the TAZ activator is at a concentration of about 0.2 nM.
Embodiment 2500. The pharmaceutical composition of any of the above enumerated embodiments wherein the TAZ activator is at a concentration of about 0.3 nM.
Embodiment 2501. The pharmaceutical composition of any of the above enumerated embodiments wherein the TAZ activator is at a concentration of about 0.4 nM.
Embodiment 2502. The pharmaceutical composition of any of the above enumerated embodiments wherein the TAZ activator is at a concentration of about 0.5 nM.
Embodiment 2503. The pharmaceutical composition of any of the above enumerated embodiments wherein the TAZ activator is at a concentration of about 0.6 nM.
Embodiment 2504. The pharmaceutical composition of any of the above enumerated embodiments wherein the TAZ activator is at a concentration of about 0.7 nM.
Embodiment 2505. The pharmaceutical composition of any of the above enumerated embodiments wherein the TAZ activator is at a concentration of about 0.8 nM.
Embodiment 2506. The pharmaceutical composition of any of the above enumerated embodiments wherein the TAZ activator is at a concentration of about 0.9 nM.
Embodiment 2507. The pharmaceutical composition of any of the above enumerated embodiments wherein the TAZ activator is at a concentration of about 1 nM.
Embodiment 2508. The pharmaceutical composition of any of the above enumerated embodiments wherein the TAZ activator is at a concentration of about 1 μM.
Embodiment 2509. The pharmaceutical composition of any of the above enumerated embodiments wherein the TAZ activator is at a concentration of about 2 μM.
Embodiment 2510. The pharmaceutical composition of any of the above enumerated embodiments wherein the TAZ activator is at a concentration of about 3 μM.
Embodiment 2511. The pharmaceutical composition of any of the above enumerated embodiments wherein the TAZ activator is at a concentration of about 4 μM.
Embodiment 2512. The pharmaceutical composition of any of the above enumerated embodiments wherein the TAZ activator is at a concentration of about 5 μM.
Embodiment 2513. The pharmaceutical composition of any of the above enumerated embodiments wherein the TAZ activator is at a concentration of about 6 μM.
Embodiment 2514. The pharmaceutical composition of any of the above enumerated embodiments wherein the TAZ activator is at a concentration of about 7 μM.
Embodiment 2515. The pharmaceutical composition of any of the above enumerated embodiments wherein the TAZ activator is at a concentration of about 8 μM.
Embodiment 2516. The pharmaceutical composition of any of the above enumerated embodiments wherein the TAZ activator is at a concentration of about 9 μM.
Embodiment 2517. The pharmaceutical composition of any of the above enumerated embodiments wherein the TAZ activator is at a concentration of about 10 μM.
Embodiment 2518. The pharmaceutical composition of any of the above enumerated embodiments wherein the TAZ activator is at a concentration of about 20 μM.
Embodiment 2519. The pharmaceutical composition of any of the above enumerated embodiments wherein the TAZ activator is at a concentration of about 30 μM.
Embodiment 2520. The pharmaceutical composition of any of the above enumerated embodiments wherein the TAZ activator is at a concentration of about 40 μM.
Embodiment 2521. The pharmaceutical composition of any of the above enumerated embodiments wherein the TAZ activator is at a concentration of about 50 μM.
Embodiment 2522. The pharmaceutical composition of any of the above enumerated embodiments wherein the TAZ activator is at a concentration of about 60 μM.
Embodiment 2523. The pharmaceutical composition of any of the above enumerated embodiments wherein the TAZ activator is at a concentration of about 70 μM.
Embodiment 2524. The pharmaceutical composition of any of the above enumerated embodiments wherein the TAZ activator is at a concentration of about 80 μM.
Embodiment 2525. The pharmaceutical composition of any of the above enumerated embodiments wherein the TAZ activator is at a concentration of about 90 μM.
Embodiment 2526. The pharmaceutical composition of any of the above enumerated embodiments wherein the TAZ activator is at a concentration of about 100 μM.
Embodiment 2527. The pharmaceutical composition of any of the above enumerated embodiments wherein the TAZ activator is at a concentration of about 200 μM.
Embodiment 2528. The pharmaceutical composition of any of the above enumerated embodiments wherein the TAZ activator is at a concentration of about 300 μM.
Embodiment 2529. The pharmaceutical composition of any of the above enumerated embodiments wherein the TAZ activator is at a concentration of about 400 μM.
Embodiment 2530. The pharmaceutical composition of any of the above enumerated embodiments wherein the TAZ activator is at a concentration of about 600 μM.
Embodiment 2531. The pharmaceutical composition of any of the above enumerated embodiments wherein the TAZ activator is at a concentration of about 700 μM.
Embodiment 2532. The pharmaceutical composition of any of the above enumerated embodiments wherein the TAZ activator is at a concentration of about 800 μM.
Embodiment 2533. The pharmaceutical composition of any of the above enumerated embodiments wherein the TAZ activator is at a concentration of about 900 μM.
Embodiment 2534. The pharmaceutical composition of any of the above enumerated embodiments wherein the TAZ activator is at a concentration of about 1 mM.
Embodiment 2535. The pharmaceutical composition of any of the above enumerated embodiments wherein the TAZ activator is at a concentration of about 2 mM.
Embodiment 2536. The pharmaceutical composition of any of the above enumerated embodiments wherein the TAZ activator is at a concentration of about 3 mM.
Embodiment 2537. The pharmaceutical composition of any of the above enumerated embodiments wherein the TAZ activator is at a concentration of about 4 mM.
Embodiment 2538. The pharmaceutical composition of any of the above enumerated embodiments wherein the TAZ activator is at a concentration of about 5 mM.
Embodiment 2539. The pharmaceutical composition of any of the above enumerated embodiments wherein the TAZ activator is at a concentration of about 6 mM.
Embodiment 2540. The pharmaceutical composition of any of the above enumerated embodiments wherein the TAZ activator is at a concentration of about 7 mM.
Embodiment 2541. The pharmaceutical composition of any of the above enumerated embodiments wherein the TAZ activator is at a concentration of about 8 mM.
Embodiment 2542. The pharmaceutical composition of any of the above enumerated embodiments wherein the TAZ activator is at a concentration of about 9 mM.
Embodiment 2543. The pharmaceutical composition of any of the above enumerated embodiments wherein the TAZ activator is at a concentration of about 10 mM.
Embodiment 2544. The pharmaceutical composition of any of the above enumerated embodiments wherein the TAZ activator is at a concentration of about 20 mM.
Embodiment 2545. The pharmaceutical composition of any of the above enumerated embodiments wherein the TAZ activator is at a concentration of about 30 mM.
Embodiment 2546. The pharmaceutical composition of any of the above enumerated embodiments wherein the TAZ activator is at a concentration of about 40 mM.
Embodiment 2547. The pharmaceutical composition of any of the above enumerated embodiments wherein the TAZ activator is at a concentration of about 50 mM.
Embodiment 2548. The pharmaceutical composition of any of the above enumerated embodiments wherein the TAZ activator is at a concentration of about 60 mM.
Embodiment 2549. The pharmaceutical composition of any of the above enumerated embodiments wherein the TAZ activator is at a concentration of about 70 mM.
Embodiment 2550. The pharmaceutical composition of any of the above enumerated embodiments wherein the TAZ activator is at a concentration of about 80 mM.
Embodiment 2551. The pharmaceutical composition of any of the above enumerated embodiments wherein the TAZ activator is at a concentration of about 90 mM.
Embodiment 2552. The pharmaceutical composition of any of the above enumerated embodiments wherein the TAZ activator is at a concentration of about 100 mM.
Embodiment 2553. The pharmaceutical composition of any of the above enumerated embodiments wherein the TAZ activator is at a concentration of about 150 mM.
Embodiment 2554. The pharmaceutical composition of any of the above enumerated embodiments wherein the TAZ activator is at a concentration of about 200 mM.
Embodiment 2555. The pharmaceutical composition of any of the above enumerated embodiments wherein the TAZ activator is at a concentration of about 250 mM.
Embodiment 2556. The pharmaceutical composition of any of the above enumerated embodiments wherein the TAZ activator is at a concentration of about 500 mM.
Embodiment 2557. The pharmaceutical composition of any of the above enumerated embodiments wherein the TAZ activator is at a concentration of about 100 mM.
Embodiment 2558. The pharmaceutical composition of any of the above enumerated embodiments wherein the TAZ activator is at a unit dose of about 0.01 mg to 5000 mg.
Embodiment 2559. The pharmaceutical composition of any of the above enumerated embodiments wherein the TAZ activator is at a unit dose of about 0.1 mg to 1000 mg.
Embodiment 2560. The pharmaceutical composition of any of the above enumerated embodiments wherein the TAZ activator is at a unit dose of about 1 mg to 500 mg.
Embodiment 2561. The pharmaceutical composition of any of the above enumerated embodiments wherein the TAZ activator is at a unit dose of about 1 mg to 250 mg.
Embodiment 2562. The pharmaceutical composition of any of the above enumerated embodiments wherein the TAZ activator is at a unit dose of about 1 mg to 100 mg.
Embodiment 2563. The pharmaceutical composition of any of the above enumerated embodiments wherein the TAZ activator is at a unit dose of about 1 mg to 50 mg.
Embodiment 2564. The pharmaceutical composition of any of the above enumerated embodiments wherein the TAZ activator is at a unit dose of about 0.01 mg to 1 mg.
Embodiment 2565. The pharmaceutical composition of any of the above enumerated embodiments wherein the TAZ activator is at a unit dose of about 0.1 mg to 10 mg.
Embodiment 2566. The pharmaceutical composition of any of the above enumerated embodiments wherein the TAZ activator is at a unit dose of about 1 mg to 100 mg.
Embodiment 2567. The pharmaceutical composition of any of the above enumerated embodiments wherein the TAZ activator is at a unit dose of about 100 mg to 1000 mg.
Embodiment 2568. The pharmaceutical composition of any of the above enumerated embodiments wherein the TAZ activator is at a unit dose of about 1000 mg to 5000 mg.
Embodiment 2569. The pharmaceutical composition of any of the above enumerated embodiments wherein the TAZ activator is at a unit dose of about 0.5 mg to 1 mg.
Embodiment 2570. The pharmaceutical composition of any of the above enumerated embodiments wherein the TAZ activator is at a unit dose of about 1 mg to 2 mg.
Embodiment 2571. The pharmaceutical composition of any of the above enumerated embodiments wherein the TAZ activator is at a unit dose of about 2 mg to 3 mg.
Embodiment 2572. The pharmaceutical composition of any of the above enumerated embodiments wherein the TAZ activator is at a unit dose of about 3 mg to 4 mg.
Embodiment 2573. The pharmaceutical composition of any of the above enumerated embodiments wherein the TAZ activator is at a unit dose of about 4 mg to 5 mg.
Embodiment 2574. The pharmaceutical composition of any of the above enumerated embodiments wherein the TAZ activator is at a unit dose of about 5-10 mg.
Embodiment 2575. The pharmaceutical composition of any of the above enumerated embodiments wherein the TAZ activator is at a unit dose of about 10-25 mg.
Embodiment 2576. The pharmaceutical composition of any of the above enumerated embodiments wherein the TAZ activator is at a unit dose of about 25-50 mg.
Embodiment 2577. The pharmaceutical composition of any of the above enumerated embodiments wherein the TAZ activator is at a unit dose of about 50-100 mg.
Embodiment 2578. The pharmaceutical composition of any of the above enumerated embodiments wherein the TAZ activator is at a concentration of about 0.001 μM to 10 mM.
Embodiment 2579. The pharmaceutical composition of any of the above enumerated embodiments wherein the TAZ activator is at a concentration of about 0.01 μM to 1 mM.
Embodiment 2580. The pharmaceutical composition of any of the above enumerated embodiments wherein the TAZ activator is at a concentration of about 0.1 μM to 100 μM.
Embodiment 2581. The pharmaceutical composition of any of the above enumerated embodiments wherein the TAZ activator is at a concentration of about 0.001 μM to 0.01 μM.
Embodiment 2582. The pharmaceutical composition of any of the above enumerated embodiments wherein the TAZ activator is at a concentration of about 0.01 μM to 0.1 μM.
Embodiment 2583. The pharmaceutical composition of any of the above enumerated embodiments wherein the TAZ activator is at a concentration of about 0.1 μM to 1 μM.
Embodiment 2584. The pharmaceutical composition of any of the above enumerated embodiments wherein the TAZ activator is at a concentration of about 1 μM to 10 μM.
Embodiment 2585. The pharmaceutical composition of any of the above enumerated embodiments wherein the TAZ activator is at a concentration of about 10 μM to 100 μM.
Embodiment 2586. The pharmaceutical composition of any of the above enumerated embodiments wherein the TAZ activator is at a concentration of about 100 μM to 1,000 μM.
Embodiment 2587. The pharmaceutical composition of any of the above enumerated embodiments wherein the TAZ activator is at a concentration of about 0.001 mM to 10,000 mM.
Embodiment 2588. The pharmaceutical composition of any of the above enumerated embodiments wherein the TAZ activator is at a concentration of about 0.01 mM to 1,000 mM.
Embodiment 2589. The pharmaceutical composition of any of the above enumerated embodiments wherein the TAZ activator is at a concentration of about 0.1 mM to 100 mM.
Embodiment 2590. The pharmaceutical composition of any of the above enumerated embodiments wherein the TAZ activator is at a concentration of about 0.001 mM to 0.01 mM.
Embodiment 2591. The pharmaceutical composition of any of the above enumerated embodiments wherein the TAZ activator is at a concentration of about 0.01 mM to 0.1 mM.
Embodiment 2592. The pharmaceutical composition of any of the above enumerated embodiments wherein the TAZ activator is at a concentration of about 0.1 mM to 1 mM
Embodiment 2593. The pharmaceutical composition of any of the above enumerated embodiments wherein the TAZ activator is at a concentration of about 1 mM to 10 mM.
Embodiment 2594. The pharmaceutical composition of any of the above enumerated embodiments wherein the TAZ activator is at a concentration of about 10 mM to 100 mM.
Embodiment 2595. The pharmaceutical composition of any of the above enumerated embodiments wherein the TAZ activator is at a concentration of about 100 mM to 1,000 mM
Embodiment 2596. The pharmaceutical composition of any of the above enumerated embodiments wherein the TAZ activator is at a concentration of about 1,000 mM to 10,000 mM.
Embodiment 2597. The pharmaceutical composition of any of the above enumerated embodiments wherein the TAZ activator is at a concentration of about 0.1 mM.
Embodiment 2598. The pharmaceutical composition of any of the above enumerated embodiments wherein the TAZ activator is at a concentration of about 0.2 mM.
Embodiment 2599. The pharmaceutical composition of any of the above enumerated embodiments wherein the TAZ activator is at a concentration of about 0.3 mM.
Embodiment 2600. The pharmaceutical composition of any of the above enumerated embodiments wherein the TAZ activator is at a concentration of about 0.4 mM.
Embodiment 2601. The pharmaceutical composition of any of the above enumerated embodiments wherein the TAZ activator is at a concentration of about 0.5 mM.
Embodiment 2602. The pharmaceutical composition of any of the above enumerated embodiments wherein the TAZ activator is at a concentration of about 0.6 mM.
Embodiment 2603. The pharmaceutical composition of any of the above enumerated embodiments wherein the TAZ activator is at a concentration of about 0.7 mM.
Embodiment 2604. The pharmaceutical composition of any of the above enumerated embodiments wherein the TAZ activator is at a concentration of about 0.8 mM.
Embodiment 2605. The pharmaceutical composition of any of the above enumerated embodiments wherein the TAZ activator is at a concentration of about 0.9 mM.
Embodiment 2606. The pharmaceutical composition of any of the above enumerated embodiments wherein the TAZ activator is at a concentration of about 1 mM.
Embodiment 2607. The pharmaceutical composition of any of the above enumerated embodiments wherein the TAZ activator is at a concentration of about 2 mM.
Embodiment 2608. The pharmaceutical composition of any of the above enumerated embodiments wherein the TAZ activator is at a concentration of about 3 mM.
Embodiment 2609. The pharmaceutical composition of any of the above enumerated embodiments wherein the TAZ activator is at a concentration of about 4 mM.
Embodiment 2610. The pharmaceutical composition of any of the above enumerated embodiments wherein the TAZ activator is at a concentration of about 5 mM.
Embodiment 2611. The pharmaceutical composition of any of the above enumerated embodiments wherein the TAZ activator is at a concentration of about 6 mM.
Embodiment 2612. The pharmaceutical composition of any of the above enumerated embodiments wherein the TAZ activator is at a concentration of about 7 mM.
Embodiment 2613. The pharmaceutical composition of any of the above enumerated embodiments wherein the TAZ activator is at a concentration of about 8 mM.
Embodiment 2614. The pharmaceutical composition of any of the above enumerated embodiments wherein the TAZ activator is at a concentration of about 9 mM.
Embodiment 2615. The pharmaceutical composition of any of the above enumerated embodiments wherein the TAZ activator is at a concentration of about 10 mM.
Embodiment 2616. The pharmaceutical composition of any of the above enumerated embodiments wherein the TAZ activator is at a concentration of about 20 mM.
Embodiment 2617. The pharmaceutical composition of any of the above enumerated embodiments wherein the TAZ activator is at a concentration of about 30 mM.
Embodiment 2618. The pharmaceutical composition of any of the above enumerated embodiments wherein the TAZ activator is at a concentration of about 40 mM.
Embodiment 2619. The pharmaceutical composition of any of the above enumerated embodiments wherein the TAZ activator is at a concentration of about 50 mM.
Embodiment 2620. The pharmaceutical composition of any of the above enumerated embodiments wherein the TAZ activator is at a concentration of about 60 mM.
Embodiment 2621. The pharmaceutical composition of any of the above enumerated embodiments wherein the TAZ activator is at a concentration of about 70 mM.
Embodiment 2622. The pharmaceutical composition of any of the above enumerated embodiments wherein the TAZ activator is at a concentration of about 80 mM.
Embodiment 2623. The pharmaceutical composition of any of the above enumerated embodiments wherein the TAZ activator is at a concentration of about 90 mM.
Embodiment 2624. The pharmaceutical composition of any of the above enumerated embodiments wherein the TAZ activator is at a concentration of about 100 mM.
Embodiment 2625. The pharmaceutical composition of any of the above enumerated embodiments wherein the TAZ activator is at a concentration of about 500 mM.
Embodiment 2626. The pharmaceutical composition of any of the above enumerated embodiments wherein the TAZ activator is at a unit dose of about 1.5 mg to 750 mg.
Embodiment 2627. The pharmaceutical composition of any of the above enumerated embodiments wherein the TAZ activator is at a unit dose of about 5 mg to 500 mg.
Embodiment 2628. The pharmaceutical composition of any of the above enumerated embodiments wherein the TAZ activator is at a unit dose of about 10 mg to 250 mg.
Embodiment 2629. The pharmaceutical composition of any of the above enumerated embodiments wherein the TAZ activator is at a unit dose of about 15 mg to 150 mg.
Embodiment 2630. The pharmaceutical composition of any of the above enumerated embodiments wherein the TAZ activator is at a unit dose of about 1.5 mg to 10 mg.
Embodiment 2631. The pharmaceutical composition of any of the above enumerated embodiments wherein the TAZ activator is at a unit dose of about 10 mg to 20 mg.
Embodiment 2632. The pharmaceutical composition of any of the above enumerated embodiments wherein the TAZ activator is at a unit dose of about 20 mg to 30 mg.
Embodiment 2633. The pharmaceutical composition of any of the above enumerated embodiments wherein the TAZ activator is at a unit dose of about 30 mg to 40 mg.
Embodiment 2634. The pharmaceutical composition of any of the above enumerated embodiments wherein the TAZ activator is at a unit dose of about 40 mg to 50 mg.
Embodiment 2635. The pharmaceutical composition of any of the above enumerated embodiments wherein the TAZ activator is at a unit dose of about 50 mg to 60 mg.
Embodiment 2636. The pharmaceutical composition of any of the above enumerated embodiments wherein the TAZ activator is at a unit dose of about 60 mg to 70 mg.
Embodiment 2637. The pharmaceutical composition of any of the above enumerated embodiments wherein the TAZ activator is at a unit dose of about 70 mg to 80 mg.
Embodiment 2638. The pharmaceutical composition of any of the above enumerated embodiments wherein the TAZ activator is at a unit dose of about 90 mg to 100 mg.
Embodiment 2639. The pharmaceutical composition of any of the above enumerated embodiments wherein the TAZ activator is at a unit dose of about 100 mg to 120 mg.
Embodiment 2640. The pharmaceutical composition of any of the above enumerated embodiments wherein the TAZ activator is at a unit dose of about 120 mg to 150 mg.
Embodiment 2641. The pharmaceutical composition of any of the above enumerated embodiments wherein the TAZ activator is at a concentration of about 0.1 mM to 100,000 mM.
Embodiment 2642. The pharmaceutical composition of any of the above enumerated embodiments wherein the TAZ activator is at a concentration of about 0.01 mM to 10,000 mM.
Embodiment 2643. The pharmaceutical composition of any of the above enumerated embodiments wherein the TAZ activator is at a concentration of about 0.1 mM to 1,000 mM.
Embodiment 2644. The pharmaceutical composition of any of the above enumerated embodiments wherein the TAZ activator is at a concentration of about 0.1 mM to 100 mM.
Embodiment 2645. The pharmaceutical composition of any of the above enumerated embodiments wherein the TAZ activator is at a concentration of about 0.001 mM to 0.01 mM.
Embodiment 2646. The pharmaceutical composition of any of the above enumerated embodiments wherein the TAZ activator is at a concentration of about 0.01 mM to 0.1 mM
Embodiment 2647. The pharmaceutical composition of any of the above enumerated embodiments wherein the TAZ activator is at a concentration of about 0.1 mM to 1 mM.
Embodiment 2648. The pharmaceutical composition of any of the above enumerated embodiments wherein the TAZ activator is at a concentration of about 1 mM to 10 mM.
Embodiment 2649. The pharmaceutical composition of any of the above enumerated embodiments wherein the TAZ activator is at a concentration of about 10 mM to 100 mM.
Embodiment 2650. The pharmaceutical composition of any of the above enumerated embodiments wherein the TAZ activator is at a concentration of about 100 mM to 1,000 mM.
Embodiment 2651. The pharmaceutical composition of any of the above enumerated embodiments wherein the TAZ activator is at a concentration of about 1,000 mM to 10,000 mM.
Embodiment 2652. The pharmaceutical composition of any of the above enumerated embodiments wherein the Wnt agonist is a Wnt agonist listed in Table 3.
Embodiment 2653. The pharmaceutical composition of any of the above enumerated embodiments wherein the Wnt agonist is AZD1080.
Embodiment 2654. The pharmaceutical composition of any of the above enumerated embodiments wherein the Wnt agonist is LY2090314.
Embodiment 2655. The pharmaceutical composition of any of the above enumerated embodiments wherein the Wnt agonist is GSK3 inhibitor XXII.
Embodiment 2656. The pharmaceutical composition of any of the above enumerated embodiments wherein the Wnt agonist is CHIR99021.
Embodiment 2657. The pharmaceutical composition of any of the above enumerated embodiments wherein the Wnt agonist is an analogue of LY2090314 as known in the art
Embodiment 2658. The pharmaceutical composition of any of the above enumerated embodiments wherein the Wnt agonist at a concentration of about 0.001 mM to 10,000 mM.
Embodiment 2659. The pharmaceutical composition of any of the above enumerated embodiments wherein the Wnt agonist at a concentration of about 0.01 mM to 1,000 mM.
Embodiment 2660. The pharmaceutical composition of any of the above enumerated embodiments wherein the Wnt agonist at a concentration of about 0.1 mM to 100 mM.
Embodiment 2661. The pharmaceutical composition of any of the above enumerated embodiments wherein the Wnt agonist at a concentration of about 0.001 mM to 0.01 mM.
Embodiment 2662. The pharmaceutical composition of any of the above enumerated embodiments wherein the Wnt agonist at a concentration of about 0.01 mM to 0.1 mM.
Embodiment 2663. The pharmaceutical composition of any of the above enumerated embodiments wherein the Wnt agonist at a concentration of about 0.1 mM to 1 mM.
Embodiment 2664. The pharmaceutical composition of any of the above enumerated embodiments wherein the Wnt agonist at a concentration of about 1 mM to 10 mM.
Embodiment 2665. The pharmaceutical composition of any of the above enumerated embodiments wherein the Wnt agonist at a concentration of about 10 mM to 100 mM.
Embodiment 2666. The pharmaceutical composition of any of the above enumerated embodiments wherein the Wnt agonist at a concentration of about 100 mM to 1,000 mM.
Embodiment 2667. The pharmaceutical composition of any of the above enumerated embodiments wherein the Wnt agonist at a concentration of about 1,000 mM to 10,000 mM.
Embodiment 2668. The pharmaceutical composition of any of the above enumerated embodiments wherein the Wnt agonist at a concentration of about 1 mM.
Embodiment 2669. The pharmaceutical composition of any of the above enumerated embodiments wherein the Wnt agonist at a concentration of about 2 mM.
Embodiment 2670. The pharmaceutical composition of any of the above enumerated embodiments wherein the Wnt agonist at a concentration of about 3 mM.
Embodiment 2671. The pharmaceutical composition of any of the above enumerated embodiments wherein the Wnt agonist at a concentration of about 4 mM.
Embodiment 2672. The pharmaceutical composition of any of the above enumerated embodiments wherein the Wnt agonist at a concentration of about 5 mM.
Embodiment 2673. The pharmaceutical composition of any of the above enumerated embodiments wherein the Wnt agonist at a concentration of about 6 mM.
Embodiment 2674. The pharmaceutical composition of any of the above enumerated embodiments wherein the Wnt agonist at a concentration of about 7 mM.
Embodiment 2675. The pharmaceutical composition of any of the above enumerated embodiments wherein the Wnt agonist at a concentration of about 8 mM.
Embodiment 2676. The pharmaceutical composition of any of the above enumerated embodiments wherein the Wnt agonist at a concentration of about 9 mM.
Embodiment 2677. The pharmaceutical composition of any of the above enumerated embodiments wherein the Wnt agonist at a concentration of about 10 mM.
Embodiment 2678. The pharmaceutical composition of any of the above enumerated embodiments wherein the Wnt agonist at a concentration of about 0.001 μM to 10 mM.
Embodiment 2679. The pharmaceutical composition of any of the above enumerated embodiments wherein the Wnt agonist at a concentration of about 0.01 μM to 1 mM.
Embodiment 2680. The pharmaceutical composition of any of the above enumerated embodiments wherein the Wnt agonist at a concentration of about 0.1 μM to 100 μM.
Embodiment 2681. The pharmaceutical composition of any of the above enumerated embodiments wherein the Wnt agonist at a concentration of about 0.001 μM to 0.01 μM.
Embodiment 2682. The pharmaceutical composition of any of the above enumerated embodiments wherein the Wnt agonist at a concentration of about 0.01 μM to 0.1 μM.
Embodiment 2683. The pharmaceutical composition of any of the above enumerated embodiments wherein the Wnt agonist at a concentration of about 0.1 μM to 1 μM.
Embodiment 2684. The pharmaceutical composition of any of the above enumerated embodiments wherein the Wnt agonist at a concentration of about 1 μM to 10 μM.
Embodiment 2685. The pharmaceutical composition of any of the above enumerated embodiments wherein the Wnt agonist at a concentration of about 10 μM to 100 μM.
Embodiment 2686. The pharmaceutical composition of any of the above enumerated embodiments wherein the Wnt agonist at a concentration of about 100 μM to 1 mM.
Embodiment 2687. The pharmaceutical composition of any of the above enumerated embodiments wherein the Wnt agonist at a concentration of about 1 mM to 10 mM.
Embodiment 2688. The pharmaceutical composition of any of the above enumerated embodiments wherein the Wnt agonist at a concentration of about 1 μM.
Embodiment 2689. The pharmaceutical composition of any of the above enumerated embodiments wherein the Wnt agonist at a concentration of about 5 μM.
Embodiment 2690. The pharmaceutical composition of any of the above enumerated embodiments wherein the Wnt agonist at a concentration of about 10 μM.
Embodiment 2691. The pharmaceutical composition of any of the above enumerated embodiments wherein the Wnt agonist at a concentration of about 15 μM.
Embodiment 2692. The pharmaceutical composition of any of the above enumerated embodiments wherein the Wnt agonist at a concentration of about 20 μM.
Embodiment 2693. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pvrrole-2,5-dione at a concentration of about 0.001 μM to 10 mM.
Embodiment 2694. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.01 μM to 1 mM.
Embodiment 2695. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.1 μM to 100 μM.
Embodiment 2696. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.001 μM to 0.01 μM.
Embodiment 2697. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.01 μM to 0.1 μM.
Embodiment 2698. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 0.1 μM to 1 μM.
Embodiment 2699. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 1 μM to 10 μM.
Embodiment 2700. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pvrrole-2,5-dione at a concentration of about 10 μM to 100 μM.
Embodiment 2701. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 100 μM to 1 mM.
Embodiment 2702. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 1 mM to 10 mM.
Embodiment 2703. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 1 μM.
Embodiment 2704. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 5 μM.
Embodiment 2705. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 10 μM.
Embodiment 2706. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 15 μM.
Embodiment 2707. The pharmaceutical composition of any of the above enumerated embodiments wherein the Wnt agonist is a substituted 3-Imidazo[1,2-a]pyridin-3-yl-4-(1,2,3,4-tetrahydro-[1,4]diazepino-[6,7,1-hi]indol-7-yl)pyrrole-2,5-dione at a concentration of about 20 μM.
Embodiment 2708. The pharmaceutical composition of any of the above enumerated embodiments wherein the Wnt agonist is GSK3-inhibitor XXII.
Embodiment 2709. The pharmaceutical composition of any of the above enumerated embodiments wherein the Wnt agonist is at a concentration of about 0.1 μM to 1,000 mM.
Embodiment 2710. The pharmaceutical composition of any of the above enumerated embodiments wherein the Wnt agonist is at a concentration of about 1 μM to 100 mM.
Embodiment 2711. The pharmaceutical composition of any of the above enumerated embodiments wherein the Wnt agonist is at a concentration of about 10 μM to 10 mM.
Embodiment 2712. The pharmaceutical composition of any of the above enumerated embodiments wherein the Wnt agonist is at a concentration of about 0.1 μM to 1 μM.
Embodiment 2713. The pharmaceutical composition of any of the above enumerated embodiments wherein the Wnt agonist is at a concentration of about 1 μM to 10 μM.
Embodiment 2714. The pharmaceutical composition of any of the above enumerated embodiments wherein the Wnt agonist is at a concentration of about 10 μM to 100 μM.
Embodiment 2715. The pharmaceutical composition of any of the above enumerated embodiments wherein the Wnt agonist is at a concentration of about 100 μM to 1 mM.
Embodiment 2716. The pharmaceutical composition of any of the above enumerated embodiments wherein the Wnt agonist is at a concentration of about 1 mM to 10 mM.
Embodiment 2717. The pharmaceutical composition of any of the above enumerated embodiments wherein the Wnt agonist is at a concentration of about 10 mM to 100 mM.
Embodiment 2718. The pharmaceutical composition of any of the above enumerated embodiments wherein the Wnt agonist is at a concentration of about 100 mM to 1000 mM.
Embodiment 2719. The pharmaceutical composition of any of the above enumerated embodiments wherein the Wnt agonist is at a concentration of about 0.1 mM.
Embodiment 2720. The pharmaceutical composition of any of the above enumerated embodiments wherein the Wnt agonist is at a concentration of about 0.2 mM.
Embodiment 2721. The pharmaceutical composition of any of the above enumerated embodiments wherein the Wnt agonist is at a concentration of about 0.3 mM.
Embodiment 2722. The pharmaceutical composition of any of the above enumerated embodiments wherein the Wnt agonist is at a concentration of about 0.4 mM.
Embodiment 2723. The pharmaceutical composition of any of the above enumerated embodiments wherein the Wnt agonist is at a concentration of about 0.5 mM.
Embodiment 2724. The pharmaceutical composition of any of the above enumerated embodiments wherein the Wnt agonist is at a concentration of about 0.6 mM
Embodiment 2725. The pharmaceutical composition of any of the above enumerated embodiments wherein the Wnt agonist is at a concentration of about 0.7 mM
Embodiment 2726. The pharmaceutical composition of any of the above enumerated embodiments wherein the Wnt agonist is at a concentration of about 0.8 mM
Embodiment 2727. The pharmaceutical composition of any of the above enumerated embodiments wherein the Wnt agonist is at a concentration of about 0.9 mM
Embodiment 2728. The pharmaceutical composition of any of the above enumerated embodiments wherein the Wnt agonist is at a concentration of about 1.0 mM.
Embodiment 2729. The pharmaceutical composition of any of the above enumerated embodiments wherein the Wnt agonist is a GSK3 Inhibitor.
Embodiment 2730. The pharmaceutical composition of any of the above enumerated embodiments wherein the Wnt agonist is CHIR99021.
Embodiment 2731. The pharmaceutical composition of any of the above enumerated embodiments further comprising an epigenetic agent.
Embodiment 2732. The pharmaceutical composition of any of the above enumerated embodiments wherein the epigenetic agent is an HDAC inhibitor.
Embodiment 2733. The pharmaceutical composition of any of the above enumerated embodiments wherein the HDAC inhibitor is at a concentration of about 10 μM to 1,000,000 mM.
Embodiment 2734. The pharmaceutical composition of any of the above enumerated embodiments wherein the HDAC inhibitor is at a concentration of about 1000 μM to 100,000 mM.
Embodiment 2735. The pharmaceutical composition of any of the above enumerated embodiments wherein the HDAC inhibitor is at a concentration of about 10,000 μM to 10,000 mM.
Embodiment 2736. The pharmaceutical composition of any of the above enumerated embodiments wherein the HDAC inhibitor is at a concentration of about 1000 μM to 10,000 μM.
Embodiment 2737. The pharmaceutical composition of any of the above enumerated embodiments wherein the HDAC inhibitor is at a concentration of about 10,000 μM to 100,000 μM.
Embodiment 2738. The pharmaceutical composition of any of the above enumerated embodiments wherein the HDAC inhibitor is at a concentration of about 100,000 μM to 1,000,000 μM.
Embodiment 2739. The pharmaceutical composition of any of the above enumerated embodiments wherein the HDAC inhibitor is at a concentration of about 1,000 mM to 10,000 mM.
Embodiment 2740. The pharmaceutical composition of any of the above enumerated embodiments wherein the HDAC inhibitor is at a concentration of about 10,000 mM to 100,000 mM.
Embodiment 2741. The pharmaceutical composition of any of the above enumerated embodiments wherein the HDAC inhibitor is VPA.
Embodiment 2742. The pharmaceutical composition of any of the above enumerated embodiments wherein the HDAC inhibitor is at a unit dose of about 50 mg.
Embodiment 2743. The pharmaceutical composition of any of the above enumerated embodiments wherein the HDAC inhibitor is at a unit dose of about 100 mg.
Embodiment 2744. The pharmaceutical composition of any of the above enumerated embodiments wherein the HDAC inhibitor is at a unit dose of about 125 mg.
Embodiment 2745. The pharmaceutical composition of any of the above enumerated embodiments wherein the HDAC inhibitor is at a unit dose of about 250 mg.
Embodiment 2746. The pharmaceutical composition of any of the above enumerated embodiments wherein the HDAC inhibitor is at a unit dose of about 500 mg.
Embodiment 2747. The pharmaceutical composition of any of the above enumerated embodiments wherein the HDAC inhibitor is at a unit dose of about 1000 mg.
Embodiment 2748. The pharmaceutical composition of any of the above enumerated embodiments wherein the HDAC inhibitor is at a unit dose of about 2000 mg.
Embodiment 2749. The pharmaceutical composition of any of the above enumerated embodiments wherein the HDAC inhibitor is at a unit dose of about 3000 mg.
Embodiment 2750. The pharmaceutical composition of any of the above enumerated embodiments wherein the HDAC inhibitor is at a unit dose of about 4000 mg.
Embodiment 2751. The pharmaceutical composition of any of the above enumerated embodiments wherein the HDAC inhibitor is at a unit dose of about 5000 mg
Embodiment 2752. The pharmaceutical composition of any of the above enumerated embodiments wherein the unit dose of the HDAC inhibitor is an oral dosage form.
Embodiment 2753. A TAZ activator for use in treating or preventing an inner ear hearing or balance disorder in a subject, wherein the subject has been, or will be, administered a Wnt agonist.
Embodiment 2754. A Wnt agonist for use in treating or preventing an inner ear hearing or balance disorder in a subject, wherein the subject has been, or will be, administered a TAZ activator.
Embodiment 2755. An epigenetic agent for use in treating or preventing an inner ear hearing or balance disorder in a subject, wherein the subject has been, or will be, administered a TAZ activator and a Wnt agonist.
Embodiment 2756. The TAZ activator, Wnt agonist or epigenetic agent for use according to any preceding embodiment, wherein the treatment is as defined in any preceding embodiment that describes a method of treatment.
Embodiment 2757. A container comprising a TAZ activator and instructions, where those instructions describe the TAZ activator's use for treating or preventing an inner ear hearing or balance disorder in a subject, wherein the instructions require that the subject has been, or will be, administered a Wnt agonist.
Embodiment 2758. A container comprising a Wnt agonist and instructions, where those instructions describe the Wnt agonist's use in treating or preventing an inner ear hearing or balance disorder in a subject, wherein the instructions require that the subject has been, or will be, administered a TAZ activator.
Embodiment 2759. A container comprising an epigenetic agent and instructions, where those instructions describe the epigenetic agent's use in treating or preventing an inner ear hearing or balance disorder in a subject, wherein the instructions require that the subject has been, or will be, administered a TAZ activator and a Wnt agonist.
Embodiment 2760. The container according to any preceding embodiment, wherein the treatment described in the instructions is as defined in any preceding embodiment that describes a method of treatment.

### Definitions

In this application, the use of “or” includes “and/or” unless stated otherwise. As used in this application, the term “comprise” and variations of the term, such as “comprising” and “comprises,” are not intended to exclude other additives, components, integers or steps. By “consisting of” is meant including, and limited to, whatever follows the phrase “consisting of.” Thus, the phrase “consisting of” indicates that the listed elements are required or mandatory, and that no other elements are present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether they materially affect the activity or action of the listed elements.

The terms “about” and “approximately” are used as equivalents. Any numerals used in this disclosure with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Any reference to a compound is also a reference to a pharmaceutically acceptable salt of that compound (regardless of whether or not pharmaceutically acceptable salts are explicitly mentioned). Any compound can be provided for use in the invention in any pharmaceutically acceptable solid form, e.g. salt, solvate, hydrate, polymorph, amorphous material form etc. Any references to a compound also include references to artificially deuterated forms of that compound.

“Activity” refers to biological function mediated by proteins of a cell measured by methods known in the art such as immunostaining and western blotting in conjunction with cellular effects such as proliferation, cellular growth, or cellular gene expression.

“Administration” refers to introducing a substance into a subject. In some embodiments, administration is auricular, intrarticular, intracochlear, intravestibular, or transtympanically, e.g. by injection. In some embodiments, administration is directly to the inner ear, e.g. injection through the round window, otic capsule, or vestibular canals. In some embodiments, administration is directly into the inner ear via a cochlear implant delivery system. In some embodiments, the substance is injected transtympanically to the middle ear. In certain embodiments the substance is administered systemically (e.g. orally or parenterally). In certain embodiments “causing to be administered” refers to administration of a second component after a first component has already been administered (e.g. at a different time and/or by a different actor).

“Agonist” refers to an agent that causes an increase in the expression, levels, and/or activity of a target gene, protein, and/or pathway. In some instances, an agonist directly binds to and activates a target protein. In some instances, an agonist increases the activity of a pathway by binding to and modulating the activity of one or more pathway components, for example, by inhibiting the activity of negative regulator(s) of the pathway, or by activating upstream or downstream regulator(s) of the pathway.

“Auricular administration” refers to a method of using a catheter or wick device to administer a composition across the tympanic membrane to the inner ear of the subject. To facilitate insertion of the wick or catheter, the tympanic membrane is pierced using a suitably sized syringe or pipette. The devices could also be inserted using any other methods known to those of skill in the art, e.g. surgical implantation of the device. In particular embodiments, the wick or catheter device is a stand-alone device, meaning that it is inserted into the ear of the subject and then the composition is controllably released to the inner ear. In other particular embodiments, the wick or catheter device is attached or coupled to a pump or other device that allows for the administration of additional compositions. The pump is automatically programmed to deliver dosage units or is controlled by the subject or medical professional.

“Cell Aggregate” as used herein refers to a body cells in the organ of Corti that have proliferated to form a cluster of a given cell type that is greater than 40 microns in diameter and/or produced a morphology in which greater than 3 cell layers reside perpendicular to the basilar membrane.

“Cell Aggregate” can also refer a process in which cell division creates a body of cells that cause one or more cell types to breach the reticular lamina, or the boundary between endolymph and perilymph.

“Cell Density” as used herein in connection with a specific cell type is the mean number of that cell type per area in a Representative Microscopy Sample. The cell types may include but are not limited to Lgr5+ cells, hair cells, or supporting cells. The Cell Density is assessed with a given cell type in a given organ or tissue, including but not limited to the cochlea or organ of Corti. For instance, the Lgr5+ Cell Density in the organ of Corti is the Cell Density of Lgr5+ cells as measured across the organ of Corti. Typically, supporting cells and Lgr5+ cells will be enumerated by taking cross sections of the organ of Corti. Typically, hair cells will be enumerated by looking down at the surface of the organ of Corti, though cross sections are used in some instances, as described in a Representative Microscopy Sample. Typically, Cell Density of Lgr5+ cells will be measured by analyzing whole mount preparations of the Organ of Corti and counting the number of Lgr5 cells across a given distance along the surface of the epithelia, as described in a Representative Microscopy Sample. Hair cells are identified by their morphological features such as bundles or hair cell specific stains (e.g. Myosin VIIa, Prestin, vGlut3, Pou4f3, Espin, conjugated-Phalloidin, PMCA2, Ribeye, Atoh1, etc.). Lgr5+ cells are identified by specific stains or antibodies (e.g. Lgr5-GFP transgenic reporter, anti-Lgr5 antibody, etc.)

“Cochlear Concentration” as used herein will be the concentration of a given agent as measured through sampling cochlear fluid or tissue. Unless otherwise noted, the sample should contain a substantial enough portion of the cochlear fluid or tissue so that it is approximately representative of the average concentration of the agent in the cochlea. For example, samples are drawn from a vestibular canal, and a series of fluid samples drawn in series such that individual samples are comprised of cochlear fluid in specified portions of the cochlea

“Complementary nucleic acid sequence” refers to a nucleic acid sequence capable of hybridizing with another nucleic acid sequence comprised of complementary nucleotide base pairs.

“Cross-Sectional Cell Density” as used herein in connection with a specific cell type is the mean number of that cell type per area of cross section through a tissue in a Representative Microscopy Sample. Cross sections of the organ of Corti can also be used to determine the number of cells in a given plane. Typically, hair cells Cross-sectional Cell Density will be measured by analyzing whole mount preparations of the organ of Corti and counting the number of hair cells across a given distance in cross sections taken along a portion of the epithelia, as described in a Representative Microscopy Sample. Typically, Cross-sectional Cell Density of Lgr5+ cells will be measured by analyzing whole mount preparations of the organ of Corti and counting the number of Lgr5+ cells across a given distance in cross sections taken along a portion of the epithelia, as described in a Representative Microscopy Sample. Hair cells are identified by their morphological features such as bundles or hair cell specific stains (suitable stains include e.g. Myosin VIIa, Prestin, vGlut3, Pou4f3, conjugated-Phalloidin, PMCA2, Atoh1, etc.). Lgr5+ cells are identified by specific stains or antibodies (suitable stains and antibodies include fluorescence in situ hybridization of Lgr5 mRNA, Lgr5-GFP transgenic reporter system, anti-Lgr5 antibodies, etc.).

“Decreasing” or “decreases” refers to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference or control.

“Decreasing” or “decreases” also includes decreasing by at least about 1.1-fold, for example, at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more, for example, as compared to the level of a reference or control.

“Effective Concentration” is the minimum concentration of a compound that induces at least an 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more in gene expression and/or about a 1.5-fold increase in number of Lgr5+ cells in a Stem Cell Proliferation Assay compared to the number of Lgr5+ cells in a Stem Cell Proliferation Assay performed without the compound.

“Effective Release Rate” (mass/time) as used herein is the Effective Concentration (mass/volume)*30 uL/1 hour.

“Eliminate” means to decrease to a level that is undetectable.

“Engraft” or “engraftment” refers to the process of stem or progenitor cell incorporation into a tissue of interest in vivo through contact with existing cells of the tissue. “Epithelial progenitor cell” refers to a multipotent cell which has the potential to become restricted to cell lineages resulting in epithelial cells.

“Epithelial stem cell” refers to a multipotent cell which has the potential to become committed to multiple cell lineages, including cell lineages resulting in epithelial cells.

“Expression” refers to gene levels as measured by the amount of RNA

“HDAC inhibitor” refers to any compound that inhibits the cellular activity of Histone Deacetylase classes I-IV

“Hybridize” refers to pairing to form a double-stranded molecule between complementary nucleotide bases (e.g. adenine (A) forms a base pair with thymine (T), as does guanine (G) with cytosine (C) in DNA) under suitable conditions of stringency. (See, e.g. Wahl, G. M. and S. L. Berger (1987) Methods Enzymol. 152:399; Kimmel, A. R (1987) Methods Enzymol. 152:507).

An “inhibitor” refers to an agent that causes a decrease in the expression, levels, and/or activity of a target gene, protein, and/or pathway. An “antagonist” is one example of an “inhibitor”.

As used herein, an “inhibitory nucleic acid” is a double-stranded RNA, RNA interference, miRNA, siRNA, shRNA, or antisense molecule, or a portion thereof, or a mimetic thereof, that when administered to a mammalian cell results in a decrease in the expression of a target gene. Typically, a nucleic acid inhibitor comprises at least a portion of a target nucleic acid molecule, or an ortholog thereof, or comprises at least a portion of the complementary strand of a target nucleic acid molecule. In some instances, expression of a target gene is reduced by 10%, 25%, 50%, 75%, or even 90-100%.

“In vitro Lgr5 activity” refers to the level of expression or activity of Lgr5 in an in vitro population of cells. It is measured, for example, in cells derived from a Lgr5-GFP expressing mouse such as a B6.129P2-Lgr5tm1(cre/ERT2)Cle/J mouse (also known as Lgr5-EGFP-IRES-creERT2 or Lgr5-GFP mouse, Jackson Lab Stock No: 008875) by dissociating cells to single cells, staining with propidium iodide (PI), and analyzing the cells using a flow cytometer for Lgr5-GFP expression. Inner ear epithelial cells from wild-type (non-Lgr5-GFP) mice that passing the same culturing and analyzing procedures can be used as a negative control. Typically, two population of cells are shown in the bivariate plot with GFP/FITC as one variable, which include both GFP positive and GFP negative populations. Lgr5+ cells can be identified by gating GFP positive cell population. The percentage of Lgr5+ cells can be measured by gating GFP positive cell population against both GFP negative population and the negative control. The number of Lgr5+ cells can be calculated by multiplying the total number of cells by the percentage of Lgr5-positive cells. For cells derived from non-Lgr5-GFP mice, Lgr5 activity can be measured using an anti-Lgr5 antibody or quantitative-PCR on the Lgr5 gene.

“In vivo Lgr5 activity” as used herein is the level of expression or activity of Lgr5 in a subject. It is measured, for example, by removing an animal's inner ear and measuring Lgr5 protein or Lgr5 mRNA. Lgr5 protein production can be measured using an anti-Lgr5 antibody to measure fluorescence intensity as determined by imaging cochlear samples, where fluorescence intensity is used as a measure of Lgr5 presence. Western blots can be used with an anti-Lgr5 antibody, where cells can be harvested from the treated organ to determine increases in Lgr5 protein. Quantitative-PCR or RNA in situ hybridization can be used to measure relative changes in Lgr5 mRNA production, where cells can be harvested from the inner ear to determine changes in Lgr5 mRNA. Alternatively, Lgr5 expression can be measured using an Lgr5 promoter driven GFP reporter transgenic system, where the presence or intensity GFP fluoresce can be directly detected using flow cytometry, imaging, or indirectly using an anti-GFP antibody.

“Increasing” or “increases” refers to increasing by at least 5%, for example, 5, 6, 7, 8, 9, 10, 15, 20, 25, 30, 35, 40, 45, 50, 55, 60, 65, 70, 75, 80, 85, 90, 95, 99, 100, 150, 200, 250, 300, 350, 400, 450, or 500% or more, for example, as compared to the level of a reference.

“Increasing” or “increases” also means increases by at least about 1.1-fold, for example, at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more, for example, as compared to the level of a reference standard.

“Intrarticular administration” refers to administration of a composition to the middle or inner ear of a subject by directly injecting the composition.

“Intracochlear” administration refers to direct injection of a composition across the tympanic membrane and across the round window membrane into the cochlea.

“Intravestibular” administration refers to direct injection of a composition across the tympanic membrane and across the round window or oval window membrane into the vestibular organs.

“Isolated” refers to a material that is free to varying degrees from components which normally accompany it as found in its native state. “Isolate” denotes a degree of separation from original source or surroundings.

“Lgr5” is an acronym for the Leucine-rich repeat-containing G-protein coupled receptor 5, also known as G-protein coupled receptor 49 (GPR49) or G-protein coupled receptor 67 (GPR67). It is a protein that in humans is encoded by the Lgr5 gene.

“Lgr5 Activity” is defined as the level of activity of Lgr5 in a population of cells. In an in vitro cell population, Lgr5 activity is measured in an in vitro Lgr5 Activity assay. In an in vivo cell population, Lgr5 activity is measured in an in vivo Lgr5 Activity assay.

“Lgr5+ cell” or “Lgr5-positive cell” as used herein is a cell that expresses Lgr5. “Lgr5-cell” or “Lgr5-negative” as used herein is a cell that is not Lgr5+.

“Lineage Tracing” as used herein is using a mouse line that enables fate tracing of any cell that expresses a target gene at the time of reporter induction. This can include hair cell or supporting cells genes (Sox2, Lgr5, MyosinVIIa, Pou4f3, etc.). For example, lineage tracing may use an Lgr5-EGFP-IRES-creERT2 mouse crossed with a reporter mouse, which upon induction, allows one to trace the fate of cells that expressed Lgr5 at the time of induction. By further example, Lgr5 cells can be isolated into single cells and cultured in a Stem Cell Proliferation Assay to generate colonies, then subsequently differentiated in a Differentiation Assay and analyzed for cell fate by staining for hair cell and/or supporting cell proteins and determining the reporter co-localization with either hair cell or supporting cell staining to determine the Lgr5 cells' fate. In addition, lineage tracing can be performed in cochlear explants to track supporting cell or hair cell fate within the intact organ after treatment. For example, Lgr5 cell fate can be determined by isolating the cochlea from a Lgr5-EGFP-IRES-creERT2 mouse crossed with a reporter mouse and inducing the reporter in Lgr5 cells before or during treatment. The organ can then be analyzed for cell fate by staining for hair cell and/or supporting cell proteins and determining the reporter co-localization with either hair cell or supporting cell staining to determine the Lgr5 cells' fate. In addition, lineage tracing can be performed in vivo track supporting cell or hair cell fate within the intact organ after treatment. For example, Lgr5 cell fate can be determined inducing a reporter in an Lgr5-EGFP-IRES-creERT2 mouse crossed with a reporter mouse, treating the animal, then isolating the cochlea. The organ can then be analyzed for cell fate by staining for hair cell and/or supporting cell proteins and determining the reporter co-localization with either hair cell or supporting cell staining to determine the Lgr5 cells' fate. Lineage tracing is performed using alternative reporters of interest as is standard in the art.

“Mammal” refers to any mammal including but not limited to human, mouse, rat, sheep, monkey, goat, rabbit, hamster, horse, cow or pig.

“Mean Release Time” as used herein is the time in which one-half of an agent is released into phosphate buffered saline from a carrier in a Release Assay.

“Native Morphology” as used herein is means that tissue organization largely reflects the organization in a healthy tissue.

“Non-human mammal”, as used herein, refers to any mammal that is not a human.

As used in relevant context herein, the term “number” of cells can be 0, 1, or more cells.

“Organ of Corti” as used herein refers to the sensory epithelia of the cochlea where the sensory cells (inner and outer hair cells) and supporting cells reside.

“Organoid” or “epithelial organoid” refers to a cell cluster or aggregate that resembles an organ, or part of an organ, and possesses cell types relevant to that particular organ.

“Pharmaceutically-acceptable salt” includes both acid and base addition salts.

“Pharmaceutically-acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline, and caffeine.

“Population” of cells refers to any number of cells greater than 1, but can be at least 1×103 cells, at least 1×104 cells, at least at least 1×105 cells, at least 1×106 cells, at least 1×107 cells, at least 1×108 cells, at least 1×109 cells, or at least 1×1010 cells.

“Progenitor cell” as used herein refers to a cell that, like a stem cell, has the tendency to differentiate into a specific type of cell, but is already more specific than a stem cell and is pushed to differentiate into its “target” cell.

“Proliferation Period” as used herein is the duration of time in which tissue or cells are exposed to a TAZ activator alone or in combination with a Wnt agonist.

In certain embodiments, the “purity” of any given agent or compound in a composition is specifically defined. For instance, certain compositions may comprise an agent that is at least 80, 85, 90, 91, 92, 93, 94, 95, 96, 97, 98, 99, or 100% pure, including all decimals in between, as measured, for example and by no means limiting, by high performance liquid chromatography (HPLC), a well-known form of column chromatography used frequently in biochemistry and analytical chemistry to separate, identify, and quantify compounds.

“Reference” means a standard or control condition (e.g. untreated with a test agent or combination of test agents).

“Release Assay” as used herein is a test in which the rate of release of an agent from a Biocompatible Matrix through dialysis membrane to a saline environment. An exemplary Release Assay is performed by placing 30 microliters of a composition in 1 ml Phosphate Buffered Saline inside saline dialysis bag with a suitable cutoff, and placing the dialysis bag within 10 mL of Phosphate Buffered Saline at 37° C. The dialysis membrane size is chosen based on agent size in order to allow the agent being assessed to exit the membrane. For small molecule release, a 3.5-5 kDa cutoff is used. The Release Rate for a composition may change over time and is measured in 1 hour increments.

“Representative Microscopy Sample” as used herein describes a sufficient number of fields of view within a cell culture system, a portion of extracted tissue, or an entire extracted organ that the average feature size or number being measured can reasonably be said to represent the average feature size or number if all relevant fields were measured. For example, in order to assess the hair cell counts at a frequency range on the Organ of Corti, ImageJ software (NIH) can used to measure the total length of cochlear whole mounts and the length of individual counted segments. The total number of inner hair cells, outer hair cells, and supporting cells can be counted in the entire or fraction of any of the four cochlear segments of 1200-1400 μm (apical, mid-apical, mid-basal, and basal) at least 3 fields of view at 100 μm field size would be reasonably considered a Representative Microscopy Sample. A Representative Microscopy sample can include measurements within a field of view, which can be measured as cells per a given distance. A Representative Microscopy sample can be used to assess morphology, such as cell-cell contacts, cochlear architecture, and cellular components (e.g. bundles, synapses).

“Rosette Patterning” is a characteristic cell arrangement in the cochlea in which <5% hair cells are adjacent to other hair cells.

The term “sample” refers to a volume or mass obtained, provided, and/or subjected to analysis. In some embodiments, a sample is or comprises a tissue sample, cell sample, a fluid sample, and the like. In some embodiments, a sample is taken from (or is) a subject (e.g. a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid is, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g. a subject); in some embodiments, a “sample” is the result of processing of a primary sample, for example to remove certain potentially contaminating components and/or to isolate or purify certain components of interest.

“Self-renewal” refers to the process by which a stem cell divides to generate one (asymmetric division) or two (symmetric division) daughter cells with development potentials that are indistinguishable from those of the mother cell. Self-renewal involves both proliferation and the maintenance of an undifferentiated state.

“siRNA” refers to a double stranded RNA. Optimally, an siRNA is 18, 19, 20, 21, 22, 23 or 24 nucleotides in length and has a 2 base overhang at its 3′ end. These dsRNAs can be introduced to an individual cell or culture system. Such siRNAs are used to downregulate mRNA levels or promoter activity.

“Stem cell” refers to a multipotent cell having the capacity to self-renew and to differentiate into multiple cell lineages.

“Stem Cell Differentiation Assay” as used herein is an assay to determine the differentiation capacity of stem cells. In an exemplary Stem Cell Differentiation Assay, the number of cells for an initial cell population is harvested from a Atoh1-GFP mouse between the age of 3 to 7 days, by isolating the Organ of Corti sensory epithelium, dissociating the epithelium into single cells, and passing the cells through a 40 um cell strainer. Approximately 5000 cells are entrapped in 40 μl of culture substrate (for example: Matrigel® (Corning, Growth Factor Reduced)) and placed at the center of wells in a 24-well plate with 500 μl of an appropriate culture media, growth factors and agent being tested. Appropriate culture media and growth factors include Advanced DMEM/F12 with media Supplements (1× N2, 1× B27, 2 mM Glutamax, 10 mM HEPES, 1 mM N-acetylcysteine, and 100 U/ml penicillin/100 pg/ml streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C. and 5% CO2, with media change every 2 days. These cells are then cultured by removing the Stem Cell Proliferation Assay agents and replacing with Basal culture media and molecules to drive differentiation. An appropriate Basal culture medium is Advanced DMEM/F12 supplemented with 1× N2, 1× B27, 2 mM GlutaMax™, 10 mM HEPES, 1 mM N-acetylcysteine, and 100 U/ml penicillin/100 pg/ml streptomycin and appropriate molecules to drive differentiation are 3 μM CHIR99021 and 5 μM DAPT for 10 days, with media change every 2 days. The number of hair cells in a population is measured using flow cytometry for GFP. Hair cell differentiation level can further be assessed using qPCR to measure hair cell marker (e.g. Myo7a) expression level normalized using suitable and unregulated references or housekeeping genes (e.g. Hprt). Hair cell differentiation level can also be assessed by immunostaining for hair cell markers (e.g. Myosin7a, vGlut3, Espin, PMCAs, Ribeye, conjugated-phalloidin, Atoh1, Pou4f3, etc.). Hair cell differentiation level can also be assessed by Western Blot for Myosin7a, vGlut3, Espin, PMCAs, Prestin, Ribeye, Atoh1, Pou4f3.

“Stem Cell Assay” as used herein is an assay in which a cell or a cell population are tested for a series of criteria to determine whether the cell or cell population are stem cells or enriched in stem cells or stem cell markers. In a stem cell assay, the cell/cell population are tested for stem cell characteristics such as expression of Stem Cell Markers, and further optionally are tested for stem cell function, including the capacity of self-renewal and differentiation. Gene expression is measured using methods known in the art such as by PCR, Nanostring, immunostaining, RNAseq, RNA hybridization, or Western blot analysis.

“Stem Cell Proliferation Assay” as used herein is an assay to determine the capacity for agent(s) to induce the creation of stem cells from a starting cell population. In an exemplary Stem Cell Proliferation Assay, the number of cells for an initial cell population is harvested from a Lgr5-GFP mouse such as a B6.129P2-Lgr5tm1(cre/ERT2)Cle/J mouse (also known as Lgr5-EGFP-IRES-creERT2 or Lgr5-GFP mouse, Jackson Lab Stock No: 008875) between the age of 0 to 5 days, by isolating the organ of Corti sensory epithelium and dissociating the epithelium into single cells. Approximately 5000 cells are entrapped in 40 μl of culture substrate (for example: Matrigel (Corning, Growth Factor Reduced)) and placed at the center of wells in a 24-well plate with 500 μl of an appropriate culture media, growth factors and agent being tested. Appropriate culture media and growth factors include Advanced DMEM/F12 with media Supplements (1× N2, 1× B27, 2 mM Glutamax, 10 mM HEPES, 1 mM N-acetylcysteine, and 100 U/ml penicillin/100 pg/ml streptomycin) and growth factors (50 ng/ml EGF, 50 ng/ml bFGF, and 50 ng/ml IGF-1) as well as the agent(s) being assessed are added into each well. Cells are cultured for 10 days in a standard cell culture incubator at 37° C. and 5% CO2, with media change every 2 days. The number of Lgr5+ cells is quantified by counting the number of cells identified as Lgr5+ in an In vitro Lgr5 activity assay. The fraction of cells that are Lgr5+ is quantified by dividing the number of cells identified as Lgr5+ in a cell population by the total number of cells present in the cell population. The number of hair cells in a population is measured by staining with hair cell marker (e.g. MyosinVIIa), or using an endogenous reporter of hair cell genes (e.g. Pou4f3-GFP, Atoh1-nGFP) and analyzing using flow cytometry. The fraction of cells that are hair cells is quantified by dividing the number of cells identified as hair cells in a cell population by the total number of cells present in the cell population. Gene and/or protein expression and/or activity is measured in this assay using methods known in the art such as by PCR, Nanostring, immunostaining, RNAseq, RNA hybridization, or Western blot analysis.

“Stem Cell Markers” as used herein can be defined as gene products (e.g. protein, RNA, etc.) that specifically expressed in stem cells. One type of stem cell marker is gene products that are directly and specifically support the maintenance of stem cell identity. Examples include Lgr5 and Sox2. Additional stem cell markers can be identified using assays that were described in the literatures. To determine whether a gene is required for maintenance of stem cell identity, gain-of-function and loss-of-function studies can be used. In gain-of-function studies, over expression of specific gene product (the stem cell marker) would help maintain the stem cell identity. While in loss-of-function studies, removal of the stem cell marker would cause loss of the stem cell identity or induced the differentiation of stem cells. Another type of stem cell marker is gene that only expressed in stem cells but does not necessary to have specific function to maintain the identity of stem cells. This type of markers can be identified by comparing the gene expression signature of sorted stem cells and non-stem cells by assays such as micro-array and qPCR. This type of stem cell marker can be found in the literature. (e.g. Liu Q. et al., Int J Biochem Cell Biol. 2015 March; 60:99-111. http://www.ncbi.nlm.nih.gov/pubmed/25582750). Potential stem cell markers include Ccdc121, Gdf10, Opcm1, Phex, etc. The expression of stem cell markers such as Lgr5 or Sox2 in a given cell or cell population can be measure using assays such as qPCR, immunohistochemistry, western blot, and RNA hybridization. The expression of stem cell markers can also be measured using transgenic cells express reporters which can indicate the expression of the given stem cell markers, e.g. Lgr5-GFP or Sox2-GFP. Flow cytometry analysis can then be used to measure the activity of reporter expression. Fluorescence microscopy can also be used to directly visualize the expression of reporters. The expression of stem cell markers may further be determined using microarray analysis for global gene expression profile analysis. The gene expression profile of a given cell population or purified cell population can be compared with the gene expression profile of the stem cell to determine similarity between the 2 cell populations. Stem cell function can be measured by colony forming assay or sphere forming assay, self-renewal assay and differentiation assay. In colony (or sphere) forming assay, when cultured in appropriate culture media, the stem cell should be able to form colonies, on cell culture surface (e.g. cell culture dish) or embedded in cell culture substrate (e.g. Matrigel) or be able to form spheres when cultured in suspension. In colony/sphere forming assay, single stem cells are seeded at low cell density in appropriate culture media and allowed to proliferate for a given period of time (7-10 days). Colony formed are then counted and scored for stem cell marker expression as an indicator of sternness of the original cell. Optionally, the colonies that formed are then picked and passaged to test its self-renewal and differentiation potential. In self-renewal assay, when cultured in appropriate culture media, the cells should maintain stem cell marker (e.g. Lgr5) expression over at least one (e.g. 1, 2, 3, 4, 5, 10, 20, etc.) cell divisions. In a Stem Cell Differentiation Assay, when cultured in appropriate differentiation media, the cells should be able to generate hair cell which can be identified by hair cell marker expression measured by qPCR, immunostaining, western blot, RNA hybridization or flow cytometry.

“Subject” includes humans and mammals (e.g. mice, rats, pigs, cats, dogs, and horses). In some embodiments, subjects are be mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g. particularly in research contexts) subject mammals will be, for example, rodents (e.g. mice, rats, hamsters), rabbits, primates, or swine such as inbred pigs and the like.

“Supporting Cell” as used herein in connection with a cochlear epithelium comprises epithelial cells within the organ of Corti that are not hair cells. This includes inner pillar cells, outer pillar cells, inner phalangeal cells, Deiter cells, Hensen cells, Boettcher cells, and/or Claudius cells.

By “statistically significant”, it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Substantially” or “essentially” means nearly totally or completely, for instance, 95% or greater of some given quantity.

“Synergist” refers to a compound that causes a more than additive increase in target gene expression or protein levels by 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold more than the additive value of each compound used individually.

“Tissue” is an ensemble of similar cells from the same origin that together carry out a specific function including, for example, tissue of cochlear, such as the organ of Corti.

“Transtympanic” administration refers to direct injection of a composition across the tympanic membrane into the middle ear.

“Treating” as used herein in connection with a cell population means delivering a substance to the population to affect an outcome. In the case of in vitro populations, the substance is directly (or even indirectly) delivered to the population. In the case of in vivo populations, the substance is delivered by administration to the host subject.

“Vehicle Control” or “Control” refers to treatment with the carrier that is absent of drug, such as DMSO for in vitro assays, poloxamer for middle ear delivery, and/or carrier or solution used to deliver drug compounds to cochlear cells describe here.

It is to be appreciated that references to “treating” or “treatment” include the alleviation of established symptoms of a condition. “Treating” or “treatment” of a state, disorder or condition therefore includes: (1) preventing or delaying the appearance of clinical symptoms of the state, disorder or condition developing in a human that is afflicted with or predisposed to the state, disorder or condition but does not yet experience or display clinical or subclinical symptoms of the state, disorder or condition, (2) inhibiting the state, disorder or condition, i.e. arresting, reducing or delaying the development of the disease or a relapse thereof (in case of maintenance treatment) or at least one clinical or subclinical symptom thereof, or (3) relieving or attenuating the disease, i.e. causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Wnt activation” as used herein is an activation of the Wnt signaling pathway.

“Wnt alone” as used herein means when the activity as described herein of another agent or combination of agents is compared the activity of “Wnt alone” it is meant comparison is made using the same the Wnt agent at the same concentration.

The term “alkyl” as used herein refers to a straight or branched saturated hydrocarbon. For example, an alkyl group can have 1 to 8 carbon atoms (i.e. (C₁-C₈)alkyl) or 1 to 6 carbon atoms (i.e. (C₁-C₆ alkyl) or 1 to 4 carbon atoms.

The term “alkenyl” as used herein refers to a linear or branched hydrocarbon radical which includes one or more double bonds and can include divalent radicals, having from 2 to about 15 carbon atoms. Examples of alkenyl groups include but are not limited to, ethenyl, propenyl, butenyl, and higher homologs and isomers.

The term “alkynyl” as used herein refers to a linear or branched hydrocarbon radical which includes one or more triple bonds and can include divalent radicals, having from 2 to about 15 carbon atoms. Examples of alkynyl groups include but are not limited to, ethynyl, propynyl, butynyl, and higher homologs and isomers.

The term “halo” or “halogen” as used herein refers to fluoro, chloro, bromo and iodo.

The term “aryl” as used herein refers to a single all carbon aromatic ring or a multiple condensed all carbon ring system wherein at least one of the rings is aromatic. For example, an aryl group can have 6 to 20 carbon atoms, 6 to 14 carbon atoms, or 6 to 12 carbon atoms. Aryl includes a phenyl radical. Aryl also includes multiple condensed ring systems (e.g. ring systems comprising 2, 3 or 4 rings) having about 9 to 20 carbon atoms in which at least one ring is aromatic and wherein the other rings are aromatic or not aromatic (i.e. carbocycle). Such multiple condensed ring systems are optionally substituted with one or more (e.g. 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring.

The term “heteroaryl” as used herein refers to a single aromatic ring that has at least one atom other than carbon in the ring, wherein the atom is selected from the group consisting of oxygen, nitrogen and sulfur; the term also includes multiple condensed ring systems that have at least one such aromatic ring, which multiple condensed ring systems are further described below. Thus, the term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. The term also includes multiple condensed ring systems (e.g. ring systems comprising 2, 3 or 4 rings) wherein a heteroaryl group, as defined above, can be condensed with one or more rings selected from heteroaryls (to form for example a naphthyridinyl such as 1,8-naphthyridinyl), heterocycles, (to form for example a 1, 2, 3, 4-tetrahydronaphthyridinyl such as 1, 2, 3, 4-tetrahydro-1,8-naphthyridinyl), carbocycles (to form for example 5,6,7, 8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems are optionally substituted with one or more (e.g. 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system are connected in any order relative to one another. It is also to be understood that the point of attachment of a multiple condensed ring system (as defined above for a heteroaryl) can be at any position of the multiple condensed ring system including a heteroaryl, heterocycle, aryl or carbocycle portion of the multiple condensed ring system and at any suitable atom of the multiple condensed ring system including a carbon atom and heteroatom (e.g. a nitrogen).

The term “cycloalkyl” as used herein refers to a saturated or partially saturated ring structure having about 3 to about 8 ring members that has only carbon atoms as ring atoms and can include divalent radicals. Examples of cycloalkyl groups include but are not limited to, cyclopropyl, cyclobutyl, cyclopentyl, cyclohexyl, cyclohexene, cyclopentenyl, cyclohexenyl.

The terms “heterocyclyl” or “heterocyclic” refer to monocyclic or polycyclic 3 to 24-membered rings containing carbon and heteroatoms selected from oxygen, phosphorous, nitrogen, or sulfur and wherein there are no delocalized n electrons (aromaticity) shared among the ring carbon or heteroatoms. Examples of heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, and homotropanyl. A heterocyclyl or heterocycloalkyl ring can also be fused or bridged, e.g. can be a bicyclic ring. Examples of heterocyclyl also include, but are not limited to, fused rings, bridged rings (e.g. 2,5-diazabicyclo[2,2,1]heptane), and spirocyclic rings, (e.g. 2,8-diazaspiro[4,5]decane).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intends to include C₁, C₂, C₃, C₄, C₅, and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl or n-hexyl. In some embodiments, a straight chain or branched alkyl has six or fewer carbon atoms (e.g. C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g. ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g. C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g. ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g. C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g. fused, bridged, or spiro rings) system having 3 to 30 carbon atoms (e.g. C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic. In some embodiments, the cycloalkyl is hexahydroindacenyl. In some embodiments, the cycloalkyl is

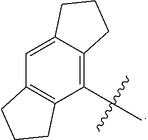 [SMILES: *c1c2c(cc3c1CCC3)CCC2]

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g. 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic heterocycloalkyl, only one of the rings in the heterocycloalkyl needs to be non-aromatic (e.g. 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g. 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulphur. The nitrogen atom is substituted or unsubstituted (i.e. N or NR wherein R is H or other substituents, as defined). The nitrogen and sulphur heteroatoms may optionally be oxidised (i.e. N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g. 4,5,6,7-tetrahydrobenzo[c]isoxazolyl).

Furthermore, the terms “aryl” and “heteroaryl” include multicyclic aryl and heteroaryl groups, e.g. tricyclic, bicyclic, e.g. naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g. the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g. tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e. ═O), then 2 hydrogen atoms on the atom are replaced. Keto substituents are not present on aromatic moieties. Ring double bonds, as used herein, are double bonds that are formed between two adjacent ring atoms (e.g. C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent is bonded to any atom in the ring. When a substituent is listed without indicating the atom via which such substituent is bonded to the rest of the compound of a given formula, then such substituent is bonded via any atom in such formula. Combinations of substituents and/or variables are permissible, but only if such combinations result in stable compounds.

When any variable (e.g. R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulphhydryl, alkylthio, arylthio, thiocarboxylate, sulphates, alkylsulphinyl, sulphonato, sulphamoyl, sulphonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

### Examples

### Example 1: Materials and Methods

Mice for Cell Screening

Neonatal Lgr5-EGFP-IRES-Cre-ER mice (The Jackson Laboratory, strain 8875) were used to analyze the effects of small molecules on cochlear stem cell expansion (see Barker et al., Nature 449, 1003-7 (2007). This strain allowed for visualization and quantification of EGFP cells.

Cell Assays

All animal studies were conducted under an approved institutional protocol per National Institutes of Health guidelines. Using neonatal animals, cochleae were dissected and the organ of Corti (sensory epithelium) was separated from the stria vascularis (ion transport epithelium) and the modiolus (nerve tissue). Epithelia were then collected and treated with TrypLE for 15-20 minutes to obtain single cells. The cells were then filtered (40 mm) and suspended in a Matrigel (Corning) dome for 3D culture seeded at 0.5 cochlea per well.

Expansion of Lgr5 Cells: Cells were cultured in a 3D system and bathed in a serum free 1:1 mixture of DMEM and F12, supplemented with Glutamax (GIBCO), N2, B27 (Invitrogen), EGF (50 ng/mL; Chemicon), bFGF (50 ng/mL; Chemicon), IGF-1 (50 ng/mL; Chemicon), and small molecules for seven days. Media was changed every other day. Treatments were run in triplicate or quadruplicate.

Quantification of Cell Proliferation: Lgr5 cells were quantified after 7-10 days. Cell colonies were dissociated into single cells using TrypLE. The cells were then stained with propidium iodide (PI) and analyzed using a flow cytometer to count Lgr5-EGFP cells. The percentage of viable Lgr5 cells was plotted against the concentration in GraphPad Prism.

Quantification of Cell Proliferation, Expansion and Enrichment

Organ of Corti are dissected from Lgr5 GFP(+) mice and dissociated as single cells as described above. Background media contains the same supplements and growth factors at the same concentrations as described above. Assays for image quantification are run in 96 well black plates with clear bottom with cells embedded in 50% Matrigel at cell density of 500 k cells/mL with 50 uL applied to each well. Cells are cultured for 7 days, with media change every 3-4 days. After 7 days of exposure to experimental conditions (e.g. small molecules), media is then removed from culture and replaced with media containing Hoescht at a 1:2000 dilution for a final concentration of 5 ug/mL (200 uL/well). The plate is then placed in a cell culture incubator at 37C for 1 hr. The media containing Hoescht is then removed and 200 uL/well of Cell Recovery Solution is added. The plate is then incubated on a plastic-wrapped (e.g. Saran wrap) CoolRack™ on ice for 80 minutes. Next, the plate is centrifuged for 5 minutes at 2300 RPMs (Beckman Coulter Allegra 6R centrifuge; GH 3.8A plate rotor; ambient temperature). Cells are then imaged on Celigo using 3 channels for brightfield, blue (Hoescht), and green (Lgr5 GFP). Proliferated cell colonies are captured as summed objects in the blue channel and the green channel. The green Lgr5 GFP(+) cell colonies are quantified for total GFP(+) cell area, while the blue hoescht stained colonies are quantified as total cell area. The % GFP(+) Cell Area is calculated using the total GFP(+) cell area divided by the total cell area multiplied by 100. All results are compiled and utilized to determine the effects of experimental conditions (e.g. small molecules) on the expansion and enrichment of the Lgr5 cell population.

Lateral Canal Sampling

Animals were initially anesthetized with 100 mg/kg sodium thiobutabarbital (Inactin, Sigma, St Louis, Mo.) and maintained on 0.8 to 1.2% isofluorane in oxygen. Animals were mechanically ventilated through a tracheal cannula. Tidal volume was set to maintain a 5% end-tidal CO₂ level. Heart rate and blood oxygen saturation were monitored with a pulse-oximeter (Surgivet. Waukesha, Wis.). Body temperature was maintained near 38° C. with a thermistor-controlled heating pad.

Access to the LSCC was obtained with a post-auricular incision and a lateral opening in the auditory bulla. To prepare the LSCC for injection and sampling, the bone over the canal was thinned with a dental burr, where necessary removing a branch of the facial nerve that in some animals runs parallel to the LSCC for a short distance. When the canal was visible through the thinned bone, a layer of thin cyanoacrylate glue was applied to the dry bone followed by layers of two-part silicone adhesive (Kwik-Cast, World Precision Instruments, Sarasota, Fla.). The silicone was applied thinly over the canal but multiple layers were built up at the periphery to form a hydrophobic cup structure. A 30-40 μm fenestration into the canal wall was made through the adhesives and bone using a 30° House stapes pick (N1705 80, Bausch and Lomb Inc.). The pick was sharp at the tip, but rapidly widened so that entry into the canal, and potential damage to the endolymphatic system, was minimized.

At times varied from 15 min to 4 h after the end of injection, multiple perilymph samples were taken from the LSCC. The injection pipette was first removed and the drop of cyanoacrylate glue that sealed it in place was broken up with the pick, taking care to leave the silicone cup intact. The fenestration was widened to 50-70 μm to allow perilymph leakage and the emerging perilymph was collected in blunt-tipped capillaries (#53432-706, 5 μL, VWR International, Radnor, Pa.). Each capillary was marked at a nominal volume of 1 μL. Sixteen to twenty individual 1 μL perilymph samples were collected sequentially, over a 20-30 min time period. The length of each sample was immediately measured with a calibrated dissecting microscope. Samples were expelled into dilutent (25 uL of 50:50 acetonitrile), with pairs of samples pooled, resulting in 8-10 measurements each. All data are presented as the 8-10 measured samples from each experiment. Analysis of compound concentration was determined by LCMS

Apical Sampling

Gradients of drug along the perilymphatic spaces were measured directly from multiple samples obtained by a technique called “sequential sampling”. When the apex is perforated, perilymph is driven out by cerebrospinal fluid (CSF) entering the basal turn of ST through the cochlear aqueduct, pushing perilymph in an apical direction along the scala. The first sample collected originates from perilymph near the apex and each following sample from perilymph that originated from a scala location progressively closer to the base. After all ST perilymph has been pushed out, subsequent samples contain CSF that has passed through the scala. Samples collected in this manner allow drug gradients along the length of ST to be quantified. Perilymph was collected from the cochlear apex as a series of individual 1 μL samples collected over a 10-20 min period. To prepare the cochlea for sample collection the middle ear mucosa overlying the cochlear apex was first removed and the bone was allowed to dry. A thin layer of cyanoacrylate glue (Permabond 101; Permabond, Pottstown, Pa.) was applied to the dry bone, followed by layers of two-part silicone adhesive (Kwik-Cast, World Precision Instruments, Sarasota, Fla.), built up at the edges to form a hydrophobic cup. At the time of sampling a 30-40 μm fenestration was made at the apex through the adhesives using a 30° House stapes pick (N1705 80, Bausch and Lomb Inc.). Clear, uncontaminated fluid flows from the fenestration, accumulating on the hydrophobic surface. Fluid was collected with hand-held, blunt tipped capillary tubes (VWR 53432-706: VWR Radnor, Pa.), each marked for a nominal volume of 1 μL and taking 1-2 min to collect. The length of each sample in its capillary tube was measured with a calibrated dissecting microscope, from which the exact sample volume was established. Ten individual samples were collected in this manner, with the first sample representing the apex and each subsequent sample representing further towards the base and eventually the CSF. Samples were expelled into dilutent (25 uL of 50:50 acetonitrile) and analysis of compound concentration was determined by LCMS

### Example 2: TAZ Activation does not Promote the Expansion of Cochlear Progenitor Cells

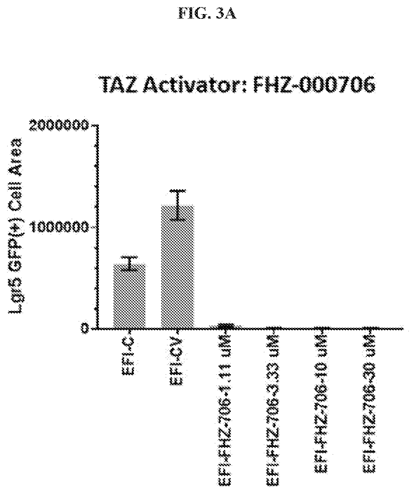

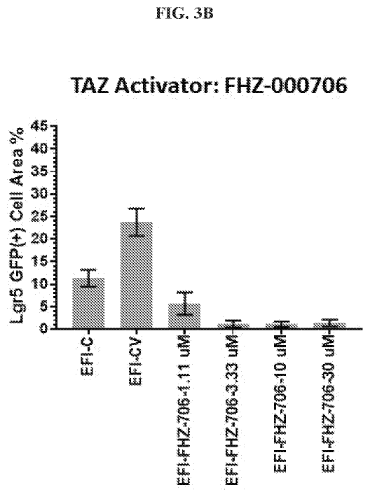

Cellular assays were carried about as described in Example 1 to determine the effect of TAZ activation on the expansion of cochlear progenitor cells. As shown in FIG. 3A and FIG. 3B, TAZ activation with FHZ-000706 does not promote the proliferation or enrichment of cochlear progenitor cells.

### Example 3: TAZ Activation in Combination with a Wnt Agonist Enhances the Expansion of Cochlear Progenitor Cells

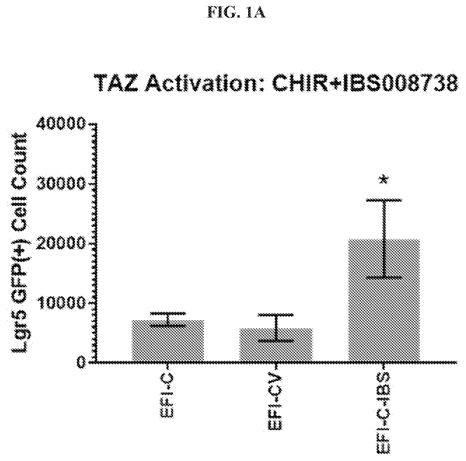

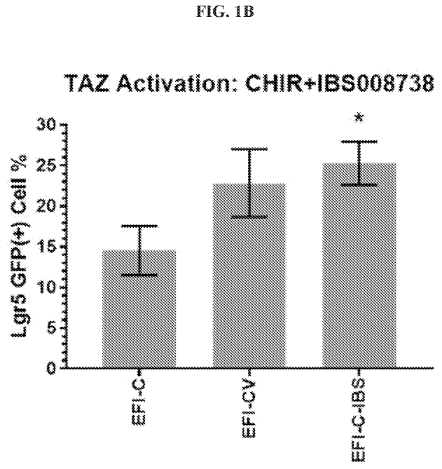

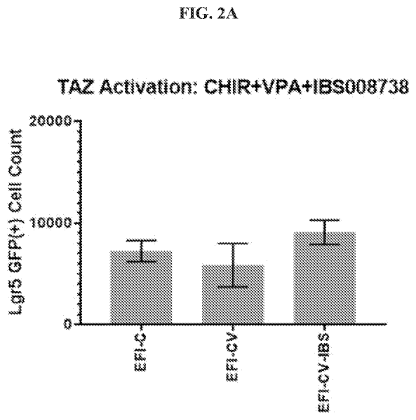

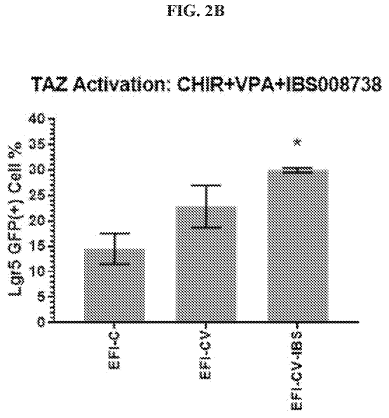

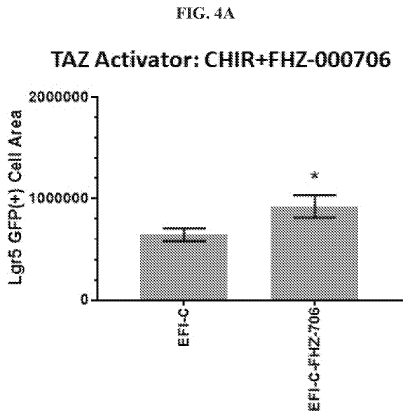

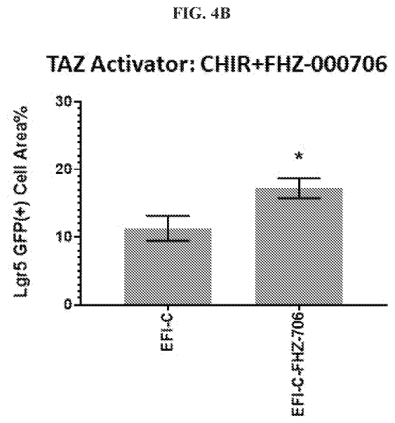

Cellular assays were carried about as described in Example 1 to determine the effect of TAZ activation in combination with a Wnt agonist on the expansion of cochlear progenitor cells. As shown in FIG. 1A, FIG. 1B, FIG. 2A, and FIG. 2B, Lgr5+ progentitor cells proliferate and are enriched when TAZ activator IBS008738 (10 μM) is combined with Wnt Agonist CHIR99021 (4 μM). As shown in FIG. 4A and FIG. 4B, Lgr5+ progentitor cells proliferate and are enriched when TAZ activator FHZ-000706 (10 μM) is combined with Wnt Agonist CHIR99021 (4 μM).

We claim:

1. A method for increasing proliferation of a cochlear supporting cell or a vestibular supporting cell, comprising contacting the supporting cell with:
a) a transcriptional coactivator with PDZ-binding motif (TAZ) activator; and
b) a Wnt agonist;
wherein (a) and (b) can occur in any order or simultaneously, thereby increasing cochlear supporting cell or vestibular supporting cell proliferation compared to a vehicle control.

2. A method for producing an expanded population of cochlear or vestibular cells, comprising contacting a population of cochlear supporting cells or vestibular supporting cells with:
a) a transcriptional coactivator with PDZ-binding motif (TAZ) activator and;
b) a Wnt agonist
wherein (a) and (b) can occur in any order or simultaneously, thereby producing an expanded population of cochlear or vestibular cells compared to a vehicle control.

3. The method of any preceding claim, further comprising contacting the cochlear or vestibular supporting cell(s) with an epigenetic agent.

4. The method of claim 3, wherein the epigenetic agent is an HDAC inhibitor, an EZH2 inhibitor, a DOT1L inhibitor a KDM inhibitor, or an LSD1 inhibitor.

5. The method of claim 1 or claim 2, wherein the cochlear supporting cell(s) or vestibular supporting cell(s) express(es) leucine-rich repeat-containing G-protein coupled receptor 5 (Lgr5).

6. The method of any preceding claim, wherein the cochlear supporting cell(s) or vestibular supporting cell(s) is/are a mature cell(s).

7. The method of any of claims 2-4, wherein the expanded population of cochlear or vestibular cells expresses leucine-rich repeat-containing G-protein coupled receptor 5 (Lgr5).

8. The method of any preceding claim, wherein the TAZ activator in combination with the Wnt agonist increases Lgr5 Activity of the expanded population of cochlear or vestibular cells by a factor of at least 10, 20, 30, 40, 50, 75, 100 or 200% compared to a Wnt agonist alone or a Wnt agonist in combination with valproic acid, wherein the Lgr5 Activity is measured in a Stem Cell Proliferation Assay

9. A method of treating a subject who has, or is at risk of, developing an inner ear hearing or balance disorder, comprising administering to the subject:
a) a transcriptional coactivator with PDZ-binding motif (TAZ) activator; and
b) a Wnt agonist wherein (a) and (b) can occur in any order or simultaneously.

10. The method of claim 9, wherein the subject has an inner ear hearing or balance disorder.

11. The method of any of claims 9-10, wherein the inner ear hearing or balance disorder is sensorineural hearing loss.

12. The method of any of claims 9-11, wherein the treatment results in improved auditory function when assessed by behavioural audiometry or auditory brainstem response (ABR) testing.

13. The method of any preceding claim, wherein the TAZ activator is IBS008738, TM-25659, FHZ-000706, or TT10.

14. The method of any preceding claim, wherein the Wnt agonist is a GSK3 inhibitor.

15. The method of any one of claims 9-14, further comprising administering to the subject, an epigenetic agent.

16. The method of claim 15, wherein the epigenetic agent is an HDAC inhibitor, an EZH2 inhibitor, a DOT1L inhibitor a KDM inhibitor or an LSD1 inhibitor.

17. The method of claim 16, wherein the HDAC inhibitor is Valproic Acid (VPA)

18. The method of claim 16, wherein the EZH2 inhibitor is selected from the group consisting of; CPI-1205, CPI-169, E11, PF-06821497, tazemetostat, valemetostat, CPI-360, EPZ011989, UNC 2399, and PF 06726304.

19. The method of claim 16, wherein the KDM inhibitor is AS 8351, TC-E 5002 or EPT-103182.

20. The method of claim 16, wherein the LSD1 inhibitor is selected from the group consisting of GSK-2879552, GSK-LSD1, Tranylcypromine, Phenelzine sulfate, ORY-1001, and RN-1.

21. The method of claim 16, wherein the DOT1L inhibitor is selected from the group consisting of EPZ004777, pinometostat and SGC0946.

22. The method of any preceding claim, wherein the TAZ activator is administered locally and/or systemically.

23. The method of any preceding claim, wherein the Wnt agonist is administered locally and/or systemically.

24. The method of any preceding claim, wherein the epigenetic agent is administered locally and/or systemically.

25. The method of any of claims 22-24, wherein the local administration is to the tympanic membrane, the middle ear or the inner ear.

26. The method of any of claims 22-25, wherein the systemic administration is oral or parenteral.

27. The method of any of claims 22-26, wherein the TAZ activator is IBS008738, TM-25659, FHZ-00706-1, or TT10.

28. A pharmaceutical composition comprising a TAZ activator, a Wnt agonist and a pharmaceutically acceptable carrier.

29. The pharmaceutical composition of claim 28, wherein the TAZ activator is IBS008738, TM-25659, FHZ-000706, or TT10.

30. The pharmaceutical composition of any of claims 28-29, wherein the Wnt agonist is a GSK3 inhibitor.

31. The pharmaceutical composition of claim 30, wherein the GSK3 inhibitor is selected from the group consisting of: AZD1080, LY2090314, a substituted 3-Imidazo[1,2-a]pyridin-3-yl-4-(1,2,3,4-tetrahydro-[1,4]diazepino-[6,7,1-hi]indol-7-yl)pyrrole-2,5-dione, GSK3 inhibitor XXII or CHIR99021.

32. The pharmaceutical composition of any of claims 28-31 further comprising further comprising an epigenetic agent.

33. The pharmaceutical composition of claim 32, wherein the epigenetic agent is an HDAC inhibitor, an EZH2 inhibitor, a DOT1L inhibitor a KDM inhibitor or a LSD1 inhibitor.

34. The pharmaceutical composition of claim 33, wherein the HDAC inhibitor is Valproic Acid (VPA)

35. The pharmaceutical composition of claim 33, wherein the EZH2 inhibitor is selected from the group consisting of: CPI-1205, CPI-169, E11, PF-06821497, tazemetostat, valemetostat, CPI-360, EPZ011989, UNC 2399, and PF 06726304.

36. The pharmaceutical composition of claim 33, wherein the DOT1L inhibitor is selected from the group consisting of EPZ004777, pinometostat, and SGC0946.

37. The pharmaceutical composition of claim 33, wherein the KDM inhibitor is selected from the group consisting of AS 8351, TC-E 5002 and EPT-103182.

38. The pharmaceutical composition of claim 33, wherein the LSD1 inhibitor is selected from the group consisting of GSK-2879552, GSK-LSD1, Tranylcypromine, Phenelzine sulfate, RN-1, and ORY-1001.

39. The pharmaceutical composition of any of claims 28-38, wherein the pharmaceutical composition is in a biocompatible matrix.

40. The pharmaceutical composition of claim 39, wherein the biocompatible matrix comprises hyaluronic acid, hyaluronates, lecithin gels, pluronics, poly(ethyleneglycol), poloxamers, chitosans, xyloglucans, collagens, fibrins, polyesters, poly(lactides), poly(glycolide), poly(lactic-co-glycolic acid (PLGA), sucrose acetate isobutyrate, glycerol monooleate, poly anhydrides, poly caprolactone sucrose, glycerol monooleate, silk materials, or a combination thereof.

41. The pharmaceutical composition of any of claims 28-40, wherein the pharmaceutical composition is formulated for systemic and/or local administration.

42. The pharmaceutical composition any of claims 28-41, for use in treating or preventing an inner ear hearing or balance disorder.

43. The pharmaceutical composition for use according to claim 42, wherein the inner ear hearing or balance disorder is sensorineural hearing loss.

44. Use of the pharmaceutical composition of any of claims 28-43 in the manufacture of a medicament for the treatment or prevention of an inner ear hearing or balance disorder.

45. Use of the pharmaceutical composition according to claim 44, wherein the inner ear hearing or balance disorder is sensorineural hearing loss.

46. A transcriptional coactivator with PDZ-binding motif (TAZ) activator for use in treating or preventing an inner ear hearing or balance disorder in a subject, wherein the subject has been, or will be, administered a Wnt agonist.

47. A Wnt agonist for use in treating or preventing an inner ear hearing or balance disorder in a subject, wherein the subject has been, or will be, administered a transcriptional coactivator with PDZ-binding motif (TAZ) activator.

48. An epigenetic agent for use in treating or preventing an inner ear hearing or balance disorder in a subject, wherein the subject has been, or will be, administered a transcriptional coactivator with PDZ-binding motif (TAZ) activator and a Wnt agonist.

49. The TAZ activator, Wnt agonist or epigenetic agent for use according to any of claims 43-47, wherein the inner ear hearing or balance disorder is sensorineural hearing loss.

50. The TAZ activator, Wnt agonist or epigenetic agent for use according to any of claims 44-49, wherein the treatment is as defined in any of claims 9-26.

51. A container comprising a transcriptional coactivator with PDZ-binding motif (TAZ) activator and instructions, where those instructions describe the TAZ activator use for treating or preventing an inner ear hearing or balance disorder in a subject, wherein the instructions require that the subject has been, or will be, administered a Wnt agonist.

52. A container comprising a Wnt agonist and instructions, where the instructions describe the Wnt agonist's use in treating or preventing an inner ear hearing or balance disorder in a subject, wherein the instructions require that the subject has been, or will be, administered a transcriptional coactivator with PDZ-binding motif (TAZ) activator.

53. A container comprising an epigenetic agent and instructions, where those instructions describe the epigenetic agent's use in treating or preventing an inner ear hearing or balance disorder in a subject, and wherein the instructions require that the subject has been, or will be, administered a transcriptional coactivator with PDZ-binding motif (TAZ) activator and a Wnt agonist.

54. The container according to any of claims 51-53, wherein the inner ear hearing or balance disorder is sensorineural hearing loss.

55. The container according to any of claims 51-53, wherein the treatment is as defined in any of claims 9-26.
